SEQUENCE LISTING (17 sequences)

SEQ ID NO: 1 - protein, 133 aa, Homo sapiens
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 2 - protein, 133 aa, Homo sapiens
MPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 3 - protein, 133 aa, Homo sapiens
APTSSSTKKTQLQLEHLLLDLQMILNGINNCKNPKLTRMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 4 - protein, 738 aa, synthetic construct
RGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAGGGGSGGGGSGGGGSAPTSSSTKKTQLQLEHLLLDLQMILNGINNCKNPKLTRMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 5 - protein, 733 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAPTSSSTKKTQLQLEHLLLDLQMILNGINNCKNPKLTRMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 6 - protein, 590 aa, Mus musculus
RGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 7 - protein, 585 aa, Homo sapiens [sequence omitted]

SEQ ID NO: 8 - DNA, 399 bp, Homo sapiens
  source 1..399: mol_type = genomic DNA
gcccccacaagcagcagcaccaagaagacacagctgcagctggagcacctgctgctggacctgcagatgatcctgaacggcatcaacaactgcaagaaccccaagctgacaagaatgctgaccaagaagttctacatgcccaagaaggccaccgagctgaagcacctgcagtgcctggaggaggagctgaagcccctggaagaggtgctgaacctggctcagagcaagaacttccacctgagacctagagacctgatcagcaacatcaacgtgatcgtgctggagctgaagggcagcgagaccacctttatgtgcgagtacgccgacgagaccgccaccatcgtggagttcctgaacagatggatcaccttttctcagagcatcattagcaccctcacc

SEQ ID NO: 9 - DNA, 2220 bp, synthetic construct
  source 1..2220: mol_type = other DNA
agaggcgtgttcagaagagaggcccacaagagcgagatcgcccacagatacaacgacctgggcgagcagcacttcaagggcctggtgctgatcgccttctctcagtacctgcagaagtgcagctacgacgagcacgccaagctggtgcaagaggtgaccgacttcgccaagacctgcgtggccgacgagagcgccgccaactgcgacaagagcctgcacaccctgttcggcgacaagctgtgcgccatccccaacctgagagagaactacggcgagctggccgactgctgcaccaagcaagagcccgagagaaacgagtgcttcctgcagcacaaggacgacaaccctagcctgccccccttcgagagacccgaggccgaggccatgtgcacaagcttcaaggagaaccccaccacctttatgggacactacctgcacgaggtggcccgtagacatccctatttttacgcccccgagctgctgtactacgccgagcagtacaacgagatcctgacccaatgctgtgccgaagctgacaaggagagctgcctgacccccaagctggacggcgtgaaggagaaggccctggtgagcagcgtgagacagagaatgaagtgcagcagcatgcagaagttcggcgagagagccttcaaggcctgggccgtggctagactgtctcagaccttccccaacgccgacttcgccgagatcaccaagctggccaccgacctgaccaaggtgaacaaggagtgctgccacggcgacctgctggagtgcgccgacgacagagccgagctggccaagtacatgtgcgagaaccaagccaccatcagcagcaagctgcagacctgctgcgacaagcccctgctgaagaaggcccactgcctgtcggaggtggaacacgacaccatgcccgcagatctgcccgccatcgccgccgacttcgtggaggaccaagaggtgtgcaagaactacgccgaagccaaggatgtgttcctgggcaccttcctgtacgagtacagcagacggcatcccgactacagcgtgagcctgctgctgagactggccaagaagtatgaggccactctggagaaatgctgtgcggaagccaacccgccggcctgctacggtaccgtcctggccgagtttcagcccctggtggaggagcccaagaacctggtgaagaccaactgcgacctgtacgagaagctgggcgagtacggctttcagaacgccatcctggtgagatacacacagaaggccccccaagtgagcacccccaccctagtggaggccgcaagaaacctgggcagagtgggcacaaaatgctgcacgctccccgaggatcagagactgccctgcgtggaggactacctgagcgccatcctgaacagagtgtgcctgctgcacgagaagacccccgtgagcgagcacgtgaccaaatgctgcagcggaagcctggtggagagaagaccctgcttcagcgccctgaccgtggacgagacctacgtgcccaaggagttcaaggccgagaccttcaccttccacagcgacatctgcaccctgcccgagaaggaaaagcagatcaagaagcagaccgccctggccgagctggtgaagcacaagcccaaggccaccgccgagcagctgaagaccgtgatggacgacttcgctcagttcctggacacctgctgcaaggccgccgacaaggacacctgcttcagcaccgagggacccaatttagtaacaagatgcaaagatgcattggccggtggtggcggctcgggcgggggcgggtccggtggtgggggtagcgcccccacaagcagcagcaccaagaagacacagctgcagctggagcacctgctgctggacctgcagatgatcctgaacggcatcaacaactgcaagaaccccaagctgacaagaatgctgaccaagaagttctacatgcccaagaaggccaccgagctgaagcacctgcagtgcctggaggaggagctgaagcccctggaagaggtgctgaacctggctcagagcaagaacttccacctgagacctagagacctgatcagcaacatcaacgtgatcgtgctggagctgaagggcagcgagactaccttcatgtgcgaatacgccgacgagaccgccaccatcgtggagttcctgaacagatggatcaccttctctcagagcatcatcagcaccctgacctgataa

SEQ ID NO: 10 - DNA, 2199 bp, synthetic construct
  source 1..2199: mol_type = other DNA
gacgcccacaagagcgaggtggcccacagattcaaggacctgggcgaggagaacttcaaggccctggtgctgatcgccttcgctcagtacctgcagcagtgccccttcgaggaccacgtgaagctggtgaacgaggtgaccgagttcgccaagacctgcgtggccgacgagagcgccgagaactgcgacaagagcctgcacaccctgttcggcgacaagctgtgcaccgtggccaccctgagagagacctacggcgagatggccgactgctgcgccaagcaagagcccgagagaaacgagtgcttcctgcagcacaaggacgacaaccccaacctgcctagactggtgagacccgaggtggacgtgatgtgcaccgccttccacgacaacgaggagaccttcctgaagaagtacctgtacgagatcgcaagacggcacccgtatttctacgcccccgagctgctgttcttcgccaagagatacaaggcggcttttaccgaatgctgtcaagccgccgacaaagcggcgtgtctgctgcccaagctggacgagctgagagacgagggcaaggctagcagcgccaagcagagactgaagtgcgctagcctgcagaagttcggcgagagagccttcaaggcctgggccgtggctagactgtctcagagattccccaaggccgagttcgccgaggtgagcaagctggtgaccgacctgaccaaggtgcacaccgagtgctgccacggcgacctgctggagtgcgccgacgacagagccgacctggccaagtacatctgcgagaaccaagacagcatcagcagcaagctgaaggagtgctgcgagaagcccctgctggagaagagccactgcatcgccgaggtggagaacgacgagatgcccgccgacctgcctagcctggccgccgacttcgtggagagcaaggacgtgtgcaagaactacgccgaggccaaggacgtgttcctgggcatgttcctgtacgagtacgctagaagacatccggactacagcgtggtgctgttactgagactggccaagacctacgaaactaccttagagaagtgttgtgccgctgccgacccccatgagtgctatgccaaggtgttcgacgagttcaagcccctagtggaagagcctcagaacctgatcaagcagaactgcgagctgttcgagcagctgggcgagtacaagtttcagaacgccctgctggtgagatacaccaagaaggtgccccaagtgagcacccccacgttggtggaggtgagcagaaacctgggcaaggtgggcagcaagtgctgcaagcaccccgaggccaagagaatgccctgcgccgaggactacctgtccgtggttctgaatcagctgtgcgtgctgcacgagaagacccccgtgagcgacagagtgaccaagtgctgcaccgagagcctggtgaacagaagaccctgcttcagcgccctggaggtggacgagacctacgtgcccaaggagttcaacgccgagaccttcaccttccacgccgacatctgcaccctgagcgagaaggagagacagatcaagaagcagaccgccctggtggagctggtgaagcacaagcccaaggccaccaaggagcagctgaaggccgtgatggacgacttcgccgccttcgtggaaaaatgttgtaaagccgatgacaaggagacctgcttcgccgaggagggcaagaagctggtggccgctagccaagccgccctgggcctggggggtgggggaagcggcggtggcggtagcgggggaggcggtagcgcccccacaagcagcagcaccaagaagacacagctgcagctggagcacctgctgctggacctgcagatgatcctgaacggcatcaacaactgcaagaaccccaagctgacaagaatgctgaccaagaagttctacatgcccaagaaggccaccgagctgaagcacctgcagtgcctggaggaggagctgaagcccctggaagaggtgctgaacctggctcagagcaagaacttccacctgagacctagagacctgatcagcaacatcaacgtgatcgtgctggagctgaagggcagcgagaccacctttatgtgcgagtacgccgacgagaccgccaccatcgtggagttcctgaacagatggatcaccttttctcagagcatcattagcaccctcacc

SEQ ID NO: 11 - RNA, 2214 nt, synthetic construct
  source 1..2214: mol_type = other RNA
agaggcgtgttcagaagagaggcccacaagagcgagatcgcccacagatacaacgacctgggcgagcagcacttcaagggcctggtgctgatcgccttctctcagtacctgcagaagtgcagctacgacgagcacgccaagctggtgcaagaggtgaccgacttcgccaagacctgcgtggccgacgagagcgccgccaactgcgacaagagcctgcacaccctgttcggcgacaagctgtgcgccatccccaacctgagagagaactacggcgagctggccgactgctgcaccaagcaagagcccgagagaaacgagtgcttcctgcagcacaaggacgacaaccctagcctgccccccttcgagagacccgaggccgaggccatgtgcacaagcttcaaggagaaccccaccacctttatgggacactacctgcacgaggtggcccgtagacatccctatttttacgcccccgagctgctgtactacgccgagcagtacaacgagatcctgacccaatgctgtgccgaagctgacaaggagagctgcctgacccccaagctggacggcgtgaaggagaaggccctggtgagcagcgtgagacagagaatgaagtgcagcagcatgcagaagttcggcgagagagccttcaaggcctgggccgtggctagactgtctcagaccttccccaacgccgacttcgccgagatcaccaagctggccaccgacctgaccaaggtgaacaaggagtgctgccacggcgacctgctggagtgcgccgacgacagagccgagctggccaagtacatgtgcgagaaccaagccaccatcagcagcaagctgcagacctgctgcgacaagcccctgctgaagaaggcccactgcctgtcggaggtggaacacgacaccatgcccgcagatctgcccgccatcgccgccgacttcgtggaggaccaagaggtgtgcaagaactacgccgaagccaaggatgtgttcctgggcaccttcctgtacgagtacagcagacggcatcccgactacagcgtgagcctgctgctgagactggccaagaagtatgaggccactctggagaaatgctgtgcggaagccaacccgccggcctgctacggtaccgtcctggccgagtttcagcccctggtggaggagcccaagaacctggtgaagaccaactgcgacctgtacgagaagctgggcgagtacggctttcagaacgccatcctggtgagatacacacagaaggccccccaagtgagcacccccaccctagtggaggccgcaagaaacctgggcagagtgggcacaaaatgctgcacgctccccgaggatcagagactgccctgcgtggaggactacctgagcgccatcctgaacagagtgtgcctgctgcacgagaagacccccgtgagcgagcacgtgaccaaatgctgcagcggaagcctggtggagagaagaccctgcttcagcgccctgaccgtggacgagacctacgtgcccaaggagttcaaggccgagaccttcaccttccacagcgacatctgcaccctgcccgagaaggaaaagcagatcaagaagcagaccgccctggccgagctggtgaagcacaagcccaaggccaccgccgagcagctgaagaccgtgatggacgacttcgctcagttcctggacacctgctgcaaggccgccgacaaggacacctgcttcagcaccgagggacccaatttagtaacaagatgcaaagatgcattggccggtggtggcggctcgggcgggggcgggtccggtggtgggggtagcgcccccacaagcagcagcaccaagaagacacagctgcagctggagcacctgctgctggacctgcagatgatcctgaacggcatcaacaactgcaagaaccccaagctgacaagaatgctgaccaagaagttctacatgcccaagaaggccaccgagctgaagcacctgcagtgcctggaggaggagctgaagcccctggaagaggtgctgaacctggctcagagcaagaacttccacctgagacctagagacctgatcagcaacatcaacgtgatcgtgctggagctgaagggcagcgagactaccttcatgtgcgaatacgccgacgagaccgccaccatcgtggagttcctgaacagatggatcaccttctctcagagcatcatcagcaccctgacc

SEQ ID NO: 12 - RNA, 2814 nt, synthetic construct
  source 1..2814: mol_type = other RNA
aggagacccaagctggctagcgtttaaacttaagcttggtaccgagctcggatccactagtccagtgtggtggaattctgcagatactcttctggtccccacagactcagagagaagccaccatggagttcggcctgagctgggtgttcctggtggccctgttcagaggcgtgcagagcagaggcgtgttcagaagagaggcccacaagagcgagatcgcccacagatacaacgacctgggcgagcagcacttcaagggcctggtgctgatcgccttctctcagtacctgcagaagtgcagctacgacgagcacgccaagctggtgcaagaggtgaccgacttcgccaagacctgcgtggccgacgagagcgccgccaactgcgacaagagcctgcacaccctgttcggcgacaagctgtgcgccatccccaacctgagagagaactacggcgagctggccgactgctgcaccaagcaagagcccgagagaaacgagtgcttcctgcagcacaaggacgacaaccctagcctgccccccttcgagagacccgaggccgaggccatgtgcacaagcttcaaggagaaccccaccacctttatgggacactacctgcacgaggtggcccgtagacatccctatttttacgcccccgagctgctgtactacgccgagcagtacaacgagatcctgacccaatgctgtgccgaagctgacaaggagagctgcctgacccccaagctggacggcgtgaaggagaaggccctggtgagcagcgtgagacagagaatgaagtgcagcagcatgcagaagttcggcgagagagccttcaaggcctgggccgtggctagactgtctcagaccttccccaacgccgacttcgccgagatcaccaagctggccaccgacctgaccaaggtgaacaaggagtgctgccacggcgacctgctggagtgcgccgacgacagagccgagctggccaagtacatgtgcgagaaccaagccaccatcagcagcaagctgcagacctgctgcgacaagcccctgctgaagaaggcccactgcctgtcggaggtggaacacgacaccatgcccgcagatctgcccgccatcgccgccgacttcgtggaggaccaagaggtgtgcaagaactacgccgaagccaaggatgtgttcctgggcaccttcctgtacgagtacagcagacggcatcccgactacagcgtgagcctgctgctgagactggccaagaagtatgaggccactctggagaaatgctgtgcggaagccaacccgccggcctgctacggtaccgtcctggccgagtttcagcccctggtggaggagcccaagaacctggtgaagaccaactgcgacctgtacgagaagctgggcgagtacggctttcagaacgccatcctggtgagatacacacagaaggccccccaagtgagcacccccaccctagtggaggccgcaagaaacctgggcagagtgggcacaaaatgctgcacgctccccgaggatcagagactgccctgcgtggaggactacctgagcgccatcctgaacagagtgtgcctgctgcacgagaagacccccgtgagcgagcacgtgaccaaatgctgcagcggaagcctggtggagagaagaccctgcttcagcgccctgaccgtggacgagacctacgtgcccaaggagttcaaggccgagaccttcaccttccacagcgacatctgcaccctgcccgagaaggaaaagcagatcaagaagcagaccgccctggccgagctggtgaagcacaagcccaaggccaccgccgagcagctgaagaccgtgatggacgacttcgctcagttcctggacacctgctgcaaggccgccgacaaggacacctgcttcagcaccgagggacccaatttagtaacaagatgcaaagatgcattggccggtggtggcggctcgggcgggggcgggtccggtggtgggggtagcgcccccacaagcagcagcaccaagaagacacagctgcagctggagcacctgctgctggacctgcagatgatcctgaacggcatcaacaactgcaagaaccccaagctgacaagaatgctgaccaagaagttctacatgcccaagaaggccaccgagctgaagcacctgcagtgcctggaggaggagctgaagcccctggaagaggtgctgaacctggctcagagcaagaacttccacctgagacctagagacctgatcagcaacatcaacgtgatcgtgctggagctgaagggcagcgagactaccttcatgtgcgaatacgccgacgagaccgccaccatcgtggagttcctgaacagatggatcaccttctctcagagcatcatcagcaccctgacctgataaatccagcacagtggcggccctggtactgcatgcacgcaatgctagctgcccctttcccgtcctgggcaccccgagtctcccccgaccccgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaat

SEQ ID NO: 13 - RNA, 2199 nt, synthetic construct
  source 1..2199: mol_type = other RNA
gacgcccacaagagcgaggtggcccacagattcaaggacctgggcgaggagaacttcaaggccctggtgctgatcgccttcgctcagtacctgcagcagtgccccttcgaggaccacgtgaagctggtgaacgaggtgaccgagttcgccaagacctgcgtggccgacgagagcgccgagaactgcgacaagagcctgcacaccctgttcggcgacaagctgtgcaccgtggccaccctgagagagacctacggcgagatggccgactgctgcgccaagcaagagcccgagagaaacgagtgcttcctgcagcacaaggacgacaaccccaacctgcctagactggtgagacccgaggtggacgtgatgtgcaccgccttccacgacaacgaggagaccttcctgaagaagtacctgtacgagatcgcaagacggcacccgtatttctacgcccccgagctgctgttcttcgccaagagatacaaggcggcttttaccgaatgctgtcaagccgccgacaaagcggcgtgtctgctgcccaagctggacgagctgagagacgagggcaaggctagcagcgccaagcagagactgaagtgcgctagcctgcagaagttcggcgagagagccttcaaggcctgggccgtggctagactgtctcagagattccccaaggccgagttcgccgaggtgagcaagctggtgaccgacctgaccaaggtgcacaccgagtgctgccacggcgacctgctggagtgcgccgacgacagagccgacctggccaagtacatctgcgagaaccaagacagcatcagcagcaagctgaaggagtgctgcgagaagcccctgctggagaagagccactgcatcgccgaggtggagaacgacgagatgcccgccgacctgcctagcctggccgccgacttcgtggagagcaaggacgtgtgcaagaactacgccgaggccaaggacgtgttcctgggcatgttcctgtacgagtacgctagaagacatccggactacagcgtggtgctgttactgagactggccaagacctacgaaactaccttagagaagtgttgtgccgctgccgacccccatgagtgctatgccaaggtgttcgacgagttcaagcccctagtggaagagcctcagaacctgatcaagcagaactgcgagctgttcgagcagctgggcgagtacaagtttcagaacgccctgctggtgagatacaccaagaaggtgccccaagtgagcacccccacgttggtggaggtgagcagaaacctgggcaaggtgggcagcaagtgctgcaagcaccccgaggccaagagaatgccctgcgccgaggactacctgtccgtggttctgaatcagctgtgcgtgctgcacgagaagacccccgtgagcgacagagtgaccaagtgctgcaccgagagcctggtgaacagaagaccctgcttcagcgccctggaggtggacgagacctacgtgcccaaggagttcaacgccgagaccttcaccttccacgccgacatctgcaccctgagcgagaaggagagacagatcaagaagcagaccgccctggtggagctggtgaagcacaagcccaaggccaccaaggagcagctgaaggccgtgatggacgacttcgccgccttcgtggaaaaatgttgtaaagccgatgacaaggagacctgcttcgccgaggagggcaagaagctggtggccgctagccaagccgccctgggcctggggggtgggggaagcggcggtggcggtagcgggggaggcggtagcgcccccacaagcagcagcaccaagaagacacagctgcagctggagcacctgctgctggacctgcagatgatcctgaacggcatcaacaactgcaagaaccccaagctgacaagaatgctgaccaagaagttctacatgcccaagaaggccaccgagctgaagcacctgcagtgcctggaggaggagctgaagcccctggaagaggtgctgaacctggctcagagcaagaacttccacctgagacctagagacctgatcagcaacatcaacgtgatcgtgctggagctgaagggcagcgagaccacctttatgtgcgagtacgccgacgagaccgccaccatcgtggagttcctgaacagatggatcaccttttctcagagcatcattagcaccctcacc

SEQ ID NO: 14 - RNA, 2799 nt, synthetic construct
  source 1..2799: mol_type = other RNA
aggagacccaagctggctagcgtttaaacttaagcttggtaccgagctcggatccactagtccagtgtggtggaattctgcagatactcttctggtccccacagactcagagagaagccaccatggagttcggcctgagctgggtgttcctggtggccctgttcagaggcgtgcagagcgacgcccacaagagcgaggtggcccacagattcaaggacctgggcgaggagaacttcaaggccctggtgctgatcgccttcgctcagtacctgcagcagtgccccttcgaggaccacgtgaagctggtgaacgaggtgaccgagttcgccaagacctgcgtggccgacgagagcgccgagaactgcgacaagagcctgcacaccctgttcggcgacaagctgtgcaccgtggccaccctgagagagacctacggcgagatggccgactgctgcgccaagcaagagcccgagagaaacgagtgcttcctgcagcacaaggacgacaaccccaacctgcctagactggtgagacccgaggtggacgtgatgtgcaccgccttccacgacaacgaggagaccttcctgaagaagtacctgtacgagatcgcaagacggcacccgtatttctacgcccccgagctgctgttcttcgccaagagatacaaggcggcttttaccgaatgctgtcaagccgccgacaaagcggcgtgtctgctgcccaagctggacgagctgagagacgagggcaaggctagcagcgccaagcagagactgaagtgcgctagcctgcagaagttcggcgagagagccttcaaggcctgggccgtggctagactgtctcagagattccccaaggccgagttcgccgaggtgagcaagctggtgaccgacctgaccaaggtgcacaccgagtgctgccacggcgacctgctggagtgcgccgacgacagagccgacctggccaagtacatctgcgagaaccaagacagcatcagcagcaagctgaaggagtgctgcgagaagcccctgctggagaagagccactgcatcgccgaggtggagaacgacgagatgcccgccgacctgcctagcctggccgccgacttcgtggagagcaaggacgtgtgcaagaactacgccgaggccaaggacgtgttcctgggcatgttcctgtacgagtacgctagaagacatccggactacagcgtggtgctgttactgagactggccaagacctacgaaactaccttagagaagtgttgtgccgctgccgacccccatgagtgctatgccaaggtgttcgacgagttcaagcccctagtggaagagcctcagaacctgatcaagcagaactgcgagctgttcgagcagctgggcgagtacaagtttcagaacgccctgctggtgagatacaccaagaaggtgccccaagtgagcacccccacgttggtggaggtgagcagaaacctgggcaaggtgggcagcaagtgctgcaagcaccccgaggccaagagaatgccctgcgccgaggactacctgtccgtggttctgaatcagctgtgcgtgctgcacgagaagacccccgtgagcgacagagtgaccaagtgctgcaccgagagcctggtgaacagaagaccctgcttcagcgccctggaggtggacgagacctacgtgcccaaggagttcaacgccgagaccttcaccttccacgccgacatctgcaccctgagcgagaaggagagacagatcaagaagcagaccgccctggtggagctggtgaagcacaagcccaaggccaccaaggagcagctgaaggccgtgatggacgacttcgccgccttcgtggaaaaatgttgtaaagccgatgacaaggagacctgcttcgccgaggagggcaagaagctggtggccgctagccaagccgccctgggcctggggggtgggggaagcggcggtggcggtagcgggggaggcggtagcgcccccacaagcagcagcaccaagaagacacagctgcagctggagcacctgctgctggacctgcagatgatcctgaacggcatcaacaactgcaagaaccccaagctgacaagaatgctgaccaagaagttctacatgcccaagaaggccaccgagctgaagcacctgcagtgcctggaggaggagctgaagcccctggaagaggtgctgaacctggctcagagcaagaacttccacctgagacctagagacctgatcagcaacatcaacgtgatcgtgctggagctgaagggcagcgagaccacctttatgtgcgagtacgccgacgagaccgccaccatcgtggagttcctgaacagatggatcaccttttctcagagcatcattagcaccctcacctgataaatccagcacagtggcggccctggtactgcatgcacgcaatgctagctgcccctttcccgtcctgggcaccccgagtctcccccgaccccgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaat

SEQ ID NO: 15 - protein, 15 aa, synthetic construct
GGGGSGGGGSGGGGS

SEQ ID NO: 16 - DNA, 45 bp, synthetic construct
  source 1..45: mol_type = other DNA
gggggtgggggaagcggcggtggcggtagcgggggaggcggtagc

SEQ ID NO: 17 - RNA, 45 nt, synthetic construct
  source 1..45: mol_type = other RNA
gggggtgggggaagcggcggtggcggtagcgggggaggcggtagc

(19) United States
(12) Patent Application Publication
(10) Pub. No.: US 2026/0258108 A1
(43) Pub. Date: Sep. 3, 2026

(54) POLYNUCLEOTIDES ENCODING MODIFIED INTERLEUKIN 2 (IL2) POLYPEPTIDES, AND METHODS OF MAKING AND USING THE SAME

(71) Applicant: ILEUKON THERAPEUTICS, INC., San Diego, CA (US)

(72) Inventors: Haining HUANG, San Diego, CA (US); Runqiang CHEN, San Diego, CA (US)

(73) Assignee: ILEUKON THERAPEUTICS, INC., San Diego, CA (US)

(21) Appl. No.: 19/157,022

(22) PCT Filed: Feb. 20, 2024
(86) PCT No.: PCT/US2024/016521

§ 371 Date: Aug. 15, 2025

Related U.S. Application Data
(60) Provisional application No. 63/447,207, filed on Feb. 21, 2023.

(51) Int. Cl. C07K 14/55; A61K 38/00

(52) U.S. Cl. CPC C07K 14/55; A61K 38/00; C07K 2319/31

(57) ABSTRACT

This disclosure relates to, inter alia, polynucleotides encoding modified interleukin 2 (IL-2) polypeptides and fusion proteins comprising such modified IL-2 polypeptides, vectors comprising such polynucleotides, pharmaceutical compositions comprising such polynucleotides and vectors, and methods of treating diseases and disorders by administering such polynucleotides, vectors, and/or pharmaceutical compositions.

### TECHNICAL FIELD

This disclosure relates to, inter alia, polynucleotides encoding modified interleukin 2 (IL-2) polypeptides and fusion proteins comprising such modified IL-2 polypeptides, vectors comprising such polynucleotides, pharmaceutical compositions comprising such polynucleotides and vectors, and methods of treating diseases and disorders by administering such polynucleotides, vectors, and pharmaceutical compositions.

### BACKGROUND

Cytokines comprise a family of cell signaling proteins such as chemokines, interferons, interleukins, lymphokines, tumor necrosis factors, and other growth factors playing roles in innate and adaptive immune cell homeostasis. Cytokines are produced by immune cells such as macrophages, B lymphocytes, T lymphocytes and mast cells, endothelial cells, fibroblasts, and different stromal cells. In some instances, cytokines modulate the balance between humoral and cell-based immune responses.

Interleukins are signaling proteins which modulate the development and differentiation of T and B lymphocytes, cell of the monocytic lineage, neutrophils, basophils, eosinophils, megakaryocytes, and hematopoietic cells. Interleukins are produced by helper CD4 T and B lymphocytes, monocytes, macrophages, endothelial cells, and other tissue residents. Interleukin 2 (IL-2) is a pleiotropic type-1 cytokine whose structure comprises a 15.5 kDa four a-helix bundle. The precursor form of IL-2 is 153 amino acid residues in length, with the first 20 amino acids forming a signal peptide and residues 21-153 forming the mature form. IL-2 is produced primarily by CD4+ T cells post antigen stimulation and, to a lesser extent, by CD8+ cells, Natural Killer (NK) cells, and NK T (NKT) cells, activated dendritic cells (DCs), and mast cells. IL-2 signaling occurs through interaction with specific combinations of IL-2 receptor (IL-2R) subunits, IL-2Rα (also known as CD25), IL-2Rβ (also known as CD122), and IL-2Rγ (also known as CD 132). Interaction of IL-2 with the IL-2Rα forms the “low-affinity” IL-2 receptor complex with a K_{d} of about 10⁻⁸ M. Interaction of IL-2 with IL-2Rβ and IL-2Rγ forms the “intermediate-affinity” IL-2 receptor complex with a K_{d} of about 10⁻⁹ M. Interaction of IL-2 with all three subunits, IL-2Rα, IL-2Rβ, and IL-2Rγ, forms the “high-affinity” IL-2 receptor complex with a K_{d} of about >10⁻¹¹ M.

In some instances, IL-2 signaling via the “high-affinity” IL-2Rαβγ complex modulates the activation and proliferation of regulatory T cells. Regulatory T cells, such as CD4+CD25+ Foxp3+ regulatory T (Treg) cells, mediate maintenance of immune homeostasis by suppression of effector cells such as CD8+ T cells, helper cells such as CD4+ Thl, Th2, and Thl 7 cells, B cells, NK cells, and NK T cells. In some instances, Treg cells are generated from the thymus (tTreg cells) or are induced from naive T cells in the periphery (pTreg cells). In some cases, Treg cells are considered as a predominant mediator of peripheral tolerance. Indeed, in one study, transfer of CD25− depleted peripheral CD4+ T cells produced a variety of autoimmune diseases in nude mice, whereas cotransfer of CD4+CD25+ T cells suppressed the development of autoimmunity (Sakaguchi et al., J. Immunol. (1995)). Augmentation of the Treg cell population down-regulates effector T cell proliferation and suppresses autoimmunity and T cell anti-tumor responses.

Clinical use of interleukin-2 (IL-2) for treatment of many disease and disorders, such as cancers and autoimmune and inflammatory diseases has been mainly limited by toxicity and short half-life in vivo (Pachella et al., J Adv Pract Oncol (2015); Lotze et al. (1985) J. Immunol (1985)). It has been observed that toxicity was markedly reduced in animals deficient in CD25 (IL-2 receptor α unit, IL-2Rα) Boyman, et al., J Immunol(2009)) PEGylation, the covalent attachment of Polyethylene glycol (PEG) to therapeutics, has also been shown to overcome certain obstacles such as rapid body clearance, aggregation and enzymatic degradation, in certain instances Maiser et al., Biotechnol Bioeng (2014)). WO 2019/028419 A1 and WO 2019/028425 A1 disclose certain interleukin (IL) conjugates (e.g., IL-2 conjugates) and use in the treatment of certain indications. Also described in WO 2019/028419 A1 and WO 2019/028425 A1 are pharmaceutical compositions and kits comprising one or more of the interleukin conjugates (e.g., IL-2 conjugates).

Despite the existence of certain IL-2 muteins as potential therapeutic molecules, there remains a need for alternative modified IL-2 chemical modalities that provide reduced toxicity, enhanced pharmacokinetic and pharmacodynamic characteristics, and other attributes relative to peptide-based counterparts.

### SUMMARY

Provided are, inter alia: polynucleotides encoding modified interleukin 2 (IL-2) polypeptides and polynucleotides encoding fusions proteins comprising modified IL-2 polypeptides; vectors, such as, for example, mRNA vectors, DNA/RNA vectors, DNA vectors, viral vectors, and non-viral vectors harboring such polynucleotides; pharmaceutical compositions comprising such polynucleotides and vectors; and methods of treating or preventing diseases or disorders, such as proliferation diseases or disorders, immune deficiency diseases or disorders, inflammatory diseases or disorders; autoimmune diseases or disorders; and/or infectious diseases or disorders.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide comprises an amino acid having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100% sequence identity to the amino acid sequence set forth in SEQ ID NO:1 or SEQ ID NO:2, wherein the modified IL-2 polypeptide comprises at least one substitution with a natural amino acid or an unnatural amino acid at one or more positions selected from the group consisting of Q13, L19, N29, N30, Y31, K32, N33, P34, K35, T37, R38, T41, F42, K43, Y45, K48, K49, E62, K64, P65, N71, Q74, K76, R81, L85, S87, V91, 192, V93, and combinations thereof.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide: a) is configured to be unconjugated or is conjugated to a water-soluble polymer, a lipid, a polypeptide, a protein or a peptide; and/or
b) has reduced binding to an interleukin 2 receptor α (IL-2Rα) compared to a comparable IL-2 polypeptide comprising an amino acid sequence set forth in SEQ ID NO:1 or SEQ ID NO:2 without the at least one substitution; and/or
c) has reduced receptor signaling potency to IL-2Rαβγ compared to a comparable IL-2 polypeptide comprising an amino acid sequence set forth in SEQ ID NO:1 or SEQ ID NO:2 without the at least one substitution, and/or
d) has increased ratio of signaling potency to IL-2Rβγ over signaling potency to IL-2Rαβγ (increased ratio of signaling potency to IL-2Rβγ/signaling potency to IL-2Rααβγ) compared to a comparable IL-2 polypeptide comprising an amino acid sequence set forth in SEQ ID NO:1 or SEQ ID NO:2 without the at least one substitution, and/or
e) has enhanced receptor signaling potency to IL-2Rβγ compared to a comparable IL-2 polypeptide comprising an amino acid sequence set forth in SEQ ID NO:1 or SEQ ID NO:2 without the at least one substitution, and/or
provided that when the modified IL-2 polypeptide comprises at least one substitution with an unnatural amino acid, the modified IL-2 polypeptide comprises at least one substitution at a position selected from the group consisting of N29, N30, Y31, K32, N33, P34, K35, R38, T41, F42, K43, Y45, K48, K49, E62, K64, P65, N71, Q74, K76, and combinations thereof, and/or at least one substitution with a natural amino acid or an unnatural amino acid at at least one position within IL-2Rα interaction region, IL-2Rβ interaction region and/or IL-2Rγ interaction region;
f) combinations of a) through e).

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide: has at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity in one or more regions of amino acid positions 10-25, 80-100 and/or 100-134 to the corresponding one or more regions of an IL-2 polypeptide comprising an amino acid sequence set forth in SEQ ID NO:1 or SEQ ID NO:2.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100% sequence identity sequence identity to an IL-2 polypeptide comprising the amino acid sequence set forth in SEQ ID NO:1 or SEQ ID NO:2.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide comprises at least one substitution with lysine, cysteine, histidine, arginine, aspartic acid, glutamic acid, serine, threonine, alanine, tryptophan, isoleucine, phenylalanine, or tyrosine at a position selected from the group consisting of Q13, L19, N29, N30, Y31, K32, N33, P34, K35, T37, R38, T41, F42, K43, Y45, K48, K49, E62, K64, P65, N71, Q74, K76, R81, L85, S87, V91, I92, V93, and combinations thereof.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide comprises: a) at least one substitution with a natural amino acid at a position selected from the group consisting of N29, N30, Y31, K32, N33, P34, K35, R38, T41, F42, K43, Y45, K48, K49, E62, K64, P65, N71, Q74, K76 and combinations thereof, and is configured to be conjugated to a water-soluble polymer, a lipid, a protein, or a peptide at at least one position selected from the group consisting of N29, N30, Y31, K32, N33, P34, K35, R38, T41, F42, K43, Y45, K48, K49, E62, K64, P65, N71, Q74, K76, and combinations thereof; and/or b) at least one substitution with a natural amino acid at at least one position selected from the group consisting of N29, N30, Y31, K32, N33, P34, K35, R38, T41, F42, K43, Y45, K48, K49, E62, K64, P65, N71, Q74, K76 and combinations thereof, and is configured to be conjugated to a water-soluble polymer, a lipid, a protein, or a peptide at the N terminal and/or C terminal of the modified IL-2 polypeptide.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide comprises: a) at least one substitution with lysine, cysteine, histidine, arginine, aspartic acid, glutamic acid, serine, threonine, alanine, tryptophan, isoleucine, phenylalanine, or tyrosine at at least one position selected from the group consisting of N29, N30, Y31, K32, N33, P34, K35, R38, T41, F42, K43, Y45, K48, K49, E62, K64, P65, N71, Q74, K76, and combinations thereof; and/or b) comprises at least one substitution with lysine, cysteine, histidine, arginine, aspartic acid, glutamic acid, serine, threonine, alanine, tryptophan, isoleucine, phenylalanine, or tyrosine at a position selected from the group consisting of N30, Y31, N33, P34, K35, R38, T41, K43, K48, K49, K64, P65, N71, Q74, K76, and combinations thereof.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide comprises: a) at least one substitution with cysteine at a position selected from the group consisting of N29, N30, Y31, N33, P34, K35, R38, T41, K43, K48, K49, K64, P65, N71, Q74, K76 and a combination thereof; b) at least one substitution with cysteine at a position selected from the group consisting of N29, Y31, K35, P65, N71, Q74 and a combination thereof; c) at least one substitution with any amino acid at a position of Y31, N29 or a combination thereof, d) at least one substitution with cysteine, serine or alanine at a position of Y31, N29 or a combination thereof, e) at least one substitution with cysteine at a position of Y31; f) at least one substitution with cysteine at a position of N29; and/or g) at least one substitution with cysteine at a position of P65.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide comprises at least one substitution with a natural amino acid at at least one position selected from the group consisting of R38, F42, Y45, E62, P65, and combinations thereof.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide comprises at least one substitution with lysine, cysteine, histidine, arginine, aspartic acid, glutamic acid, serine, threonine, alanine, tryptophan, isoleucine, phenylalanine, or tyrosine at at least one position selected from the group consisting of R38, F42, Y45, E62, P65 and a combination thereof.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide comprises: a) at least one substitution with cysteine at a position selected from the group consisting of R38, F42, Y45, E62, P65, and combinations thereof, and/or b) a substitution with alanine, lysine or serine at position F42; and/or c) a substitution with alanine at position F42; and/or d) a substitution with serine at position F42; and/or e) a substitution with lysine at position F42; and/or f) a substitution with alanine, histidine or serine at position Y45; and/or g) a substitution with alanine at position Y45; and/or h) a substitution with histidine at position Y45; and/or i) a substitution with alanine, aspartic acid or serine at position R38; and/or j) a substitution with aspartic acid at position R38; and/or k) a substitution with alanine at position P65; and/or l) a substitution with serine at position P65; and/or m) a substitution with alanine at position E62; and/or n) a substitution with lysine at position F42, and/or o) a substitution with cysteine at position Y31, and/or p) combinations of a) through o).

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide comprises at least one substitution with a natural amino acid at at least one position selected from the group consisting of Q13, L19, R81, L⁸⁵, S87, V91, I92, V93, and combinations thereof.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide comprises at least one substitution with lysine, cysteine, histidine, arginine, aspartic acid, glutamic acid, serine, threonine, alanine, tryptophan, isoleucine, phenylalanine, or tyrosine at a position selected from the group consisting of Q13, L19, R81, L85, S87, V91, I92, V93, and combinations thereof.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide comprises at least one substitution with cysteine at a position selected from the group consisting of Q13, L19, R81, L85, S87, V91, I92, V93, and combinations thereof.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide comprises: a) a substitution at position Y31; and/or b) a substitution at position F42; and/or c) a substitution at position C125; and/or d) a substitution at positions Y31, F42, and C125.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide comprises: a) a substitution with cysteine at position Y31; and/or b) a substitution with phenylalanine at position F42; and/or c) a substitution with serine at position C125; and/or d) a substitution with cysteine at position Y31, a substitution with phenylalanine at position Y31, and a substitution with serine at position C125.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide comprises at least one substitution with a natural amino acid or an unnatural amino acid at at least one position within IL-2Rα interaction region, IL-2Rβ interaction region and/or IL-2Rγ interaction region.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide comprises at least one substitution with a natural amino acid at a position within IL-2Rα interaction region.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide comprises at least one substitution with a natural amino acid at a position within IL-2Rβ interaction region.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide comprises: a) a substitution with a natural amino acid at a position within IL-2Rα interaction region and a substitution with a natural amino acid at a position within IL-2Rβ interaction region; b) a substitution with a natural amino acid at a position within IL-2Rα interaction region and a substitution with a natural amino acid at a position within IL-2Rγ interaction region; or c) a substitution with a natural amino acid at a position within IL-2Rα interaction region, a substitution with a natural amino acid at a position within IL-2Rβ interaction region and a substitution with a natural amino acid at a position within IL-2Rγ interaction region.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide has reduced binding to an IL-2Rα compared to an IL-2 polypeptide comprising an amino acid sequence set forth in SEQ ID NO:1 or SEQ ID NO:2 without the at least one substitution.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide has an N-terminal deletion and/or a C-terminal deletion.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide has an N-terminal deletion of amino acid residues 1-30, and/or a C terminal deletion of amino acid residues 114-134.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the fusion protein comprises the modified IL-2 polypeptide and an additional amino acid sequence.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the fusion protein comprises the modified IL-2 polypeptide linked to an additional amino acid sequence via a linker.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the fusion protein comprises the modified IL-2 polypeptide linked to an additional amino acid sequence, wherein the additional amino acid sequence confers an enhanced and/or extended pharmacokinetic (PK) profile on the modified IL-2 polypeptide or on the fusion protein compared to an IL-2 polypeptide comprising an amino acid sequence set forth in SEQ ID NO:1 or SEQ ID NO:2 without the at least one substitution

n certain embodiments, which may be combined with other embodiments provided herein and throughout, are provided polynucleotides comprising a nucleic acid sequence encoding a fusion protein comprising a modified interleukin 2 (IL-2) polypeptide, wherein the fusion protein comprises the modified IL-2 polypeptide linked to an additional amino acid sequence, wherein additional amino acid sequence is fused to the additional amino acid sequence via the N-terminus of the modified IL-2 polypeptide or via the C-terminus of the modified IL-2 polypeptide.

n certain embodiments, which may be combined with other embodiments provided herein and throughout, are provided polynucleotides comprising a nucleic acid sequence encoding a fusion protein comprising a modified interleukin 2 (IL-2) polypeptide, wherein the fusion protein comprises the modified IL-2 polypeptide linked to an additional amino acid sequence, wherein the additional amino acid sequence comprises an antibody sequence or a portion or a fragment thereof.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the fusion protein comprises the modified IL-2 polypeptide linked to an additional amino acid sequence, wherein the additional amino acid sequence comprises an Fc portion of an antibody.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the fusion protein comprises the modified IL-2 polypeptide linked to an additional amino acid sequence, wherein the additional amino acid sequence comprises a serum albumin or a PK-extending fragment or analog thereof.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the fusion protein comprises the modified IL-2 polypeptide linked to an additional amino acid sequence, wherein the additional amino acid sequence comprises: a) a human serum albumin (HSA); b) a murine serum albumin (MSA); c) or a PK-extending fragment or analog of a) or b).

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide comprises the amino acid sequence set forth in SEQ ID NO: 3

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide and/or the additional amino acid sequence is further configured to be conjugated to a water-soluble polymer, a lipid, a protein, or a peptide via a single amino acid residue of the modified IL-2 polypeptide.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide and/or the additional amino acid sequence is further configured to be conjugated to a water-soluble polymer, a lipid, a protein, or a peptide via: i) the alpha amino group of the N-terminal amino acid residue of the fusion polypeptide; ii) the epsilon amino group of a lysine amino acid residue of the fusion polypeptide; or iii) an N-glycosylation site or O-glycosylation site of the fusion polypeptide.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the IL-2 polypeptide or the fusion protein has increased binding to an IL-2Rβγ compared to an IL-2 polypeptide comprising an amino acid sequence set forth in SEQ ID NO:1 or SEQ ID NO:2 without the at least one substitution.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the IL-2 polypeptide or the fusion protein has: (i) reduced binding to an IL-2Rα and (ii) increased binding to an IL-2Rβγ; compared to an IL-2 polypeptide comprising an amino acid sequence set forth in SEQ ID NO:1 or SEQ ID NO:2 without the at least one substitution.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the binding affinity of the IL-2 polypeptide or the fusion protein to an IL-2Rα is decreased: from about 10%, about 20%, about 30%, about 40%, about 50%, about 60, about 70%, about 80%, about 90%, about 100 about 100%; or is decreased from about 1 fold to about 100,000 fold or more compared to the binding affinity of an IL-2 polypeptide comprising an amino acid sequence set forth in SEQ ID NO:1 or SEQ ID NO:2 without the at least one substitution.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the IL-2 polypeptide or the fusion protein has no detectable binding to an IL-2Rα.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the IL-2 polypeptide or the fusion protein has reduced receptor signaling potency to IL-2Rαβγ compared to an IL-2 polypeptide comprising an amino acid sequence set forth in SEQ ID NO:1 or SEQ ID NO:2 without the at least one substitution.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the ratio between the signaling potency of the IL-2 polypeptide or the fusion protein to IL-2Rαβγ and the signaling potency to IL-2Rαβγ of the an IL-2 polypeptide comprising an amino acid sequence set forth in SEQ ID NO:1 or SEQ ID NO:2 without the at least one substitution is from about 1/2 to about 1/100,000.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the IL-2 polypeptide or the fusion protein has no detectable receptor signaling potency to IL-2Rαβγ.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the IL-2 polypeptide or the fusion protein: (i) has reduced binding to an IL-2Rα compared to an IL-2 polypeptide comprising an amino acid sequence set forth in SEQ ID NO:1 or SEQ ID NO:2 without the at least one substitution; and (ii) has reduced receptor signaling potency to IL-2Rαβγ compared to an IL-2 polypeptide comprising an amino acid sequence set forth in SEQ ID NO:1 or SEQ ID NO:2 without the at least one substitution.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the IL-2 polypeptide or the fusion protein has no detectable binding to an IL-2Rα and has no detectable receptor signaling potency to IL-2Rαβγ.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide or the fusion protein: has comparable has increased binding to an interleukin 2 receptor β (IL-2R β) and/or an interleukin 2 receptor γ (IL-2R γ) compared to an IL-2 polypeptide comprising an amino acid sequence set forth in SEQ ID NO:1 or SEQ ID NO:2 without the at least one substitution; and/or has comparable or has increased receptor signaling potency to IL-2R γ compared to an IL-2 polypeptide comprising an amino acid sequence set forth in SEQ ID NO:1 or SEQ ID NO:2 without the at least one substitution.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the IL-2 polypeptide or the fusion protein has comparable or has higher binding level to an IL-2R β or an IL-2R γ compared to an n IL-2 polypeptide comprising an amino acid sequence set forth in SEQ ID NO:1 or SEQ ID NO:2 without the at least one substitution.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the IL-2 polypeptide or the fusion protein has comparable or has higher receptor signaling potency to IL-2R γ compared to an IL-2 polypeptide comprising an amino acid sequence set forth in SEQ ID NO:1 or SEQ ID NO:2 without the at least one substitution.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the IL-2 polypeptide or the fusion protein has comparable or has higher binding level to an IL-2R β or an IL-2R γ compared to an comparable IL-2 polypeptide comprising an amino acid sequence set forth in SEQ ID NO:1 or SEQ ID NO:2 without the at least one substitution, and has comparable or has higher receptor signaling potency to IL-2R βγ compared to an IL-2 polypeptide comprising an amino acid sequence set forth in SEQ ID NO:1 or SEQ ID NO:2 without the at least one substitution.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the IL-2 polypeptide or the fusion protein has increased ratio of signaling potency to IL-2Rβγ over signaling potency to IL-2Rαβγ (increased ratio of signaling potency to IL-2Rβγ/signaling potency to IL-2Rαβγ) compared to an IL-2 polypeptide comprising an amino acid sequence set forth in SEQ ID NO:1 or SEQ ID NO:2 without the at least one substitution.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide further comprises a promoter operably linked to the nucleic acid sequence encoding the fusion protein.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide further comprises a promoter operably linked to the nucleic acid sequence encoding the fusion protein, wherein the promoter drives tissue-specific expression of the fusion protein.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide further comprises a promoter operably linked to the nucleic acid sequence encoding the fusion protein, wherein the promoter drives muscle-specific expression of the fusion protein.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide further comprises a promoter operably linked to the nucleic acid sequence encoding the fusion protein, wherein the promoter drives cancer-specific expression or tumor-specific expression of the fusion protein.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide further comprises a promoter operably linked to the nucleic acid sequence encoding the fusion protein, wherein the promoter drives liver-specific expression of the fusion protein.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide further comprises a promoter operably linked to the nucleic acid sequence encoding the fusion protein, wherein the promoter is constitutively active.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide further comprises a promoter operably linked to the nucleic acid sequence encoding the fusion protein, wherein the promoter is conditionally active.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide further comprises a promoter operably linked to the nucleic acid sequence, wherein the promoter comprises an SP6, T3, or T7 promoter sequence.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide further comprises wherein at least a portion of the nucleic acid sequence encoding the fusion protein has been codon-optimized.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide further comprises, wherein the polynucleotide includes at least one modified nucleotide.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide further comprises, wherein the polynucleotide includes at least one modified nucleotide, wherein the at least one modified nucleotide is pseudouridine, N1-methyl-pseudouridine, or 2-thiouridine.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide further comprises a 5′ cap structure.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide further comprises a 3′ polyA sequence.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide further comprises: DNA; RNA; or a DNA/RNA hybrid.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide further comprises RNA.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide further comprises: a viral vector; a nonviral vector; a plasmid; or a Nanoplasmid™ vector.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide is configured to express the fusion protein in vitro.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide is configured to express the fusion protein in vivo.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide is in an isolated form.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the modified IL-2 polypeptide or the fusion protein has a half-life in vivo from about 5 minutes to about 10 days, from about 5 minutes to about 9 days, from about 5 minutes to about 8 days, from about 5 minutes to about 8 days, from about 5 minutes to about 7 days, from about 5 minutes to about 6 days, from about 5 minutes to about 5 days, from about 5 minutes to about 4 days, from about 5 minutes to about 3 days, from about 5 minutes to about 2 days, from about 5 minutes to about 1 day, from about 1 hour to about 10 days, about 1 hour to about 9 days, from out 1 hour to about 8 days, from about 1 hour to about 7 days, from out 1 hours to about 6 days, from about 1 hour to about 5 days, from about 1 hour to about 4 days, about 1 hour to about 3 days, from out 1 hour to about 2 days, from about 1 hour to about 1 day, from about 5 minutes, from about 10 minutes, from about 20 minutes, from about 30 minutes, from about 40 minutes, from about 50 minutes, from about 1 hour, from about 2 hours, from about 3 hours, from about 4 hours, from about 5 hours, from about 6 hours, from about 7 hours, from about 8 hours, from about 9 hours, from about 10 hours, from about 11 hours, from about 12 hours, from about 13 hours, from about 14 hours, from about 15 hours, from about 16 hours, from about 17 hours, from about 18 hours, from about 19 hours, from about 20 hours, from about 21 hours, from about 22 hours, from about 23 hours, from about 1 day, from about 2 days, from about 3 days, from about 4 days, from about 5 days, from about 6 days, from about 7 days, from about 8 days, from about 9 days, from about 10 days or a value or a range in between.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutically acceptable carrier comprises a lipid.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutically acceptable carrier comprises a lipid, wherein the lipid comprises a cationic lipid of formula (I):

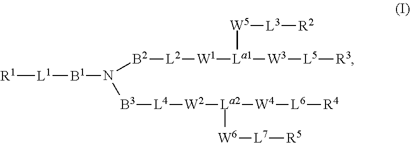

or a pharmaceutically acceptable salt, solvate, hydrate, stereoisomer, or prodrug thereof, wherein:
R¹ is H, —OR^{1A}, —YOR^{1A}, —NR^{1A}R^{1B}, —YNR^{1A}R^{1B}, —SR^{1A}, —YSR^{1A}, —(C═O)R^{1A}, —Y(C═O)R^{1A}, —(C═O)OR^{1A}, —Y(C═O)OR^{1A}, —O(C═O)R^{1A}, —YO(C═O)R^{1A}, —O(C═O)OR^{1A}, —YO(C═O)OR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Y is substituted or unsubstituted C₀-C₁₂ alkylene or substituted or unsubstituted 0 to 12 membered heteroalkylene;
R² is H, —OR^{2A}, —SR^{2A}, —(C═O)R^{2A}, —(C═O)OR^{2A}, —O(C═O)R^{2A}, —O(C═O)OR^{2A}, —(C═O)NHR^{2A}, —NH(C═O)R^{2A}, substituted or unsubstituted alkyl, or substituted or unsubstituted heteroalkyl;
R³ is H, —OR^{3A}, —SR^{3A}, —(C═O)R^{3A}, —(C═O)OR^{3A}, —O(C═O)R^{3A}, —O(C═O)OR^{3A}, —(C═O)NHR^{3A}, —NH(C═O)R^{3A}, substituted or unsubstituted alkyl, or substituted or unsubstituted heteroalkyl;
R⁴ is H, —OR^{4A}, —SR^{4A}, —(C═O)R^{4A}, —(C═O)OR^{4A}, —O(C═O)R^{4A}, —O(C═O)OR^{4A}, —(C═O)NHR^{4A}, —NH(C═O)R^{4A}, substituted or unsubstituted alkyl, or substituted or unsubstituted heteroalkyl;
R⁵ is H, —OR^{5A}, —SR^{5A}, —(C═O)R^{5A}, —(C═O)OR^{5A}, —O(C═O)R^{5A}, —O(C═O)OR^{5A}, —(C═O)NHR^{5A}, —NH(C═O)R^{5A}, substituted or unsubstituted alkyl, or substituted or unsubstituted heteroalkyl;
B¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
B² and B³ are each independently a bond, substituted or unsubstituted alkylene, or substituted or unsubstituted heteroalkylene;
L¹ is a bond, —O(C═O)—, —(C═O)O—, —O(C═O)O—, —C(═O)—, —O—, —O(CR¹⁰¹R¹⁰²)ₛO—, —S—, —C(═O)S—, —SC(═O)—, —NR¹⁰¹C(═O)—, —C(═O)NR¹⁰¹—, —NR¹⁰¹C(═S)—, —C(═S)NR¹⁰¹—, —NR¹⁰¹C(═O)NR¹⁰²—, —NR¹⁰¹C(═S)NR¹⁰²—, —OC(═O)NR¹⁰¹—, NR¹⁰¹C(═O)O—, —SC(═O)NR¹⁰¹— or —NR¹⁰¹C(═O)S—;
L² is a bond, —O(C═O)—, —(C═O)O—, —O(C═O)O—, —C(═O)—, —O—, —O(CR²⁰¹R²⁰²)ₛO—, —S—, —C(═O)S—, —SC(═O)—, —NR²⁰¹C(═O)—, —C(═O)NR²⁰¹—, —NR²⁰¹C(═O)NR²⁰²—, —NR²⁰¹C(═S)—, —C(═S)NR²⁰¹—, —NR²⁰¹C(═S)NR²⁰²—, —OC(═O)NR²⁰¹—, —NR²⁰¹C(═O)O—, —SC(═O)NR²⁰¹— or —NR²⁰¹C(═O)S—;
L³ is a bond, —O(C═O)—, —(C═O)O—, —O(C═O)O—, —C(═O)—, —O—, —O(CR³⁰¹R³⁰²)ₛO—, —S—, —C(═O)S—, —SC(═O)—, —NR³⁰¹C(═O)—, —C(═O)NR³⁰¹—, —NR³⁰¹C(═O)NR³⁰²—, —NR³⁰¹C(═S)—, —C(═S)NR³⁰¹—, —NR³⁰¹C(═S)NR³⁰²—, —OC(═O)NR³⁰¹—, —NR³⁰¹C(═O)O—, —SC(═O)NR³⁰¹— or —NR³⁰¹C(═O)S—;
L⁴ is a bond, —O(C═O)—, —(C═O)O—, —O(C═O)O—, —C(═O)—, —O—, —O(CR⁴⁰¹R⁴⁰²)ₛO—, —S—, —C(═O)S—, —SC(═O)—, —NR⁴⁰¹C(═O)—, —C(═O)NR⁴⁰¹—, —NR⁴⁰¹C(═O)NR⁴⁰²—, —NR⁴⁰¹C(═S)—, —C(═S)NR⁴⁰¹—, —NR⁴⁰¹C(═S)NR⁴⁰²—, —OC(═O)NR⁴⁰¹—, —NR⁴⁰¹C(═O)O—, —SC(═O)NR⁴⁰¹— or —NR⁴⁰¹C(═O)S—;
L⁵ is a bond, —O(C═O)—, —(C═O)O—, —O(C═O)O—, —C(═O)—, —O—, —O(CR⁵⁰¹R⁵⁰²)ₛO—, —S—, —C(═O)S—, —SC(═O)—, —NR⁵⁰¹C(═O)—, —C(═O)NR⁵⁰¹—, —NR⁵⁰¹C(═O))NR⁵⁰²—, —NR⁵⁰¹C(═S)—, —C(═S)NR⁵⁰¹—, —NR⁵⁰¹C(═S)NR⁵⁰²—, —OC(═O)NR⁵⁰¹—, —NR⁵⁰¹C(═O)O—, —SC(═O)NR⁵⁰¹— or —NR⁵⁰¹C(═O)S—;
L⁶ is a bond, —O(C═O)—, —(C═O)O—, —O(C═O)O—, —C(═O)—, —O—, —O(CR⁶⁰¹R⁶⁰²)ₛO—, —S—, —C(═O)S—, —SC(═O)—, —NR⁶⁰¹C(═O)—, —C(═O)NR⁶⁰¹—, —NR⁶⁰¹C(═O)NR⁶⁰²—, —NR⁶⁰¹C(═S)—, —C(═S)NR⁶⁰¹—, —NR⁶⁰¹C(═S)NR⁶⁰²—, —OC(═O)NR⁶⁰¹—, —NR⁶⁰¹C(═O)O—, —SC(═O)NR⁶⁰¹— or —NR⁶⁰¹C(═O)S—;
L⁷ is a bond, —O(C═O)—, —(C═O)O—, —O(C═O)O—, —C(═O)—, —O—, —O(CR⁷⁰¹R⁷⁰²)ₛO—, —S—, —C(═O)S—, —SC(═O)—, —NR⁷⁰¹C(═O)—, —C(═O)NR⁷⁰¹—, —NR⁷⁰¹C(═O)NR⁷⁰²—, —NR⁷⁰¹C(═S)—, —C(═S)NR⁷⁰¹—, —NR⁷⁰¹C(═S)NR⁷⁰²—, —OC(═O)NR⁷⁰¹—, —NR⁷⁰¹C(═O)O—, —SC(═O)NR⁷⁰¹— or —NR⁷⁰¹C(═O)S—;
L^{a1} and L^{a2} are each independently

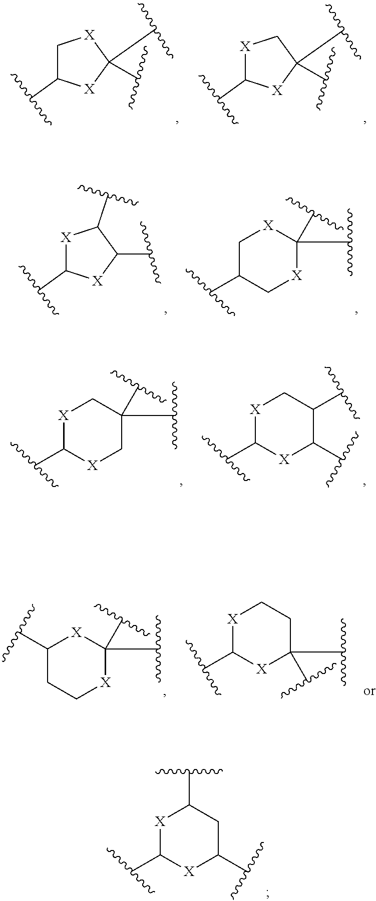

each X is independently O, S, or CH₂;
W¹, W², W³, W⁴, W⁵, and W⁶ are each independently a bond, substituted or unsubstituted C₁-C₁₂ alkylene, or substituted or unsubstituted 2 to 12 membered heteroalkylene;
each R^{1A} and R^{1B} is independently H, substituted or unsubstituted C₁-C₁₂ alkyl, or
substituted or unsubstituted 2 to 12 membered heteroalkyl;
each R^{2A}, R^{3A}, R^{4A}, and R^{5A} is independently H, substituted or unsubstituted C₁-C₃₀ alkyl, or substituted or unsubstituted 2 to 30 membered heteroalkyl;
each R¹⁰¹, R¹⁰², R²⁰¹, R²², R³⁰¹, R³⁰², R⁴⁰¹, R⁴⁰², R⁵⁰¹, R⁵⁰², R⁶⁰¹, R⁶⁰², R⁷⁰¹, and R⁷⁰² is independently H, substituted or unsubstituted C₁-C₁₂ alkyl, or substituted or unsubstituted 2 to 12 membered heteroalkyl; and
each s is independently an integer from 1 to 4.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutically acceptable carrier comprises a lipid, wherein the lipid comprises a cationic lipid of formula (I):

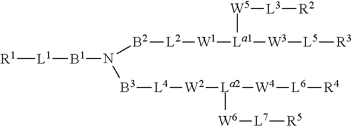

R¹ is H, —OR^{1A} or substituted or unsubstituted heteroalkyl;
L¹ is a bond, —NR¹⁰¹C(═S)—, —C(═S)NR¹⁰¹—, —O(C═O)—, —(C═O)O—, or —O—;
B¹ is a bond or a substituted or unsubstituted alkylene;
B² and B³ are each independently a bond or substituted or unsubstituted alkylene;
L² is a bond, —O(C═O)—, —(C═O)O—, —O(C═O)O—, —C(═O)—, —O—, or —S—;
L⁴ is a bond, —O(C═O)—, —(C═O)O—, —O(C═O)O—, —C(═O)—, —O—, or —S—;
W¹, W², W³, W⁴, W⁵, and W⁶ are each independently a bond or substituted or unsubstituted C₁-C₁₂ alkylene;
L^{a1} and L^{a2} are each independently

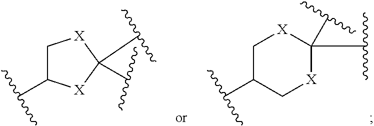

each X is independently O or S;
L³ is a bond, —O(C═O)—, —(C═O)O—, —O(C═O)O—, —C(═O)—, —O—, or —S—;
L⁵ is a bond, —O(C═O)—, —(C═O)O—, —O(C═O)O—, —C(═O)—, —O—, or —S—;
L⁶ is a bond, —O(C═O)—, —(C═O)O—, —O(C═O)O—, —C(═O)—, —O—, or —S—;
L⁷ is a bond, —O(C═O)—, —(C═O)O—, —O(C═O)O—, —C(═O)—, —O—, or —S—;
R² is H or substituted or unsubstituted alkyl;
R³ is H or substituted or unsubstituted alkyl;
R⁴ is H or substituted or unsubstituted alkyl;
R⁵ is H or substituted or unsubstituted alkyl;
each R^{1A} is independently H or substituted or unsubstituted C₁-C₁₂ alkyl; and
each R¹⁰¹ is independently H or substituted or unsubstituted 2 to 12 membered heteroalkyl.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutically acceptable carrier comprises a lipid, wherein the lipid comprises a cationic lipid of formula (I):

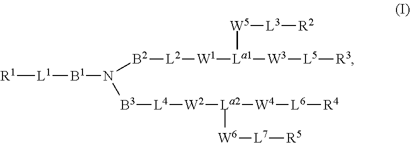

or a pharmaceutically acceptable salt, solvate, hydrate, stereoisomer, or prodrug thereof, wherein, wherein the cationic lipid is a lipid wherein: R¹ is H, —OH, methoxy, ethoxy, or substituted or unsubstituted heteroalkyl;
L¹ is a bond, —NR¹⁰¹C(═S)—, or —C(═S)NR¹⁰¹—;
B¹ is a bond or an unsubstituted C₁-C₈ alkylene;
B² and B³ are each independently a bond or substituted or unsubstituted C₁-C₈ alkylene;
L² is a bond, —O(C═O)—, or —(C═O)O—;
L⁴ is a bond, —O(C═O)—, or —(C═O)O—;
W¹, W², W³, W⁴, W⁵, and W⁶ are each independently a bond or substituted or unsubstituted C₁-C₁₂ alkylene;
L^{a1} and L^{a2} are each independently;

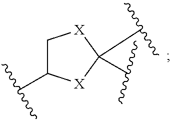

each X is independently O or S;
L³ is a bond, —O(C═O)—, or —(C═O)O—;
L⁵ is a bond, —O(C═O)—, or —(C═O)O—;
L⁶ is a bond, —O(C═O)—, or —(C═O)O—;
L⁷ is a bond, —O(C═O)—, or —(C═O)O—;
R² is H or substituted or unsubstituted C₁-C₁₂ alkyl;
R³ is H or substituted or unsubstituted C₁-C₁₂ alkyl;
R⁴ is H or substituted or unsubstituted C₁-C₁₂ alkyl;
R⁵ is H or substituted or unsubstituted C₁-C₁₂ alkyl; and
each R¹⁰¹ is independently substituted or unsubstituted 2 to 12 membered heteroalkyl.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutically acceptable carrier comprises a lipid, wherein the lipid comprises a cationic lipid of formula (I):

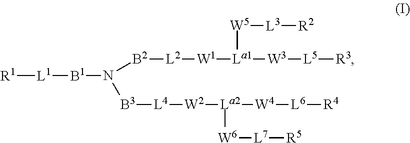

or a pharmaceutically acceptable salt, solvate, hydrate, stereoisomer, or prodrug thereof, wherein, wherein the cationic lipid is a lipid wherein: R¹ is —OH or methoxy;
L¹ is a bond;
B¹ is an unsubstituted C₁-C₈ alkylene;
B² and B³ are each independently a bond or substituted or unsubstituted C₁-C₈ alkylene;
L² is a bond;
L⁴ is a bond;
W¹, W², W³, W⁴, W⁵, and W⁶ are each independently a bond or substituted or unsubstituted C₁-C₁₂ alkylene;
L^{a1} and L^{a2} are each independently;

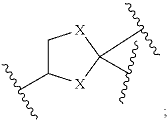

each X is independently O;
L³ is a bond;
L⁵ is a bond;
L⁶ is a bond;
L⁷ is a bond;
R² is H or substituted or unsubstituted C₁-C₁₂ alkyl;
R³ is H or substituted or unsubstituted C₁-C₁₂ alkyl;
R⁴ is H or substituted or unsubstituted C₁-C₁₂ alkyl; and
R⁵ is H or substituted or unsubstituted C₁-C₁₂ alkyl.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutically acceptable carrier comprises a lipid, wherein the lipid comprises a cationic lipid of formula (I):

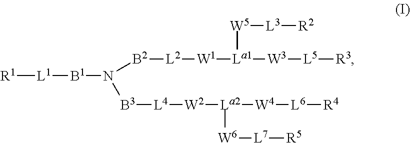

or a pharmaceutically acceptable salt, solvate, hydrate, stereoisomer, or prodrug thereof, wherein, wherein the cationic lipid is a lipid wherein: R¹ is substituted or unsubstituted heteroalkyl;
L¹ is —C(═S)NR¹⁰¹—, where the carbon atom is connected to the nitrogen atom in formula (I);
B¹ is a bond;
B² and B³ are each independently a bond or substituted or unsubstituted C₁-C₈ alkylene;
L² is a bond, —O(C═O)—, or —(C═O)O—;
L⁴ is a bond, —O(C═O)—, or —(C═O)O—;
W¹, W², W³, W⁴, W⁵, and W⁶ are each independently a bond or substituted or unsubstituted C₁-C₁₂ alkylene;
L^{a1} and L^{a2} are each independently;

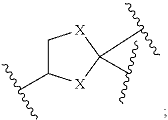

each X is independently O;
L³ is a bond;
L⁵ is a bond;
L⁶ is a bond;
L⁷ is a bond;
R² is H or substituted or unsubstituted C₁-C₁₂ alkyl;
R³ is H or substituted or unsubstituted C₁-C₁₂ alkyl;
R⁴ is H or substituted or unsubstituted C₁-C₁₂ alkyl; and
R⁵ is H or substituted or unsubstituted C₁-C₁₂ alkyl.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutically acceptable carrier comprises a lipid, wherein the lipid comprises a cationic lipid of formula (I):

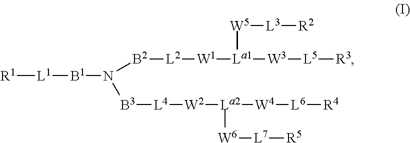

or a pharmaceutically acceptable salt, solvate, hydrate, stereoisomer, or prodrug thereof, wherein, wherein the cationic lipid is:

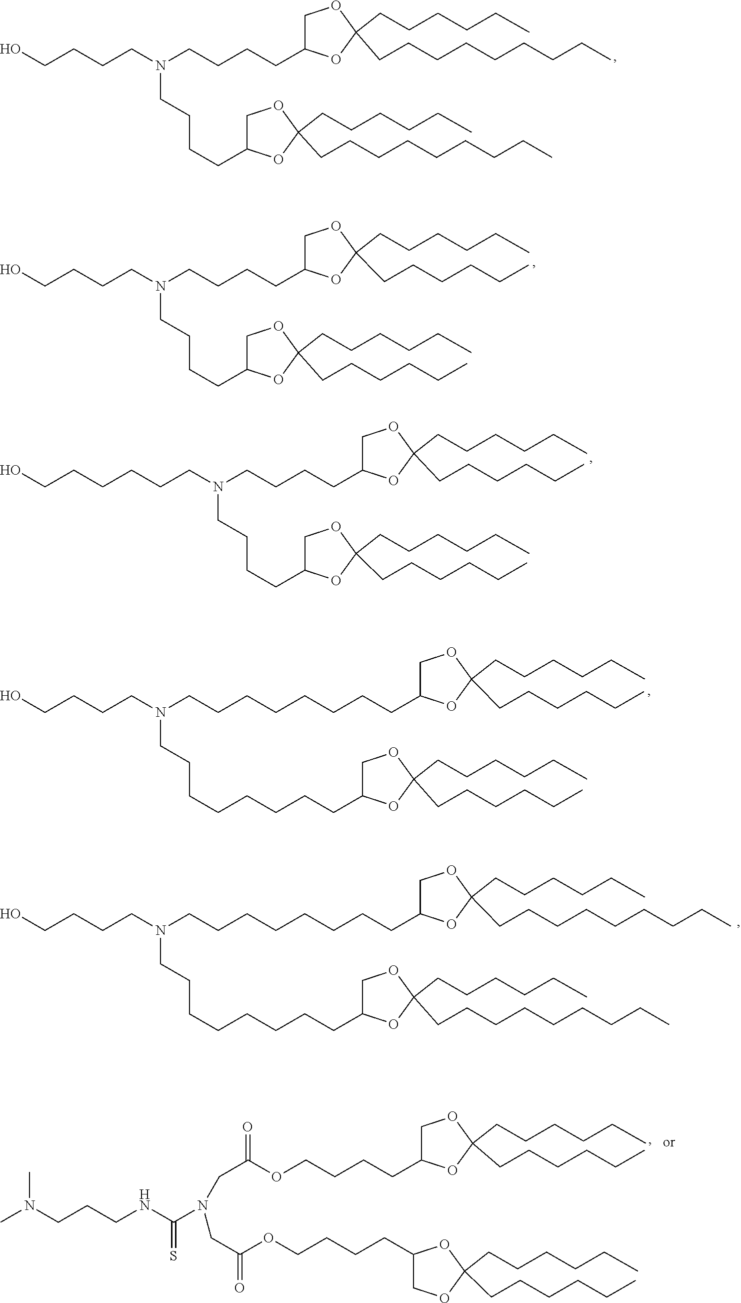

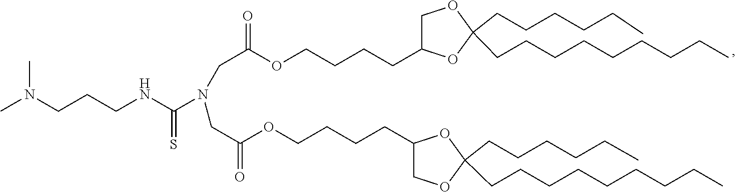 [SMILES: CCCCCCCCCC1(CCCCCC)OCC(CCCCOC(=O)CN(CC(=O)OCCCCC2COC(CCCCCC)(CCCCCCCCC)O2)C(=S)NCCCN(C)C)O1]

or a pharmaceutically acceptable salt thereof.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutically acceptable carrier comprises a lipid, wherein the lipid comprises a cationic lipid of formula (II):

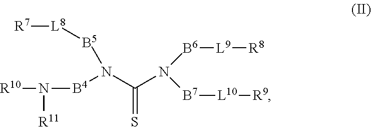

or a pharmaceutically acceptable salt, solvate, hydrate, stereoisomer, or prodrug thereof, wherein:
B⁴ is W⁷-L^{a3}-W⁸;
W⁷ and W⁸ are each independently a bond, substituted or unsubstituted alkylene, or substituted or unsubstituted heteroalkylene;
L^{a3} is a bond, —O(C═O)—, —(C═O)O—, —O(C═O)O—, —C(═O)—, —O—, —O(CR^{a31}R^{a32})ₛO—, —S—, —C(═O)S—, —SC(═O)—, —NR^{a31}C(═O)—, —C(═O)NR^{a31}—, —NR^{a31}C(═O)NR^{a32}—, —NR^{a31}C(═S)—, —C(═S)NR^{a31}—, —NR^{a31}C(═S)NR^{a32}—, —OC(═O)NR^{a31}—, —NR^{a31}C(═O)O—, —SC(═O)NR^{a31}— or —NR^{a31}C(═O)S—;
R¹⁰ and R¹¹ are each independently H, substituted or unsubstituted alkyl, substituted or unsubstituted heteroalkyl, or R¹⁰ and R¹¹ together with the nitrogen atom to which they are connected form a substituted or unsubstituted heterocycloalkyl or substituted or unsubstituted heteroaryl;
B⁵, B⁶, and B⁷ are each independently a bond, substituted or unsubstituted alkylene, or substituted or unsubstituted heteroalkylene;
L⁸ is a bond, —O(C═O)—, —(C═O)O—, —O(C═O)O—, —C(═O)—, —O—, —O(CR⁸⁰¹R⁸⁰²)ₛO—, —S—, —C(═O)S—, —SC(═O)—, —NR⁸⁰¹C(═O)—, —C(═O)NR⁸⁰¹—, —NR⁸⁰¹C(═O)NR⁸⁰²—, —NR⁸⁰¹C(═S)—, —C(═S)NR⁸⁰¹—, —NR⁸⁰¹C(═S)NR⁸⁰²—, —OC(═O)NR⁸⁰²—, —NR⁸⁰¹C(═O)O—, —SC(═O)NR⁸⁰¹— or —NR⁸⁰¹C(═O)S—;
L⁹ is a bond, —O(C═O)—, —(C═O)O—, —O(C═O)O—, —C(═O)—, —O—, —O(CR⁹⁰¹R⁹⁰²)ₛO—, —S—, —C(═O)S—, —SC(═O)—, —NR⁹⁰¹C(═O)—, —C(═O)NR⁹⁰¹—, —NR⁹⁰¹C(═O)NR⁹⁰²—, —NR⁹⁰¹C(═S)—, —C(═S)NR⁹⁰¹—, —NR⁹⁰¹C(═S)NR⁹⁰²—, —OC(═O)NR⁹⁰¹—, —NR⁹⁰¹C(═O)O—, —SC(═O)NR⁹⁰¹— or —NR⁹⁰¹C(═O)S—;
L¹⁰ is a bond, —O(C═O)—, —(C═O)O—, —O(C═O)O—, —C(═O)—, —O—, —O(CR¹¹⁰R¹¹⁰)ₛO—, —S—, —C(═O)S—, —SC(═O)—, —NR¹¹⁰C(═O)—, —C(═O)NR¹¹⁰—, —NR¹¹⁰C(═O)NR¹¹¹—, —NR¹¹⁰C(═S)—, —C(═S)NR¹¹⁰—, —NR¹¹⁰C(═S)NR¹¹¹—, —OC(═O)NR¹¹⁰—, —NR¹¹⁰C(═O)O—, —SC(═O)NR¹¹⁰— or —NR¹¹⁰C(═O)S—;
R⁷, R⁸, and R⁹ are each independently H, substituted or unsubstituted C₁-C₃₀ alkyl, or substituted or unsubstituted 2 to 30 membered heteroalkyl;
each R^{a31} and R^{a32} is independently H, substituted or unsubstituted C₁-C₁₂ alkyl, or substituted or unsubstituted 2 to 12 membered heteroalkyl;
each R⁸⁰¹, R⁸⁰², R⁹⁰¹, R⁹⁰², R¹¹⁰, and R¹¹¹ is independently H, substituted or unsubstituted C₁-C₁₂ alkyl, or substituted or unsubstituted 2 to 12 membered heteroalkyl; and
each s is independently an integer from 1 to 4.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutically acceptable carrier comprises a lipid, wherein the lipid comprises a cationic lipid of formula (II):

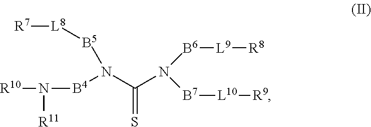

or a pharmaceutically acceptable salt, solvate, hydrate, stereoisomer, or prodrug thereof, wherein, wherein the cationic lipid is a lipid wherein:
W⁷ and W⁸ are each independently a bond or substituted or unsubstituted alkylene;
L^{a3} is a bond;
R¹⁰ and R¹¹ are each independently H, substituted or unsubstituted alkyl or R¹⁰ and R¹¹ together with the nitrogen atom to which they are connected form a substituted or unsubstituted heterocycloalkyl;
B⁵ is a bond;
B⁶ and B⁷ are each independently a bond or substituted or unsubstituted alkylene;
L⁸ is a bond;
L⁹ is a bond, —O(C═O)—, —(C═O)O—, or —C(═O)—;
L¹⁰ is a bond, —O(C═O)—, —(C═O)O—, or —C(═O)—; and
R⁷, R⁸, and R⁹ are each independently H or substituted or unsubstituted C₁-C₃₀ alkyl.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutically acceptable carrier comprises a lipid, wherein the lipid comprises a cationic lipid of formula (II):

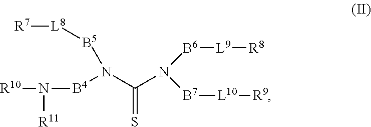

or a pharmaceutically acceptable salt, solvate, hydrate, stereoisomer, or prodrug thereof, wherein, wherein W⁷ and W⁸ are each independently a bond or substituted or unsubstituted C₁-C₈ alkylene;
L^{a3} is a bond;
R¹⁰ and R¹¹ are each independently substituted or unsubstituted alkyl or R¹⁰ and R¹¹ together with the nitrogen atom to which they are connected form a substituted or unsubstituted heterocycloalkyl;
B⁵ is a bond;
B⁶ and B⁷ are each independently a bond or substituted or unsubstituted C₁-C₈ alkylene;
L⁸ is a bond;
L⁹ is —O(C═O)— or —(C═O)O—;
L¹⁰ —O(C═O)— or —(C═O)O—; and
R⁷, R⁸, and R⁹ are each independently substituted or unsubstituted C₁-C₂₀ alkyl.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutically acceptable carrier comprises a lipid, wherein the lipid comprises a cationic lipid of formula (II):

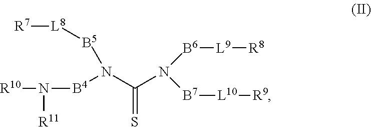

or a pharmaceutically acceptable salt, solvate, hydrate, stereoisomer, or prodrug thereof, wherein: W⁷ and W⁸ are each independently a bond or substituted or unsubstituted C₂-C₄ alkylene;
L^{a3} is a bond;
R¹⁰ and R¹¹ are each independently substituted or unsubstituted methyl, ethyl, propyl, isopropyl, or R¹⁰ and R¹¹ together with the nitrogen atom to which they are connected form a substituted or unsubstituted 3 to 8 membered heterocycloalkyl;
B⁵ is a bond;
B⁶ and B⁷ are each independently a bond or substituted or unsubstituted C₂-C₄ alkylene;
L¹ is a bond;
L⁹ is —O(C═O)— or —(C═O)O—;
L¹⁰ —O(C═O)— or —(C═O)O—;
R⁷ is H or methyl; and
R⁸, and R⁹ are each independently substituted or unsubstituted C₁-C₂₀ alkyl.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutically acceptable carrier comprises a lipid, wherein the lipid comprises a cationic lipid of formula (II):

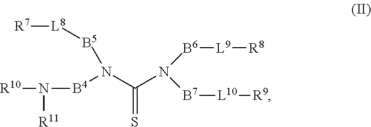

or a pharmaceutically acceptable salt, solvate, hydrate, stereoisomer, or prodrug thereof, wherein: W⁷ and W⁸ are each independently a bond or unsubstituted C₂-C₄ alkylene;
L^{a3} is a bond;
R¹⁰ and R¹¹ are each independently substituted or unsubstituted methyl, ethyl, propyl, isopropyl, or R¹⁰ and R¹¹ together with the nitrogen atom to which they are connected form a substituted or unsubstituted 5 to 6 membered heterocycloalkyl;
B⁵ is a bond;
B⁶ and B⁷ are each independently a bond or unsubstituted C₂-C₄ alkylene;
L¹ is a bond;
L⁹ is —O(C═O)— or —(C═O)O—;
L¹⁰ is —O(C═O)— or —(C═O)O—;
R⁷ is H or methyl; and
R⁸ and R⁹ are each independently substituted or unsubstituted C₁-C₂₀ alkyl.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutically acceptable carrier comprises a lipid, wherein the lipid comprises a cationic lipid of formula (II):

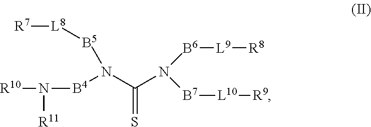

or a pharmaceutically acceptable salt, solvate, hydrate, stereoisomer, or prodrug thereof, wherein: W⁷ and W⁸ are each independently a bond or unsubstituted C₂-C₄ alkylene;
L^{a3} is a bond;
R¹⁰ and R¹¹ are each independently substituted or unsubstituted methyl, ethyl, propyl, isopropyl, or R¹⁰ and R¹¹ together with the nitrogen atom to which they are connected form a substituted or unsubstituted 5 to 6 membered heterocycloalkyl;
B⁵, B⁶, and B⁷ are each independently a bond;
L¹ is a bond;
L⁹ is a bond;
L¹⁰ is a bond;
R⁷ is H or methyl; and
R⁸ and R⁹ are each independently substituted or unsubstituted C₁-C₃₀ alkyl.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutically acceptable carrier comprises a lipid, wherein the lipid comprises a cationic lipid of formula (II):

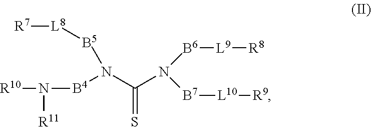

or a pharmaceutically acceptable salt, solvate, hydrate, stereoisomer, or prodrug thereof, wherein the cationic lipid is:

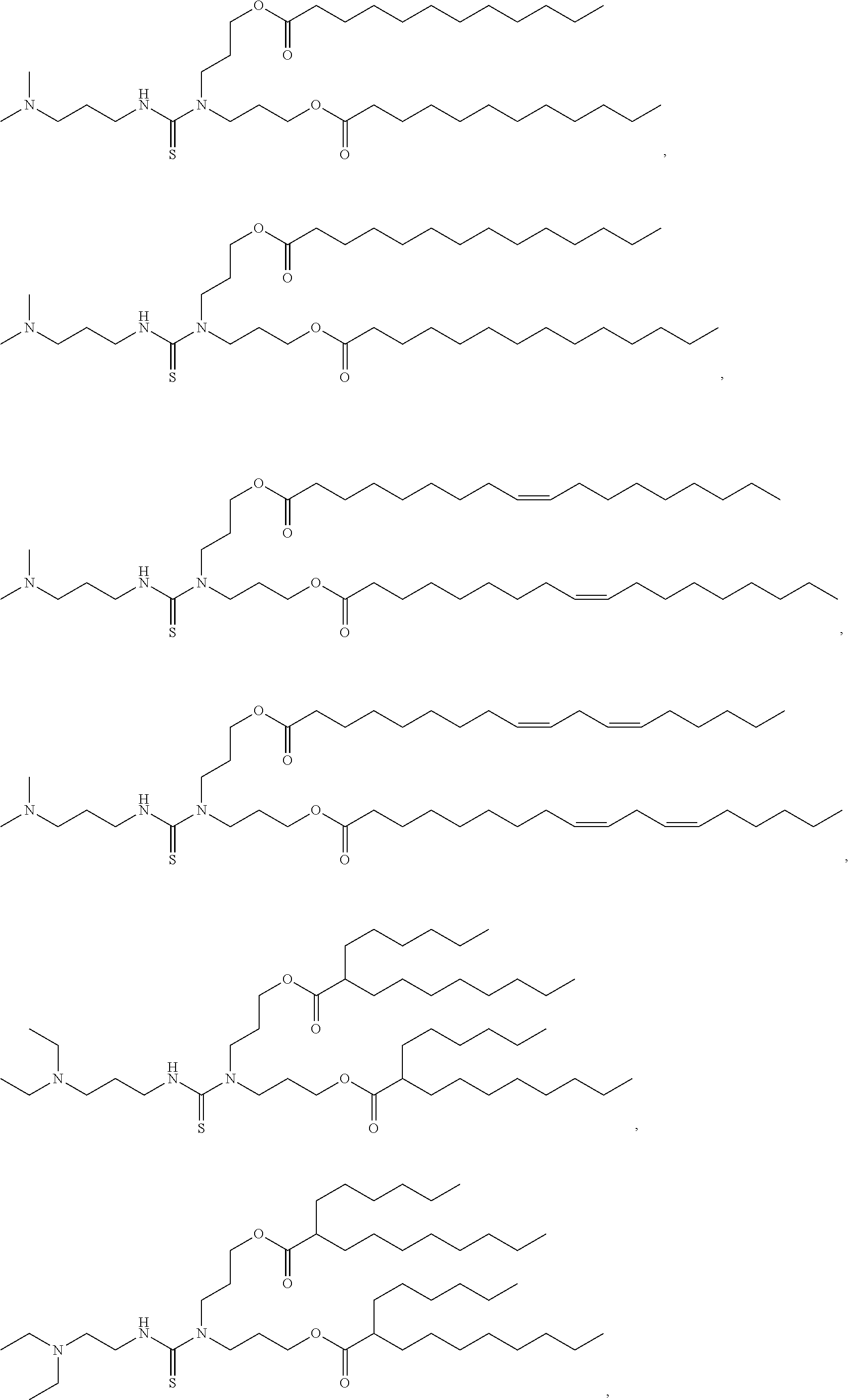

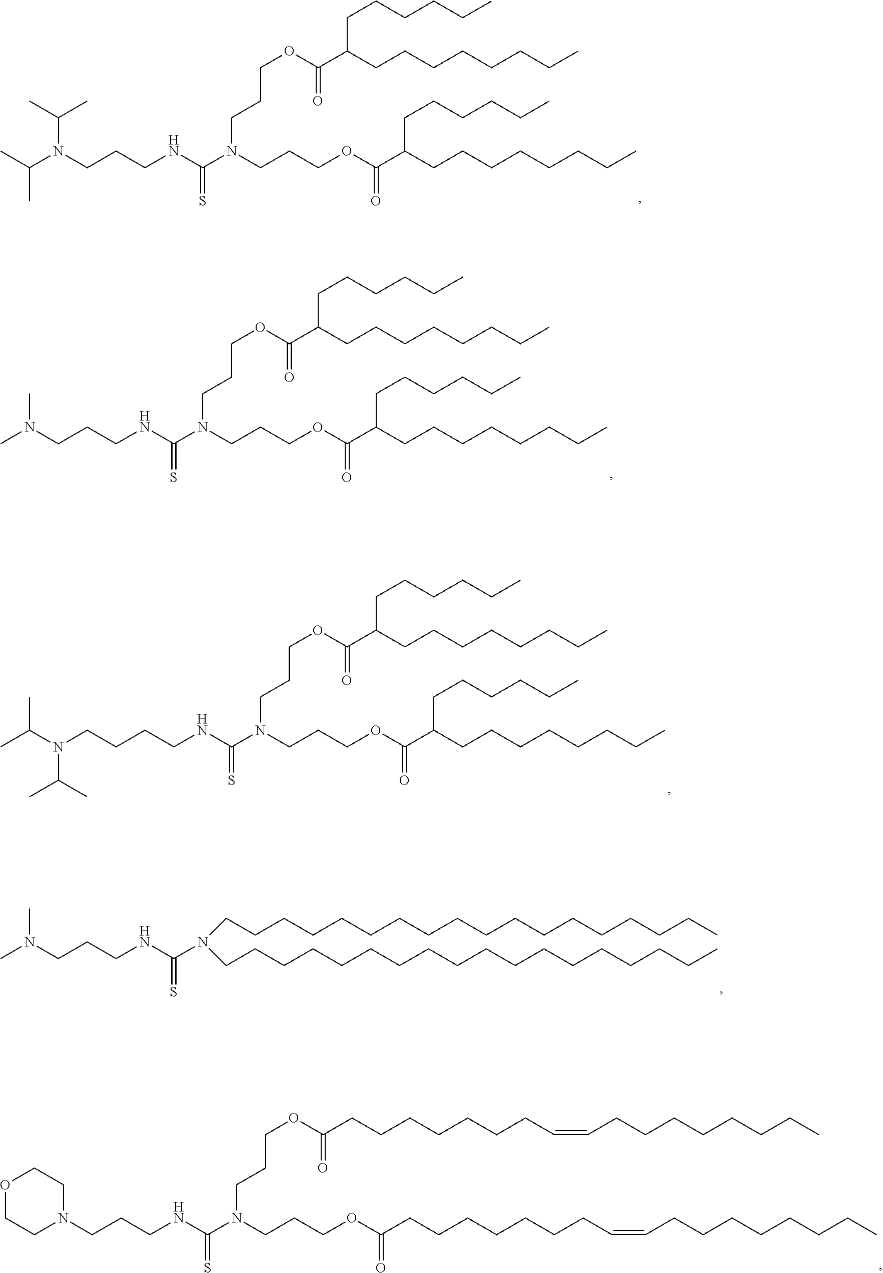

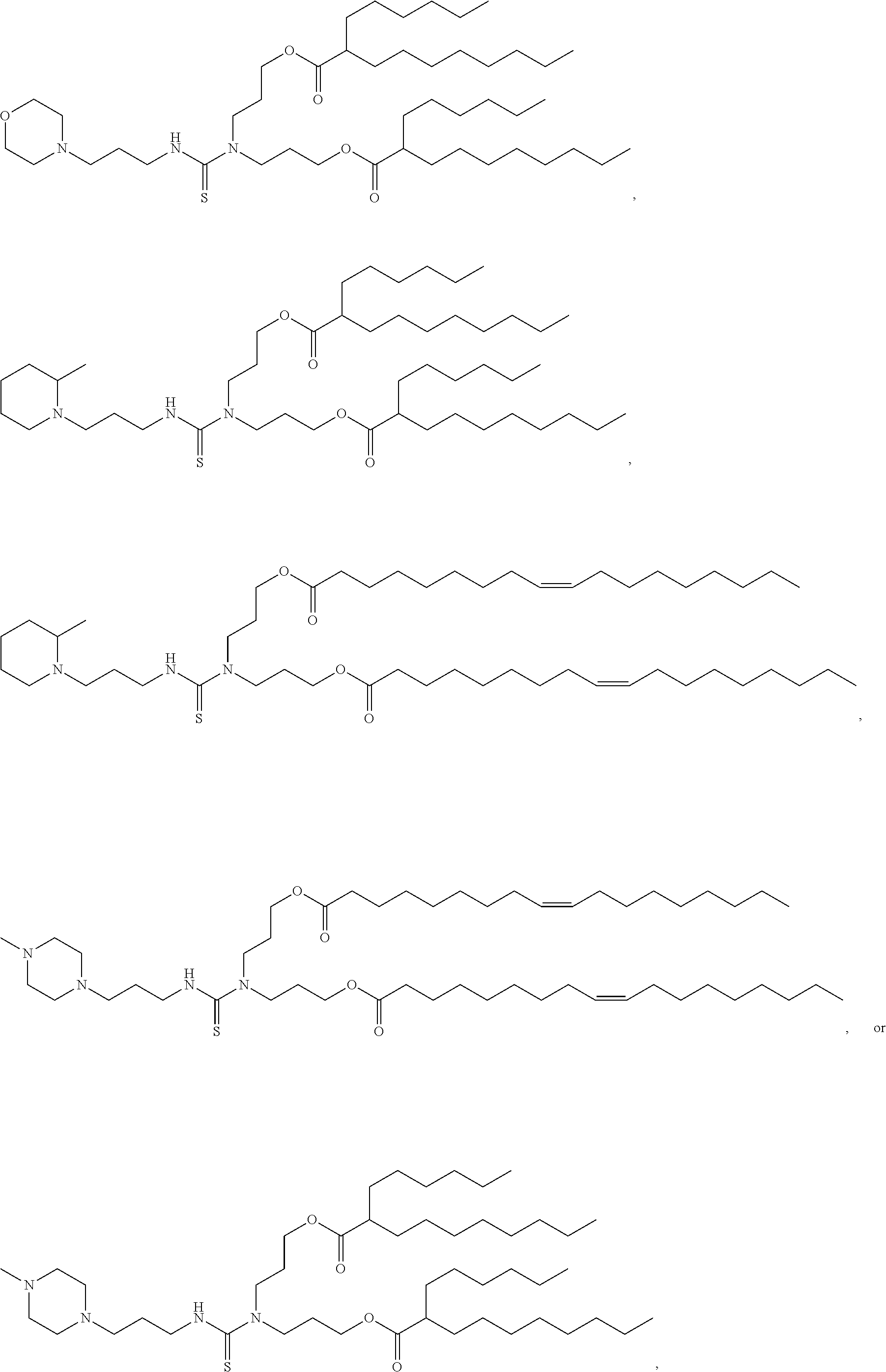

or a pharmaceutically acceptable salt thereof.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (EL-2) polypeptide as described herein and throughout, and a pharmaceutically acceptable carrier, wherein the pharmaceutically acceptable carrier comprises a lipid, wherein the lipid comprises a cationic lipid of formula (III):

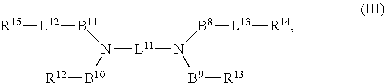

or a pharmaceutically acceptable salt, solvate, hydrate, stereoisomer, or prodrug thereof, wherein:
L¹¹ is

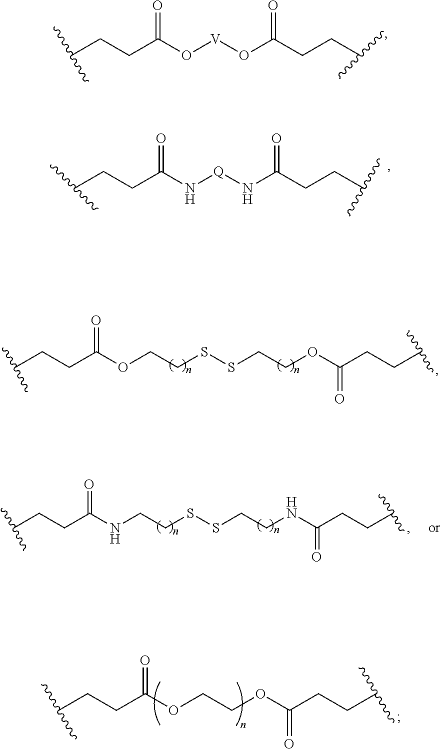

Q is substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene;
V is substituted or unsubstituted alkylene, substituted or unsubstituted cycloalkylene, substituted or unsubstituted arylene;
B⁸, B⁹, B¹⁰, and B¹¹ are each independently a bond, substituted or unsubstituted alkylene, or substituted or unsubstituted heteroalkylene;
L¹² is a bond, —O(C═O)—, —(C═O)O—, —O(C═O)O—, —C(═O)—, —O—, —O(CR²¹⁰R²¹¹)ₛO—, —S—, —C(═O)S—, —SC(═O)—, —NR²¹⁰C(═O)—, —C(═O)NR²¹⁰—, —NR²¹⁰C(═O)NR²¹¹—, —NR²¹⁰C(═S)—, —C(═S)NR²¹⁰—, —NR²¹⁰C(═S)NR²¹¹—, —OC(═O)NR²¹⁰—, —NR²¹⁰C(═O)O—, —SC(═O)NR²¹⁰— or —NR²¹⁰C(═O)S—;
L¹³ is a bond, —O(C═O)—, —(C═O)O—, —O(C═O)O—, —C(═O)—, —O—, —O(CR³¹⁰R³¹¹)ₛO—, —S—, —C(═O)S—, —SC(═O)—, —NR³¹⁰C(═O)—, —C(═O)NR³¹⁰—, —NR³¹⁰C(═O)NR³¹¹—, —NR³¹⁰C(═S)—, —C(═S)NR³¹⁰—, —NR³¹⁰C(═S)NR³¹¹—, —OC(═O)NR³¹⁰—, —NR³¹⁰C(═O)O—, —SC(═O)NR³¹⁰— or —NR³¹⁰C(═O)S—;
R¹² is H, —OR^{12A}, —SR^{12A}, —NR^{12A}, —CN, —(C═O)R^{12A}, —O(C═O)R^{12A}, —(C═O)OR^{12A}, —NR^{12A}(C═O)—R^{12B}, —(C═O)NR^{12A}R^{12B};
R¹³ is H, —OR^{13A}, —SR^{13A}, —NR^{13A}, —CN, —(C═O)R^{13A}, —O(C═O)R^{13A}, —(C═O)OR^{13A}, —NR^{13A}(C═O)—R^{13B}, —(C═O)NR^{13A}R^{13B};
R¹⁴ and R¹⁵ are each independently substituted or unsubstituted C₂-C₃₀ alkyl, or substituted or unsubstituted 2 to 30 membered heteroalkyl;
R^{12A}, R^{12B}, R^{13A}, and R^{13B} are each independently H, substituted or unsubstituted C₁-C₂₀ alkyl, or substituted or unsubstituted 2 to 20 membered heteroalkyl;
each R²¹⁰, R²¹¹, R³¹⁰, and R³¹¹ is independently H, substituted or unsubstituted C₁-C₁₂ alkyl, or substituted or unsubstituted 2 to 12 membered heteroalkyl;
each n is independently an integer from 0 to 8; and
each s is independently an integer from 1 to 4.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutically acceptable carrier comprises a lipid, wherein the lipid comprises a cationic lipid of formula (III):

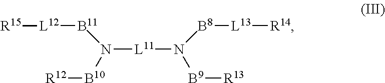

or a pharmaceutically acceptable salt, solvate, hydrate, stereoisomer, or prodrug thereof, wherein:
L¹¹ is

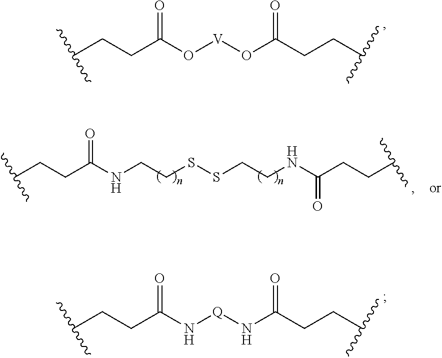

Q is substituted or unsubstituted alkylene;
V is substituted or unsubstituted alkylene;
B⁸, B⁹, B¹⁰, and B¹¹ are each independently substituted or unsubstituted alkylene;
L¹² is —O(C═O)— or —(C═O)O—;
L¹³ is —O(C═O)— or —(C═O)O—;
R¹² is H, —OR^{12A}, or —NR^{12A};
R¹³ is H, —OR^{13A}, or —NR^{13A};
R¹⁴ and R¹⁵ are each independently substituted or unsubstituted C₂-C₃₀ alkyl;
R^{12A} and R^{13A} are each independently H, substituted or unsubstituted C₁-C₂₀ alkyl; and
each n is independently an integer from 0 to 8.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutically acceptable carrier comprises a lipid, wherein the lipid comprises a cationic lipid of formula (III):

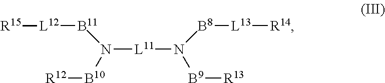

or a pharmaceutically acceptable salt, solvate, hydrate, stereoisomer, or prodrug thereof, wherein:
L¹¹ is or

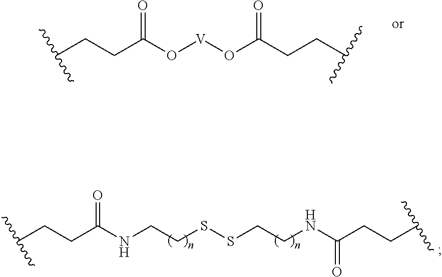

V is substituted or unsubstituted alkylene;
B⁸, B⁹, B¹⁰, and B¹¹ are each independently substituted or unsubstituted C₁-C₂₀ alkylene;
L¹² is —O(C═O)— or —(C═O)O—;
L¹³ is —O(C═O)— or —(C═O)O—;
R¹² is H or —OR^{2A};
R¹³ is H or —OR^{13A};
R¹⁴ and R¹⁵ are each independently substituted or unsubstituted C₂-C₂₀ alkyl;
R^{12A} and R^{13A} are each independently H, substituted or unsubstituted C₁-C₈ alkyl; and
each n is independently an integer from 0 to 4.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutically acceptable carrier comprises a lipid, wherein the lipid comprises a cationic lipid of formula (III):

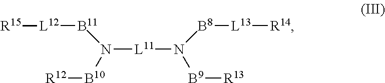

or a pharmaceutically acceptable salt, solvate, hydrate, stereoisomer, or prodrug thereof, wherein:
L¹¹ is

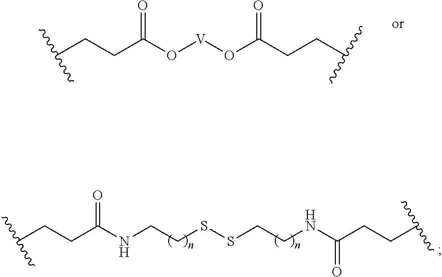

V is unsubstituted alkylene;
B⁸, B⁹, B¹⁰, and B¹¹ are each independently substituted or unsubstituted C₁-C₈ alkylene;
L¹² is —O(C═O)— or —(C═O)O—;
L¹³ is —O(C═O)— or —(C═O)O—;
R¹² is —OH, methoxy, or ethoxy;
R¹³ is —OH, methoxy, or ethoxy;
R¹⁴ and R¹⁵ are each independently substituted or unsubstituted C₂-C₂₀ alkyl; and
each n is independently an integer from 0 to 4.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (EL-2) polypeptide as described herein and throughout, and a pharmaceutically acceptable carrier, wherein the pharmaceutically acceptable carrier comprises a lipid, wherein the lipid comprises a cationic lipid of formula (III):

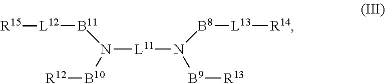

or a pharmaceutically acceptable salt, solvate, hydrate, stereoisomer, or prodrug thereof, wherein the cationic lipid is:

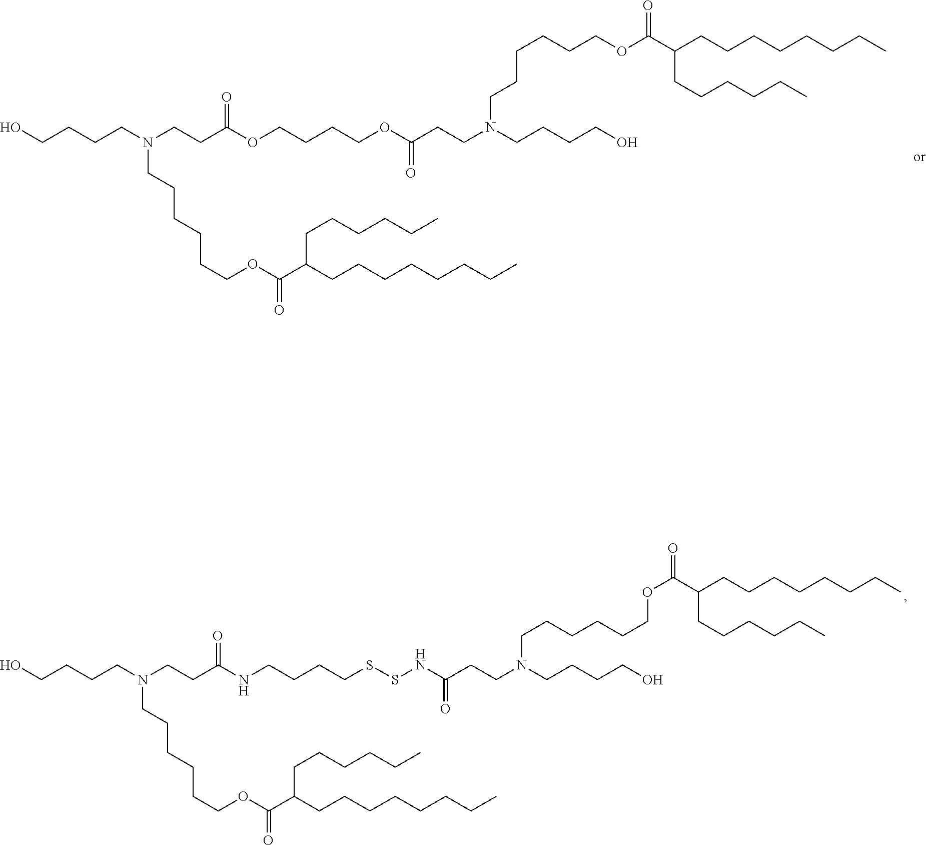 [SMILES: CCCCCCCCC(CCCCCC)C(=O)OCCCCCCN(CCCCO)CCC(=O)OCCCCOC(=O)CCN(CCCCO)CCCCCCOC(=O)C(CCCCCC)CCCCCCCC ; CCCCCCCCC(CCCCCC)C(=O)OCCCCCCN(CCCCO)CCC(=O)NCCCCSSNC(=O)CCN(CCCCO)CCCCCCOC(=O)C(CCCCCC)CCCCCCCC]

or a pharmaceutically acceptable salt thereof.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutically acceptable carrier comprises a lipid, wherein the lipid comprises a cationic lipid of formula (IV):

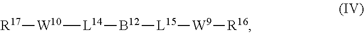

or a pharmaceutically acceptable salt, solvate, hydrate, stereoisomer, or prodrug thereof, wherein:
   B¹² is —W⁷-L^{a3}-W⁸—;
W⁷ and W⁸ are each independently a bond, substituted or unsubstituted C₁-C₁₂ alkylene, or substituted or unsubstituted 2 to 12 membered heteroalkylene;
L^{a3} is a bond, —S—S—, —O—(CH₂O)ₘ—,

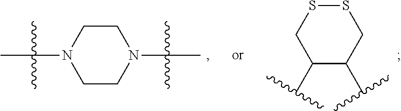 [SMILES: *N1CCN(*)CC1 ; *C1CSSCC1*]

W⁹ and W¹⁰ are each independently a bond, substituted or unsubstituted C₁-C₁₂ alkylene, substituted or unsubstituted 2 to 12 membered heteroalkylene, substituted or unsubstituted cycloalkylene, substituted or unsubstituted heterocycloalkylene, or any combination thereof,
L¹⁴ is a bond, —O(C═O)—, —(C═O)O—, —O(C═O)O—, —C(═O)—, —O—, —O(CR⁴¹⁰R⁴¹¹)O—, —S—, —C(═O)S—, —SC(═O)—, —NR⁴¹⁰C(═O)—, —C(═O)NR⁴¹⁰—, —NR⁴¹⁰C(═O)NR⁴¹¹—, —NR⁴¹⁰C(═S)—, —C(═S)NR⁴¹⁰—, —NR⁴¹⁰C(═S)NR⁴¹¹—, —OC(═O)NR⁴¹⁰—, —NR⁴¹⁰C(═O)O—, —SC(═O)NR⁴¹⁰— or —NR⁴¹⁰C(═O)S—;
L¹⁵ is a bond, —O(C═O)—, —(C═O)O—, —O(C═O)O—, —C(═O)—, —O—, —O(CR⁵¹⁰R⁵¹¹)ₛO—, —S—, —C(═O)S—, —SC(═O)—, —NR⁵¹⁰C(═O)—, —C(═O)NR⁵¹⁰—, —NR⁵¹⁰C(═O)NR⁵¹¹—, —NR⁵¹⁰C(═S)—, —C(═S)NR⁵¹⁰—, —NR⁵¹⁰C(═S)NR⁵¹¹—, —OC(═O)NR⁵¹⁰—, —NR⁵¹⁰C(═O)O—, —SC(═O)NR⁵¹⁰— or —NR⁵¹⁰C(═O)S—;
R¹⁶ and R¹⁷ are each independently

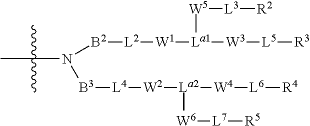

a fragment of cationic lipid of formula (I),

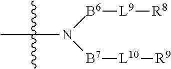

a fragment of cationic lipid of formula (II),

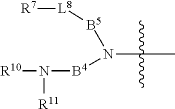

a fragment of cationic lipid of formula (II),

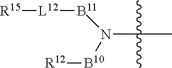

a fragment of cationic lipid of formula (III), or

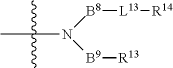

a fragment of cationic lipid of formula (III);
each R⁴¹⁰, R⁴¹¹, R⁵¹⁰, and R⁵¹¹ is independently H, substituted or unsubstituted C₁-C₁₂ alkyl, or substituted or unsubstituted 2 to 12 membered heteroalkyl;
each m is independently an integer from 0 to 8; and
each s is independently an integer from 1 to 4.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutically acceptable carrier comprises a lipid, wherein the lipid comprises a cationic lipid of formula (IV):

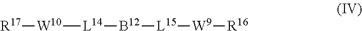

wherein:
L^{a3} is a bond, —S—S—,

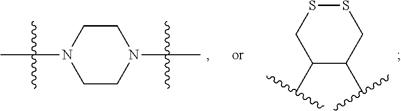 [SMILES: *N1CCN(*)CC1 ; *C1CSSCC1*]

W⁷ and W⁸ are each independently a bond or substituted or unsubstituted C₁-C₁₂ alkylene;
L¹⁴ is —O(C═O)—, —(C═O)O—, —C(═O)—, —NR⁴¹⁰C(═O)—, —C(═O)NR⁴¹⁰—, —NR⁴¹⁰C(═S)—, —C(═S)NR⁴¹⁰—, —OC(═O)NR⁴¹⁰—, or —NR⁴¹⁰C(═O)O—;
L¹⁵ is —O(C═O)—, —(C═O)O—, —C(═O)—, —NR⁵¹⁰C(═O)—, —C(═O)NR⁵¹⁰—, —NR⁵¹⁰C(═S)—, —C(═S)NR⁵¹⁰—, —OC(═O)NR⁵¹⁰—, or —NR⁵¹⁰C(═O)O—;
W⁹ and W¹⁰ are each independently a bond or substituted or unsubstituted C₁-C₁₂ alkylene;
R¹⁶ and R¹⁷ are each independently

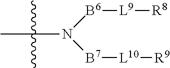

a fragment of cationic lipid of formula (II); and
each R⁴¹⁰ and R⁵¹⁰ is independently H or substituted or unsubstituted C₁-C₁₂ alkyl.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutically acceptable carrier comprises a lipid, wherein the lipid comprises a cationic lipid of formula (IV):

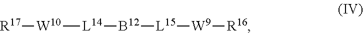

wherein the cationic lipid is a lipid wherein:
L^{a3} is a bond, —S—S—,

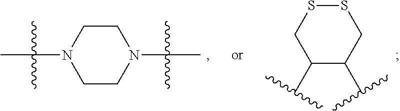 [SMILES: *N1CCN(*)CC1 ; *C1CSSCC1*]

W⁷ and W⁸ are each independently a bond or unsubstituted C₁-C₁₂ alkylene;
L¹⁴ is —O(C═O)—, —(C═O)O—, —NR⁴¹⁰C(═S)—, —C(═S)NR⁴¹⁰—, —OC(═O)NR⁴¹⁰—, or —NR⁴¹⁰C(═O)O—;
L¹⁵ is —O(C═O)—, —(C═O)O—, —NR⁵¹⁰C(═S)—, —C(═S)NR⁵¹⁰—, —OC(═O)NR⁵¹⁰—, or —NR⁵¹⁰C(═O)O—;
W⁹ and W¹⁰ are each independently a bond or substituted or unsubstituted C₁-C₁₂ alkylene;
R¹⁶ and R¹⁷ are each independently

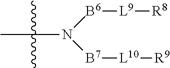

a fragment of cationic lipid of formula (II); and
each R⁴¹⁰ and R⁵¹⁰ is independently H or substituted or unsubstituted C₁-C₁₂ alkyl.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutically acceptable carrier comprises a lipid, wherein the lipid comprises a cationic lipid of formula (IV):

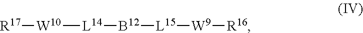

wherein the cationic lipid is a lipid wherein:
L^{a3} is a bond, —S—S—,

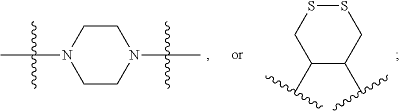 [SMILES: *N1CCN(*)CC1 ; *C1CSSCC1*]

W⁷ and W⁸ are each independently a bond or unsubstituted C₁-C₈ alkylene;
L¹⁴ is —O(C═O)—, —(C═O)O—, —NR⁴¹⁰C(═S)—, —C(═S)NR⁴¹⁰—, —OC(═O)NR⁴¹⁰—, or —NR⁴¹⁰C(═O)O—;
L¹⁵ is —O(C═O)—, —(C═O)O—, —NR⁵¹⁰C(═S)—, —C(═S)NR⁵¹⁰—, —OC(═O)NR⁵¹⁰—, or —NR⁵¹⁰C(═O)O—;
W⁹ and W¹⁰ are each independently a bond or unsubstituted C₁-C₈ alkylene;
R¹⁶ and R¹⁷ are each independently

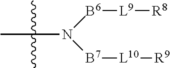

a fragment of cationic lipid of formula (II); and
each R⁴¹⁰ and R¹⁰⁰ is independently H or unsubstituted C₁-C₈ alkyl.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutically acceptable carrier comprises a lipid, wherein the lipid comprises a cationic lipid of formula (IV):

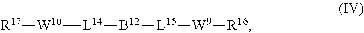

wherein:
L^{a3} is a bond, —S—S—,

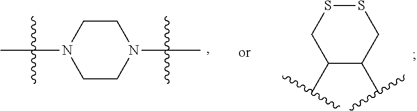 [SMILES: *N1CCN(*)CC1 ; *C1CSSCC1*]

W⁷ and W⁸ are each independently a bond or unsubstituted C₁-C₈ alkylene;
L¹⁴ is —O(C═O)—, —(C═O)O—, —NR⁴¹⁰C(═S)—, —C(═S)NR⁴¹⁰—, —OC(═O)NR⁴¹⁰—, or —NR⁴¹⁰C(═O)O—;
L¹⁵ is —O(C═O)—, —(C═O)O—, —NR⁵¹⁰C(═S)—, —C(═S)NR⁵¹⁰—, —OC(═O)NR⁵¹⁰—, or —NR⁵¹⁰C(═O)O—;
W⁹ and W¹⁰ are each independently a bond or unsubstituted C₁-C₈ alkylene;
R¹⁶ and R¹⁷ are each independently

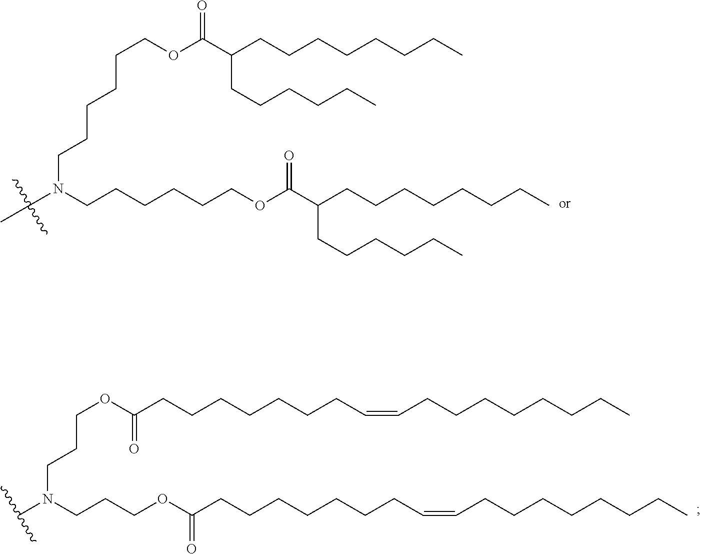 [SMILES: *N(CCCCCCOC(=O)C(CCCCCC)CCCCCCCC)CCCCCCOC(=O)C(CCCCCC)CCCCCCCC ; *N(CCCOC(=O)CCCCCCC/C=C\CCCCCCCC)CCCOC(=O)CCCCCCC/C=C\CCCCCCCC]

and
each R⁴¹⁰ and R¹⁰⁰ is independently H or methyl.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutically acceptable carrier comprises a lipid, wherein the lipid comprises a cationic lipid of formula (IV):

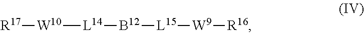

wherein the cationic lipids:

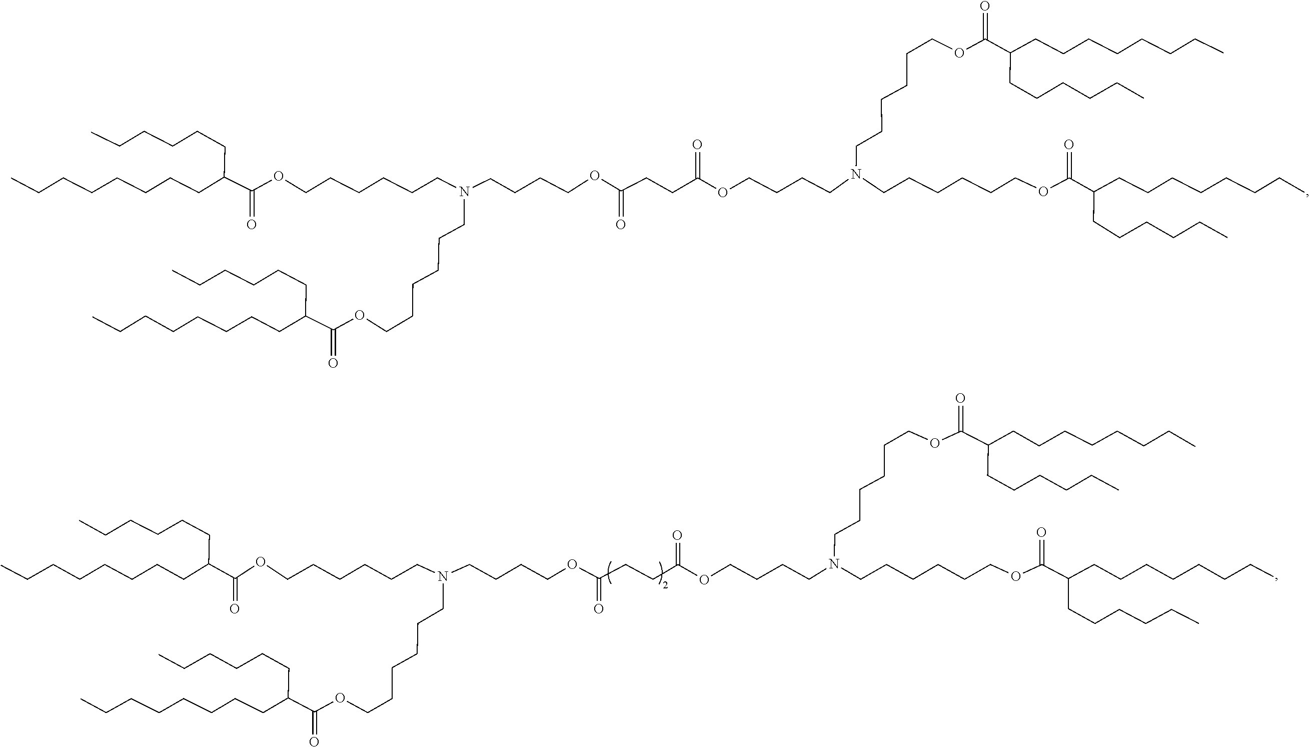

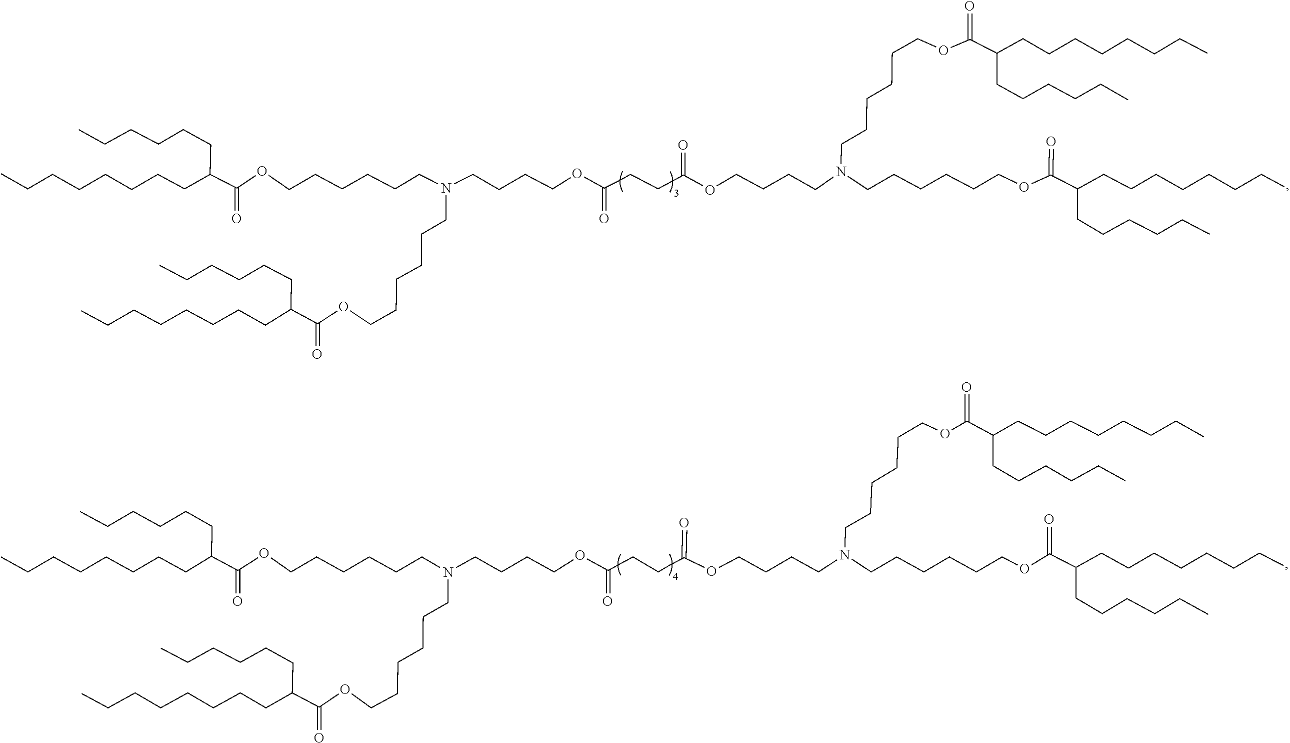

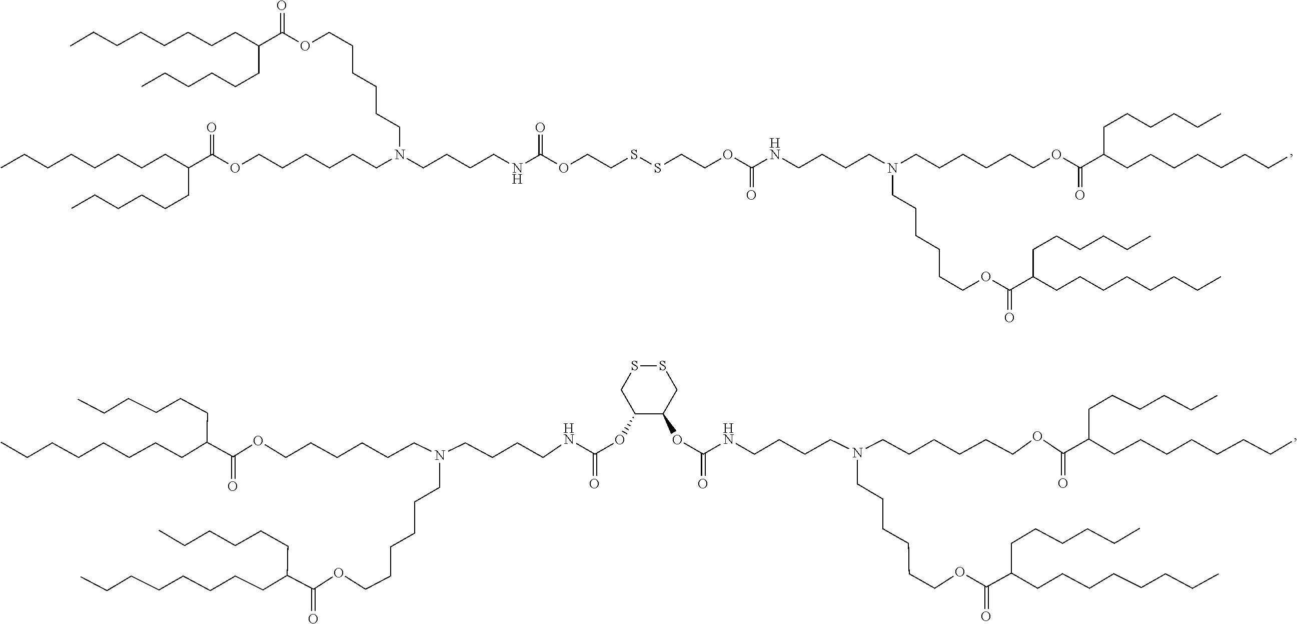

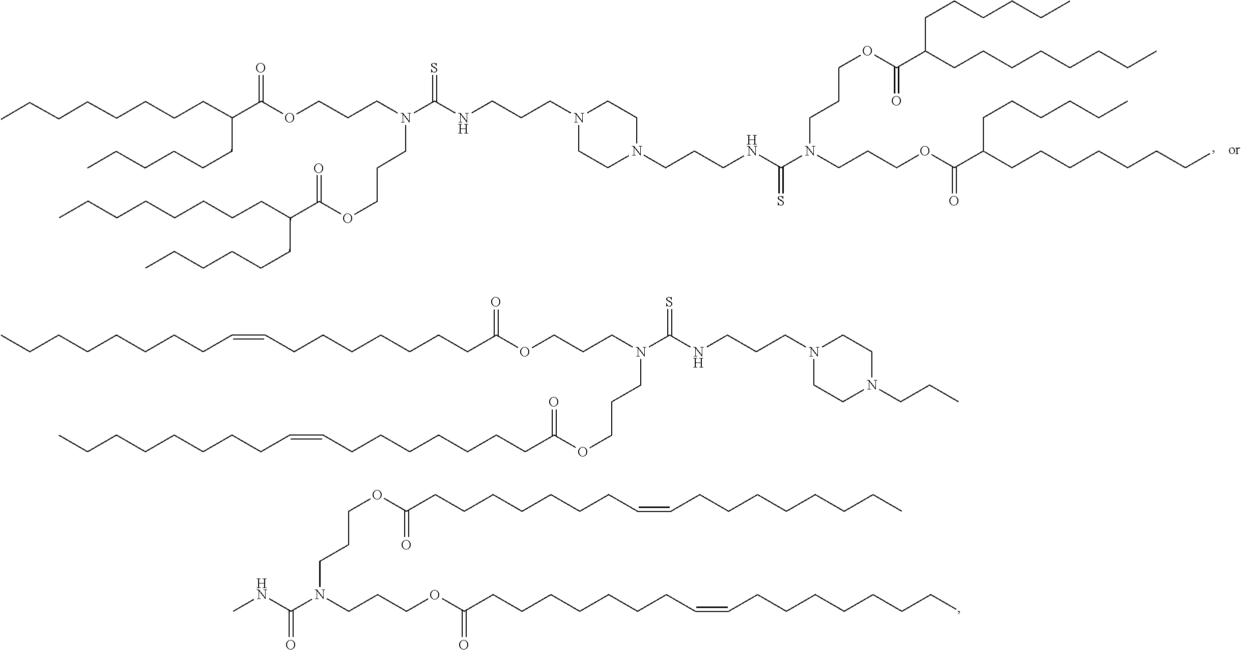

or a pharmaceutically acceptable salt thereof.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutical composition comprises the following compound:

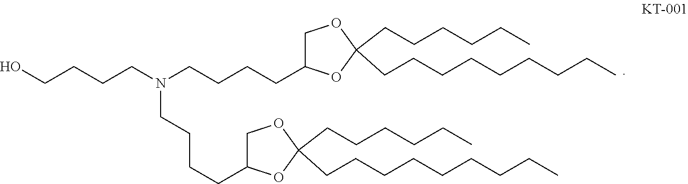 [SMILES: CCCCCCCCCC1(CCCCCC)OCC(CCCCN(CCCCO)CCCCC2COC(CCCCCC)(CCCCCCCCC)O2)O1]

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutical composition further comprises lipid nanoparticles.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout, and a pharmaceutically acceptable carrier, wherein the pharmaceutical composition further comprises at least a second therapeutic agent.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout and a pharmaceutically acceptable carrier as described herein and throughout, wherein the pharmaceutical composition further comprises at least a second therapeutic agent, wherein the at least a second therapeutic agent comprises an anti-neoplasm agent or substance, immune-oncology (I-O) agent, an immune checkpoint inhibitor, and/or an antibody-dependent cell-mediated cytotoxicity agent, and/or a cell therapy agent.

In certain embodiments, which may be combined with other embodiments provided herein and throughout, are provided a pharmaceutical composition comprising an effective amount of a polynucleotide comprising a nucleic acid sequence encoding a fusion protein comprising a modified interleukin 2 (IL-2) polypeptide as described herein and throughout a pharmaceutically acceptable carrier as described herein and throughout, wherein the pharmaceutical composition further comprises at least a second therapeutic agent, wherein the at least a second therapeutic agent comprises an anti-PD1 antibody, and anti-PDL1 antibody, an anti-EGFR antibody, and anti-Her2 antibody, and anti-CD20 antibody, an anti-CD38 antibody, an anti-CD47 antibody, an anti-CD123 antibody, an anti-cMET antibody, an anti-CCR4 antibody, an anti-CTLA4 antibody, a chimeric antigen receptor T cell (CAR-t), or a dimeric antigen receptor T cell (DAR-T).

In certain embodiments, which may be combined with other embodiments provided herein and throughout, are provided polynucleotides comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier as described herein and throughout, wherein the polynucleotide or the pharmaceutical composition is for use in a method of treating or preventing a proliferation disorder in a subject.

In certain embodiments, which may be combined with other embodiments provided herein and throughout, are provided methods for treating or preventing a proliferation disease or disorder in a subject having, suspected of having, or at risk of having the proliferation disease or disorder, the method comprising administering to the subject an effective amount of a polynucleotide comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier as described herein and throughout.

In certain embodiments, which may be combined with other embodiments provided herein and throughout, are provided methods for treating or preventing a proliferation disease or disorder in a subject having, suspected of having, or at risk of having the proliferation disease or disorder, the method comprising administering to the subject an effective amount of a polynucleotide comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier as described herein and throughout, wherein the proliferation disease or disorder comprises a tumor.

In certain embodiments, which may be combined with other embodiments provided herein and throughout, are provided methods for treating or preventing a proliferation disease or disorder in a subject having, suspected of having, or at risk of having the proliferation disease or disorder, the method comprising administering to the subject an effective amount of a polynucleotide comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier as described herein and throughout, wherein the proliferation disease or disorder comprises a cancer.

In certain embodiments, which may be combined with other embodiments provided herein and throughout, are provided methods for treating or preventing a proliferation disease or disorder in a subject having, suspected of having, or at risk of having the proliferation disease or disorder, the method comprising administering to the subject an effective amount of a polynucleotide comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier as described herein and throughout, wherein the proliferation disease or disorder comprises a solid tumor.

In certain embodiments, which may be combined with other embodiments provided herein and throughout, are provided methods for treating or preventing a proliferation disease or disorder in a subject having, suspected of having, or at risk of having the proliferation disease or disorder, the method comprising administering to the subject an effective amount of a polynucleotide comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier as described herein and throughout, wherein the proliferation disease or disorder is selected from the group consisting of: Chondrosarcoma, Ewing's sarcoma, Malignant fibrous histiocytoma of bone/osteosarcoma, Osteosarcoma, Rhabdomyosarcoma, Heart cancer, Astrocytoma, Brainstem glioma, Pilocytic astrocytoma, Ependymoma, Primitive neuroectodermal tumor, Cerebellar astrocytoma, Cerebral astrocytoma, Glioma, Medulloblastoma, Neuroblastoma, Oligodendroglioma, Pineal astrocytoma, Pituitary adenoma, Visual pathway and hypothalamic glioma, Breast cancer, Invasive lobular carcinoma, Tubular carcinoma, Invasive cribriform carcinoma, Medullary carcinoma, Male breast cancer, Phyllodes tumor, Inflammatory Breast Cancer, Adrenocortical carcinoma, Islet cell carcinoma (endocrine pancreas), Multiple endocrine neoplasia syndrome, Parathyroid cancer, Pheochromocytoma, Thyroid cancer, Merkel cell carcinoma, Uveal melanoma, Retinoblastoma, Anal cancer, Appendix cancer, cholangiocarcinoma, Carcinoid tumor, gastrointestinal, Colon cancer, Extrahepatic bile duct cancer, Gallbladder cancer, Gastric (stomach) cancer, Gastrointestinal carcinoid tumor, Gastrointestinal stromal tumor (GIST), Hepatocellular cancer, Pancreatic cancer, Islet cell cancer, Rectal cancer, Colorectal cancer, Bladder cancer, Cervical cancer, Endometrial cancer, Extragonadal germ cell tumor, Ovarian cancer, Ovarian epithelial cancer (surface epithelial-stromal tumor), Ovarian germ cell tumor, Fallopian Tube cancer, Peritoneal cancer, Penile cancer, Renal cell carcinoma (RCC), Renal pelvis and ureter cancer, transitional cell cancer, Prostate cancer, Testicular cancer, Gestational trophoblastic tumor, Ureter and renal pelvis, transitional cell cancer, Urethral cancer, Uterine sarcoma, Vaginal cancer, Vulvar cancer, Wilms tumor, Esophageal cancer, Head and neck cancer, Nasopharyngeal carcinoma, Oral cancer, Oropharyngeal cancer, Paranasal sinus and nasal cavity cancer, Pharyngeal cancer, Salivary gland cancer, Hypopharyngeal cancer, Basal-cell carcinoma, Melanoma, Skin cancer (non-melanoma), Bronchial adenomas/carcinoids, Small cell lung cancer, Mesothelioma, Non-small cell lung cancer (NSCLC), Pleuropulmonary blastoma, Laryngeal cancer, Thymoma and thymic carcinoma, AIDS-related cancers, Kaposi sarcoma, Epithelioid hemangioendothelioma (EHE), Desmoplastic small round cell tumor, and Liposarcoma.

In certain embodiments, which may be combined with other embodiments provided herein and throughout, are provided methods for treating or preventing a proliferation disease or disorder in a subject having, suspected of having, or at risk of having the proliferation disease or disorder, the method comprising administering to the subject an effective amount of a polynucleotide comprising a nucleic acid sequence encoding a fusion protein comprising a modified interleukin 2 (IL-2) polypeptide as described herein and throughout, or an effective amount of a pharmaceutical composition comprising such a polynucleotide a pharmaceutically acceptable carrier, wherein the proliferation disease or disorder comprises a tumor, wherein the tumor or the cancer comprises a hematological malignancy.

In certain embodiments, which may be combined with other embodiments provided herein and throughout, are provided methods for treating or preventing a proliferation disease or disorder in a subject having, suspected of having, or at risk of having the proliferation disease or disorder, the method comprising administering to the subject an effective amount of a polynucleotide comprising a nucleic acid sequence encoding a fusion protein comprising a modified interleukin 2 (IL-2) polypeptide as described herein and throughout, or an effective amount of a pharmaceutical composition comprising such a polynucleotide a pharmaceutically acceptable carrier, wherein the proliferation disease or disorder comprises a hematological malignancy selected from the group consisting of: myeloid neoplasms, Leukemias, Lymphomas, Hodgkin lymphoma, classic Hodgkin Lymphoma, Non-Hodgkin lymphoma, Anaplastic large cell lymphoma, Angioimmunoblastic T-cell lymphoma, Hepatosplenic T-cell lymphoma, B-cell lymphoma reticuloendotheliosis, Reticulosis, Microglioma, Diffuse large B-cell lymphoma, Follicular lymphoma, Mucosa-associated lymphatic tissue lymphoma, B-cell chronic lymphocytic leukemia, Mantle cell lymphoma, Burkitt lymphoma, Mediastinal large B cell lymphoma, Waldenstrom's macroglobulinemia, Nodal marginal zone B cell lymphoma, Splenic marginal zone lymphoma, Intravascular large B-cell lymphoma, Primary effusion lymphoma, Lymphomatoid granulomatosis, Nodular lymphocyte predominant Hodgkin's lymphoma, plasma cell leukemia, Acute erythraemia and erythroleukaemia, Acute erythremic myelosis, Acute erythroid leukemia, Heilmeyer-Schoner disease, Acute megakaryoblastic leukemia, Mast cell leukemia, Panmyelosis, Acute panmyelosis with myelofibrosis, Lymphosarcoma cell leukemia, Acute leukaemia of unspecified cell type, Blastic phase chronic myelogenous leukemia, Stem cell leukemia, Chronic leukaemia of unspecified cell type, Subacute leukaemia of unspecified cell type, Accelerated phase chronic myelogenous leukemia, Acute myeloid leukemia, Polycythemia vera, Acute promyelocytic leukemia, Acute basophilic leukemia, Acute eosinophilic leukemia, Acute lymphoblastic leukemia, Acute monocytic leukemia, Acute myeloblastic leukemia with maturation, Acute myeloid dendritic cell leukemia, Adult T-cell leukemia/lymphoma, Aggressive NK-cell leukemia, B-cell prolymphocytic leukemia, B-cell chronic lymphocytic leukemia, B-cell leukemia, Chronic myelogenous leukemia, Chronic myelomonocytic leukemia, Chronic neutrophilic leukemia, Chronic lymphocytic leukemia, Hairy cell leukemia, Chronic idiopathic myelofibrosis, Multiple myeloma, Kahler's disease, Myelomatosis, Solitary myeloma, Plasma cell leukemia, Plasmacytoma, extramedullary, Malignant plasma cell tumour NOS, Plasmacytoma NOS, Monoclonal gammopathy, Multiple Myeloma, Angiocentric immunoproliferative lesion, Lymphoid granulomatosis, Angioimmunoblastic lymphadenopathy, T-gamma lymphoproliferative disease, Waldenstrom's macroglobulinaemia, Alpha heavy chain disease, Gamma heavy chain disease, Franklin's disease, Immunoproliferative small intestinal disease, Mediterranean disease, Malignant immunoproliferative disease, unspecified, and Immunoproliferative disease NOS.

In certain embodiments, which may be combined with other embodiments provided herein and throughout, are provided methods for treating or preventing an immune deficiency disease or disorder in a subject having, suspected of having, or at risk of having the immune deficiency disease or disorder, the method comprising administering to the subject an effective amount of a polynucleotide comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier as described herein and throughout.

In certain embodiments, which may be combined with other embodiments provided herein and throughout, are provided methods for treating or preventing an immune deficiency disease or disorder in a subject having, suspected of having, or at risk of having the immune deficiency disease or disorder, the method comprising administering to the subject an effective amount of a polynucleotide comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier, wherein the immune deficiency disease or disorder is selected from the group consisting of: Agammaglobulinemia: X-Linked and Autosomal Recessive, Ataxia Telangiectasia, Chronic Granulomatous Disease and Other Phagocytic Cell Disorders, Common Variable Immune Deficiency, Complement Deficiencies, DiGeorge Syndrome, Hemophagocytic Lymphohistiocytosis (HLH), Hyper IgE Syndrome, Hyper IgM Syndromes, IgG Subclass Deficiency, Innate Immune Defects, NEMO Deficiency Syndrome, Selective IgA Deficiency, Selective IgM Deficiency, Severe Combined Immune, Deficiency and Combined Immune Deficiency, Specific Antibody Deficiency, Transient Hypogammaglobulinemia of Infancy, WHIM Syndrome (Warts, Hypogammaglobulinemia, Infections, and Myelokathexis), Wiskott-Aldrich Syndrome, Other Antibody Deficiency Disorders, Other Primary Cellular Immunodeficiencies, Severe combined immune deficiency (SCID), Common variable immune deficiency (CVID), Human immunodeficiency virus/acquired immune deficiency syndrome (HIV/AIDS), Drug-induced immune deficiency, Graft versus host syndrome, Primary Immune Deficiency Diseases (PIDDs), and Lymphopenia.

In certain embodiments, which may be combined with other embodiments provided herein and throughout, are provided methods for treating or preventing an autoimmune or inflammatory disease or disorder in a subject having, suspected of having, or at risk of having the autoimmune or inflammatory disease or disorder, the method comprising administering to the subject an effective amount of a polynucleotide comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier as described herein and throughout.

In certain embodiments, which may be combined with other embodiments provided herein and throughout, are provided methods for treating or preventing an autoimmune or inflammatory disease or disorder in a subject having, suspected of having, or at risk of having the autoimmune or inflammatory disease or disorder, the method comprising administering to the subject an effective amount of a polynucleotide comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier as described herein and throughout, wherein the autoimmune or inflammatory disease or disorder is selected from the group consisting of: inflammation, autoimmune disease, paraneoplastic autoimmune diseases, cartilage inflammation, fibrotic disease and/or bone degradation, arthritis, rheumatoid arthritis, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reter's Syndrome, SEA Syndrome (Seronegativity, Enthesopathy, Arthropathy Syndrome), juvenile dermatomyositis, juvenile psoriatic arthritis, juvenile scleroderma, juvenile systemic lupus erythematosus, juvenile vasculitis, pauciarticular rheumatoid arthritis, polyarticular rheumatoid arthritis, systemic onset rheumatoid arthritis, ankylosing spondylitis, enteropathic arthritis, reactive arthritis, Reter's Syndrome, SEA Syndrome (Seronegativity, Enthesopathy, Arthropathy Syndrome), dermatomyositis, psoriatic arthritis, scleroderma, systemic lupus erythematosus, vasculitis, myolitis, polymyolitis, dermatomyolitis, osteoarthritis, polyarteritis nodossa, Wegener's granulomatosis, arteritis, ploymyalgia rheumatica, sarcoidosis, scleroderma, sclerosis, primary biliary sclerosis, sclerosing cholangitis, Sjogren's syndrome, psoriasis, plaque psoriasis, guttate psoriasis, inverse psoriasis, pustular psoriasis, erythrodermic psoriasis, dermatitis, atopic dermatitis, atherosclerosis, lupus, Still's disease, Systemic Lupus Erythematosus (SLE), myasthenia gravis, inflammatory bowel disease (IBD), Crohn's disease, ulcerative colitis, celiac disease, multiple schlerosis (MS), asthma, COPD, Guillain-Barre disease, Type I diabetes mellitus, thyroiditis (e.g., Graves' disease), Addison's disease, Raynaud's phenomenon, autoimmune hepatitis, GVHD, and transplantation rejection.

In certain embodiments, which may be combined with other embodiments provided herein and throughout, are provided methods for treating or preventing an infectious disease or disorder in a subject in a subject having, suspected of having, or at risk of having an infectious disease or disorder, the method comprising administering to the subject an effective amount of a polynucleotide comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier as described herein and throughout.

In certain embodiments, which may be combined with other embodiments provided herein and throughout, are provided methods for treating or preventing an infectious disease or disorder in a subject in a subject having, suspected of having, or at risk of having an infectious disease or disorder, the method comprising administering to the subject an effective amount of a polynucleotide comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier as described herein and throughout, wherein the infectious disease or disorder is selected from the group consisting of: Acinetobacter infections, Actinomycosis, African sleeping sickness (African trypanosomiasis), AIDS (acquired immunodeficiency syndrome), Amoebiasis, Anaplasmosis, Angiostrongyliasis Anisakiasis, Anthrax, Arcanobacterium haemolyticum infection, Argentine hemorrhagic fever, Ascariasis, Aspergillosis Astrovirus infection, Babesiosis, Bacillus cereus infection, Bacterial meningitis, Bacterial pneumonia, Bacterial vaginosis, Bacteroides infection, Balantidiasis, Bartonellosis, Baylisascaris infection, BK virus infection, Black piedra, Blastocystosis, Blastomycosis, Bolivian hemorrhagic fever, Botulism (and Infant botulism), Brazilian hemorrhagic fever, Brucellosis, Bubonic plague, Burkholderia infection, Buruli ulcer, Calicivirus infection (Norovirus and Sapovirus), Campylobacteriosis, Candidiasis (Moniliasis; Thrush), Capillariasis, Carrion's disease, Cat-scratch disease, Cellulitis, Chagas disease (American trypanosomiasis), Chancroid, Chickenpox, Chikungunya, Chlamydia, Chlamydophila pneumoniae infection (Taiwan acute respiratory agent or TWAR), Cholera, Chromoblastomycosis, Chytridiomycosis, Clonorchiasis, Clostridium difficile colitis, Coccidioidomycosis, Colorado tick fever (CTF), Common cold (Acute viral rhinopharyngitis; Acute coryza), Coronavirus disease 2019 (COVID-19), Creutzfeldt-Jakob disease (CJD), Crimean-Congo hemorrhagic fever (CCHF), Cryptococcosis, Cryptosporidiosis, Cutaneous larva migrans (CLM), Cyclosporiasis, Cysticercosis, Cytomegalovirus infection, Dengue fever, Desmodesmus infection, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum (Sixth disease), Fasciolasis, Fasciolopsiasis, Fatal familial insomnia (FFI), Filariasis, Food poisoning by Clostridium perfringens, Free-living amebic infection, Fusobacterium infection, Gas gangrene (Clostridial myonecrosis), Geotrichosis, Gerstmann-Striussler-Scheinker syndrome (GSS), Giardiasis, Glanders, Gnathostomiasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome (HPS), Heartland virus disease, Helicobacter pylori infection, Hemolytic-uremic syndrome (HUS), Hemorrhagic fever with renal syndrome (HFRS), Hendra virus infection, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis (HGA), Human metapneumovirus infection, Human monocytic ehrlichiosis, Human papillomavirus (HPV) infection, Human parainfluenza virus infection, Hymenolepiasis, Epstein-Barr virus infectious mononucleosis (Mono), Influenza (flu), Isosporiasis, Kawasaki disease, Keratitis, Kingella kingae infection, Kuru, Lassa fever, Legionellosis (Legionnaires' disease), Pontiac fever, Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever (MHF), Measles, Middle East respiratory syndrome (MERS), Melioidosis (Whitmore's disease), Meningitis, Meningococcal disease, Metagonimiasis, Microsporidiosis, Molluscum contagiosum (MC), Monkeypox, Mumps, Murine typhus (Endemic typhus), Mycoplasma pneumonia, Mycoplasma genitalium infection, Mycetoma, Myiasis, Neonatal conjunctivitis (Ophthalmia neonatorum), Nipah virus infection, Norovirus (children and babies), (New) Variant Creutzfeldt-Jakob disease (vCJD, nvCJD), Nocardiosis, Onchocerciasis (River blindness), Opisthorchiasis, Paracoccidioidomycosis (South American blastomycosis), Paragonimiasis, Pasteurellosis, Pediculosis capitis (Head lice), Pediculosis corporis (Body lice), Pediculosis pubis (pubic lice, crab lice), Pelvic inflammatory disease (PID), Pertussis (whooping cough), Plague, Pneumococcal infection, Pneumocystis pneumonia (PCP), Pneumonia, Poliomyelitis, Prevotella infection, Primary amoebic meningoencephalitis (PAM), Progressive multifocal leukoencephalopathy, Psittacosis, Q fever, Rabies, Relapsing fever, Respiratory syncytial virus infection, Rhinosporidiosis, Rhinovirus infection, Rickettsial infection, Rickettsialpox, Rift Valley fever (RVF), Rocky Mountain spotted fever (RMSF), Rotavirus infection, Rubella, Salmonellosis, SARS (severe acute respiratory syndrome), Scabies, Scarlet fever, Schistosomiasis, Sepsis, Shigellosis (bacillary dysentery), Shingles (Herpes zoster), Smallpox (variola), Sporotrichosis, Staphylococcal food poisoning, Staphylococcal infection, Strongyloidiasis, Subacute sclerosing panencephalitis, Bejel, Syphilis, and Yaws, Taeniasis, Tetanus (lockjaw), Tinea barbae (barber's itch), Tinea capitis (ringworm of the scalp), Tinea corporis (ringworm of the body), Tinea cruris (Jock itch), Tinea manum (ringworm of the hand), Tinea nigra, Tinea pedis (athlete's foot), Tinea unguium (onychomycosis), Tinea versicolor (Pityriasis versicolor), Toxocariasis (ocular larva migrans (OLM)), Toxocariasis (visceral larva migrans (VLM)), Toxoplasmosis, Trachoma, Trichinosis, Trichomoniasis, Trichuriasis (whipworm infection), Tuberculosis, Tularemia, Typhoid fever, Typhus fever, Ureaplasma urealyticum infection, Valley fever, Venezuelan equine encephalitis, Venezuelan hemorrhagic fever, Vibrio vulnificus infection, Vibrio parahaemolyticus enteritis, Viral pneumonia, West Nile fever, White piedra (tinea blanca), Yersinia pseudotuberculosis infection, Yersiniosis, Yellow fever, Zeaspora, Zika fever, and Zygomycosis.

In certain embodiments, which may be combined with other embodiments provided herein and throughout, are provided uses of a polynucleotide comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier as described herein and throughout, for the manufacture of a medicament for treating or preventing a proliferation disease or disorder, an autoimmune or inflammatory disease or disorder, or an infectious disease or disorder, in a subject.

In certain embodiments, which may be combined with other embodiments provided herein and throughout, are provided methods and uses as described herein and throughout, wherein said methods and said uses independently comprise administering to a subject an effective amount of a polynucleotide comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier as described herein and throughout, wherein the subject is a human.

In certain embodiments, which may be combined with other embodiments provided herein and throughout, are provided methods and uses as described herein and throughout, wherein said methods and said uses independently comprise administering to a subject an effective amount of a polynucleotide comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier as described herein and throughout, wherein the subject is a non-human mammal.

In certain embodiments, which may be combined with other embodiments provided herein and throughout, are provided methods of expanding a CD4⁺ helper cell, CD8⁺ effector naive and memory cell, T regulatory (Treg) cell, Natural Killer (NK) cell, or Natural killer T (NKT) cell population, the methods comprising contacting a cell population with a polynucleotide comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier as described herein and throughout, for a time sufficient to induce formation of a complex with an IL-2R βγ, thereby stimulating the expansion of the T cell, NK cell, and/or NKT cell population.

In certain embodiments, which may be combined with other embodiments provided herein and throughout, are provided methods of expanding a CD4⁺ helper cell, CD8⁺ effector naive and memory cell, T regulatory (Treg) cell, Natural Killer (NK) cell, or Natural killer T (NKT) cell population, the methods comprising contacting a cell population with a polynucleotide comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier as described herein and throughout, for a time sufficient to induce formation of a complex with an IL-2R βγ, thereby stimulating the expansion of the T cell, Treg cell, NK cell, and/or NKT cell population with reduced cell death by 10% to 100%.

In certain embodiments, which may be combined with other embodiments provided herein and throughout, are provided methods of expanding a CD4⁺ helper cell, CD8⁺ effector naive and memory cell, T regulatory (Treg) cell, Natural Killer (NK) cell, or Natural killer T (NKT) cell population, the methods comprising contacting a cell population with a polynucleotide comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier as described herein and throughout, wherein the polynucleotide or the pharmaceutical composition expands CD4⁺ T Treg cells by less than 20%, 15%, 10%, 5%, 1% or less in the CD3⁺ cell population compared to an expansion of CD4⁺ Treg cells in the CD3⁺ cell population contacted with a comparable IL-2 polypeptide comprising an amino acid sequence set forth in SEQ ID NO:1 or SEQ ID NO:2 without the at least one substitution.

In certain embodiments, which may be combined with other embodiments provided herein and throughout, are provided methods of expanding a CD4⁺ helper cell, CD8⁺ effector naive and memory cell, T regulatory (Treg) cell, Natural Killer (NK) cell, or Natural killer T (NKT) cell population, the methods comprising contacting a cell population with a polynucleotide comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier as described herein and throughout, wherein the polynucleotide or the pharmaceutical composition does not substantially expand CD4⁺ Treg cells in the cell population.

In certain embodiments, which may be combined with other embodiments provided herein and throughout, are provided methods of expanding a CD4⁺ helper cell, CD8⁺ effector naive and memory cell, T regulatory (Treg) cell, Natural Killer (NK) cell, or Natural killer T (NKT) cell population, the methods comprising contacting a cell population with a polynucleotide comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier as described herein and throughout, wherein the ratio of effector T cells to Treg cells in the cell population after incubation with the polynucleotide of or the pharmaceutical composition is about or at least 2:1, 3:1, 4:1, 5:1, 6:1, 7:1, 8:1, 9:1, 10:1, 20:1, 50:1, 100:1 or more.

In certain embodiments, which may be combined with other embodiments provided herein and throughout, are provided methods of expanding a CD4⁺ helper cell, CD8⁺ effector naive and memory cell, T regulatory (Treg) cell, Natural Killer (NK) cell, or Natural killer T (NKT) cell population, the methods comprising contacting a cell population with a polynucleotide comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier as described herein and throughout, wherein the method is conducted in vivo.

In certain embodiments, which may be combined with other embodiments provided herein and throughout, are provided methods of expanding a CD4⁺ helper cell, CD8⁺ effector naive and memory cell, T regulatory (Treg) cell, Natural Killer (NK) cell, or Natural killer T (NKT) cell population, the methods comprising contacting a cell population with a polynucleotide comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier as described herein and throughout, wherein the method is conducted in vitro.

In certain embodiments, which may be combined with other embodiments provided herein and throughout, are provided methods of expanding a CD4⁺ helper cell, CD8⁺ effector naive and memory cell, T regulatory (Treg) cell, Natural Killer (NK) cell, or Natural killer T (NKT) cell population, the methods comprising contacting a cell population with a polynucleotide comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier as described herein and throughout, wherein the method is conducted ex vivo.

In certain embodiments, which may be combined with other embodiments provided herein and throughout, are provided uses of an effective amount of comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier as described herein and throughout, for the manufacture of a medicament for expanding a CD4⁺ helper cell, CD8⁺ effector naive and memory cell, Treg cell, Natural Killer (NK) cell, or Natural killer T (NKT) cell population in a cell population.

In certain embodiments, which may be combined with other embodiments provided herein and throughout, are provided uses of an effective amount of comprising a nucleic acid sequence encoding a fusion protein comprising a modified interleukin 2 (IL-2) polypeptide as described herein and throughout, or an effective amount of a pharmaceutical composition comprising such a polynucleotide and a pharmaceutically acceptable carrier as described herein and throughout, for expanding a CD4⁺ helper cell, CD8⁺ effector naive and memory cell, Treg cell, Natural Killer (NK) cell, or Natural killer T (NKT) cell population in a subject.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide as described herein and throughout, and pharmaceutical compositions comprising such a polynucleotide and a pharmaceutically acceptable carrier as described herein and throughout, wherein the fusion protein comprises the murine serum albumin (MSA) amino acid sequence set forth in SEQ ID NO:6.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide as described herein and throughout, and pharmaceutical compositions comprising such a polynucleotide and a pharmaceutically acceptable carrier as described herein and throughout, wherein the fusion protein comprises the human serum albumin (HSA) amino acid sequence set forth in SEQ ID NO:7.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide as described herein and throughout, and pharmaceutical compositions comprising such a polynucleotide and a pharmaceutically acceptable carrier as described herein and throughout, wherein the fusion protein comprises the murine serum albumin (MSA) amino acid sequence set forth in SEQ ID NO:6 linked to the N-terminus of the amino acid sequence set forth in SEQ ID NO:3.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide as described herein and throughout, and pharmaceutical compositions comprising such a polynucleotide and a pharmaceutically acceptable carrier as described herein and throughout, wherein the fusion protein comprises the amino acid sequence set forth in SEQ ID NO:3 linked to the N-terminus of the amino acid sequence set forth in the murine serum albumin (MSA) amino acid sequence set forth in SEQ ID NO:6.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide as described herein and throughout, and pharmaceutical compositions comprising such a polynucleotide and a pharmaceutically acceptable carrier as described herein and throughout, wherein the fusion protein comprises the amino acid sequence set forth in the murine serum albumin (MSA) amino acid sequence set forth in SEQ ID NO:6 linked via a linker to the N-terminus of the amino acid sequence set forth in SEQ ID NO:3.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide as described herein and throughout, and pharmaceutical compositions comprising such a polynucleotide and a pharmaceutically acceptable carrier as described herein and throughout, wherein the fusion protein comprises the amino acid sequence set forth in SEQ ID NO:3 linked via a linker to the N-terminus of the amino acid sequence set forth in the murine serum albumin (MSA) amino acid sequence set forth in SEQ ID NO:6.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide as described herein and throughout, and pharmaceutical compositions comprising such a polynucleotide and a pharmaceutically acceptable carrier as described herein and throughout, wherein the fusion protein comprises the amino acid sequence set forth in SEQ ID NO:7 linked to the N-terminus of the amino acid sequence set forth in SEQ ID NO:3.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide as described herein and throughout, and pharmaceutical compositions comprising such a polynucleotide and a pharmaceutically acceptable carrier as described herein and throughout, wherein the fusion protein comprises the amino acid sequence set forth in SEQ ID NO:3 linked to the N-terminus of the amino acid sequence set forth in in SEQ ID NO:7.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide as described herein and throughout, and pharmaceutical compositions comprising such a polynucleotide and a pharmaceutically acceptable carrier as described herein and throughout, wherein the fusion protein comprises the amino acid sequence set forth in in SEQ ID NO:7 linked to the N-terminus of the amino acid sequence set forth in SEQ ID NO:3.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide as described herein and throughout, and pharmaceutical compositions comprising such a polynucleotide and a pharmaceutically acceptable carrier as described herein and throughout, wherein the fusion protein comprises the amino acid sequence set forth in SEQ ID NO:3 linked via a linker to the N-terminus of the amino acid sequence set forth in in SEQ ID NO:7.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide as described herein and throughout, and pharmaceutical compositions comprising such a polynucleotide and a pharmaceutically acceptable carrier as described herein and throughout, wherein the fusion protein comprises the amino acid sequence set forth in SEQ ID NO:4.

In certain embodiments, which may be combined with other embodiments provided herein and throughout, are provided polynucleotides comprising a nucleic acid sequence encoding a fusion protein comprising a modified interleukin 2 (IL-2) polypeptide, wherein the polynucleotide as described herein and throughout, a pharmaceutical composition comprising such a polynucleotide and a pharmaceutically acceptable carrier as described herein and throughout, wherein the fusion protein comprises the amino acid sequence set forth in SEQ ID NO:5.

In certain embodiments, which may be combined with other embodiments provided herein and throughout, are provided pharmaceutical compositions comprising such a polynucleotide comprising a nucleic acid sequence encoding a fusion protein comprising a modified interleukin 2 (IL-2) polypeptide as described herein and throughout, and a pharmaceutically acceptable carrier as described herein and throughout, wherein the polynucleotide comprises the nucleic acid sequence set forth in SEQ ID NO:8, SEQ ID NO:9, SEQ ID NO:10, SEQ ID NO:11, SEQ ID NO:12, SEQ ID NO:13, or SEQ ID NO:14.

In certain embodiments, which may be combined with other embodiments provided herein and throughout, are provided pharmaceutical compositions comprising such a polynucleotide comprising a nucleic acid sequence encoding a fusion protein comprising a modified interleukin 2 (IL-2) polypeptide as described herein and throughout, and a pharmaceutically acceptable carrier as described herein and throughout, wherein the polynucleotide comprises the nucleic acid sequence set forth in SEQ ID NO:11, SEQ ID NO:12, SEQ ID NO:13, or SEQ ID NO:14.

In certain embodiments, which may be combined with other embodiments provided herein and throughout, are provided pharmaceutical compositions comprising such a polynucleotide comprising a nucleic acid sequence encoding a fusion protein comprising a modified interleukin 2 (IL-2) polypeptide as described herein and throughout, and a pharmaceutically acceptable carrier as described herein and throughout, wherein the pharmaceutical composition comprises: a) the nucleic acid sequence set forth in SEQ ID NO:8, SEQ ID NO:9, SEQ ID NO:10, SEQ ID NO:11, SEQ ID NO:12, SEQ ID NO:13, or SEQ ID NO:14; and
b) the following compound:

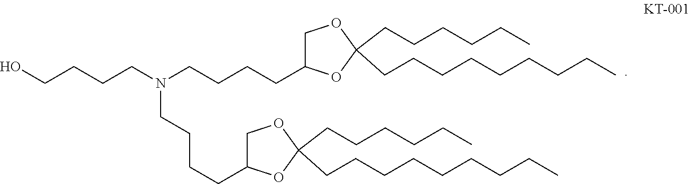 [SMILES: CCCCCCCCCC1(CCCCCC)OCC(CCCCN(CCCCO)CCCCC2COC(CCCCCC)(CCCCCCCCC)O2)O1]

In certain embodiments, which may be combined with other embodiments provided herein and throughout, are provided pharmaceutical compositions comprising such a polynucleotide comprising a nucleic acid sequence encoding a fusion protein comprising a modified interleukin 2 (IL-2) polypeptide as described herein and throughout, and a pharmaceutically acceptable carrier as described herein and throughout, wherein the pharmaceutical composition comprises: a) the nucleic acid sequence set forth in SEQ ID NO:8, SEQ ID NO:9, SEQ ID NO:10, SEQ ID NO:11, SEQ ID NO:12, SEQ ID NO:13, or SEQ ID NO:14; b) the following compound:

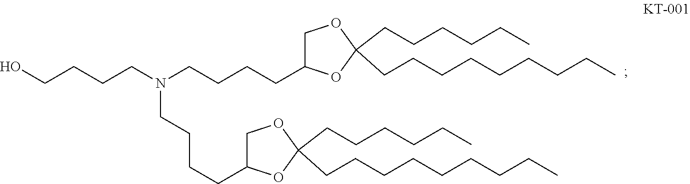 [SMILES: CCCCCCCCCC1(CCCCCC)OCC(CCCCN(CCCCO)CCCCC2COC(CCCCCC)(CCCCCCCCC)O2)O1]

and
lipid nanoparticles.

In certain embodiments, which may be combined with other embodiments provided herein and throughout, are provided pharmaceutical compositions comprising such a polynucleotide comprising a nucleic acid sequence encoding a fusion protein comprising a modified interleukin 2 (IL-2) polypeptide as described herein and throughout, and a pharmaceutically acceptable carrier as described herein and throughout, wherein the pharmaceutical composition comprises:
a) the nucleic acid sequence set forth in SEQ ID NO:11, SEQ ID NO:12, SEQ ID NO:13, or SEQ ID NO:14;
b) the following compound:

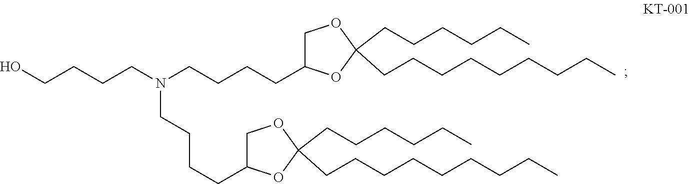 [SMILES: CCCCCCCCCC1(CCCCCC)OCC(CCCCN(CCCCO)CCCCC2COC(CCCCCC)(CCCCCCCCC)O2)O1]

and
lipid nanoparticles.

Other aspects and advantages of the present invention will be apparent from the embodiments and examples provided herein.

For the sake of brevity, the disclosures of the publications cited in this specification, including patents, are herein incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C provide results of an ELISA assay using post-transfection supernatants from 293 cells that were transfected with mRNA encoding the indicated fusion proteins.

FIG. 2 illustrates reporter assay results obtained using a HEK-Blue IL-2 receptor (IL-2R) reporter cell line (invivoGen) that has been transfected with mRNA encoding the indicated proteins. 4210=ACT4210 alone (i.e., not fused or linked to another protein) HSA-4210=fusion protein comprising, in N- to C-terminal order, human serum albumin linked to ACT4210; MSA-4210=fusion protein comprising, in N- to C-terminal order, murine serum albumin linked to ACT4210; 4210-HSA=fusion protein comprising, in N- to C-terminal order, ACT4210 linked to human serum albumin; 4210-MSA=fusion protein comprising, in N- to C-terminal order, ACT4210 linked to murine serum albumin.

FIG. 3 illustrates binding assay results obtained from the indicated fusion proteins expressed by cells transfected with mRNA encoding the indicated fusion proteins towards either IL-2Rα (left panel) or IL-2R βγ (right panel). 5210-MSA=fusion protein comprising, in N- to C-terminal order, murine serum albumin linked to ACT5210, an IL-2 mutein that is different from the IL-2 mutein known as 4210.

FIG. 4 illustrates in vivo expression results obtained from mice injected with the LNP-comprising pharmaceutical compositions, each pharmaceutical composition also comprising mRNA encoding the indicated fusion proteins. ACT421H=LNP-mRNA-HSA-4210; ACT421M=LNP-mRNA-MSA-4210.

FIG. 5 illustrates binding assay results obtained from the indicated fusion proteins expressed in vivo in mice that were injected with LNP-comprising pharmaceutical compositions, each pharmaceutical composition also comprising mRNA encoding the indicated fusion proteins, towards either IL-2Rα (left panel) or IL-2R βγ (right panel).

FIG. 6 illustrates binding assay results obtained from HSA-4210 fusion protein expressed in vivo in mice that were injected with LNP-comprising pharmaceutical compositions also comprising mRNA encoding the HSA-4210 fusion protein, towards either cynomolgus IL-2R βγ (left panel) or rat IL-2R βγ (right panel).

FIG. 7 illustrates binding assay results obtained from MSA-4210 fusion protein expressed in vivo in mice that were injected with LNP-comprising pharmaceutical compositions also comprising mRNA encoding the HSA-4210 fusion protein, towards either cynomolgus IL-2R βγ (left panel) or rat IL-2R βγ (right panel).

FIG. 8 illustrates reporter assay results obtained using a HEK-Blue IL-2 CD122/CD132 reporter cell line (invivoGen) that has been transfected with LNP-comprising pharmaceutical composition, also comprising mRNA encoding HSA-4210 fusion protein.

FIG. 9 illustrates results of a proliferation assay using CTLL-2 cells that were treated with indicated fusion proteins.

FIGS. 10A-10C illustrate concentration measured for each of the indicated proteins obtained from mice injected with LNP-comprising pharmaceutical compositions, each pharmaceutical composition also comprising mRNA encoding the indicated protein.

FIGS. 11A-11D illustrates results of a pharmacodynamic studies obtained from mice injected with LNP-comprising pharmaceutical compositions, each pharmaceutical composition also comprising mRNA encoding the indicated protein.

FIG. 12 illustrates flow cytometry results obtained from mice injected with LNP-comprising pharmaceutical composition, also comprising mRNA MSA-4210 fusion protein.

FIG. 13 illustrates number of CD8, CD4, and Treg cells obtained from mice injected with LNP-comprising pharmaceutical composition, also comprising mRNA MSA-4210 fusion protein.

FIGS. 14A-14C illustrates results demonstrating inhibition of lung metastasis obtained using B16F10 mice (a lung metastasis mouse model) injected with LNP-comprising pharmaceutical composition, also comprising mRNA MSA-4210 fusion protein.

FIG. 15 illustrates overall survival results obtained using B16F10 mice (a lung metastasis mouse model) injected with LNP-comprising pharmaceutical composition, also comprising mRNA MSA-4210 fusion protein, or with mice injected with IL-2 1 mg/kg once per day for five days, as indicated.

### DETAILED DESCRIPTION

The practice of the present invention will employ, unless otherwise indicated, conventional techniques of molecular biology (including recombinant techniques), microbiology, cell biology, biochemistry, immunology, and pharmacology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); and Remington, The Science and Practice of Pharmacy, 2^{nd} ed., (Lippincott, Williams & Wilkins 2003).

Unless defined otherwise, all technical and scientific terms used herein have the same meaning as is commonly understood by one of ordinary skill in the art to which this invention belongs. All patents, patent applications (published or unpublished), and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

The term “and/or” used herein is to betaken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition. In some embodiments, “about” encompasses variation within 10%, 5%, 2%, 1%, or 0.5% of a stated value.

Numeric ranges are inclusive of the numbers defining the range. Measured and measurable values are understood to be approximate, taking into account significant digits and the error associated with the measurement. Also, all ranges are to be interpreted as encompassing the endpoints in the absence of express exclusions such as “not including the endpoints”; thus, for example, “ranging from 1 to 10” includes the values 1 and 10 and all integer and (where appropriate) non-integer values greater than 1 and less than 10.

The term “administering”, “administered” and grammatical variants refers to the physical introduction of a therapeutic agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In one embodiment, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The terms “polypeptide,” “oligopeptide,” “peptide,” and “protein” are used interchangeably herein to refer to polymers of amino acids of any length, e.g., at least 5, 6, 7, 8, 9, 10, 20, 30, 40, 50, 100, 200, 300, 400, 500, 1,000 or more amino acids. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, the terms “variant” is used in reference to polypeptides that have some degree of amino acid sequence identity to a parent polypeptide sequence. A variant is similar to a parent sequence, but has at least one substitution, deletion or insertion in their amino acid sequence that makes them different in sequence from a parent polypeptide. Additionally, a variant may retain the functional characteristics of the parent polypeptide, e.g., maintaining a biological activity that is at least 50%, 60%, 70%, 80%, 90%, 95%, 98%, or 99% of that of the parent polypeptide.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule, and can be an immunoglobulin of any class, e.g., IgG, IgM, IgA, IgD and IgE, IgY, which is the major antibody type in avian species such as chicken, is also included within the definition. As used herein, the term encompasses not only intact polyclonal or monoclonal antibodies, but also fragments thereof (such as Fab, Fab′, F(ab′)2, Fv), single chain (ScFv), mutants thereof, naturally occurring variants, fusion proteins comprising an antibody portion with an antigen recognition site of the required specificity, humanized antibodies, chimeric antibodies, and any other modified configuration of the immunoglobulin molecule that comprises an antigen recognition site of the required specificity.

As used herein, the term “antigen” refers to a target molecule that is specifically bound by an antibody through its antigen recognition site. The antigen may be monovalent or polyvalent, i.e., it may have one or more epitopes recognized by one or more antibodies. Examples of kinds of antigens that can be recognized by antibodies include polypeptides, oligosaccharides, glycoproteins, polynucleotides, lipids, etc.

As used herein, the term “epitope” refers to a portion of an antigen, e.g., a peptide sequence of at least about 3 to 5, preferably about 5 to 10 or 15, and not more than about 1,000 amino acids (or any integer there between), which define a sequence that by itself or as part of a larger sequence, binds to an antibody generated in response to such sequence. There is no critical upper limit to the length of the fragment, which may, for example, comprise nearly the full-length of the antigen sequence, or even a fusion protein comprising two or more epitopes from the target antigen. An epitope for use in the subject invention is not limited to a peptide having the exact sequence of the portion of the parent protein from which it is derived, but also encompasses sequences identical to the native sequence, as well as modifications to the native sequence, such as deletions, additions and substitutions (conservative in nature).

The term “Fc” or “Fc region” as used herein refers to the portion of an antibody heavy chain constant region beginning in or after the hinge region and ending at the C-terminus of the heavy chain. The Fc region comprises at least a portion of the CH2 and CH3 regions and may, or may not, include a portion of the hinge region. An Fc domain may bind Fc cell surface receptors and some proteins of the immune complement system. An Fc region may bind a complement component C1q. An Fc domain may exhibit effector function, including any one or any combination of two or more activities including complement-dependent cytotoxicity (CDC), antibody-dependent cell-mediated cytotoxicity (ADCC), antibody-dependent phagocytosis (ADP), opsonization and/or cell binding. An Fc domain may bind an Fc receptor, including FcγRI (e.g., CD64), FcγRII (e.g, CD32) and/or FcγRIII (e.g., CD16a). In one embodiment, the Fc region may include a mutation that increases or decreases any one or any combination of these functions. In one embodiment, the Fc domain comprises Fc region comprises one or more mutations selected from N297A, N297Q, N297D, L234A, L235A, L235E, P329A, and P329G (e.g., according to Kabat numbering). In one embodiment, the Fc domain comprises a LALA mutation (e.g., equivalent to L234A, L235A according to Kabat numbering) which reduces effector function. In one embodiment, the Fc domain comprises a LALA-PG mutation (e.g., equivalent to L234A, L235A, P329G according to Kabat numbering) which reduces effector function. In one embodiment, the Fc domain mediates serum half-life of the protein complex, and a mutation in the Fc domain can increase or decrease the serum half-life of the protein complex. In one embodiment, the Fc domain affects thermal stability of the protein complex, and mutation in the Fc domain can increase or decrease the thermal stability of the protein complex. In one embodiment, the Fc region comprises one or more mutations selected from M252Y, T256D, T307Q, T307W, M252Y, S254T, T256E, M428L, and N434S (e.g., according to Kabat numbering). In one embodiment, the Fc region comprises the mutations M252Y, S254T, and T256E (YTE) (e.g., according to Kabat numbering).

The term “isolated” refers to a polynucleotide, a nucleic acid) or a protein, (e.g., an antibody, or an antigen binding portion thereof) or that is substantially free of other cellular material. The term isolated also refers in some embodiments to protein or polynucleotides that are substantially free of other molecules, reactants, reagents, and/or impurities, such as other proteins or polynucleotides having different amino acid or nucleotide sequences, respectively. The purity or homogeneity of the desired polynucleotide or protein can be assayed using techniques well known in the art, including low resolution methods such as gel electrophoresis and high resolution methods such as HPLC or mass spectrometry.

The term “labeled” or related terms as used herein with respect to a polypeptide refers to joinder antibodies and their antigen binding portions thereof that are unlabeled or joined to a detectable label or moiety for detection, wherein the detectable label or moiety is radioactive, colorimetric, antigenic, enzymatic, a detectable bead (such as a magnetic or electrodense (e.g., gold) bead), biotin, streptavidin or protein A. A variety of labels can be employed, including, but not limited to, radionuclides, fluorescers, enzymes, enzyme substrates, enzyme cofactors, enzyme inhibitors and ligands (e.g., biotin, haptens). Any of the polynucleotides, modified IL-2 polypeptides, or fusion proteins described herein can be unlabeled or can be joined to a detectable label or moiety.

The term “labeled” or related terms as used herein with respect to a polypeptide refers to joinder thereof to a detectable label or moiety for detection. Exemplary detectable labels or moieties include radioactive, colorimetric, antigenic, enzymatic labels/moieties, a detectable bead (such as a magnetic or electrodense (e.g., gold) bead), biotin, streptavidin or protein A. A variety of labels can be employed, including, but not limited to, radionuclides, fluorescers, enzymes, enzyme substrates, enzyme cofactors, enzyme inhibitors and ligands (e.g., biotin, haptens). Any of the polynucleotides, modified IL-2 polypeptides, or fusion proteins described herein can be unlabeled or can be joined to a detectable label or detectable moiety.

The term “leader sequence” or “leader peptide” or “[peptide] signal sequence” or “signal peptide” or “secretion signal peptide” refers to a peptide sequence that is located at the N-terminus of a polypeptide. A leader sequence directs a polypeptide chain to a cellular secretory pathway and can direct integration and anchoring of the polypeptide into the lipid bilayer of the cellular membrane. Typically, a leader sequence is about 10-50 amino acids in length and is cleaved from the polypeptide upon secretion of the mature polypeptide or insertion of the mature polypeptide into the membrane. Thus, proteins provided herein such as membrane proteins and antibodies having signal peptides that are identified by their precursor sequences that include a signal peptide sequence are also intended to encompass the mature forms of the polypeptides lacking the signal peptide, and proteins provided herein such as membrane proteins and antibodies having signal peptides that are identified by their mature polypeptide sequences that lack a signal peptide sequence are also intended to encompass forms of the polypeptides that include a signal peptide, whether native to the protein or derived from another secreted or membrane-inserted protein. In one embodiment, a leader sequence includes signal sequences comprising CD8α, CD28 or CD16 leader sequences. In one embodiment, the signal sequence comprises a mammalian sequence, including for example mouse or human Ig gamma secretion signal peptide.

The terms “percent identity”, “percent homology”, and related terms used herein refers to a quantitative measurement of the similarity between two polypeptide sequences or between two polynucleotide sequences. The percent identity between two polypeptide sequences is a function of the number of identical amino acids at aligned positions that are shared between the two polypeptide sequences, taking into account the number of gaps, and the length of each gap, which may need to be introduced to optimize alignment of the two polypeptide sequences. In a similar manner, the percent identity between two polynucleotide sequences is a function of the number of identical nucleotides at aligned positions that are shared between the two polynucleotide sequences, taking into account the number of gaps, and the length of each gap, which may need to be introduced to optimize alignment of the two polynucleotide sequences. A comparison of the sequences and determination of the percent identity between two polypeptide sequences, or between two polynucleotide sequences, may be accomplished using a mathematical algorithm. For example, the “percent identity” or “percent homology” of two polypeptide or two polynucleotide sequences may be determined by comparing the sequences using the GAP computer program (a part of the GCG Wisconsin Package, version 10.3 (Accelrys, San Diego, Calif.)) using its default parameters. Expressions such as “comprises a sequence with at least X % identity to Y” with respect to a test sequence mean that, when aligned to sequence Y as described above, the test sequence comprises residues identical to at least X % of the residues of Y.

In some embodiments, polynucleotides comprising a nucleic acid sequence encoding a fusion protein comprising a modified interleukin 2 (IL-2) polypeptide are provided, wherein the polynucleotide, the modified IL-2 polypeptide, and/or the fusion protein may be similar but not necessarily identical to any given test nucleic acid sequences or test amino acid sequences. The similarities between the polynucleotides, modified IL-2 polypeptides, or fusion proteins described herein and throughout to a given test nucleic acid sequence or amino acid sequence, respectively, may comprise at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100% identity.

The term “amino acid substitution” and like terms refers to the substitution of one amino acid residue in a polypeptide with another amino acid residue having a different side chain (R group). It is understood that such amino acid substitutions may be achieved by changing or altering a nucleic acid or polynucleotide sequence encoding a given polypeptide so as encode, and thus produce when expressed, a polypeptide sequence containing the amino acid substitution.

The term “conservative amino acid substitution” and like terms refers to the substitution of one amino acid residue in a polypeptide with another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference in its entirety. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. It is understood that such conservative amino acid substitutions may be achieved by changing or altering a nucleic acid or polynucleotide sequence encoding a given polypeptide so as encode, and thus produce when expressed, a polypeptide sequence containing the conservative amino acid substitution.

As used herein, the term “specifically binds” refers to the binding specificity of a specific binding pair. Recognition by an antibody of a particular target in the presence of other potential targets is one characteristic of such binding. Specific binding involves two different molecules wherein one of the molecules specifically binds with the second molecule through chemical or physical means. The two molecules are related in the sense that their binding with each other is such that they are capable of distinguishing their binding partner from other assay constituents having similar characteristics. The members of the binding component pair are referred to as ligand and receptor (anti-ligand), specific binding pair (SBP) member and SBP partner, and the like. A molecule may also be an SBP member for an aggregation of molecules; for example an antibody raised against an immune complex of a second antibody and its corresponding antigen may be considered to be an SBP member for the immune complex.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. The term refers to a polymer containing at least two deoxyribonucleotides or ribonucleotides in either single- or double-stranded form and includes DNA, RNA, and hybrids thereof, as well as peptide nucleic acids, locked nucleic acids, and other synthetic nucleic acid analogs and hybrids thereof. Nucleic acids and polynucleotides include recombinant and chemically-synthesized forms, and may include DNA (cDNA or genomic DNA), RNA (e.g., mRNA, siRNA, dsRNA, shRNA, miRNA, tRNA, rRNA, vRNA), analogs of the DNA or RNA generated using nucleotide analogs (e.g., peptide nucleic acids and non-naturally occurring nucleotide analogs), and hybrids thereof. Nucleic acid molecule can be single-stranded or double-stranded. In some embodiments nucleic acids comprise a one type of polynucleotides or a mixture of two or more different types of polynucleotides.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR 2 (“amidate”), P(O)R, P(O)OR′, CO or CH 2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide,” as used herein, generally refers to short, generally single stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

As used herein, the term “homologue” is used to refer to a nucleic acid which differs from a naturally occurring nucleic acid (e.g., the “prototype” or “wild-type” nucleic acid) by minor modifications to the naturally occurring nucleic acid, but which maintains the basic nucleotide structure of the naturally occurring form. Such changes include, but are not limited to: changes in one or a few nucleotides, including deletions (e.g., a truncated version of the nucleic acid) insertions and/or substitutions. A homologue can have enhanced, decreased, or substantially similar properties as compared to the naturally occurring nucleic acid. A homologue can be complementary or matched to the naturally occurring nucleic acid. Homologues can be produced using techniques known in the art for the production of nucleic acids including, but not limited to, recombinant DNA techniques, chemical synthesis, etc.

As used herein, “substantially complementary or substantially matched” means that two nucleic acid sequences have at least 90% sequence identity. Preferably, the two nucleic acid sequences have at least 95%, 96%, 97%, 98%, 99% or 100% of sequence identity. Alternatively, “substantially complementary or substantially matched” means that two nucleic acid sequences can hybridize under high stringency condition(s).

In general, the stability of a hybrid is a function of the ion concentration and temperature. Typically, a hybridization reaction is performed under conditions of lower stringency, followed by washes of varying, but higher, stringency. Moderately stringent hybridization refers to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions are conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization refers to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M (EDTA). Other suitable moderate stringency and high stringency hybridization buffers and conditions are well known to those of skill in the art.

A “vector” and related terms used herein refers to a nucleic acid molecule or a polynucleotide (e.g., DNA, RNA, mRNA, and the like) which can be operably linked to foreign genetic material (e.g., nucleic acid transgene, such as a polynucleotide encoding a fusion protein comprising a modified IL-2 polypeptide as described herein and throughout). Vectors can be used as a vehicle to introduce foreign genetic material into a cell (e.g., host cell) in vivo, ex vivo, and/or in vitro. Vectors can include at least one restriction endonuclease recognition sequence for insertion of the transgene into the vector. Vectors can include at least one gene sequence that confers antibiotic resistance or a selectable characteristic to aid in selection of host cells that harbor a vector-transgene construct. Expression vectors can include one or more origin of replication sequences. Vectors can be single-stranded or double-stranded nucleic acid molecules. Vectors can be linear or circular nucleic acid molecules. One type of vector is a “plasmid,” which refers to a linear or circular double stranded extrachromosomal DNA molecule which can be linked to a transgene, and is capable of replicating in a host cell, and transcribing and/or translating the transgene. A viral vector typically contains viral RNA or DNA backbone sequences which can be linked to the transgene. The viral backbone sequences can be modified to disable infection but retain insertion of the viral backbone and the co-linked transgene into a host cell genome. Examples of viral vectors include retroviral, lentiviral, adenoviral, adeno-associated viral, baculoviral, papovaviral, vaccinia viral, herpes simplex viral and Epstein Barr viral vectors. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome.

An “expression vector” (aka a “plasmid”, used interchangeably throughout) is a type of vector that can contain one or more regulatory sequences and/or regulatory promoter systems, such as inducible and/or constitutive promoters and enhancers. Such regulatory sequences and/or regulatory promoter systems may comprise one or more of a variety of promoter of enhancer sequences obtained or derived from, for example, cytomegalovirus (CMV) promoter, elongation factor 1 (EF1) promoter, chicken β-actin promoter, β-actin promoters from other species, phosphoglycerokinase (PGK) promoter, spleen focus-forming virus (SFFV) promoter, Rous sarcoma virus (RSV) promoter, human serum albumin (SA) promoter, thyroxine binding globulin (TBG) promoter, cytochrome P450 2E1 (CYP2E1) promoter, muscle creatine kinase enhancer sequences, desmin enhancer sequences, desmin promoter sequences, MHDLKSMRTe enhancer/promoter sequences, and the like. Exemplary such vectors, such as NanoPlasmid™ vectors, regulatory elements, and the like may be found in, for example, US 2004/0175727 and WO 2022/056291, hereby incorporated by referenced in their entireties for all purposes.

Expression vectors may also comprise regulatory promoter systems comprising combinations of one or more promoters sequences and/or enhancer sequences, such: as a chicken β-actin/CMV enhancer (CAG) promoter; a human or murine CMV-derived enhancer elements combined with the elongation factor 1a (EF1a) promoters; CpG free versions of the human or murine CMV-derived enhancer elements combined with the elongation factor 1a (EF 1α) promoters; the albumin promoter combined with an α-fetoprotein MERII enhancer; one or more MCK enhancer sequences combined with a desmin enhancer sequence and/or a desmin long promoter sequence; one or more MCK enhancer sequences combined with a desmin enhancer sequence and/or a desmin short promoter sequence; a CMV enhancer sequence and an EF1α promoter sequence; one or more MCK enhancer sequences and a C5-12 promoter sequence; one or more MCK enhancer sequences; and the like. In certain embodiments, C5-12 promoter sequences, C1-26 promoter sequences, C2-26 promoter sequences, C2-27 promoter sequences, C5-5 promoter sequences, C6-5 promoter sequences, C6-16 promoter sequences, C6-39 promoter sequences, C6-5 promoter sequences, MEF-1 regulatory elements, MEF-2 regulatory elements, SRE regulatory elements, TEF-1 regulatory elements, and the like may be substituted for one or more of the promoter and/or enhancer sequences described above, and/or added to or added to one or more of the promoter and/or enhancer sequences described above, in order to generate regulatory promoter systems in accordance with the disclosure.

Such regulatory sequences and/or regulatory promoter systems, may also drive or facilitate tissue-specific transcription and/or translation of nucleic acid sequences that encode polypeptides, such as antigen-binding proteins (e.g., antibodies or antigen binding fragments thereof). Exemplary such promoters and/or enhancers may drive tissue-specific expression of polynucleotides encoding polypeptides. Exemplary such promoters and/or enhancers may drive muscle-specific expression of polynucleotides encoding polypeptides. Such tissue- and/or muscle-specific promoters and/or enhancers which may be combined to generate regulatory tissue- and/or muscle-specific promoter systems. In some embodiments, such tissue- and/or muscle-specific promoters and/or enhancers comprise for example, one or more promoter or enhance sequences disclosed in, e.g., US 2004/0175727, incorporated herein by reference in its entirety). In some embodiments, such tissue- and/or muscle-specific promoters and/or enhancers which may be employed in combinations to generate regulatory prompter systems in accordance with the disclosure comprise, for example, one or more promoter or enhance muscle creatine kinase (MCK) enhancer sequences, desmin enhancer sequences, desmin long promoter sequences, desmin short promoter sequences, C5-12 promoter sequences, C1-26 promoter sequences, C2-26 promoter sequences, C2-27 promoter sequences, C5-5 promoter sequences, C6-5 promoter sequences, C6-16 promoter sequences, C6-39 promoter sequences, C6-5 promoter sequences, MEF-1 regulatory elements, MEF-2 regulatory elements, SRE regulatory elements, TEF-1 regulatory elements, elongation factor 1 alpha (“EF1a” or “EF1α”, used interchangeably throughout) promoter sequences, MHDLKSMRTx enhancer/promoter sequences, and the like.

A regulatory promoter system operably linked to the polypeptide-encoding sequence(s) can be a eukaryotic or prokaryotic regulatory promoter system but is preferably a eukaryotic regulatory promoter system hat is active in a mammalian cell. The regulatory promoter system(s) can direct transcription and/or translation of the transgene in the host cell and can include ribosomal binding sites and/or polyadenylation sites. In certain embodiments, the regulatory promoter system(s) can independently, or collectively (e.g., in concert) direct transcription and/or translation of the transgene in the host cell and can include ribosomal binding sites and/or polyadenylation sites. In certain embodiments, the regulatory promoter system(s) can independently, or collectively (e.g., in concert) direct tissue-specific transcription and/or translation of the transgene in the host cell and can include ribosomal binding sites and/or polyadenylation sites. In certain embodiments, the regulatory promoter system(s) can independently, or collectively (e.g., in concert) direct muscle-specific transcription and/or translation of the transgene in the host cell and can include ribosomal binding sites and/or polyadenylation sites. In various embodiments, the host cell, or population of host cells, harbor one or more expression vectors that can direct transient introduction of the transgene into the host cells or stable insertion of the transgene into the host cells' genome, where the transgene comprises nucleic acids encoding any of the first and/or second polypeptides described herein. In embodiments where a nucleic acid molecule encodes two polypeptides, the two polypeptide-encoding sequences can be regulated by the same promoter and can be linked by an IRES or 2A sequence (Shao et al. (2009) Cell Research 19:296-306) or the two polypeptide-encoding sequences can be operably linked to different promoters.

Expression vectors can include ribosomal binding sites and/or polyadenylation sites. Expression vectors can include one or more origin of replication sequences. Regulatory sequences direct transcription, or transcription and translation, of a transgene linked to or inserted into the expression vector which is transduced into a host cell. The regulatory sequence(s) can control the level, timing and/or location of expression of the transgene. The regulatory sequence can, for example, exert its effects directly on the transgene, or through the action of one or more other molecules (e.g., polypeptides that bind to the regulatory sequence and/or the nucleic acid). Regulatory sequences can be part of a vector. Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-3606.

A transgene is “operably linked” to a regulatory sequence (e.g., a promoter) when the regulatory sequence affects the expression (e.g., the level, timing, or location of expression) of the transgene.

The terms “transfected” or “transformed” or “transduced” or other related terms used herein refer to a process by which exogenous nucleic acid (e.g., transgene) is transferred or introduced into a host cell. A “transfected” or “transformed” or “transduced” host cell is one which has been transfected, transformed or transduced with exogenous nucleic acid (transgene). The host cell includes the primary subject cell and its progeny.

As used herein, “vector (or plasmid)” refers to discrete elements that are used to introduce heterologous DNA into cells for either expression or replication thereof. Selection and use of such vehicles are well known within the skill of the artisan. An expression vector includes vectors capable of expressing DNA's that are operatively linked with regulatory sequences, such as promoter regions, that are capable of effecting expression of such DNA fragments. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, “a promoter region or promoter element” refers to a segment of DNA or RNA that controls transcription of the DNA or RNA to which it is operatively linked. The promoter region includes specific sequences that are sufficient for RNA polymerase recognition, binding and transcription initiation. This portion of the promoter region is referred to as the promoter. In addition, the promoter region includes sequences that modulate this recognition, binding and transcription initiation activity of RNA polymerase. These sequences may be cis acting or may be responsive to trans acting factors. Promoters, depending upon the nature of the regulation, may be constitutive or regulated. Exemplary promoters contemplated for use in prokaryotes include the bacteriophage T7 and T3 promoters, and the like.

As used herein, “operatively linked” or “operationally associated” refers to the functional relationship of DNA or RNA with regulatory and effector sequences of nucleotides, such as promoters, enhancers, transcriptional and translational start sites, transcriptional and translational stop sites, polyadenylation sites, ribosome entry sites, five prime cap sites, and other transcriptional or translational control or regulatory elements. For example, operative linkage of DNA or RNA to a promoter refers to the physical and functional relationship between the DNA or RNA and the promoter such that the transcription of such DNA is initiated from the promoter by an RNA polymerase that specifically recognizes, binds to and transcribes the DNA. In order to optimize expression and/or in vitro transcription, it may be necessary to remove, add or alter 5′ untranslated portions of the clones to eliminate extra, potential inappropriate alternative translation initiation (i.e., start) codons or other sequences that may interfere with or reduce expression, either at the level of transcription or translation. Alternatively, consensus sites can be inserted immediately 5′ of the start codon and may enhance expression. See, e.g., Kozak (1991) J. Biol. Chem. 266:19867-19870. The desirability of (or need for) such modification may be empirically determined.

“Treating” or “treatment” or “alleviation” refers to therapeutic treatment wherein the object is to slow down (lessen) if not cure the targeted pathologic condition or disorder or prevent recurrence of the condition. A subject is successfully “treated” if, after receiving a therapeutic amount of a therapeutic agent or treatment, the subject shows observable and/or measurable reduction in or absence of one or more signs and symptoms of the particular disease. Reduction of the signs or symptoms of a disease may also be felt by the patient. A patient is also considered treated if the patient experiences stable disease. In some embodiments, treatment with a therapeutic agent is effective to result in the patients being disease-free 3 months after treatment, preferably 6 months, more preferably one year, even more preferably 2 or more years post treatment. These parameters for assessing successful treatment and improvement in the disease are readily measurable by routine procedures familiar to a physician of appropriate skill in the art. In some embodiments, “treatment” means any manner in which the symptoms of a condition, disorder or disease are ameliorated or otherwise beneficially altered. Treatment also encompasses any pharmaceutical use of the compositions herein. In some embodiments, “amelioration” of the symptoms of a particular disorder by administration of a particular pharmaceutical composition refers to any lessening, whether permanent or temporary, lasting or transient that can be attributed to or associated with administration of the composition.

The term “prediction” or “prognosis” is often used herein to refer to the likelihood that a patient will respond either favorably or unfavorably to a drug or set of drugs, or the likely outcome of a disease. In one embodiment, the prediction relates to the extent of those responses or outcomes. In one embodiment, the prediction relates to whether and/or the probability that a patient will survive or improve following treatment, for example treatment with a particular therapeutic agent, and for a certain period of time without disease recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, steroid treatment, etc.

The term “prevent”, “prevention”, or “preventing” refers to the provision or administration of a therapeutic or prophylactic agent or treatment regimen, wherein the object is to diminish, reduce, or eliminate the occurrence of a disease, condition, or disorder in a subject suspected of having or at risk of having the disease, condition or disorder. Similarly, “prevent”, “prevention”, or “preventing” refers to the provision or administration of a therapeutic or prophylactic agent or treatment regimen, wherein the object is to diminish, reduce, or eliminate the occurrence of one or more symptoms or clinical manifestations of a disease, condition, or disorder in a subject suspected of having or at risk of having the disease, condition or disorder.

A subject is successfully “treated” if, after receiving a therapeutic amount of a therapeutic agent or treatment, the subject shows observable and/or measurable reduction in or absence of one or more signs and symptoms of the particular disease, disorder, or condition. Reduction of the signs or symptoms of a disease may also be felt by the patient. A patient is also considered treated if the patient experiences stable disease. In some embodiments, treatment with a therapeutic agent is effective to result in the patients being disease-free 3 months after treatment, preferably 6 months, more preferably one year, even more preferably 2 or more years post treatment. These parameters for assessing successful treatment and improvement in the disease are readily measurable by routine procedures familiar to a physician of appropriate skill in the art. In some embodiments, “treatment” means any manner in which the symptoms of a condition, disorder or disease are ameliorated or otherwise beneficially altered. Treatment also encompasses any pharmaceutical use of the compositions herein. In some embodiments, “amelioration” of the symptoms of a particular disorder by administration of a particular pharmaceutical composition refers to any lessening, whether permanent or temporary, lasting or transient that can be attributed to or associated with administration of the composition.

The term “synergistic effect” refers to a situation where the combination of two or more agents produces a greater effect than the sum of the effects of each of the individual agents. The term encompasses not only a reduction in symptoms of the disorder to be treated, but also an improved side effect profile, improved tolerability, improved patient compliance, improved efficacy, or any other improved clinical outcome.

The term a “sub-therapeutic amount” of an agent or therapy is an amount less than the effective amount for that agent or therapy as a single agent, but when combined with an effective or sub-therapeutic amount of another agent or therapy can produce a result desired by the physician, due to, for example, synergy in the resulting efficacious effects, or reduced side effects.

Combination therapy or “in combination with” refer to the use of more than one therapeutic agent to treat a particular disorder or condition. By “in combination with,” it is not intended to imply that the therapeutic agents must be administered at the same time and/or formulated for delivery together, although these methods of delivery are within the scope of this disclosure. A therapeutic agent can be administered concurrently with, prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after), one or more other additional agents. The therapeutic agents in a combination therapy can also be administered on an alternating dosing schedule, with or without a resting period (e.g., no therapeutic agent is administered on certain days of the schedule). The administration of a therapeutic agent “in combination with” another therapeutic agent includes, but is not limited to, sequential administration and concomitant administration of the two agents. In general, each therapeutic agent is administered at a dose and/or on a time schedule determined for that particular agent.

As used herein the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20^{th} ed., (Lippincott, Williams & Wilkins 2003). Except insofar as any conventional media or agent is incompatible with the active compound, such use in the compositions is contemplated.

A “pharmaceutically acceptable salt” is intended to mean a salt of a free acid or base of a compound represented herein that is non-toxic, biologically tolerable, or otherwise biologically suitable for administration to the subject. See, generally, Berge, et al., J. Pharm. Sci., 1977, 66, 1-19. Preferred pharmaceutically acceptable salts are those that are pharmacologically effective and suitable for contact with the tissues of subjects without undue toxicity, irritation, or allergic response. A modified interleukin 2 (IL-2) polypeptide or its conjugate described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates.

As used herein, the term “therapeutically effective amount” or “effective amount” refers to an amount of a therapeutic agent that when administered alone or in combination with an additional therapeutic agent to a cell, tissue, or subject is effective to prevent or ameliorate a disease or disorder, a proliferation disease or disorder, in a subject. A therapeutically effective dose further refers to that amount of the therapeutic agent sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. In some embodiments, “an effective amount of a compound for treating a particular disease” is an amount that is sufficient to ameliorate, or in some manner reduce the symptoms associated with the disease. Such amount may be administered as a single dosage or may be administered according to a regimen, whereby it is effective. The amount may cure the disease but, typically, is administered in order to ameliorate the symptoms of the disease. Repeated administration may be required to achieve the desired amelioration of symptoms.

The term “combination” refers to either a fixed combination in one dosage unit form, or a kit of parts for the combined administration where a modified interleukin 2 (IL-2) polypeptide or its conjugate and a combination partner (e.g., another drug as explained below, also referred to as “therapeutic agent” or “co-agent”) may be administered independently at the same time or separately within time intervals, especially where these time intervals allow that the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a modified interleukin 2 (IL-2) polypeptide or its conjugate and a combination partner, are both administered to a patient simultaneously in the form of a single entity or dosage. The term “non-fixed combination” means that the active ingredients, e.g., a modified interleukin 2 (IL-2) polypeptide or its conjugate and a combination partner, are both administered to a patient as separate entities either simultaneously, concurrently or sequentially with no specific time limits, wherein such administration provides therapeutically effective levels of the two substances in the body of the patient. The latter also applies to cocktail therapy, e.g., the administration of three or more active ingredients.

As used herein, “biological sample” refers to any sample obtained from a living or viral source or other source of macromolecules and biomolecules, and includes any cell type or tissue of a subject from which nucleic acid or protein or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom.

The terms “level” or “levels” are used to refer to the presence and/or amount of a target, e.g., a substance or an organism that is part of the etiology of a disease or disorder, and can be determined qualitatively or quantitatively. A “qualitative” change in the target level refers to the appearance or disappearance of a target that is not detectable or is present in samples obtained from normal controls. A “quantitative” change in the levels of one or more targets refers to a measurable increase or decrease in the target levels when compared to a healthy control.

A “healthy control” or “normal control” is a biological sample taken from an individual who does not suffer from a disease or disorder, e.g., a proliferation disease or disorder. A “negative control” is a sample that lacks any of the specific analyte the assay is designed to detect and thus provides a reference baseline for the assay.

As used herein, “mammal” refers to any of the mammalian class of species. Frequently, the term “mammal,” as used herein, refers to humans, human subjects or human patients. “Mammal” also refers to any of the non-human mammalian class of species, e.g., experimental, companion or economic non-human mammals. Exemplary non-human mammals include mice, rats, rabbits, cats, dogs, pigs, cattle, sheep, goats, horses, monkeys, Gorillas and chimpanzees.

As used herein, “production by recombinant means” refers to production methods that use recombinant nucleic acid methods that rely on well-known methods of molecular biology for expressing polypeptides or proteins encoded by cloned nucleic acids.

The terms “subject” and “patient” as used herein refer to human and non-human animals, including vertebrates, mammals and non-mammals. In one embodiment, the subject can be human, non-human primates, simian, ape, murine (e.g., mice and rats), bovine, porcine, equine, canine, feline, caprine, lupine, ranine or piscine.

As used herein, a “prodrug” is a substance that, upon in vivo administration, is metabolized or otherwise converted to the biologically, pharmaceutically or therapeutically active form of the substance. To produce a prodrug, the pharmaceutically active substance is modified such that the active substance will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

It is understood that aspects and embodiments of the invention described herein include “consisting” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Other objects, advantages and features of the present invention will become apparent from the following specification taken in conjunction with the accompanying drawings.

Polynucleotides Encoding Modified Interleukin 2 (IL-2) Polypeptides and Fusion Proteins Comprising them

In some embodiments, polynucleotides comprising a nucleic acid sequence encoding a fusion protein comprising a modified interleukin 2 (IL-2) polypeptide, wherein the modified interleukin 2 (IL-2) polypeptide comprises an amino acid having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100% sequence identity to the amino acid sequence set forth in SEQ ID NO:1 (also known as human wild-type IL-2) or SEQ ID NO:2 (also known as human rhIL-2), wherein the modified IL-2 polypeptide comprises at least one substitution with a natural amino acid or an unnatural amino acid at one or more positions selected from the group consisting of Q13, L19, N29, N30, Y31, K32, N33, P34, K35, T37, R38, T41, F42, K43, Y45, K48, K49, E62, K64, P65, N71, Q74, K76, R81, L85, S87, V91, I92, V93, and combinations thereof, are provided. In some embodiments, the modified IL-2 polypeptide: a) is configured to be conjugated to a water-soluble polymer, a lipid, or a polypeptide, e.g., a protein or a peptide; and/or b) has reduced binding to an interleukin 2 receptor α (IL-2Rα) compared to a comparable IL-2 polypeptide comprising an amino acid sequence set forth in SEQ ID NO:1 or SEQ ID NO:2 without the substitution; and/or c) has reduced receptor signaling potency to IL-2Rαβγ compared to a comparable IL-2 polypeptide comprising an amino acid sequence set forth in SEQ ID NO:1 or SEQ ID NO:2 without the at least one substitution, and/or d) has increased ratio of signaling potency to IL-2Rβγ over signaling potency to IL-2Rαβγ (increased ratio of signaling potency to IL-2Rβγ/signaling potency to IL-2Rαβγ) compared to a comparable IL-2 polypeptide comprising an amino acid sequence set forth in SEQ ID NO:1 or SEQ ID NO:2 without the at least one substitution, and/or e) has enhanced receptor signaling potency to IL-2Rβγ compared to a comparable IL-2 polypeptide comprising an amino acid sequence set forth in SEQ ID NO:1 or SEQ ID NO:2 without the at least one substitution, and/or provided that when the modified IL-2 polypeptide comprises at least one substitution with an unnatural amino acid, the modified IL-2 polypeptide comprises at least one substitution at a position selected from the group consisting of N29, N30, Y31, K32, N33, P34, K35, R38, T41, F42, K43, Y45, K48, K49, E62, K64, P65, N71, Q74, K76, and combinations thereof, and/or at least one substitution with a natural amino acid or an unnatural amino acid at at least one position within IL-2Rα interaction region, IL-2Rβ interaction region and/or IL-2Rγ interaction region; and/or f) combinations of a) through e).

The amino acid sequences of SEQ ID NO:1 or SEQ ID NO:2 are set forth below:

SEQ ID NO: 1
(¹APTSSSTKKTQL¹³QLEHLL¹⁹LDLQMILNGI²⁹N³⁰N³¹Y³²K³³
N³⁴P³⁵KLT³⁸RML⁴¹T⁴²F⁴³KF⁴⁵YMP⁴⁸K⁴⁹KATELKHLQCLEE⁶²E
L⁶⁴K⁶⁵PLEEVL⁷¹NLA⁷⁴QS⁷⁶KNFHL⁸¹RPRD⁸⁵LI⁸⁷SNIN⁹¹V⁹²
I⁹³VLELKGSETTFMCEYADETATIVEFLNRWITFSQSIIST
L¹³³T)
SEQ ID NO: 2
(¹MPTSSSTKKTQL¹³QLEHLL¹⁹LDLQMILNGI²⁹N³⁰N³¹Y³²K³³
N³⁴P³⁵KLT³⁸RML⁴¹T⁴²F⁴³KF⁴⁵YMP⁴⁸K⁴⁹KATELKHLQCLEE⁶²E
L⁶⁴K⁶⁵PLEEVL⁷¹NLA⁷⁴QS⁷⁶KNFHL⁸¹RPRD⁸⁵LI⁸⁷SNIN⁹¹V⁹²
I⁹³VLELKGSETTFMCEYADETATIVEFLNRWITFCQSIIST
L¹³³T)

In some embodiments, polynucleotides comprising a nucleic acid sequence encoding a fusion protein comprising a modified interleukin 2 (IL-2) polypeptide are provided, wherein the modified IL-2 polypeptide has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100% sequence identity in the region of amino acid residues 10-25 to the corresponding region of a comparable IL-2 polypeptide comprising an amino acid sequence set forth in SEQ ID NO:1 or SEQ ID NO:2 without the substitution.

In some embodiments, polynucleotides comprising a nucleic acid sequence encoding a fusion protein comprising a modified interleukin 2 (IL-2) polypeptide are provided, wherein the modified IL-2 polypeptide has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100% sequence identity in the region of amino acid residues 80-100 to the corresponding region of a comparable IL-2 polypeptide comprising an amino acid sequence set forth in SEQ ID NO:1 or SEQ ID NO:2 without the substitution.

In some embodiments, polynucleotides comprising a nucleic acid sequence encoding a fusion protein comprising a modified interleukin 2 (IL-2) polypeptide are provided, wherein the modified IL-2 polypeptide has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100% sequence identity in the region of amino acid residues 100-134 to the corresponding region of a comparable IL-2 polypeptide comprising an amino acid sequence set forth in SEQ ID NO:1 or SEQ ID NO:2 without the substitution.

In some embodiments, polynucleotides comprising a nucleic acid sequence encoding a fusion protein comprising a modified interleukin 2 (IL-2) polypeptide are provided, wherein the modified IL-2 polypeptide has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100% sequence identity in the regions of amino acid residues 10-25 and 80-100 to the corresponding regions of a comparable IL-2 polypeptide comprising an amino acid sequence set forth in SEQ ID NO:1 or SEQ ID NO:2 without the substitution.

In some embodiments, polynucleotides comprising a nucleic acid sequence encoding a fusion protein comprising a modified interleukin 2 (IL-2) polypeptide are provided, wherein the modified IL-2 polypeptide has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100% sequence identity in the regions of amino acid residues 10-25 and 100-134 to the corresponding regions of a comparable IL-2 polypeptide comprising an amino acid sequence set forth in SEQ ID NO:1 or SEQ ID NO:2 without the substitution.

In some embodiments, polynucleotides comprising a nucleic acid sequence encoding a fusion protein comprising a modified interleukin 2 (IL-2) polypeptide are provided, wherein the modified IL-2 polypeptide has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100% sequence identity in the regions of amino acid residues 80-100 and 100-134 to the corresponding regions of a comparable IL-2 polypeptide comprising an amino acid sequence set forth in SEQ ID NO:1 or SEQ ID NO:2 without the substitution.

In some embodiments, polynucleotides comprising a nucleic acid sequence encoding a fusion protein comprising a modified interleukin 2 (IL-2) polypeptide are provided, wherein the modified IL-2 polypeptide has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100% sequence identity in the regions of amino acid residues 10-25, 80-100 and 100-134 to the corresponding regions of a comparable IL-2 polypeptide comprising an amino acid sequence set forth in SEQ ID NO:1 or SEQ ID NO:2 without the substitution.

In some embodiments, polynucleotides comprising a nucleic acid sequence encoding a fusion protein comprising a modified interleukin 2 (IL-2) polypeptide are provided, wherein the modified IL-2 polypeptide has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100% sequence identity to a comparable IL-2 polypeptide comprising an amino acid sequence set forth in SEQ ID NO:1 or SEQ ID NO:2 without the substitution.

In some embodiments, polynucleotides comprising a nucleic acid sequence encoding a fusion protein comprising a modified interleukin 2 (IL-2) polypeptide are provided, wherein the modified interleukin 2 (IL-2) polypeptide comprises any suitable substitution with a natural amino acid. In some embodiments, polynucleotides comprising a nucleic acid sequence encoding a fusion protein comprising a modified interleukin 2 (IL-2) polypeptide are provided, wherein the modified interleukin 2 (IL-2) polypeptide comprises a substitution with lysine, cysteine, histidine, arginine, aspartic acid, glutamic acid, serine, threonine, alanine, tryptophan, isoleucine, phenylalanine, or tyrosine at a position of Q13, L19, N29, N30, Y31, K32, N33, P34, K35, T37, R38, T41, F42, K43, Y45, K48, K49, E62, K64, P65, N71, Q74, K76, R81, L85, S87, V91, I92, V93, or a combination thereof.

In some embodiments, polynucleotides comprising a nucleic acid sequence encoding a fusion protein comprising a modified interleukin 2 (IL-2) polypeptide are provided, wherein the modified interleukin 2 (IL-2) polypeptide a) comprises a substitution with a natural amino acid at a position selected from the group consisting of N29, N30, Y31, K32, N33, P34, K35, R38, T41, F42, K43, Y45, K48, K49, E62, K64, P65, N71, Q74, K76 and a combination thereof, and is configured to be conjugated to a water-soluble polymer, a lipid, a protein, or a peptide at the position selected from the group consisting of N29, N30, Y31, K32, N33, P34, K35, R38, T41, F42, K43, Y45, K48, K49, E62, K64, P65, N71, Q74, K76 and a combination thereof, and/or b) comprises a substitution with a natural amino acid at a position selected from the group consisting of N29, N30, Y31, K32, N33, P34, K35, R38, T41, F42, K43, Y45, K48, K49, E62, K64, P65, N71, Q74, K76 and a combination thereof, and is configured to be conjugated to a water-soluble polymer, a lipid, a protein, or a peptide at the N terminal and/or C terminal of the polypeptide.

In some embodiments, polynucleotides comprising a nucleic acid sequence encoding a fusion protein comprising a modified interleukin 2 (IL-2) polypeptide are provided, wherein the modified interleukin 2 (IL-2) polypeptide: a) comprises a substitution with lysine, cysteine, histidine, arginine, aspartic acid, glutamic acid, serine, threonine, alanine, tryptophan, isoleucine, phenylalanine, or tyrosine at a position selected from the group consisting of N29, N30, Y31, K32, N33, P34, K35, R38, T41, F42, K43, Y45, K48, K49, E62, K64, P65, N71, Q74, K76 and a combination thereof; and/or b) comprises a substitution with lysine, cysteine, histidine, arginine, aspartic acid, glutamic acid, serine, threonine, alanine, tryptophan, isoleucine, phenylalanine, or tyrosine at a position selected from the group consisting of N29, N30, Y31, N33, P34, K35, R38, T41, K43, K48, K49, K64, P65, N71, Q74, K76 and a combination thereof.

In some embodiments, polynucleotides comprising a nucleic acid sequence encoding a fusion protein comprising a modified interleukin 2 (IL-2) polypeptide are provided, wherein the modified interleukin 2 (IL-2) polypeptide: a) comprises a substitution with cysteine at a position selected from the group consisting of N29, N30, Y31, N33, P34, K35, R38, T41, K43, K48, K49, K64, P65, N71, Q74, K76 and a combination thereof, b) comprises a substitution with cysteine at a position selected from the group consisting of N29, Y31, K35, P65, N71, Q74 and a combination thereof; c) comprises a substitution with cysteine at a position of Y31; and/or d) comprises a substitution with cysteine at a position of P65.

In some embodiments, polynucleotides comprising a nucleic acid sequence encoding a fusion protein comprising a modified interleukin 2 (IL-2) polypeptide are provided, wherein the modified interleukin 2 (IL-2) polypeptide comprises a substitution with any amino acid at a position Y31. In some embodiments, polynucleotides comprising a nucleic acid sequence encoding a fusion protein comprising a modified interleukin 2 (IL-2) polypeptide are provided, wherein the modified interleukin 2 (IL-2) polypeptide comprises a substitution with serine or alanine at a position Y31.

In some embodiments, polynucleotides comprising a nucleic acid sequence encoding a fusion protein comprising a modified interleukin 2 (IL-2) polypeptide are provided, wherein the modified interleukin 2 (IL-2) polypeptide comprises a substitution with a natural amino acid or an unnatural amino acid at a position within IL-2Rα interaction region, IL-2Rβ interaction region and/or IL-2Rγ interaction region.

In some embodiments, polynucleotides comprising a nucleic acid sequence encoding a fusion protein comprising a modified interleukin 2 (IL-2) polypeptide are provided, wherein the modified interleukin 2 (IL-2) polypeptide comprises a substitution with a natural amino acid at a position within IL-2Rα interaction region. In some embodiments, polynucleotides comprising a nucleic acid sequence encoding a fusion protein comprising a modified interleukin 2 (IL-2) polypeptide are provided, wherein the modified interleukin 2 (IL-2) polypeptide further comprises a substitution with a natural amino acid at any suitable position within IL-2Rα interaction region. In some embodiments, polynucleotides comprising a nucleic acid sequence encoding a fusion protein comprising a modified interleukin 2 (IL-2) polypeptide are provided, wherein the modified interleukin 2 (IL-2) polypeptide further comprises a substitution with a natural amino acid at a position selected from the group consisting of R38, F42, Y45, E62, P65 and a combination thereof.

In some embodiments, polynucleotides comprising a nucleic acid sequence encoding a fusion protein comprising a modified interleukin 2 (IL-2) polypeptide are provided, wherein the modified interleukin 2 (IL-2) polypeptide comprises any suitable substitution with a natural amino acid at a position within IL-2Rα interaction region. In some embodiments, polynucleotides comprising a nucleic acid sequence encoding a fusion protein comprising a modified interleukin 2 (IL-2) polypeptide are provided, wherein the modified interleukin 2 (IL-2) polypeptide comprises a substitution with lysine, cysteine, histidine, arginine, aspartic acid, glutamic acid, serine, threonine, alanine, tryptophan, isoleucine, phenylalanine, or tyrosine at a position selected from the group consisting of R38, F42, Y45, E62, P65 and a combination thereof.

In some embodiments, polynucleotides comprising a nucleic acid sequence encoding a fusion protein comprising a modified interleukin 2 (IL-2) polypeptide are provided, wherein the modified interleukin 2 (IL-2) polypeptide: a) comprises a substitution with cysteine at a position selected from the group consisting of R38, F42, Y45, E62, P65 and a combination thereof, b) comprises a substitution with alanine, lysine or serine at a position of F42; c) comprises a substitution with alanine at a position of F42; d) comprises a substitution with serine at a position of F42; e) comprises a substitution with lysine at a position of F42; f) comprises a substitution with alanine, histidine or serine at a position of Y45; g) comprises a substitution with alanine at a position of Y45; h) comprises a substitution with histidine at a position of Y45; i) comprises a substitution with alanine, aspartic acid or serine at a position of R38; j) comprises a substitution with aspartic acid at a position of R38; k) comprises a substitution with alanine at a position of P65; 1) comprises a substitution with serine at a position of P65; m) comprises a substitution with alanine at a position of E62; and/or n) comprises a substitution with lysine at a position of F42, a substitution with cysteine at position of Y31, or a combination thereof.

In some embodiments, polynucleotides comprising a nucleic acid sequence encoding a fusion protein comprising a modified interleukin 2 (IL-2) polypeptide are provided, wherein the modified interleukin 2 (IL-2) polypeptide comprises a substitution with a natural amino acid at a position within IL-2Rβ interaction region. In some embodiments, polynucleotides comprising a nucleic acid sequence encoding a fusion protein comprising a modified interleukin 2 (IL-2) polypeptide are provided, wherein the modified interleukin 2 (IL-2) polypeptide further comprises a substitution with a natural amino acid at any suitable position within IL-2Rβ interaction region. In some embodiments, polynucleotides comprising a nucleic acid sequence encoding a fusion protein comprising a modified interleukin 2 (IL-2) polypeptide are provided, wherein the modified interleukin 2 (IL-2) polypeptide further comprises a substitution with a natural amino acid at a position selected from the group consisting of Q13, L19, R81, L85, S87, V91, I92, V93 and a combination thereof.

In some embodiments, polynucleotides comprising a nucleic acid sequence encoding a fusion protein comprising a modified interleukin 2 (IL-2) polypeptide are provided, wherein the modified interleukin 2 (IL-2) polypeptide comprises any suitable substitution with a natural amino acid at a position within IL-2Rβ interaction region. In some embodiments, polynucleotides comprising a nucleic acid sequence encoding a fusion protein comprising a modified interleukin 2 (IL-2) polypeptide are provided, wherein the modified interleukin 2 (IL-2) polypeptide comprises a substitution with lysine, cysteine, histidine, arginine, aspartic acid, glutamic acid, serine, threonine, alanine, tryptophan, isoleucine, phenylalanine, or tyrosine at a position selected from the group consisting of Q13, L19, R81, L85, S87, V91, I92, V93 and a combination thereof. In some embodiments, polynucleotides comprising a nucleic acid sequence encoding a fusion protein comprising a modified interleukin 2 (IL-2) polypeptide are provided, wherein the modified interleukin 2 (IL-2) polypeptide comprises a substitution with cysteine at a position selected from the group consisting of Q13, L19, R81, L85, S87, V91, I92, V93 and a combination thereof.

In some embodiments, polynucleotides comprising a nucleic acid sequence encoding a fusion protein comprising a modified interleukin 2 (IL-2) polypeptide are provided, wherein the modified interleukin 2 (IL-2) polypeptide comprises: a) a substitution with a natural amino acid at a position within IL-2Rα interaction region and a substitution with a natural amino acid at a position within IL-2Rβ interaction region; b) a substitution with a natural amino acid at a position within IL-2Rα interaction region and a substitution with a natural amino acid at a position within IL-2Rγ interaction region; or c) a substitution with a natural amino acid at a position within IL-2Rα interaction region, a substitution with a natural amino acid at a position within IL-2Rβ interaction region and a substitution with a natural amino acid at a position within IL-2R7 interaction region.

In some embodiments, polynucleotides comprising a nucleic acid sequence encoding a fusion protein comprising a modified interleukin 2 (IL-2) polypeptide are provided, wherein the modified interleukin 2 (IL-2) polypeptide comprises any suitable substitution with an unnatural amino acid. Exemplary unnatural amino acids that may be employed in accordance with the embodiments disclosed herein and throughout are described, for example, in WO 2019/028425 A1 and WO 2019/028419 A1, which are hereby incorporated herein in their entireties for all purposes. In some embodiments, polynucleotides comprising a nucleic acid sequence encoding a fusion protein comprising a modified interleukin 2 (IL-2) polypeptide are provided, wherein the modified interleukin 2 (IL-2) polypeptide comprises an unnatural amino acid and or an amino acid comprising an unnatural functional group such as, for example: a lysine analogue, a cysteine analogue or a histidine analogue; an aromatic side chain; an azido group; an alkyne group; and/or an aldehyde or ketone group. In some embodiments, polynucleotides comprising a nucleic acid sequence encoding a fusion protein comprising a modified interleukin 2 (IL-2) polypeptide are provided, wherein the modified interleukin 2 (IL-2) polypeptide comprises an unnatural amino acid that does not comprise an aromatic side chain. In some embodiments, polynucleotides comprising a nucleic acid sequence encoding a fusion protein comprising a modified interleukin 2 (IL-2) polypeptide are provided, wherein the modified interleukin 2 (IL-2) polypeptide comprises an unnatural amino acid that comprises N6-azidoethoxy-L-lysine (AzK), N6-propargylethoxy-L-lysine (PraK), BCN-L-lysine, norbornene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, or selenocysteine.

In some embodiments, polynucleotides comprising a nucleic acid sequence encoding a fusion protein comprising a modified interleukin 2 (IL-2) polypeptide are provided, wherein the modified interleukin 2 (IL-2) polypeptide comprises an unnatural amino acid that can be incorporated into the modified IL-2 polypeptide by any suitable means or methods. In some embodiments, polynucleotides comprising a nucleic acid sequence encoding a fusion protein comprising a modified interleukin 2 (IL-2) polypeptide are provided, wherein the modified interleukin 2 (IL-2) polypeptide comprises an unnatural amino acid that can be incorporated into the modified IL-2 polypeptide by an orthogonal tRNA synthetase/tRNA pair. Any suitable orthogonal tRNA can be used. For example, the orthogonal tRNA of the orthogonal synthetase/tRNA pair can comprise at least one unnatural nucleobase.

In some embodiments, polynucleotides comprising a nucleic acid sequence encoding a fusion protein comprising a modified interleukin 2 (IL-2) polypeptide are provided, wherein the modified interleukin 2 (IL-2) polypeptide has reduced or no detectable binding to an IL-2Rα compared to a comparable IL-2 polypeptide comprising an amino acid sequence set forth in SEQ ID NO:1 or SEQ ID NO:2 without the substitution. In some embodiments, polynucleotides comprising a nucleic acid sequence encoding a fusion protein comprising a modified interleukin 2 (IL-2) polypeptide are provided, wherein the binding affinity of the modified IL-2 polypeptide to an IL-2Rα can be decreased from about 10% to about 100%, e.g., decreased by about 10%, 20%, 30%, 40%, 50%, 60%, 17%, 80%, 90%, 100%, or a subrange thereof. In some embodiments, polynucleotides comprising a nucleic acid sequence encoding a fusion protein comprising a modified interleukin 2 (IL-2) polypeptide are provided, wherein the binding affinity of the modified IL-2 polypeptide to an IL-2Rα can be decreased from about 10% to about 100%, or can be decreased from about 1 fold to about 100,000 fold or more, e.g., decreased by about 1 fold, 10 fold, 100 fold, 1,000 fold, 10,000 fold, 100,000 fold or more, or a subrange thereof. In some embodiments, polynucleotides comprising a nucleic acid sequence encoding a fusion protein comprising a modified interleukin 2 (IL-2) polypeptide are provided, wherein the modified interleukin 2 (IL-2) polypeptide has no detectable binding to an IL-2Rα.

In some embodiments, polynucleotides comprising a nucleic acid sequence encoding a fusion protein comprising a modified interleukin 2 (IL-2) polypeptide are provided, wherein the modified interleukin 2 (IL-2) polypeptide has reduced or no detectable receptor signaling potency to IL-2Rαβγ compared to a comparable IL-2 polypeptide comprising an amino acid sequence set forth in SEQ ID NO:1 or SEQ ID NO:2 without the substitution. In some embodiments, polynucleotides comprising a nucleic acid sequence encoding a fusion protein comprising a modified interleukin 2 (IL-2) polypeptide are provided, wherein a ratio between the signaling potency to IL-2Rαβγ of the modified IL-2 polypeptide and the signaling potency to IL-2Rαβγ of the comparable IL-2 polypeptide comprising an amino acid sequence set forth in SEQ ID NO:1 or SEQ ID NO:2 without the substitution can be from about 1/2 to about 1/100,000, e.g., at about 1/2, 1/5, 1/10, 1/100, 1/1,000, 1/10,000, 1/100,000, or more, or a subrange thereof. In some embodiments, polynucleotides comprising a nucleic acid sequence encoding a fusion protein comprising a modified interleukin 2 (IL-2) polypeptide are provided, wherein, the modified IL-2 polypeptide has no detectable receptor signaling potency to IL-2Rαβγ.

In some embodiments, polynucleotides comprising a nucleic acid sequence encoding a fusion protein comprising a modified interleukin 2 (IL-2) polypeptide are provided, wherein modified the IL-2 polypeptide has reduced binding to an IL-2Rα compared to a comparable IL-2 polypeptide comprising an amino acid sequence set forth in SEQ ID NO:1 or SEQ ID NO:2 without the substitution and has reduced receptor signaling potency to IL-2Rαβγ compared to a comparable IL-2 polypeptide comprising an amino acid sequence set forth in SEQ ID NO:1 or SEQ ID NO:2 without the substitution. In some embodiments, polynucleotides comprising a nucleic acid sequence encoding a fusion protein comprising a modified interleukin 2 (IL-2) polypeptide are provided, wherein the modified IL-2 polypeptide has no detectable binding to an IL-2Rα and has no detectable receptor signaling potency to IL-2Rαβγ.

In some embodiments, polynucleotides comprising a nucleic acid sequence encoding a fusion protein comprising a modified interleukin 2 (IL-2) polypeptide are provided, wherein the modified IL-2 polypeptide can retain substantial or can have higher binding level to an interleukin 2 receptor β (IL-2R β) or an interleukin 2 receptor γ (IL-2R γ) compared to a comparable IL-2 polypeptide comprising an amino acid sequence set forth in SEQ ID NO:1 or SEQ ID NO:2 without the substitution, and/or can retain substantial or can have higher receptor signaling potency to IL-2R γ compared to a comparable IL-2 polypeptide comprising an amino acid sequence set forth in SEQ ID NO:1 or SEQ ID NO:2 without the substitution. In some embodiments, polynucleotides comprising a nucleic acid sequence encoding a fusion protein comprising a modified interleukin 2 (IL-2) polypeptide are provided, wherein the modified IL-2 polypeptide retains substantial or has higher binding level to an IL-2R β or an IL-2R γ compared to a comparable IL-2 polypeptide comprising an amino acid sequence set forth in SEQ ID NO:1 or SEQ ID NO:2 without the substitution. In some embodiments, polynucleotides comprising a nucleic acid sequence encoding a fusion protein comprising a modified interleukin 2 (IL-2) polypeptide are provided, wherein the modified IL-2 polypeptide retains substantial or has higher receptor signaling potency to IL-2R βγ compared to a comparable IL-2 polypeptide comprising an amino acid sequence set forth in SEQ ID NO:1 or SEQ ID NO:2 without the substitution. In some embodiments, polynucleotides comprising a nucleic acid sequence encoding a fusion protein comprising a modified interleukin 2 (IL-2) polypeptide are provided, wherein the modified IL-2 polypeptide retains substantial or has higher binding level to an IL-2R β or an IL-2R γ compared to a comparable IL-2 polypeptide comprising an amino acid sequence set forth in SEQ ID NO:1 or SEQ ID NO:2 without the substitution, and retains substantial or has higher receptor signaling potency to IL-2R βγ compared to a comparable IL-2 polypeptide comprising an amino acid sequence set forth in SEQ ID NO:1 or SEQ ID NO:2 without the substitution.

In some embodiments, polynucleotides comprising a nucleic acid sequence encoding a fusion protein comprising a modified interleukin 2 (IL-2) polypeptide are provided, wherein the modified IL-2 polypeptide comprises a deletion at any suitable location. In some embodiments, polynucleotides comprising a nucleic acid sequence encoding a fusion protein comprising a modified interleukin 2 (IL-2) polypeptide are provided, wherein the modified IL-2 polypeptide has a N terminal deletion, e.g., a N terminal deletion of amino acid residues 1-30 or a subrange thereof. In some embodiments, polynucleotides comprising a nucleic acid sequence encoding a fusion protein comprising a modified interleukin 2 (IL-2) polypeptide are provided, wherein the modified IL-2 polypeptide has a C terminal deletion, e.g., a C terminal deletion of amino acid residues 114-134 or a subrange thereof. In some embodiments, polynucleotides comprising a nucleic acid sequence encoding a fusion protein comprising a modified interleukin 2 (IL-2) polypeptide are provided, wherein the modified IL-2 polypeptide has a N terminal deletion and a C terminal deletion.

In some embodiments, polynucleotides comprising a nucleic acid sequence encoding a fusion protein comprising a modified interleukin 2 (IL-2) polypeptide are provided, wherein the fusion protein comprises a recombinant fusion protein. In some embodiments, polynucleotides comprising a nucleic acid sequence encoding a fusion protein comprising a modified interleukin 2 (IL-2) polypeptide are provided, wherein the fusion polypeptide further comprises an additional amino acid sequence. In some embodiments, polynucleotides comprising a nucleic acid sequence encoding a fusion protein comprising a modified interleukin 2 (IL-2) polypeptide are provided, wherein the modified IL-2 polypeptide is fused to the additional amino acid sequence in any suitable manner. In some embodiments, polynucleotides comprising a nucleic acid sequence encoding a fusion protein comprising a modified interleukin 2 (IL-2) polypeptide are provided, wherein the modified interleukin 2 (IL-2) polypeptide is fused to the additional amino acid sequence via the N-terminus of the modified IL-2 polypeptide or via the C-terminus of the modified IL-2 polypeptide. The additional amino acid sequence can comprise any suitable sequence or content. In some embodiments, polynucleotides comprising a nucleic acid sequence encoding a fusion protein comprising a modified interleukin 2 (IL-2) polypeptide are provided, wherein the additional amino acid sequence comprises an antibody sequence or a portion or a fragment thereof. In some embodiments, polynucleotides comprising a nucleic acid sequence encoding a fusion protein comprising a modified interleukin 2 (IL-2) polypeptide are provided, wherein the additional amino acid sequence comprises an Fc portion of an antibody.

In some embodiments, polynucleotides comprising a nucleic acid sequence encoding a fusion protein comprising a modified interleukin 2 (IL-2) polypeptide are provided, wherein the fusion protein further comprises an additional amino acid sequence that comprises a serum albumin or a PK-extending fragment or analog thereof. In some embodiments, polynucleotides comprising a nucleic acid sequence encoding a fusion protein comprising a modified interleukin 2 (IL-2) polypeptide are provided, wherein the fusion protein further comprises an additional amino acid sequence that comprises a human serum albumin (HSA); b) a murine serum albumin (MSA); c) or a PK-extending fragment or analog of a) or b).

In some embodiments, polynucleotides comprising a nucleic acid sequence encoding a fusion protein comprising a modified interleukin 2 (IL-2) polypeptide are provided, wherein the fusion protein further comprises an additional amino acid sequence that comprises the human serum albumin (HSA) set forth in SEQ ID NO:7; b) the murine serum albumin (MSA) set forth in SEQ ID NO:6; c) or a PK-extending fragment or analog of a) or b).

In some embodiments, polynucleotides comprising a nucleic acid sequence encoding a fusion protein comprising a modified interleukin 2 (IL-2) polypeptide are provided, wherein the polynucleotide is in an isolated form and/or in a purified form.

In some embodiments, polynucleotides comprising a nucleic acid sequence encoding a fusion protein comprising a modified interleukin 2 (IL-2) polypeptide are provided, wherein the polynucleotide is prepared using any suitable technique or process. In some embodiments, polynucleotides comprising a nucleic acid sequence encoding a fusion protein comprising a modified interleukin 2 (IL-2) polypeptide are provided, wherein the polynucleotide is prepared by recombinant production, chemical synthesis or a combination thereof.

In some embodiments, polynucleotides comprising a nucleic acid sequence encoding a fusion protein comprising a modified interleukin 2 (IL-2) polypeptide are provided, wherein the polynucleotide comprises DNA, RNA, a DNA/RNA hybrid, a viral vector, a non-viral vector, a plasmid, and/or a Nanoplasmid™. In some embodiments, polynucleotides comprising a nucleic acid sequence encoding a fusion protein comprising a modified interleukin 2 (IL-2) polypeptide are provided, wherein the polynucleotide is configured to express the fusion protein in vitro, in vivo, and/or ex vivo.

In some embodiments, polynucleotides comprising a nucleic acid sequence encoding a fusion protein comprising a modified interleukin 2 (IL-2) polypeptide are provided, wherein the modified IL-2 polypeptide and/or the fusion protein, once expressed, may form a conjugate with another moiety in vivo, ex vivo, or in vitro

In some embodiments, polynucleotides comprising a nucleic acid sequence encoding a fusion protein comprising a modified interleukin 2 (IL-2) polypeptide are provided, wherein the polynucleotide is prepared as and/or included in a pharmaceutical composition comprising one or more lipids and/or lipid nanoparticles (LNPs).

In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to another moiety, e.g., a water-soluble polymer, a lipid, a polypeptide, e.g., a protein, or a peptide. Such conjugation may be performed or may occur in vitro or in vivo.

In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to another moiety, e.g., a water-soluble polymer, a lipid, a protein, or a peptide, in any suitable manner. For example, the modified IL-2 polypeptide can be conjugated to a water-soluble polymer, a lipid, a protein, or a peptide covalently. In another example, the modified IL-2 polypeptide can be conjugated to a water-soluble polymer, a lipid, a protein, or a peptide non-covalently. In still another example, the modified IL-2 polypeptide can be conjugated to a water-soluble polymer, a lipid, a protein, or a peptide via a substituted natural amino acid or unnatural amino acid at any suitable position.

In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to another moiety, e.g., a water-soluble polymer, a lipid, a protein, or a peptide, via a substituted natural amino acid or unnatural amino acid at a position selected from the group consisting of Q13, L19, N29, N30, Y31, K32, N33, P34, K35, T37, R38, T41, F42, K43, Y45, K48, K49, E62, K64, P65, N71, Q74, K76, R81, L85, S87, V91, I92, V93 and a combination thereof. In another embodiment, the modified IL-2 polypeptide is conjugated to another moiety, e.g., a water-soluble polymer, a lipid, a protein, or a peptide, via a substituted natural amino acid at a position selected from the group consisting of Q13, L19, N29, N30, Y31, K32, N33, P34, K35, T37, R38, T41, F42, K43, Y45, K48, K49, E62, K64, P65, N71, Q74, K76, R81, L85, S87, V91, I92, V93 and a combination thereof. In still another embodiment, the modified IL-2 polypeptide is conjugated to another moiety, e.g., a water-soluble polymer, a lipid, a protein, or a peptide, via a substituted lysine, cysteine, histidine, arginine, aspartic acid, glutamic acid, serine, threonine, alanine, tryptophan, isoleucine, phenylalanine, or tyrosine at a position selected from the group consisting of Q13, L19, N29, N30, Y31, K32, N33, P34, K35, T37, R38, T41, F42, K43, Y45, K48, K49, E62, K64, P65, N71, Q74, K76, R81, L85, S87, V91, I92, V93 and a combination thereof. In yet another embodiment, the modified IL-2 polypeptide is conjugated to another moiety, e.g., a water-soluble polymer, a lipid, a protein, or a peptide, via a substituted cysteine at a position selected from the group consisting of Q13, L19, N29, N30, Y31, K32, N33, P34, K35, T37, R38, T41, F42, K43, Y45, K48, K49, E62, K64, P65, N71, Q74, K76, R81, L85, S87, V91, 192, V93 and a combination thereof.

In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to another moiety, e.g., a water-soluble polymer, a lipid, a protein, or a peptide, via a substituted natural amino acid or unnatural amino acid at a position selected from the group consisting of N29, N30, Y31, K32, N33, P34, K35, R38, T41, F42, K43, Y45, K48, K49, E62, K64, P65, N71, Q74, K76 and a combination thereof. In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to another moiety, e.g., a water-soluble polymer, a lipid, a protein, or a peptide, via a substituted natural amino acid at a position selected from the group consisting of N29, N30, Y31, K32, N33, P34, K35, R38, T41, F42, K43, Y45, K48, K49, E62, K64, P65, N71, Q74, K76 and a combination thereof. In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to another moiety, e.g., a water-soluble polymer, a lipid, a protein, or a peptide, via a substituted lysine, cysteine, histidine, arginine, aspartic acid, glutamic acid, serine, threonine, alanine, tryptophan, isoleucine, phenylalanine, or tyrosine at a position selected from the group consisting of N29, N30, Y31, K32, N33, P34, K35, R38, T41, F42, K43, Y45, K48, K49, E62, K64, P65, N71, Q74, K76 and a combination thereof. In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to another moiety, e.g., a water-soluble polymer, a lipid, a protein, or a peptide, via a substituted cysteine at a position selected from the group consisting of N29, N30, Y31, K32, N33, P34, K35, R38, T41, F42, K43, Y45, K48, K49, E62, K64, P65, N71, Q74, K76 and a combination thereof.

In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to another moiety, e.g., a water-soluble polymer, a lipid, a protein, or a peptide, via a single amino acid residue or multiple amino acid residues of the modified IL-2 polypeptide. In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to another moiety, e.g., a water-soluble polymer, a lipid, a protein, or a peptide, via: i) the alpha amino group of the N-terminal amino acid residue of the modified IL-2 polypeptide; ii) the epsilon amino group of a lysine amino acid residue of the modified IL-2 polypeptide; or iii) an N-glycosylation site or O-glycosylation site of the modified IL-2 polypeptide.

In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to another moiety, e.g., a water-soluble polymer, a lipid, a protein, or a peptide, through a linker. In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to another moiety, e.g., a water-soluble polymer, a lipid, a protein, or a peptide, directly without a linker.

In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to another moiety, e.g., a water-soluble polymer, a lipid, a protein, or a peptide, via a single amino acid residue in a fusion polypeptide that comprises the modified IL-2 polypeptide and an additional amino acid sequence. The single amino acid residue can be located at any suitable location. For example, the single amino acid residue can be located within the modified IL-2 polypeptide. In another example, the single amino acid residue can be located within the additional amino acid sequence.

The additional amino acid sequence in the present modified IL-2 polypeptide conjugate can comprise any suitable sequence or content. For example, the additional amino acid sequence in the present modified IL-2 polypeptide conjugate can comprise an antibody sequence or a portion or a fragment thereof. In another example, the additional amino acid sequence in the present modified IL-2 polypeptide conjugate can comprise a Fc portion of an antibody.

In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to another moiety, e.g., a water-soluble polymer, a lipid, a protein, or a peptide in a fusion polypeptide, in any suitable manner. In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to another moiety, e.g., a water-soluble polymer, a lipid, a protein, or a peptide, via: i) the alpha amino group of the N-terminal amino acid residue of the fusion polypeptide; ii) the epsilon amino group of a lysine amino acid residue of the fusion polypeptide; or iii) an N-glycosylation site or O-glycosylation site of the fusion polypeptide. In another example, the fusion polypeptide can be covalently conjugated to a water-soluble polymer, a lipid, a protein, or a peptide directly or through a linker.

In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to any suitable water-soluble polymer. For example, the water-soluble polymer can comprise polyethylene glycol (PEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(a-hydroxy acid), poly(vinyl alcohol), polyphosphazene, polyoxazolines (POZ), poly(N-acryloylmorpholine), or a combination thereof. See e.g., WO 2019/028425A1 and WO 2019/028419A1.

In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to a PEG molecule. The PEG molecule can be a linear PEG or a branched PEG. The branched PEG can have any suitable configuration and/or any suitable number of PEG chains. For example, the branched PEG can have about three to about ten PEG chains emanating from a central core group. In another example, the branched PEG can be a star PEG comprising from about 10 to about 100 PEG chains emanating from a central core group. In still another example, the branched PEG can be a comb PEGs comprising multiple PEG chains grafted onto a polymer backbone.

In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to a PEG molecule of any suitable molecular weight. For example, the PEG molecule can have a range of molecular weight from about 300 g/mol to about 10,000,000 g/mol, e.g., at about 300 g/mol, 500 g/mol, 1,000 g/mol, 10,000 g/mol, 100,000 g/mol, 1,000,000 g/mol, 10,000,000 g/mol or a subrange thereof. In another example, the PEG molecule can have an average molecular weight from about 5,000 Daltons to about 1,000,000 Daltons, e.g., at about 5,000 Daltons, 10,000 Daltons, 100,000 Daltons, 1,000,000 Daltons or a subrange thereof. In still another example, the PEG molecule can have an average molecular weight of from about 20,000 Daltons to about 30,000 Daltons, e.g., at about 20,000 Daltons, 21,000 Daltons, 22,000 Daltons, 23,000 Daltons, 24,000 Daltons, 25,000 Daltons, 26,000 Daltons, 27,000 Daltons, 28,000 Daltons, 29,000 Daltons, 30,000 Daltons or a subrange thereof.

In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to a PEG molecule in any suitable form. For example, the PEG molecule can be a monodisperse, uniform, or discrete PEG molecule.

In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to a polysaccharide.

In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to any suitable lipid. In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to a fatty acid.

In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to any suitable protein. In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to an antibody or a binding fragment thereof. In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to an Fc portion of an antibody.

In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to, e.g., a water-soluble polymer, a lipid, a protein, or a peptide, which can be bound to the modified IL-2 polypeptide via any suitable manner. In some embodiments, polynucleotides comprising a nucleic acid sequence encoding a fusion protein comprising a modified interleukin 2 (IL-2) polypeptide are provided, wherein the modified IL-2 polypeptide, as described above, is configured to be conjugated to, e.g., a water-soluble polymer, a lipid, a protein, or a peptide, which can be indirectly bound to the substituted natural amino acid or unnatural amino acid of the modified IL-2 polypeptide through a linker. In another example, the other moiety, e.g., a water-soluble polymer, a lipid, a protein, or a peptide, can be directly bound to the substituted natural amino acid or unnatural amino acid of the modified IL-2 polypeptide.

The present modified IL-2 polypeptide conjugate can have any suitable half-life in vivo. For example, the present modified IL-2 polypeptide conjugate can have a half-life in vivo from about 5 minutes to about 10 days, e.g., at about 5 minutes, 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, 11 hour, 12 hours, 13 hours, 14 hours, 15 hours, 16 hours, 17 hours, 18 hours, 19 hours, 20 hours, 21 hour, 22 hours, 23 hours, 1 day, 2 days, 3 days, 4 days, 5 days, 6 days, 7 days, 8 days, 9 days, 10 days or a subrange thereof.

### Pharmaceutical Compositions

In some embodiments are provided pharmaceutical compositions comprising an polynucleotide comprising a nucleic acid sequence encoding a fusion protein comprising a modified interleukin 2 (IL-2) polypeptide, as described above, and a pharmaceutically acceptable carrier or excipient.

The present pharmaceutical composition can be configured to treat or prevent any suitable disease(s), disorder(s) or condition(s). For example, the present pharmaceutical composition can be configured to treat or prevent a proliferation disorder in a subject.

In one embodiment, the present pharmaceutical composition is configured to treat or prevent a solid tumor or cancer in a subject. The solid tumor or cancer can be Chondrosarcoma, Ewing's sarcoma, Malignant fibrous histiocytoma of bone/osteosarcoma, Osteosarcoma, Rhabdomyosarcoma, Heart cancer, Astrocytoma, Brainstem glioma, Pilocytic astrocytoma, Ependymoma, Primitive neuroectodermal tumor, Cerebellar astrocytoma, Cerebral astrocytoma, Glioma, Medulloblastoma, Neuroblastoma, Oligodendroglioma, Pineal astrocytoma, Pituitary adenoma, Visual pathway and hypothalamic glioma, Breast cancer, Invasive lobular carcinoma, Tubular carcinoma, Invasive cribriform carcinoma, Medullary carcinoma, Male breast cancer, Phyllodes tumor, Inflammatory Breast Cancer, Adrenocortical carcinoma, Islet cell carcinoma (endocrine pancreas), Multiple endocrine neoplasia syndrome, Parathyroid cancer, Pheochromocytoma, Thyroid cancer, Merkel cell carcinoma, Uveal melanoma, Retinoblastoma, Anal cancer, Appendix cancer, cholangiocarcinoma, Carcinoid tumor, gastrointestinal, Colon cancer, Extrahepatic bile duct cancer, Gallbladder cancer, Gastric (stomach) cancer, Gastrointestinal carcinoid tumor, Gastrointestinal stromal tumor (GIST), Hepatocellular cancer, Pancreatic cancer islet cell, Rectal cancer, Bladder cancer, Cervical cancer, Endometrial cancer, Extragonadal germ cell tumor, Ovarian cancer, Ovarian epithelial cancer (surface epithelial-stromal tumor), Ovarian germ cell tumor, Penile cancer, Renal cell carcinoma, Renal pelvis and ureter, transitional cell cancer, Prostate cancer, Testicular cancer, Gestational trophoblastic tumor, Ureter and renal pelvis, transitional cell cancer, Urethral cancer, Uterine sarcoma, Vaginal cancer, Vulvar cancer, Wilms tumor, Esophageal cancer, Head and neck cancer, Nasopharyngeal carcinoma, Oral cancer, Oropharyngeal cancer, Paranasal sinus and nasal cavity cancer, Pharyngeal cancer, Salivary gland cancer, Hypopharyngeal cancer, Basal-cell carcinoma, Melanoma, Skin cancer (non-melanoma), Bronchial adenomas/carcinoids, Small cell lung cancer, Mesothelioma, Non-small cell lung cancer, Pleuropulmonary blastoma, Laryngeal cancer, Thymoma and thymic carcinoma, AIDS-related cancers, Kaposi sarcoma, Epithelioid hemangioendothelioma (EHE), Desmoplastic small round cell tumor or Liposarcoma.

In another embodiment, the present pharmaceutical composition is configured to treat or prevent a hematological malignancy in a subject. The hematological malignancy can be hematological malignancy including: myeloid neoplasms, Leukemias, Lymphomas, Hodgkin lymphoma, Non-Hodgkin lymphoma, Anaplastic large cell lymphoma, Angioimmunoblastic T-cell lymphoma, Hepatosplenic T-cell lymphoma, B-cell lymphoma reticuloendotheliosis, Reticulosis, Microglioma, Diffuse large B-cell lymphoma, Follicular lymphoma, Mucosa-associated lymphatic tissue lymphoma, B-cell chronic lymphocytic leukemia, Mantle cell lymphoma, Burkitt lymphoma, Mediastinal large B cell lymphoma, Waldenstrom's macroglobulinemia, Nodal marginal zone B cell lymphoma, Splenic marginal zone lymphoma, Intravascular large B-cell lymphoma, Primary effusion lymphoma, Lymphomatoid granulomatosis, Nodular lymphocyte predominant Hodgkin's lymphoma, plasma cell leukemia, Acute erythraemia and erythroleukaemia, Acute erythremic myelosis, Acute erythroid leukemia, Heilmeyer-Schoner disease, Acute megakaryoblastic leukemia, Mast cell leukemia, Panmyelosis, Acute panmyelosis with myelofibrosis, Lymphosarcoma cell leukemia, Acute leukaemia of unspecified cell type, Blastic phase chronic myelogenous leukemia, Stem cell leukemia, Chronic leukaemia of unspecified cell type, Subacute leukaemia of unspecified cell type, Accelerated phase chronic myelogenous leukemia, Acute myeloid leukemia, Polycythemia vera, Acute promyelocytic leukemia, Acute basophilic leukemia, Acute eosinophilic leukemia, Acute lymphoblastic leukemia, Acute monocytic leukemia, Acute myeloblastic leukemia with maturation, Acute myeloid dendritic cell leukemia, Adult T-cell leukemia/lymphoma, Aggressive NK-cell leukemia, B-cell prolymphocytic leukemia, B-cell chronic lymphocytic leukemia, B-cell leukemia, Chronic myelogenous leukemia, Chronic myelomonocytic leukemia, Chronic neutrophilic leukemia, Chronic lymphocytic leukemia, Hairy cell leukemia, Chronic idiopathic myelofibrosis, Multiple myeloma, Kahler's disease, Myelomatosis, Solitary myeloma, Plasma cell leukemia, Plasmacytoma, extramedullary, Malignant plasma cell tumour NOS, Plasmacytoma NOS, Monoclonal gammopathy, Multiple Myeloma, Angiocentric immunoproliferative lesion, Lymphoid granulomatosis, Angioimmunoblastic lymphadenopathy, T-gamma lymphoproliferative disease, Waldenstrom's macroglobulinaemia, Alpha heavy chain disease, Gamma heavy chain disease, Franklin's disease, Immunoproliferative small intestinal disease, Mediterranean disease, Malignant immunoproliferative disease, unspecified, or Immunoproliferative disease NOS.

In still another embodiment, the present pharmaceutical composition is configured to treat or prevent an immune deficiency disease or disorder in a subject. The immune deficiency disease or disorder can be Agammaglobulinemia: X-Linked and Autosomal Recessive, Ataxia Telangiectasia, Chronic Granulomatous Disease and Other Phagocytic Cell Disorders, Common Variable Immune Deficiency, Complement Deficiencies, DiGeorge Syndrome, Hemophagocytic Lymphohistiocytosis (HLH), Hyper IgE Syndrome, Hyper IgM Syndromes, IgG Subclass Deficiency, Innate Immune Defects, NEMO Deficiency Syndrome, Selective IgA Deficiency, Selective IgM Deficiency, Severe Combined Immune, Deficiency and Combined Immune Deficiency, Specific Antibody Deficiency, Transient Hypogammaglobulinemia of Infancy, WHIM Syndrome (Warts, Hypogammaglobulinemia, Infections, and Myelokathexis), Wiskott-Aldrich Syndrome, Other Antibody Deficiency Disorders, Other Primary Cellular Immunodeficiencies, Severe combined immune deficiency (SCID), Common variable immune deficiency (CVID), Human immunodeficiency virus/acquired immune deficiency syndrome (HIV/AIDS), Drug-induced immune deficiency, Graft versus host syndrome, Primary Immune Deficiency Diseases (PIDDs) or Lymphopenia.

The present pharmaceutical composition can further comprise another active ingredient. The another active ingredient can the active ingredient to treat or prevent any suitable any suitable disease(s), disorder(s) or condition(s). For example, the another active ingredient can be an anti-neoplasm substance.

The additional active ingredient(s) may be formulated in a separate pharmaceutical composition from at least one exemplary modified IL-2 polypeptide or modified IL-2 polypeptide conjugate of the present disclosure or may be included with at least one exemplary modified IL-2 polypeptide or modified IL-2 polypeptide conjugate of the present disclosure in a single pharmaceutical composition.

The present pharmaceutical compositions can be formulated to be administered orally, parenterally, by inhalation, topically, rectally, nasally, buccally, vaginally, via an implanted reservoir, or other drug administration methods. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

A sterile injectable composition, such as a sterile injectable aqueous or oleaginous suspension, may be formulated according to techniques known in the art using suitable dispersing or wetting agents and suspending agents. The sterile injectable preparation may also be a sterile injectable solution or suspension in a non-toxic parenterally acceptable diluent or solvent. Among the acceptable vehicles and solvents that may be employed include mannitol, water, Ringer's solution and isotonic sodium chloride solution. Suitable carriers and other pharmaceutical composition components are typically sterile.

In addition, sterile, fixed oils are conventionally employed as a solvent or suspending medium (e.g., synthetic mono- or diglycerides). Fatty acids, such as oleic acid and its glyceride derivatives, are useful in the preparation of injectables, as are pharmaceutically acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Various emulsifying agents or bioavailability enhancers which are commonly used in the manufacture of pharmaceutically acceptable solid, liquid, or other dosage forms can also be used for the purpose of formulation.

A composition for oral administration may be any orally acceptable dosage form including, but not limited to, tablets, capsules, emulsions and aqueous suspensions, dispersions and solutions. In the case of tablets for oral use, commonly used carriers include lactose and corn starch. Lubricating agents, such as magnesium stearate, can also be added. For oral administration in a capsule form, useful diluents include lactose and dried corn starch. When aqueous suspensions or emulsions are administered orally, the active ingredient can be suspended or dissolved in an oily phase combined with emulsifying or suspending agents. If needed, certain sweetening, flavoring, or coloring agents can be added. A nasal aerosol or inhalation compositions can be prepared according to techniques well-known in the art of pharmaceutical formulation and can be prepared as solutions in, for example saline, employing suitable preservatives (for example, benzyl alcohol), absorption promoters to enhance bioavailability, and/or other solubilizing or dispersing agents known in the art.

Any suitable formulation of the compounds described herein can be prepared. See generally, Remington's Pharmaceutical Sciences, (2000) Hoover, J. E. editor, 20 th edition, Lippincott Williams and Wilkins Publishing Company, Easton, Pa., pages 780-857. A formulation is selected to be suitable for an appropriate route of administration. In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts. Pharmaceutically acceptable salts are obtained using standard procedures well known in the art, for example, by a sufficiently basic compound such as an amine with a suitable acid, affording a physiologically acceptable anion. Alkali metal (e.g., sodium, potassium or lithium) or alkaline earth metal (e.g., calcium) salts of carboxylic acids also are made.

Where contemplated compounds or substances are administered in a pharmacological composition, it is contemplated that the compounds or substances can be formulated in admixture with a pharmaceutically acceptable excipient and/or carrier. For example, contemplated compounds or substances can be administered orally as neutral compounds or substances or as pharmaceutically acceptable salts, or intravenously in a physiological saline solution. Conventional buffers such as phosphates, bicarbonates or citrates can be used for this purpose. Of course, one of ordinary skill in the art may modify the formulations within the teachings of the specification to provide numerous formulations for a particular route of administration. In particular, contemplated compounds or substances may be modified to render them more soluble in water or other vehicle, which for example, may be easily accomplished with minor modifications (salt formulation, esterification, etc.) that are well within the ordinary skill in the art. It is also well within the ordinary skill of the art to modify the route of administration and dosage regimen of a particular compound or substance in order to manage the pharmacokinetics of the present compounds or substances, e.g., the present modified IL-2 polypeptide(s) or modified IL-2 polypeptide conjugate(s), for maximum beneficial effect in a patient.

The present modified IL-2 polypeptide or modified IL-2 polypeptide conjugate may be soluble in organic solvents such as chloroform, dichloromethane, ethyl acetate, ethanol, methanol, isopropanol, acetonitrile, glycerol, N,N-dimethylformamide, N,N-dimetheylaceatmide, dimethylsulfoxide, etc. In one embodiment, the present invention provides formulations prepared by mixing the present modified IL-2 polypeptide or modified IL-2 polypeptide conjugate with a pharmaceutically acceptable carrier. In one aspect, the formulation may be prepared using a method comprising: a) dissolving a described compound or substance in a water-soluble organic solvent, a non-ionic solvent, a water-soluble lipid, a cyclodextrin, a vitamin such as tocopherol, a fatty acid, a fatty acid ester, a phospholipid, or a combination thereof, to provide a solution; and b) adding saline or a buffer containing 1-10% carbohydrate solution. In one example, the carbohydrate comprises dextrose. The pharmaceutical compositions obtained using the present methods are stable and useful for animal and clinical applications.

Illustrative examples of water soluble organic solvents for use in the present pharmaceutical compositions include and are not limited to polyethylene glycol (PEG), alcohols, acetonitrile, N-methyl-2-pyrrolidone, N,N-dimethylformamide, N,N-dimethylacetamide, dimethyl sulfoxide, or a combination thereof. Examples of alcohols include but are not limited to methanol, ethanol, isopropanol, glycerol, or propylene glycol.

Illustrative examples of water soluble non-ionic surfactants for use in the present pharmaceutical compositions include and are not limited to CREMOPHOR® EL, polyethylene glycol modified CREMOPHOR® (polyoxyethyleneglyceroltriricinoleat 35), hydrogenated CREMOPHOR® RH40, hydrogenated CREMOPHOR® RH60, PEG-succinate, polysorbate 20, polysorbate 80, SOLUTOL® HS (polyethylene glycol 660 12-hydroxystearate), sorbitan monooleate, poloxamer, LABRAFIL® (ethoxylated persic oil), LABRASOL® (capryl-caproyl macrogol-8-glyceride), GELUCIRE® (glycerol ester), SOFTIGEN® (PEG 6 caprylic glyceride), glycerin, glycol-polysorbate, or a combination thereof.

Illustrative examples of water soluble lipids for use in the present pharmaceutical compositions include but are not limited to vegetable oils, triglycerides, plant oils, or a combination thereof. Examples of lipid oils include but are not limited to castor oil, polyoxyl castor oil, corn oil, olive oil, cottonseed oil, peanut oil, peppermint oil, safflower oil, sesame oil, soybean oil, hydrogenated vegetable oil, hydrogenated soybean oil, a triglyceride of coconut oil, palm seed oil, and hydrogenated forms thereof, or a combination thereof.

Illustrative examples of fatty acids and fatty acid esters for use in the present pharmaceutical compositions include but are not limited to oleic acid, monoglycerides, diglycerides, a mono- or di-fatty acid ester of PEG, or a combination thereof.

Illustrative examples of cyclodextrins for use in the present pharmaceutical compositions include but are not limited to alpha-cyclodextrin, beta-cyclodextrin, hydroxypropyl-beta-cyclodextrin, or sulfobutyl ether-beta-cyclodextrin.

Illustrative examples of phospholipids for use in the present pharmaceutical compositions include but are not limited to soy phosphatidylcholine, or distearoyl phosphatidylglycerol, and hydrogenated forms thereof, or a combination thereof.

One of ordinary skill in the art may modify the formulations within the teachings of the specification to provide numerous formulations for a particular route of administration. In particular, the compounds or substances may be modified to render them more soluble in water or other vehicle. It is also well within the ordinary skill of the art to modify the route of administration and dosage regimen of a particular compound or substance in order to manage the pharmacokinetics of the present compounds or substances for maximum beneficial effect in a patient.

Lipids, Lipid Nanoparticles, and mRNA Pharmaceutical Compositions Comprising Same

In some embodiments are provided pharmaceutical compositions comprising an polynucleotide comprising a nucleic acid sequence encoding a fusion protein comprising a modified interleukin 2 (IL-2) polypeptide, as described above, and one or more lipids. In some embodiments are provided pharmaceutical compositions comprising an polynucleotide comprising a nucleic acid sequence encoding a fusion protein comprising a modified interleukin 2 (IL-2) polypeptide, as described above, and one or more lipids and lipid-containing nanoparticles. In some embodiments are provided pharmaceutical compositions comprising an polynucleotide comprising a nucleic acid sequence encoding a fusion protein comprising a modified interleukin 2 (IL-2) polypeptide, as described above, and one or more lipids and lipid-containing nanoparticles. Lipids and lipid-containing nanoparticles suitable for use in the pharmaceutical compositions disclosed herein and throughout are disclosed, for example, in U.S. provisional patent application No. 63/313,648, filed on Feb. 24, 2022, entitled “Novel Ionizable Cationic Lipids,” the content of which is hereby incorporated by reference in its entirety.

The terms “lipid” or “lipid moiety” are used in accordance with its ordinary meaning in chemistry and refer to a hydrophobic molecule which is typically characterized by an aliphatic hydrocarbon chain. In embodiments, the lipid moiety includes a carbon chain of 3 to 100 carbons. In embodiments, the lipid moiety includes a carbon chain of 5 to 50 carbons. In embodiments, the lipid moiety includes a carbon chain of 5 to 25 carbons. In embodiments, the lipid moiety includes a carbon chain of 8 to 525 carbons. Lipid moieties may include saturated or unsaturated carbon chains, and may be optionally substituted. In embodiments, the lipid moiety is optionally substituted with a charged moiety at the terminal end. In embodiments, the lipid moiety is an alkyl or heteroalkyl optionally substituted with a carboxylic acid moiety at the terminal end. Lipids are also a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

The terms “cationic lipid” or “ionizable cationic lipid” are used interchangeably herein and refer to lipids that are protonated at low pH, which makes them positively charged, but they remain neutral at physiological pH.

The term “lipid nanoparticle” includes a lipid formulation that can be used to deliver an active agent or therapeutic agent, such as a nucleic acid (e.g., an mRNA), to a target site of interest (e.g., cell, tissue, organ, and the like). In embodiments, the lipid particle described herein is a nucleic acid-lipid particle, which is typically formed from a cationic lipid, a non-cationic lipid, and optionally a conjugated lipid that prevents aggregation of the particle. In other embodiments, the active agent or therapeutic agent, such as a nucleic acid, may be encapsulated in the lipid portion of the particle, thereby protecting it from enzymatic degradation.

The term “lipid conjugate” refers to a conjugated lipid that inhibits aggregation of lipid particles. Such lipid conjugates include, but are not limited to, PEG-lipid conjugates such as, e.g., PEG coupled to dimyristoylglycerols (e.g., PEG-DMG conjugates), PEG coupled to diacylglycerols (e.g., PEG-DAG conjugates), PEG coupled to cholesterol, PEG coupled to phosphatidylethanolamines, and PEG conjugated to ceramides.

The term “diacylglycerol” or “DAG” includes a compound having 2 fatty acyl chains, R¹ and R², both of which have independently between 2 and 30 carbons bonded to the 1- and 2-position of glycerol by ester linkages. The acyl groups can be saturated or have varying degrees of unsaturation. Suitable acyl groups include, but are not limited to, lauroyl (C₁₂), myristoyl (C₁₄), palmitoyl (C₁₆), stearoyl (C₁₈), and icosoyl (C₂₀). In preferred embodiments, R¹ and R² are the same, i.e., R¹ and R² are both myristoyl (i.e., dimyristoyl), R¹ and R² are both stearoyl (i.e., distearoyl), etc. Diacylglycerols have the following general formula:

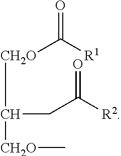

The term “dialkyloxypropyl” or “DAA” includes a compound having 2 alkyl chains, R¹ and R², both of which have independently between 2 and 30 carbons. The alkyl groups can be saturated or have varying degrees of unsaturation. Dialkyloxypropyls have the following general formula:

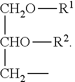

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The abbreviations used herein have their conventional meaning within the chemical and biological arts. The chemical structures and formulae set forth herein are constructed according to the standard rules of chemical valency known in the chemical arts. Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 30 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, B, Se, and S), and wherein the nitrogen and sulfur atoms may optionally be oxidized, and the nitrogen heteroatom may optionally be quaternized. The heteroatom(s) (e.g., O, N, S, Si, B, Se, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, B, Se, or P). A heteroalkyl moiety may include two optionally different heteroatoms (e.g., O, N, S, Si, B, Se, or P). A heteroalkyl moiety may include three optionally different heteroatoms (e.g., O, N, S, Si, B, Se, or P). A heteroalkyl moiety may include four optionally different heteroatoms (e.g., O, N, S, Si, B, Se, or P). A heteroalkyl moiety may include five optionally different heteroatoms (e.g., O, N, S, Si, B, Se, or P). A heteroalkyl moiety may include up to 8 optionally different heteroatoms (e.g., O, N, S, Si, B, Se, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of 0, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the [symbol] remainder of a molecule or chemical formula.

he term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In embodiments, the alkylarylene group has the formula:

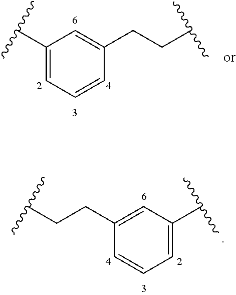 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₈ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, =N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)=NR″″, —NR—C(NR′R″)=NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)=NR″″, —NR—C(NR′R″)=NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣB—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:

(A) oxo,

halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₃₀ alkyl, C₁-C₈ alkyl, C₁-C₆ alkyl, or C₁-C₄ alkyl), unsubstituted heteroalkyl (e.g., 2 to 30 membered heteroalkyl, 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and

(B) alkyl (e.g., C₁-C₃₀ alkyl, C₁-C₈ alkyl, C₁-C₆ alkyl, or C₁-C₄ alkyl), heteroalkyl (e.g., 2 to 30 membered heteroalkyl, 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:

(i) oxo,

halogen, —CCl₃, —CBr₃, —CF₃, —C3, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₃₀ alkyl, C₁-C₈ alkyl, C₁-C₆ alkyl, or C₁-C₄ alkyl), unsubstituted heteroalkyl (e.g., 2 to 30 membered heteroalkyl, 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and

(ii) alkyl (e.g., C₁-C₃₀alkyl, C₁-C₈ alkyl, C₁-C₆ alkyl, or C₁-C₄ alkyl), heteroalkyl (e.g., 2 to 30 membered heteroalkyl, 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:

(a) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₃₀ alkyl, C₁-C₈ alkyl, C₁-C₆ alkyl, or C₁-C₄ alkyl), unsubstituted heteroalkyl (e.g., 2 to 30 membered heteroalkyl, 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and

(b) alkyl (e.g., C₁-C₃₀ alkyl, C₁-C₈ alkyl, C₁-C₆ alkyl, or C₁-C₄ alkyl), heteroalkyl (e.g., 2 to 30 membered heteroalkyl, 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₃₀ alkyl, C₁-C₈ alkyl, C₁-C₆ alkyl, or C₁-C₄ alkyl), unsubstituted heteroalkyl (e.g., 2 to 30 membered heteroalkyl, 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₃₀ alkyl, each substituted or unsubstituted heteroalkyl is a substituted or unsubstituted 2 to 30 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or“lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₃₀ alkyl, each substituted or unsubstituted heteroalkyl is a substituted or unsubstituted 2 to 30 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₃₀ alkylene, each substituted or unsubstituted heteroalkylene is a substituted or unsubstituted 2 to 30 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₃₀ alkyl, each substituted or unsubstituted heteroalkyl is a substituted or unsubstituted 2 to 30 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₃₀ alkylene, each substituted or unsubstituted heteroalkylene is a substituted or unsubstituted 2 to 30 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

### Compounds

In an aspect, provided herein is cationic lipid of formula (I):

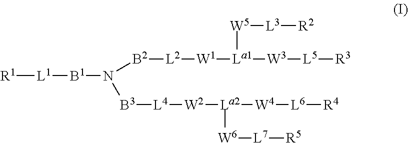

or a pharmaceutically acceptable salt, solvate, hydrate, stereoisomer, or prodrug thereof.

R¹ is H, —OR^{1A}, —YOR^{1A}, —NR^{1A}R^{1B}, —YNR^{1A}R^{1B}, —SR^{1A}, —YSR^{1A}, —(C═O)R^{1A}, —Y(C═O)R^{1A},

—(C═O)OR^{1A}, —Y(C═O)OR^{1A}, —O(C═O)R^{1A}, —YO(C═O)R^{1A}, —O(C═O)OR^{1A}, —YO(C═O)OR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Y is substituted or unsubstituted C₀-C₁₂ alkylene or substituted or unsubstituted 0 to 12 membered heteroalkylene.

R² is H, —OR^{2A}, —SR^{2A}, —(C═O)R^{2A}, —(C═O)OR^{2A}, —O(C═O)R^{2A}, —O(C═O)OR^{2A}, —(C═O)NHR^{2A},

—NH(C═O)R^{2A}, substituted or unsubstituted alkyl, or substituted or unsubstituted heteroalkyl.

R³ is H, —OR^{3A}, —SR^{3A}, —(C═O)R^{3A}, —(C═O)OR^{3A}, —O(C═O)R^{3A}, —O(C═O)OR^{3A}, —(C═O)NHR^{3A},

—NH(C═O)R^{3A}, substituted or unsubstituted alkyl, or substituted or unsubstituted heteroalkyl.

R⁴ is H, —OR^{4A}, —SR^{4A}, —(C═O)R^{4A}, —(C═O)OR^{4A}, —O(C═O)R^{4A}, —O(C═O)OR^{4A}, —(C═O)NHR^{4A},

—NH(C═O)R^{4A}, substituted or unsubstituted alkyl, or substituted or unsubstituted heteroalky.

R⁵ is H, —OR^{5A}, —SR^{5A}, —(C═O)R^{5A}, —(C═O)OR^{5A}, —O(C═O)R^{5A}, —O(C═O)OR^{5A}, —(C═O)NHR^{5A},

—NH(C═O)R^{5A}, substituted or unsubstituted alkyl, or substituted or unsubstituted heteroalkyl.

B¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

B² and B³ are each independently a bond, substituted or unsubstituted alkylene, or substituted or unsubstituted heteroalkylene.

L¹ is a bond, —O(C═O)—, —(C═O)O—, —O(C═O)O—, —C(═O)—, —O—, —O(CR¹⁰¹R¹⁰²)ₛO—, —S—, —C(═O)S—, —SC(═O)—, —NR¹⁰¹C(═O)—, —C(═O)NR¹⁰¹—, —NR¹⁰¹C(═S)—, —C(═S)NR¹⁰¹—, —NR¹⁰¹C(═O)NR¹⁰²—, —NR¹⁰¹C(═S)NR¹⁰²—, —OC(═O)NR¹⁰¹—, —NR¹⁰¹C(═O)O—, —SC(═O)NR¹⁰¹— or —NR¹⁰¹C(═O)S—.

L² is a bond, —O(C═O)—, —(C═O)O—, —O(C═O)O—, —C(═O)—, —O—, —O(CR²⁰¹R²⁰²)ₛO—, —S—, —C(═O)S—, —SC(═O)—, —NR²⁰¹C(═O)—, —C(═O)NR²⁰¹—, —NR²⁰¹C(═O)NR²⁰²—, —NR²⁰¹C(═S)—, —C(═S)NR²⁰¹—, —NR²⁰¹C(═S)NR²⁰²—, —OC(═O)NR²⁰¹—, —NR²⁰¹C(═O)O—, —SC(═O)NR²⁰¹— or —NR²⁰¹C(═O)S—.

L³ is a bond, —O(C═O)—, —(C═O)O—, —O(C═O)O—, —C(═O)—, —O—, —O(CR³⁰¹R³⁰²)ₛO—, —S—, —C(═O)S—, —SC(═O)—, —NR³⁰¹C(═O)—, —C(═O)NR³⁰¹—, —NR³⁰¹C(═O)NR³⁰²—, —NR³⁰¹C(═S)—, —C(═S)NR³⁰¹—, —NR³⁰¹C(═S)NR³⁰²—, —OC(═O)NR³⁰¹—, —NR³⁰¹C(═O)O—, —SC(═O)NR³⁰¹— or —NR³⁰¹C(═O)S—.

L⁴ is a bond, —O(C═O)—, —(C═O)O—, —O(C═O)O—, —C(═O)—, —O—, —O(CR⁴⁰¹R⁴⁰²)ₛO—, —S—, —C(═O)S—, —SC(═O)—, —NR⁴⁰¹C(═O)—, —C(═O)NR⁴⁰¹—, —NR⁴⁰¹C(═O)NR⁴⁰²—, —NR⁴⁰¹C(═S)—, —C(═S)NR⁴⁰¹—, —NR⁴⁰¹C(═S)NR⁴⁰²—, —OC(═O)NR⁴⁰¹—, —NR⁴⁰¹C(═O)O—, —SC(═O)NR⁴⁰¹— or —NR⁴⁰¹C(═O)S—.

L⁵ is a bond, —O(C═O)—, —(C═O)O—, —O(C═O)O—, —C(═O)—, —O—, —O(CR⁵⁰¹R⁵⁰²)ₛO—, —S—, —C(═O)S—, —SC(═O)—, —NR⁵⁰¹C(═O)—, —C(═O)NR⁵⁰¹—, —NR⁵⁰¹C(═O)NR⁵⁰²—, —NR⁵⁰¹C(═S)—, —C(═S)NR⁵⁰¹—, —NR⁵⁰¹C(═S)NR⁵⁰²—, —OC(═O)NR⁵⁰¹—, —NR⁵⁰¹C(═O)O—, —SC(═O)NR⁵⁰¹— or —NR⁵⁰¹C(═O)S—.

L⁶ is a bond, —O(C═O)—, —(C═O)O—, —O(C═O)O—, —C(═O)—, —O—, —O(CR⁶⁰¹R⁶⁰²)ₛO—, —S—, —C(═O)S—, —SC(═O)—, —NR⁶⁰¹C(═O)—, —C(═O)NR⁶⁰¹—, —NR⁶⁰¹C(═O)NR⁶⁰²—, —NR⁶⁰¹C(═S)—, —C(═S)NR⁶⁰¹—, —NR⁶⁰¹C(═S)NR⁶⁰²—, —OC(═O)NR⁶⁰¹—, —NR⁶⁰¹C(═O)O—, —SC(═O)NR⁶⁰¹— or —NR⁶⁰¹C(═O)S—.

L⁷ is a bond, —O(C═O)—, —(C═O)O—, —O(C═O)O—, —C(═O)—, —O—, —O(CR⁷⁰¹R⁷⁰²)ₛO—, —S—, —C(═O)S—, —SC(═O)—, —NR⁷⁰¹C(═O)—, —C(═O)NR⁷⁰¹—, —NR⁷⁰¹C(═O)NR⁷⁰²—, —NR⁷⁰¹C(═S)—, —C(═S)NR⁷⁰¹—, —NR⁷⁰¹C(═S)NR⁷⁰²—, —OC(═O)NR⁷⁰¹—, —NR⁷⁰¹C(═O)O—, —SC(═O)NR⁷⁰¹— or —NR⁷⁰¹C(═O)S—.

L^{a1} and L^{a2} are each independently

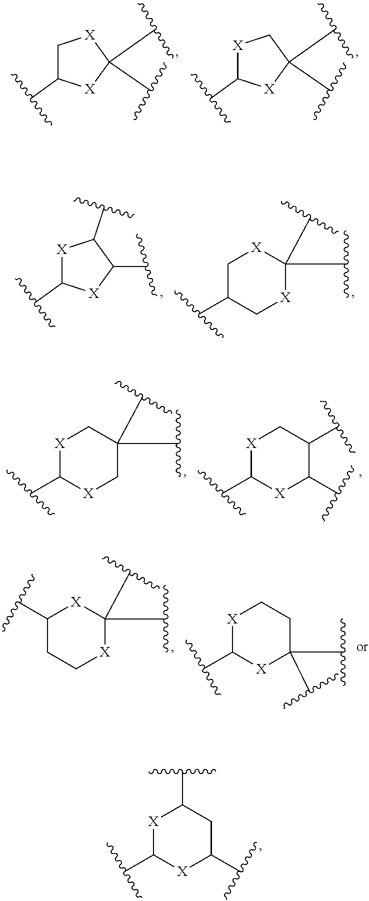

where each X is independently O, S, or CH₂.

W¹, W², W³, W⁴, W⁵, and W⁶ are each independently a bond, substituted or unsubstituted C₁-C₁₂ alkylene, or substituted or unsubstituted 2 to 12 membered heteroalkylene.

Each R^{1A} and R^{1B} is independently H, substituted or unsubstituted C₁-C₁₂ alkyl, or substituted or unsubstituted 2 to 12 membered heteroalkyl.

Each R^{2A}, R^{3A}, R^{4A}, and R^{5A} is independently H, substituted or unsubstituted C₁-C₃₀ alkyl, or substituted or unsubstituted 2 to 30 membered heteroalkyl.

Each R¹⁰¹, R¹⁰², R²⁰¹, R²⁰², R³⁰¹, R³⁰², R⁴⁰¹, R⁴⁰², R⁵⁰¹, R⁵⁰², R⁶⁰¹, R⁶⁰², R⁷⁰¹, and R⁷⁰² is independently H, substituted or unsubstituted C₁-C₁₂ alkyl, or substituted or unsubstituted 2 to 12 membered heteroalkyl.

Each s is independently an integer from 1 to 4.

In embodiments, R¹ is independently H, —OR^{1A}, —YOR^{1A}, —NR^{1A}R^{1B}, —YNR^{1A}R^{1B}, —SR^{1A},

—YSR^{1A}, —(C═O)R^{1A}, —Y(C═O)R^{1A}, —(C═O)OR^{1A}, —Y(C═O)OR^{1A}, —O(C═O)R^{1A}, —YO(C═O)R^{1A},
—O(C═O)OR^{1A}, —YO(C═O)OR^{1A}, substituted (e.g. with a substituent group, a size-limited substituent group or a lower substituent group) or unsubstituted alkyl (e.g., C₁-C₃₀ alkyl, C₁-C₈ alkyl, or C₁-C₄ alkyl), substituted (e.g. with a substituent group, a size-limited substituent group or a lower substituent group) or unsubstituted heteroalkyl (e.g., 2 to 30 membered heteroalkyl, 2 to 8 membered heteroalkyl, or 2 to 4 membered heteroalkyl), substituted (e.g. with a substituent group, a size-limited substituent group or a lower substituent group) or unsubstituted cycloalkyl (e.g., C₃-C₈ cycloalkyl, C₃-C₆ cycloalkyl, or C₅-C₆ cycloalkyl), substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl), substituted (e.g. with a substituent group, a size-limited substituent group or a lower substituent group) or unsubstituted aryl (e.g., C₆-C₁₀ aryl, C₁₀ aryl, or phenyl), or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, R¹ is substituted with one or more substituent groups. In embodiments, R¹ is substituted with one or more size-limited substituent groups. In embodiments, R¹ is substituted with one or more lower substituent groups.

In embodiments, R¹ is independently substituted (e.g. with a substituent group, a size-limited substituent group or a lower substituent group) alkyl (e.g., C₁-C₃₀ alkyl, C₁-C₈ alkyl, or C₁-C₄ alkyl). In embodiments, R¹ is independently unsubstituted alkyl (e.g., C₁-C₃₀ alkyl, C₁-C₈ alkyl, or C₁-C₄ alkyl). In embodiments, R¹ is independently substituted (e.g. with a substituent group, a size-limited substituent group or a lower substituent group) heteroalkyl (e.g., 2 to 30 membered heteroalkyl, 2 to 8 membered heteroalkyl, or 2 to 4 membered heteroalkyl). In embodiments, R¹ is independently unsubstituted heteroalkyl (e.g., 2 to 30 membered heteroalkyl, 2 to 8 membered heteroalkyl, or 2 to 4 membered heteroalkyl). In embodiments, R¹ is independently substituted (e.g. with a substituent group, a size-limited substituent group or a lower substituent group) cycloalkyl (e.g., C₃-C₈ cycloalkyl, C₃-C₆ cycloalkyl, or C₅-C₆ cycloalkyl). In embodiments, R¹ is independently unsubstituted cycloalkyl (e.g., C₃-C₈ cycloalkyl, C₃-C₆ cycloalkyl, or C₅-C₆ cycloalkyl). In embodiments, R¹ is independently substituted (e.g. with a substituent group, a size-limited substituent group or a lower substituent group) heterocycloalkyl (e.g., 3 to 8 membered heterocycloalkyl, 3 to 6 membered heterocycloalkyl, or 5 to 6 membered heterocycloalkyl). In embodiments, R¹ is independently unsubstituted heterocycloalkyl (e.g., 3 to 8 membered heterocycloalkyl, 3 to 6 membered heterocycloalkyl, or 5 to 6 membered heterocycloalkyl). In embodiments, R¹ is independently substituted (e.g. with a substituent group, a size-limited substituent group or a lower substituent group) aryl (e.g., C₆-C₁₀ aryl, C₁₀ aryl, or phenyl). In embodiments, R¹ is independently unsubstituted aryl (e.g., C₆-C₁₀ aryl, C₁₀ aryl, or phenyl). In embodiments, R¹ is independently substituted (e.g. with a substituent group, a size-limited substituent group or a lower substituent group) heteroaryl (e.g., 5 to 10 membered heteroaryl, 5 to 9 membered heteroaryl, or 5 to 6 membered heteroaryl). In embodiments, R¹ is independently unsubstituted heteroaryl (e.g., 5 to 10 membered heteroaryl, 5 to 9 membered heteroaryl, or 5 to 6 membered heteroaryl).

In embodiments, R¹ is independently H, —OR^{1A}, or substituted or unsubstituted heteroalkyl. In embodiments, R¹ is independently H, —OR^{1A}, or substituted (e.g. with a substituent group, a size-limited substituent group or a lower substituent group) or unsubstituted heteroalkyl (e.g., 2 to 30 membered heteroalkyl, 2 to 8 membered heteroalkyl, or 2 to 4 membered heteroalkyl). In embodiments, R¹ is independently H. In embodiments, R¹ is independently —OR^{1A}. In embodiments, R¹ is independently substituted or unsubstituted heteroalkyl. In embodiments, R¹ is independently substituted (e.g. with a substituent group, a size-limited substituent group or a lower substituent group) or unsubstituted heteroalkyl (e.g., 2 to 30 membered heteroalkyl, 2 to 8 membered heteroalkyl, or 2 to 4 membered heteroalkyl).

In embodiments, R¹ is independently H, —OH, methoxy, ethoxy, or substituted or unsubstituted heteroalkyl. In embodiments, R¹ is independently —OH or methoxy.

In embodiments, R¹ is independently H. In embodiments, R¹ is independently —OH. In embodiments, R¹ is independently methoxy. In embodiments, R¹ is independently ethoxy.

In embodiments, R² is H, —OR^{2A}, —SR^{2A}, —(C═O)R^{2A}, —(C═O)OR^{2A}, —O(C═O)R^{2A}, —O(C═O)OR^{2A}, —(C═O)NHR^{A2}, —NH(C═O)R^{2A}, substituted (e.g. with a substituent group, a size-limited substituent group or a lower substituent group) or unsubstituted alkyl (e.g., C₁-C₃₀ alkyl, C₁-C₈ alkyl, or C₁-C₄ alkyl), or substituted (e.g. with a substituent group, a size-limited substituent group or a lower substituent group) or unsubstituted heteroalkyl (e.g., 2 to membered heteroalkyl, 2 to 8 membered heteroalkyl, or 2 to 4 membered heteroalkyl). In embodiments, R² is substituted with one or more substituent groups. In embodiments, R² is substituted with one or more size-limited substituent groups. In embodiments, R² is substituted with one or more lower substituent groups.

In embodiments, R² is substituted (e.g. with a substituent group, a size-limited substituent group or a lower substituent group) alkyl (e.g., C₁-C₃₀ alkyl, C₁-C₈ alkyl, or C₁-C₄ alkyl). In embodiments, R² is unsubstituted alkyl (e.g., C₁-C₃₀ alkyl, C₁-C₈ alkyl, or C₁-C₄ alkyl). In embodiments, R² is substituted (e.g. with a substituent group, a size-limited substituent group or a lower substituent group) heteroalkyl (e.g., 2 to 30 membered heteroalkyl, 2 to 8 membered heteroalkyl, or 2 to 4 membered heteroalkyl). In embodiments, R² is unsubstituted heteroalkyl (e.g., 2 to 30 membered heteroalkyl, 2 to 8 membered heteroalkyl, or 2 to 4 membered heteroalkyl).

In embodiments, R² is H or substituted or unsubstituted alkyl. In embodiments, R² is H or substituted (e.g. with a substituent group, a size-limited substituent group or a lower substituent group) or unsubstituted alkyl (e.g., C₁-C₁₂ alkyl, C₁-C₈ alkyl, or C₁-C₄ alkyl). In embodiments, R² is H. In embodiments, R² is substituted (e.g. with a substituent group, a size-limited substituent group or a lower substituent group) alkyl (e.g., C₁-C₁₂ alkyl, C₁-C₈ alkyl, or C₁-C₄ alkyl). In embodiments, R² is unsubstituted alkyl (e.g., C₁-C₁₂ alkyl, C₁-C₈ alkyl, or C₁-C₄ alkyl).

In embodiments, R² is H or substituted or unsubstituted C₁-C₁₂ alkyl. In embodiments, R² is substituted (e.g. with a substituent group, a size-limited substituent group or a lower substituent group) C₁-C₁₂ alkyl. In embodiments, R² is unsubstituted C₁-C₁₂ alkyl.

In embodiments, R³ is H, —OR^{3A}, —SR^{3A}, —(C═O)R^{3A}, —(C═O)OR^{3A}, —O(C═O)R^{3A}, —O(C═O)OR^{3A}, —(C═O)NH^{A3}, —NH(C═O)R^{3A}, substituted (e.g. with a substituent group, a size-limited substituent group or a lower substituent group) or unsubstituted alkyl (e.g., C₁-C₃₀ alkyl, C₁-C₈ alkyl, or C₁-C₄ alkyl), or substituted (e.g. with a substituent group, a size-limited substituent group or a lower substituent group) or unsubstituted heteroalkyl (e.g., 2 to membered heteroalkyl, 2 to 8 membered heteroalkyl, or 2 to 4 membered heteroalkyl). In embodiments, R³ is substituted with one or more substituent groups. In embodiments, R³ is substituted with one or more size-limited substituent groups. In embodiments, R³ is substituted with one or more lower substituent groups.

In embodiments, R³ is substituted (e.g. with a substituent group, a size-limited substituent group or a lower substituent group) alkyl (e.g., C₁-C₃₀ alkyl, C₁-C₈ alkyl, or C₁-C₄ alkyl). In embodiments, R³ is unsubstituted alkyl (e.g., C₁-C₃₀ alkyl, C₁-C₈ alkyl, or C₁-C₄ alkyl). In embodiments, R³ is substituted (e.g. with a substituent group, a size-limited substituent group or a lower substituent group) heteroalkyl (e.g., 2 to 30 membered heteroalkyl, 2 to 8 membered heteroalkyl, or 2 to 4 membered heteroalkyl). In embodiments, R³ is unsubstituted heteroalkyl (e.g., 2 to 30 membered heteroalkyl, 2 to 8 membered heteroalkyl, or 2 to 4 membered heteroalkyl).

In embodiments, R³ is H or substituted or unsubstituted alkyl. In embodiments, R³ is H or substituted (e.g. with a substituent group, a size-limited substituent group or a lower substituent group) or unsubstituted alkyl (e.g., C₁-C₁₂ alkyl, C₁-C₈ alkyl, or C₁-C₄ alkyl). In embodiments, R³ is H. In embodiments, R³ is substituted (e.g. with a substituent group, a size-limited substituent group or a lower substituent group) alkyl (e.g., C₁-C₁₂ alkyl, C₁-C₈ alkyl, or C₁-C₄ alkyl). In embodiments, R³ is unsubstituted alkyl (e.g., C₁-C₁₂ alkyl, C₁-C₈ alkyl, or C₁-C₄ alkyl).

In embodiments, R³ is H or substituted or unsubstituted C₁-C₁₂ alkyl. In embodiments, R³ is substituted (e.g. with a substituent group, a size-limited substituent group or a lower substituent group) C₁-C₁₂ alkyl. In embodiments, R³ is unsubstituted C₁-C₁₂ alkyl.

In embodiments, R⁴ is H, —OR^{4A}, —SR^{4A}, —(C═O)R^{4A}, —(C═O)OR^{4A}, —O(C═O)R^{4A}, —O(C═O)OR^{4A}, —(C═O)NHR^{4A}, —NH(C═O)R^{4A}, substituted (e.g. with a substituent group, a size-limited substituent group or a lower substituent group) or unsubstituted alkyl (e.g., C₁-C₃₀ alkyl, C₁-C₈ alkyl, or C₁-C₄ alkyl), or substituted (e.g. with a substituent group, a size-limited substituent group or a lower substituent group) or unsubstituted heteroalkyl (e.g., 2 to membered heteroalkyl, 2 to 8 membered heteroalkyl, or 2 to 4 membered heteroalkyl). In embodiments, R⁴ is substituted with one or more substituent groups. In embodiments, R⁴ is substituted with one or more size-limited substituent groups. In embodiments, R⁴ is substituted with one or more lower substituent groups.

In embodiments, R⁴ is substituted (e.g. with a substituent group, a size-limited substituent group or a lower substituent group) alkyl (e.g., C₁-C₃₀ alkyl, C₁-C₈ alkyl, or C₁-C₄ alkyl). In embodiments, R⁴ is unsubstituted alkyl (e.g., C₁-C₃₀ alkyl, C₁-C₈ alkyl, or C₁-C₄ alkyl). In embodiments, R⁴ is substituted (e.g. with a substituent group, a size-limited substituent group or a lower substituent group) heteroalkyl (e.g., 2 to 30 membered heteroalkyl, 2 to 8 membered heteroalkyl, or 2 to 4 membered heteroalkyl). In embodiments, R⁴ is unsubstituted heteroalkyl (e.g., 2 to 30 membered heteroalkyl, 2 to 8 membered heteroalkyl, or 2 to 4 membered heteroalkyl).

In embodiments, R⁴ is H or substituted or unsubstituted alkyl. In embodiments, R⁴ is H or substituted (e.g. with a substituent group, a size-limited substituent group or a lower substituent group) or unsubstituted alkyl (e.g., C₁-C₁₂ alkyl, C₁-C₈ alkyl, or C₁-C₄ alkyl). In embodiments, R⁴ is H. In embodiments, R⁴ is substituted (e.g. with a substituent group, a size-limited substituent group or a lower substituent group) alkyl (e.g., C₁-C₁₂ alkyl, C₁-C₈ alkyl, or C₁-C₄ alkyl). In embodiments, R⁴ is unsubstituted alkyl (e.g., C₁-C₁₂ alkyl, C₁-C₈ alkyl, or C₁-C₄ alkyl).

In embodiments, R⁴ is H or substituted or unsubstituted C₁-C₁₂ alkyl. In embodiments, R⁴ is substituted (e.g. with a substituent group, a size-limited substituent group or a lower substituent group) C₁-C₁₂ alkyl. In embodiments, R⁴ is unsubstituted C₁-C₁₂ alkyl.

In embodiments, R⁵ is H, —OR^{5A}, —SR^{5A}, —(C═O)R^{5A}, —(C═O)OR^{5A}, —O(C═O)R^{5A}, —O(C═O)OR^{5A}, —(C═O)NHR^{5A}, —NH(C═O)R^{5A}, substituted (e.g. with a substituent group, a size-limited substituent group or a lower substituent group) or unsubstituted alkyl (e.g., C₁-C₃₀ alkyl, C₁-C₈ alkyl, or C₁-C₄ alkyl), or substituted (e.g. with a substituent group, a size-limited substituent group or a lower substituent group) or unsubstituted heteroalkyl (e.g., 2 to membered heteroalkyl, 2 to 8 membered heteroalkyl, or 2 to 4 membered heteroalkyl). In embodiments, R⁵ is substituted with one or more substituent groups. In embodiments, R⁵ is substituted with one or more size-limited substituent groups. In embodiments, R⁵ is substituted with one or more lower substituent groups.

In embodiments, R⁵ is substituted (e.g. with a substituent group, a size-limited substituent group or a lower substituent group) alkyl (e.g., C₁-C₃₀ alkyl, C₁-C₈ alkyl, or C₁-C₄ alkyl). In embodiments, R⁵ is unsubstituted alkyl (e.g., C₁-C₃₀ alkyl, C₁-C₈ alkyl, or C₁-C₄ alkyl). In embodiments, R⁵ is substituted (e.g. with a substituent group, a size-limited substituent group or a lower substituent group) heteroalkyl (e.g., 2 to 30 membered heteroalkyl, 2 to 8 membered heteroalkyl, or 2 to 4 membered heteroalkyl). In embodiments, R⁵ is unsubstituted heteroalkyl (e.g., 2 to 30 membered heteroalkyl, 2 to 8 membered heteroalkyl, or 2 to 4 membered heteroalkyl).

In embodiments, R⁵ is H or substituted or unsubstituted alkyl. In embodiments, R⁵ is H or substituted (e.g. with a substituent group, a size-limited substituent group or a lower substituent group) or unsubstituted alkyl (e.g., C₁-C₁₂ alkyl, C₁-C₈ alkyl, or C₁-C₄ alkyl). In embodiments, R⁵ is H. In embodiments, R⁵ is substituted (e.g. with a substituent group, a size-limited substituent group or a lower substituent group) alkyl (e.g., C₁-C₁₂ alkyl, C₁-C₈ alkyl, or C₁-C₄ alkyl). In embodiments, R⁵ is unsubstituted alkyl (e.g., C₁-C₁₂ alkyl, C₁-C₈ alkyl, or C₁-C₄ alkyl).

In embodiments, R⁵ is H or substituted or unsubstituted C₁-C₁₂ alkyl. In embodiments, R⁵ is substituted (e.g. with a substituent group, a size-limited substituent group or a lower substituent group) C₁-C₁₂ alkyl. In embodiments, R⁵ is unsubstituted C₁-C₁₂ alkyl.

In embodiments, Y is substituted (e.g. with a substituent group, a size-limited substituent group or a lower substituent group) or unsubstituted C₀-C₁₂ alkylene or substituted (e.g. with a substituent group, a size-limited substituent group or a lower substituent group) or unsubstituted 0 to 12 membered heteroalkylene. In embodiments, Y is substituted with one or more substituent groups. In embodiments, Y is substituted with one or more size-limited substituent groups. In embodiments, Y is substituted with one or more lower substituent groups.

In embodiments, Y is substituted (e.g. with a substituent group, a size-limited substituent group or a lower substituent group) C₀-C₁₂ alkylene. In embodiments, Y is unsubstituted C₀-C₁₂ alkylene. In embodiments, Y is substituted (e.g. with a substituent group, a size-limited substituent group or a lower substituent group) 0 to 12 membered heteroalkylene. In embodiments, Y is unsubstituted 0 to 12 membered heteroalkylene.

In embodiments, Y is substituted (e.g. with a substituent group, a size-limited substituent group or a lower substituent group) C₁-C₈ alkylene. In embodiments, Y is unsubstituted C₁-C₈ alkylene. In embodiments, Y is substituted (e.g. with a substituent group, a size-limited substituent group or a lower substituent group) 1 to 8 membered heteroalkylene. In embodiments, Y is unsubstituted 1 to 8 membered heteroalkylene.

In embodiments, Y is substituted (e.g. with a substituent group, a size-limited substituent group or a lower substituent group) C₁-C₄ alkylene. In embodiments, Y is unsubstituted C₁-C₄ alkylene. In embodiments, Y is substituted (e.g. with a substituent group, a size-limited substituent group or a lower substituent group) 1 to 4 membered heteroalkylene. In embodiments, Y is unsubstituted 1 to 4 membered heteroalkylene.

In embodiments, Y is substituted (e.g. with a substituent group, a size-limited substituent group or a lower substituent group) methylene, ethylene or propylene. In embodiments, Y is unsubstituted methylene, ethylene or propylene.

In embodiments, B¹ is a bond, substituted (e.g. with a substituent group, a size-limited substituent group or a lower substituent group) or unsubstituted alkylene (e.g., C₁-C₃₀ alkylene, C₁-C₈ alkylene, or C₁-C₄ alkylene), substituted (e.g. with a substituent group, a size-limited substituent group or a lower substituent group) or unsubstituted heteroalkylene (e.g., 2 to 30 membered heteroalkylene, 2 to 8 membered heteroalkylene, or 2 to 4 membered heteroalkylene), substituted (e.g. with a substituent group, a size-limited substituent group or a lower substituent group) or unsubstituted cycloalkylene (e.g., C₃-C₈ cycloalkylene, C₃-C₆ cycloalkylene, or C₅-C₆ cycloalkylene), substituted (e.g. with a substituent group, a size-limited substituent group or a lower substituent group) or unsubstituted heterocycloalkylene (e.g., 3 to 8 membered heterocycloalkylene, 3 to 6 membered heterocycloalkylene, or 5 to 6 membered heterocycloalkylene), substituted (e.g. with a substituent group, a size-limited substituent group or a lower substituent group) or unsubstituted arylene (e.g., C₆-C₁₀ arylene, C₁₀ arylene, or phenylene), or substituted (e.g. with a substituent group, a size-limited substituent group or a lower substituent group) or unsubstituted heteroarylene (e.g., 5 to 10 membered heteroarylene, 5 to 9 membered heteroarylene, or 5 to 6 membered heteroarylene). In embodiments, B¹ is substituted with one or more substituent groups. In embodiments, B 1 is substituted with one or more size-limited substituent groups. In embodiments, B 1 is substituted with one or more lower substituent groups. In embodiments, B¹ is a bond.

In embodiments, B¹ is substituted (e.g. with a substituent group, a size-limited substituent group or a lower substituent group) alkylene (e.g., C₁-C₃₀ alkylene, C₁-C₈ alkylene, or C₁-C₄ alkylene). In embodiments, B¹ is unsubstituted alkylene (e.g., C₁-C₃₀ alkylene, C₁-C₈ alkylene, or C₁-C₄ alkylene). In embodiments, B¹ is substituted (e.g. with a substituent group, a size-limited substituent group or a lower substituent group) heteroalkylene (e.g., 2 to 30 membered heteroalkylene, 2 to 8 membered heteroalkylene, or 2 to 4 membered heteroalkylene). In embodiments, B¹ is unsubstituted heteroalkylene (e.g., 2 to 30 membered heteroalkylene, 2 to 8 membered heteroalkylene, or 2 to 4 membered heteroalkylene). In embodiments, B¹ is substituted (e.g. with a substituent group, a size-limited substituent group or a lower substituent group) cycloalkylene (e.g., C₃-C₈ cycloalkylene, C₃-C₆ cycloalkylene, or C₅-C₆ cycloalkylene). In embodiments, B¹ is unsubstituted cycloalkylene (e.g., C₃-C₈ cycloalkylene, C₃-C₆ cycloalkylene, or C₅-C₆ cycloalkylene). In embodiments, B¹ is substituted (e.g. with a substituent group, a size-limited substituent group or a lower substituent group) heterocycloalkylene (e.g., 3 to 8 membered heterocycloalkylene, 3 to 6 membered heterocycloalkylene, or 5 to 6 membered heterocycloalkylene). In embodiments, B¹ is unsubstituted heterocycloalkylene (e.g., 3 to 8 membered heterocycloalkylene, 3 to 6 membered heterocycloalkylene, or 5 to 6 membered heterocycloalkylene). In embodiments, B¹ is substituted (e.g. with a substituent group, a size-limited substituent group or a lower substituent group) arylene (e.g., C₆-C₁₀ arylene, C₁₀ arylene, or phenylene). In embodiments, B¹ is unsubstituted arylene (e.g., C₆-C₁₀ arylene, C₁₀ arylene, or phenylene). In embodiments, B¹ is substituted (e.g. with a substituent group, a size-limited substituent group or a lower substituent group) heteroarylene (e.g., 5 to 10 membered heteroarylene, 5 to 9 membered heteroarylene, or 5 to 6 membered heteroarylene). In embodiments, B¹ is unsubstituted heteroarylene (e.g., 5 to 10 membered heteroarylene, 5 to 9 membered heteroarylene, or 5 to 6 membered heteroarylene).

In embodiments, B¹ is a bond or a substituted or unsubstituted alkylene. In embodiments, B¹ is a bond or substituted (e.g. with a substituent group, a size-limited substituent group or a lower substituent group) or unsubstituted alkylene (e.g., C₁-C₃₀ alkylene, C₁-C₈ alkylene, or C₁-C₄ alkylene).

In embodiments, B¹ is a bond or unsubstituted alkylene. In embodiments, B¹ is a bond or unsubstituted C₁-C₈ alkylene. In embodiments, B¹ is unsubstituted alkylene. In embodiments, B¹ is unsubstituted C₁-C₈ alkylene. In embodiments, B¹ is a bond.

In embodiments, B² and B³ are each independently a bond, substituted or unsubstituted alkylene, or substituted or unsubstituted heteroalkylene. In embodiments, B² and B³ are each independently a bond, substituted (e.g. with a substituent group, a size-limited substituent group or a lower substituent group) or unsubstituted alkylene (e.g., C₁-C₃₀ alkylene, C₁-C₈ alkylene, or C₁-C₄ alkylene), or substituted (e.g. with a substituent group, a size-limited substituent group or a lower substituent group) or unsubstituted heteroalkylene (e.g., 2 to 30 membered heteroalkylene, 2 to 8 membered heteroalkylene, or 2 to 4 membered heteroalkylene). In embodiments, B² is substituted with one or more substituent groups. In embodiments, B² is substituted with one or more size-limited substituent groups. In embodiments, B² is substituted with one or more lower substituent groups. In embodiments, B² is a bond. In embodiments, B³ is substituted with one or more substituent groups. In embodiments, B 3 is substituted with one or more size-limited substituent groups. In embodiments, B 3 is substituted with one or more lower substituent groups. In embodiments, B³ is a bond.

In embodiments, B² is substituted (e.g. with a substituent group, a size-limited substituent group or a lower substituent group) alkylene (e.g., C₁-C₃₀ alkylene, C₁-C₈ alkylene, or C₁-C₄ alkylene). In embodiments, B² is unsubstituted alkylene (e.g., C₁-C₃₀ alkylene, C₁-C₈ alkylene, or C₁-C₄ alkylene). In embodiments, B² is substituted (e.g. with a substituent group, a size-limited substituent group or a lower substituent group) heteroalkylene (e.g., 2 to 30 membered heteroalkylene, 2 to 8 membered heteroalkylene, or 2 to 4 membered heteroalkylene). In embodiments, B² is unsubstituted heteroalkylene (e.g., 2 to 30 membered heteroalkylene, 2 to 8 membered heteroalkylene, or 2 to 4 membered heteroalkylene).

In embodiments, B³ is substituted (e.g. with a substituent group, a size-limited substituent group or a lower substituent group) alkylene (e.g., C₁-C₃₀ alkylene, C₁-C₈ alkylene, or C₁-C₄ alkylene). In embodiments, B³ is unsubstituted alkylene (e.g., C₁-C₃₀ alkylene, C₁-C₈ alkylene, or C₁-C₄ alkylene). In embodiments, B³ is substituted (e.g. with a substituent group, a size-limited substituent group or a lower substituent group) heteroalkylene (e.g., 2 to 30 membered heteroalkylene, 2 to 8 membered heteroalkylene, or 2 to 4 membered heteroalkylene). In embodiments, B³ is unsubstituted heteroalkylene (e.g., 2 to 30 membered heteroalkylene, 2 to 8 membered heteroalkylene, or 2 to 4 membered heteroalkylene).

In embodiments, B² and B³ are each independently a bond or substituted or unsubstituted alkylene. In embodiments, B² and B³ are each independently a bond or substituted (e.g. with a substituent group, a size-limited substituent group or a lower substituent group) or unsubstituted alkylene (e.g., C₁-C₃₀ alkylene, C₁-C₈ alkylene, or C₁-C₄ alkylene).

In embodiments, B² and B³ are each independently a bond or substituted or unsubstituted C₁-C₈ alkylene. In embodiments, B² and B³ are each independently a bond or substituted (e.g. with a substituent group, a size-limited substituent group or a lower substituent group) or unsubstituted C₁-C₈ alkylene.

In embodiments, B² is a bond. In embodiments, B² is substituted C₁-C₈ alkylene. In embodiments, B² is unsubstituted C₁-C₈ alkylene. In embodiments, B³ is a bond. In embodiments, B³ is substituted C₁-C₈ alkylene. In embodiments, B³ is unsubstituted C₁-C₈ alkylene.

In embodiments, B² is butylene. In embodiments, B² is propylene. In embodiments, B² is ethylene. In embodiments, B² is methylene. In embodiments, B³ is butylene. In embodiments, B³ is propylene. In embodiments, B³ is ethylene. In embodiments, B³ is methylene.

In embodiments, L² is a bond, —O(C═O)—, —(C═O)O—, —O(C═O)O—, —C(═O)—, —O—, or —S—. In embodiments, L² is a bond, —O(C═O)—, —(C═O)O—, or —C(═O)—. In embodiments, L² is a bond,

### —O(C═O)— or —(C═O)O—.

In embodiments, L¹ is a bond, —NR¹⁰¹C(═S)—, —C(═S)NR¹⁰¹—, —O(C═O)—, —(C═O)O—, or —O—. In embodiments, L¹ is a bond, —NR¹⁰¹C(═S)—, or —C(═S)NR¹⁰¹—.

In embodiments, L¹ is a bond. In embodiments, L¹ is —NR¹⁰¹C(═S)—. In embodiments, L¹ is —C(═S)NR¹⁰¹. In embodiments, L¹ is —O(C═O)—. In embodiments, L¹ is —(C═O)O—. In embodiments, L¹ is —O—. In embodiments, L¹ is —C(═S)NR¹⁰¹, where the carbon atom is connected to the nitrogen atom in formula (I). In embodiments, L¹ is —C(═S)NH, where the carbon atom is connected to the nitrogen atom in formula (I).

In embodiments, each R¹⁰¹ is independently H, substituted or unsubstituted C₁-C₁₂ alkyl, or substituted or unsubstituted 2 to 12 membered heteroalkyl. In embodiments, each R¹⁰¹ is independently H, substituted (e.g. with a substituent group, a size-limited substituent group or a lower substituent group) or unsubstituted C₁-C₁₂ alkyl, or substituted or unsubstituted 2 to 12 membered heteroalkyl. In embodiments, each R¹⁰¹ is substituted with one or more substituent groups. In embodiments, each R¹⁰¹ is substituted with one or more size-limited substituent groups. In embodiments, each R¹⁰¹ is substituted with one or more lower substituent groups.

In embodiments, each R¹⁰¹ is independently H or substituted (e.g. with a substituent group, a size-limited substituent group or a lower substituent group) or unsubstituted 2 to 12 membered heteroalkyl. In embodiments, each R¹⁰¹ is independently substituted (e.g. with a substituent group, a size-limited substituent group or a lower substituent group) or unsubstituted 2 to 12 membered heteroalkyl.

In embodiments, each R¹⁰¹ is independently H. In embodiments, each R¹⁰¹ is independently substituted (e.g. with a substituent group, a size-limited substituent group or a lower substituent group) 2 to 12 membered heteroalkyl. In embodiments, each R¹⁰¹ is independently unsubstituted 2 to 12 membered heteroalkyl.

In embodiments, L² is a bond. In embodiments, L² is —O(C═O)—. In embodiments, L² is

—(C═O)O—. In embodiments, L² is —C(═O)—. In embodiments, L² is —O(C═O)O—. In embodiments, L² is —S—. In embodiments, L² is —O—.

In embodiments, L³ is a bond, —O(C═O)—, —(C═O)O—, —O(C═O)O—, —C(═O)—, —O—, or —S—. In embodiments, L³ is a bond, —O(C═O)—, —(C═O)O—, or —C(═O)—. In embodiments, L³ is a bond,

### —O(C═O)— or —(C═O)O—.

In embodiments, L³ is a bond. In embodiments, L³ is —O(C═O)—. In embodiments, L³ is

—(C═O)O—. In embodiments, L³ is —C(═O)—. In embodiments, L³ is —O(C═O)O—. In embodiments, L³ is —S—. In embodiments, L³ is —O—.

In embodiments, L⁴ is a bond, —O(C═O)—, —(C═O)O—, —O(C═O)O—, —C(═O)—, —O—, or —S—. In embodiments, L⁴ is a bond, —O(C═O)—, —(C═O)O—, or —C(═O)—. In embodiments, L⁴ is a bond,

### —O(C═O)— or —(C═O)O—.

In embodiments, L⁴ is a bond. In embodiments, L⁴ is —O(C═O)—. In embodiments, L⁴ is

—(C═O)O—. In embodiments, L⁴ is —C(═O)—. In embodiments, L⁴ is —O(C═O)O—. In embodiments, L⁴ is —S—. In embodiments, L⁴ is —O—.

In embodiments, L⁵ is a bond, —O(C═O)—, —(C═O)O—, —O(C═O)O—, —C(═O)—, —O—, or —S—. In embodiments, L⁵ is a bond, —O(C═O)—, —(C═O)O—, or —C(═O)—. In embodiments, L⁵ is a bond,

### —O(C═O)— or —(C═O)O—.

In embodiments, L⁵ is a bond. In embodiments, L⁵ is —O(C═O)—. In embodiments, L⁵ is

—(C═O)O—. In embodiments, L⁵ is —C(═O)—. In embodiments, L⁵ is —O(C═O)O—. In embodiments, L⁵ is —S—. In embodiments, L⁵ is —O—.

In embodiments, L⁶ is a bond, —O(C═O)—, —(C═O)O—, —O(C═O)O—, —C(═O)—, —O—, or —S—. In embodiments, L⁶ is a bond, —O(C═O)—, —(C═O)O—, or —C(═O)—. In embodiments, L⁶ is a bond,

### —O(C═O)— or —(C═O)O—.

In embodiments, L⁶ is a bond. In embodiments, L⁶ is —O(C═O)—. In embodiments, L⁶ is

—(C═O)O—. In embodiments, L⁶ is —C(═O)—. In embodiments, L⁶ is —O(C═O)O—. In embodiments, L⁶ is —S—. In embodiments, L⁶ is —O—.

In embodiments, L⁷ is a bond, —O(C═O)—, —(C═O)O—, —O(C═O)O—, —C(═O)—, —O—, or —S—. In embodiments, L⁷ is a bond, —O(C═O)—, —(C═O)O—, or —C(═O)—. In embodiments, L⁷ is a bond,

### —O(C═O)— or —(C═O)O—.

In embodiments, L⁷ is a bond. In embodiments, L⁷ is —O(C═O)—. In embodiments, L⁷ is

—(C═O)O—. In embodiments, L⁷ is —C(═O)—. In embodiments, L⁷ is —O(C═O)O—. In embodiments, L⁷ is —S—. In embodiments, L⁷ is —O—.

In embodiments, L^{a1} and L^{a2} are each independently

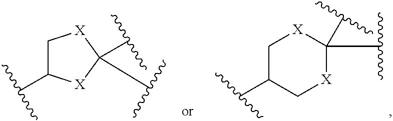

where each X is independently 0 or S. In embodiments, L^{a1} and L^{a2} are each independently

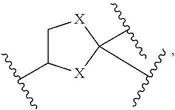

where each X is independently O or S. In embodiments, L^{a1} and L^{a2} are each independently

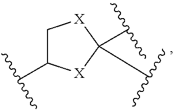

where each X is independently O.

In embodiments, L^{a1} and L^{a2} are each independently

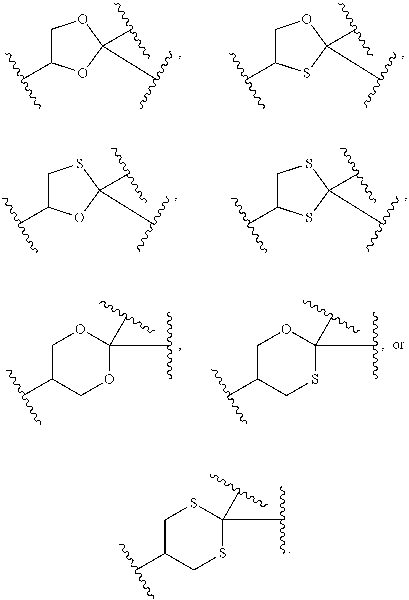

embodiments, L^{a1} and L^{a2} are each independently

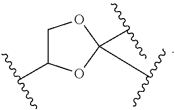 [SMILES: *C1COC(*)(*)O1]

In embodiments, L^{a1} and L^{a2} are each independently

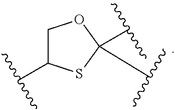 [SMILES: *C1COC(*)(*)S1]

In embodiments, L^{a1} and L^{a2} are each independently

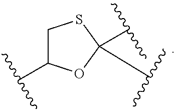 [SMILES: *C1CSC(*)(*)O1]

In embodiments, L^{a1} and L^{a2} are each independently

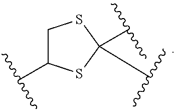 [SMILES: *C1CSC(*)(*)S1]

In embodiments, L^{a1} and L^{a2} are each independently

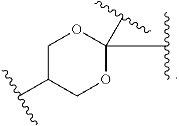 [SMILES: *C1COC(*)(*)OC1]

In embodiments, L^{a1} and L^{a2} are each independently

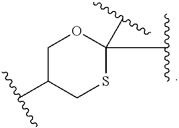 [SMILES: *C1COC(*)(*)SC1]

In embodiments, L^{a1} and L^{a2} are each independently

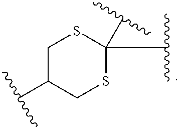 [SMILES: *C1CSC(*)(*)SC1]

In embodiments, W¹, W², W³, W⁴, W⁵, and W⁶ are each independently a bond, substituted (e.g. with a substituent group, a size-limited substituent group or a lower substituent group) or unsubstituted C₁-C₁₂ alkylene, or substituted (e.g. with a substituent group, a size-limited substituent group or a lower substituent group) or unsubstituted 2 to 12 membered heteroalkylene. In embodiments, W¹, W², W³, W⁴, W⁵, and W⁶ are each independently substituted with one or more substituent groups. In embodiments, W¹, W², W³, W⁴, W⁵, and W⁶ are each independently substituted with one or more size-limited substituent groups. In embodiments, W¹, W², W³, W⁴, W⁵, and W⁶ are each independently substituted with one or more lower substituent groups.

In embodiments, W¹, W², W³, W⁴, W⁵, and W⁶ are each independently a bond or substituted (e.g. with a substituent group, a size-limited substituent group or a lower substituent group) or unsubstituted C₁-C₁₂ alkylene. In embodiments, W¹, W², W³, W⁴, W⁵, and W⁶ are each independently a bond. In embodiments, W¹, W², W³, W⁴, W⁵, and W⁶ are each independently substituted (e.g. with a substituent group, a size-limited substituent group or a lower substituent group) C₁-C₁₂ alkylene. In embodiments, W¹, W², W³, W⁴, W⁵, and W⁶ are each independently unsubstituted C₁-C₁₂ alkylene.

In embodiments, each R^{1A} and R^{1B} is independently H, substituted (e.g. with a substituent group, a size-limited substituent group or a lower substituent group) or unsubstituted C₁-C₁₂ alkyl, or substituted (e.g. with a substituent group, a size-limited substituent group or a lower substituent group) or unsubstituted 2 to 12 membered heteroalkyl. In embodiments, each R^{1A} and R^{1B} is independently substituted with is independently substituted with one or more substituent groups. In embodiments, each R^{1A} and R^{1B} is independently substituted with one or more size-limited substituent groups. In embodiments, each R^{1A} and R^{1B} is independently substituted with one or more lower substituent groups.

In embodiments, R^{1A} and R^{1B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 12 membered, 5 to 10 membered, 5 to 9 membered, or 5 to 6 membered). In embodiments, a substituted heterocycloalkyl or substituted heteroaryl formed by the joining of R^{1A} and R^{1B} substituents bonded to the same nitrogen atom is substituted with at least one substituent group, size-limited substituent group, or lower substituent group; wherein if the substituted heterocycloalkyl or substituted heteroaryl is substituted with a plurality of groups selected from substituent groups, size-limited substituent groups, and lower substituent groups; each substituent group, size-limited substituent group, and/or lower substituent group may optionally be different. In embodiments, when a heterocycloalkyl formed by the joining of R^{1A} and R^{1B} substituents bonded to the same nitrogen atom is substituted, it is substituted with at least one substituent group. In embodiments, when a heterocycloalkyl formed by the joining of R^{1A} and R^{1B} substituents bonded to the same nitrogen atom is substituted, it is substituted with at least one size-limited substituent group. In embodiments, when a heterocycloalkyl formed by the joining of R^{1A} and R^{1B} substituents bonded to the same nitrogen atom is substituted, it is substituted with at least one lower substituent group. In embodiments, when a heteroaryl formed by the joining of R^{1A} and R^{1B} substituents bonded to the same nitrogen atom is substituted, it is substituted with at least one substituent group. In embodiments, when a heteroaryl formed by the joining of R^{1A} and R^{1B} substituents bonded to the same nitrogen atom is substituted, it is substituted with at least one size-limited substituent group. In embodiments, when a heteroaryl formed by the joining of R^{1A} and R^{1B} substituents bonded to the same nitrogen atom is substituted, it is substituted with at least one lower substituent group.

In embodiments, each R^{1A} is independently H or substituted or unsubstituted C₁-C₁₂ alkyl. In embodiments, each R^{1A} is independently H or substituted (e.g. with a substituent group, a size-limited substituent group or a lower substituent group) or unsubstituted C₁-C₁₂ alkyl. In embodiments, each R^{1A} is independently H. In embodiments, each R^{1A} is independently substituted (e.g. with a substituent group, a size-limited substituent group or a lower substituent group) C₁-C₁₂ alkyl. In embodiments, each R^{1A} is independently unsubstituted C₁-C₁₂ alkyl.

In embodiments, R¹ is H, —OR^{1A} or substituted or unsubstituted heteroalkyl.

L¹ is a bond, —NR¹⁰¹C(═S)—, —C(═S)NR¹⁰¹—, —O(C═O)—, —(C═O)O—, or —O—.

B¹ is a bond or a substituted or unsubstituted alkylene.

B² and B³ are each independently a bond or substituted or unsubstituted alkylene.

L² is a bond, —O(C═O)—, —(C═O)O—, —O(C═O)O—, —C(═O)—, —O—, or —S—.

L⁴ is a bond, —O(C═O)—, —(C═O)O—, —O(C═O)O—, —C(═O)—, —O—, or —S—.

W¹, W², W³, W⁴, W⁵, and W⁶ are each independently a bond or substituted or unsubstituted C₁-C₁₂ alkylene.

L^{a1} and L^{a2} are each independently

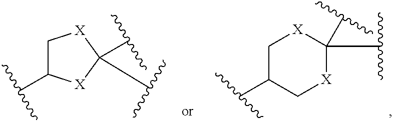

where each X is independently O or S.

L³ is a bond, —O(C═O)—, —(C═O)O—, —O(C═O)O—, —C(═O)—, —O—, or —S—.

L⁵ is a bond, —O(C═O)—, —(C═O)O—, —O(C═O)O—, —C(═O)—, —O—, or —S—.

L⁶ is a bond, —O(C═O)—, —(C═O)O—, —O(C═O)O—, —C(═O)—, —O—, or —S—.

L⁷ is a bond, —O(C═O)—, —(C═O)O—, —O(C═O)O—, —C(═O)—, —O—, or —S—.

R² is H or substituted or unsubstituted alkyl.

R³ is H or substituted or unsubstituted alkyl.

R⁴ is H or substituted or unsubstituted alkyl.

R⁵ is H or substituted or unsubstituted alkyl.

each R^{1A} is independently H or substituted or unsubstituted C₁-C₁₂ alkyl, and

each R¹⁰¹ is independently H or substituted or unsubstituted 2 to 12 membered heteroalkyl.

In embodiments, R¹ is H, —OH, methoxy, ethoxy, or substituted or unsubstituted heteroalkyl.

L¹ is a bond, —NR¹⁰¹C(═S)—, or —C(═S)NR¹⁰¹—.

B¹ is a bond or an unsubstituted C₁-C₈ alkylene.

B² and B³ are each independently a bond or substituted or unsubstituted C₁-C₈ alkylene.

L² is a bond, —O(C═O)—, or —(C═O)O—.

L⁴ is a bond, —O(C═O)—, or —(C═O)O—.

W¹, W², W³, W⁴, W⁵, and W⁶ are each independently a bond or substituted or unsubstituted C₁-C₁₂ alkylene.

L^{a1} and L^{a2} are each independently

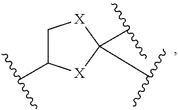

where each X is independently O or S.

L³ is a bond, —O(C═O)—, or —(C═O)O—.

L⁵ is a bond, —O(C═O)—, or —(C═O)O—.

L⁶ is a bond, —O(C═O)—, or —(C═O)O—.

L⁷ is a bond, —O(C═O)—, or —(C═O)O—.

R² is H or substituted or unsubstituted C₁-C₁₂ alkyl.

R³ is H or substituted or unsubstituted C₁-C₁₂ alkyl.

R⁴ is H or substituted or unsubstituted C₁-C₁₂ alkyl.

R⁵ is H or substituted or unsubstituted C₁-C₁₂ alkyl, and

each R¹⁰¹ is independently substituted or unsubstituted 2 to 12 membered heteroalkyl.

In embodiments, R¹ is —OH or methoxy. L¹ is a bond.

B¹ is an unsubstituted C₁-C₈ alkylene.

B² and B³ are each independently a bond or substituted or unsubstituted C₁-C₈ alkylene;

L² is a bond. L⁴ is a bond.

W¹, W², W³, W⁴, W⁵, and W⁶ are each independently a bond or substituted or unsubstituted C₁-C₁₂ alkylene.

L^{a1} and L^{a2} are each independently

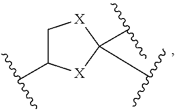

where each X is independently O.

L³ is a bond. L⁵ is a bond. L⁶ is a bond. L⁷ is a bond. R² is H or substituted or unsubstituted C₁-C₁₂ alkyl. R³ is H or substituted or unsubstituted C₁-C₁₂ alkyl.

R⁴ is H or substituted or unsubstituted C₁-C₁₂ alkyl, and R⁵ is H or substituted or unsubstituted C₁-C₁₂ alkyl;

In embodiments, R¹ is substituted or unsubstituted heteroalkyl.

L¹ is —C(═S)NR¹⁰¹—, where the carbon atom is connected to the nitrogen atom in formula (I).

B¹ is a bond. B² and B³ are each independently a bond or substituted or unsubstituted C₁-C₈ alkylene. L² is a bond, —O(C═O)—, or —(C═O)O—. L⁴ is a bond, —O(C═O)—, or —(C═O)O—.

W¹, W², W³, W⁴, W⁵, and W⁶ are each independently a bond or substituted or unsubstituted C₁-C₁₂ alkylene.

L^{a1} and L^{a2} are each independently

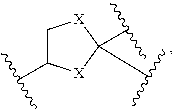

where each X is independently O. L³ is a bond.

L⁵ is a bond. L⁶ is a bond. L⁷ is a bond. R² is H or substituted or unsubstituted C₁-C₁₂ alkyl.

R³ is H or substituted or unsubstituted C₁-C₁₂ alkyl. R⁴ is H or substituted or unsubstituted C₁-C₁₂ alkyl, and R⁵ is H or substituted or unsubstituted C₁-C₁₂ alkyl.

In embodiments, each R^{2A}, R^{3A}, R^{4A}, and R^{5A} is independently H, substituted (e.g. with a substituent group, a size-limited substituent group or a lower substituent group) or unsubstituted C₁-C₃₀ alkyl, or substituted (e.g. with a substituent group, a size-limited substituent group or a lower substituent group) or unsubstituted 2 to 30 membered heteroalkyl. In embodiments, each R^{2A}, R^{3A}, R^{4A}, and R^{5A} is independently H. In embodiments, each R^{2A}, R^{3A}, R^{4A}, and R^{5A} is independently substituted (e.g. with a substituent group, a size-limited substituent group or a lower substituent group) C₁-C₃₀ alkyl. In embodiments, each R^{2A}, R^{3A}, R^{4A}, and R^{5A} is independently unsubstituted C₁-C₃₀ alkyl. In embodiments, each R^{2A}, R^{3A}, R^{4A}, and R^{5A} is independently substituted (e.g. with a substituent group, a size-limited substituent group or a lower substituent group) 2 to 30 membered heteroalkyl. In embodiments, each R^{2A}, R^{3A}, R^{4A}, and R^{5A} is independently unsubstituted 2 to 30 membered heteroalkyl.

In embodiments, each R¹⁰², R²⁰¹, R²⁰², R³⁰¹, R³⁰², R⁴⁰¹, R⁴⁰², R⁵⁰¹, R⁵⁰², R⁶⁰¹, R⁶⁰², R⁷⁰¹, and R⁷⁰² is independently H, substituted (e.g. with a substituent group, a size-limited substituent group or a lower substituent group) or unsubstituted C₁-C₁₂ alkyl, or substituted (e.g. with a substituent group, a size-limited substituent group or a lower substituent group) or unsubstituted 2 to 12 membered heteroalkyl. In embodiments, each R¹⁰¹, R²⁰¹, R²⁰², R³⁰¹, R³⁰², R⁴⁰¹, R⁴⁰², R⁵⁰¹, R⁵⁰², R⁶⁰¹, R⁶⁰², R⁷⁰¹, and R⁷⁰² is independently H. In embodiments, each R¹⁰¹, R²⁰¹, R²⁰², R³⁰¹, R³⁰², R⁴⁰¹, R⁴⁰², R⁵⁰¹, R⁵⁰², R⁶⁰¹, R⁶⁰², R⁷⁰¹, and R⁷⁰² is independently substituted (e.g. with a substituent group, a size-limited substituent group or a lower substituent group) C₁-C₁₂ alkyl. In embodiments, each R¹⁰¹, R²⁰¹, R²⁰², R³⁰¹, R³⁰², R⁴⁰¹, R⁴⁰², R⁵⁰¹, R⁵⁰², R⁶⁰¹, R⁶⁰², R⁷⁰¹, and R⁷⁰² is independently unsubstituted C₁-C₁₂ alkyl. In embodiments, each R¹⁰¹, R²⁰¹, R²⁰², R³⁰¹, R³⁰², R⁴⁰¹, R⁴⁰², R⁵⁰¹, R⁵⁰², R⁶⁰¹, R⁶⁰², R⁷⁰¹, and R⁷⁰² is independently substituted (e.g. with a substituent group, a size-limited substituent group or a lower substituent group) 2 to 12 membered heteroalkyl. In embodiments, each R¹⁰², R²⁰¹, R²⁰², R³⁰¹, R³⁰², R⁴⁰¹, R⁴⁰², R⁵⁰¹, R⁵⁰², R⁶⁰¹, R⁶⁰², R⁷⁰¹, and R⁷⁰² is independently unsubstituted 2 to 12 membered heteroalkyl.

In embodiments, each s is an integer from 1 to 4. In embodiments, each s is 1. In embodiments, each s is 2. In embodiments, each s is 3. In embodiments, each s is 4.

In embodiments, the cationic lipid of formula (I) is:

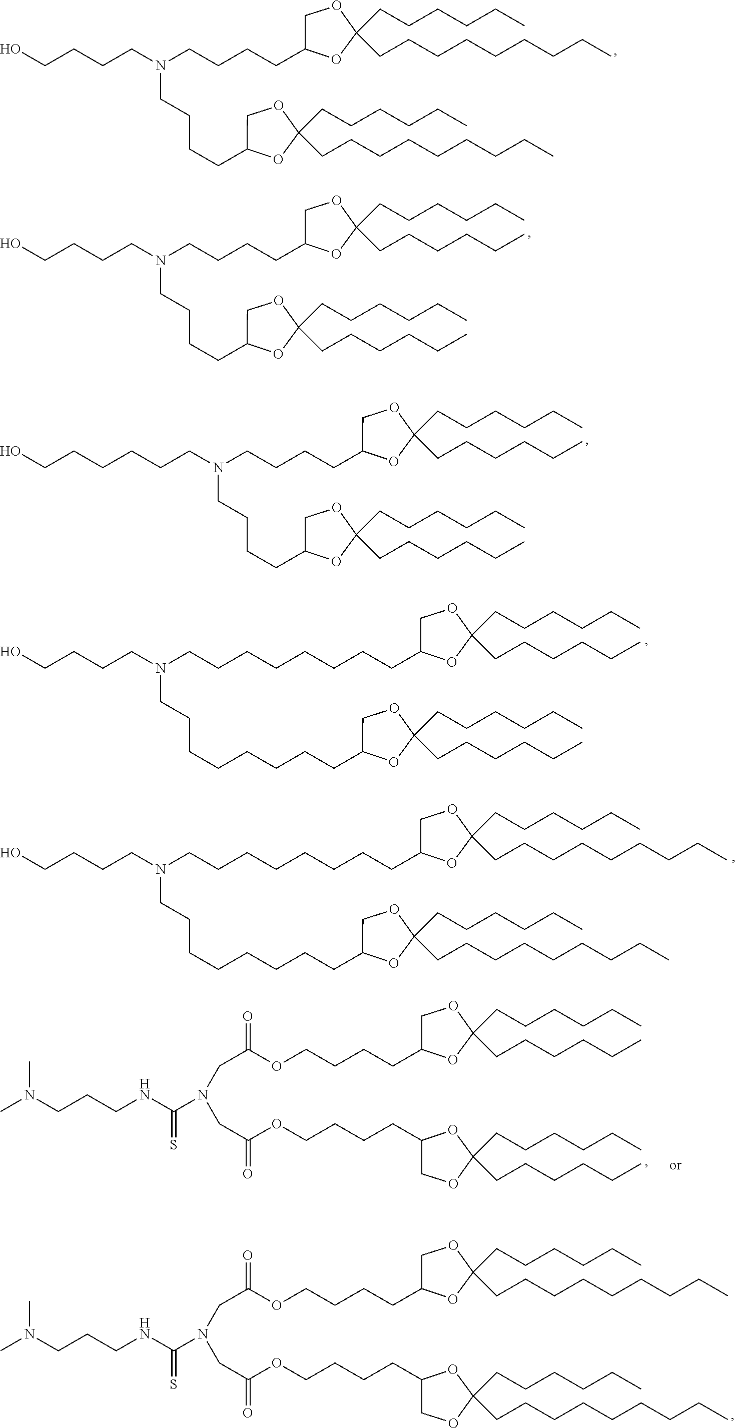

or a pharmaceutically acceptable salt thereof.

In an aspect, provided herein is cationic lipid of formula (II):

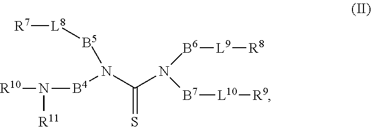

or a pharmaceutically acceptable salt, solvate, hydrate, stereoisomer, or prodrug thereof.

B⁴ is W⁷-L^{a3}-W⁸, where W⁷ and W⁸ are each independently a bond, substituted or unsubstituted alkylene, or substituted or unsubstituted heteroalkylene, and L^{a3} is a bond, —O(C═O)—, —(C═O)O—, —O(C═O)O—, —C(═O)—, —O—, —O(CR^{a31}R^{a32})ₛO—, —S—, —C(═O)S—, —SC(═O)—, —NR^{a31}C(═O)—, —C(═O)NR^{a31}—, —NR^{a31}C(═O)NR^{a32}—, —NR^{a31}C(═S)—, —C(═S)NR^{a31}—, —NR^{a31}C(═S)NR^{a32}—, —OC(═O)NR^{a31}—, —NR^{a31}C(═O)O—, —SC(═O)NR^{a31}— or —NR^{a31}C(═O)S—.

R¹⁰ and R¹¹ are each independently H, substituted or unsubstituted alkyl, substituted or unsubstituted heteroalkyl, or R¹⁰ and R¹¹ together with the nitrogen atom to which they are connected form a substituted or unsubstituted heterocycloalkyl or substituted or unsubstituted heteroaryl.

B⁵, B⁶, and B⁷ are each independently a bond, substituted or unsubstituted alkylene, or substituted or unsubstituted heteroalkylene.

L⁸ is a bond, —O(C═O)—, —(C═O)O—, —O(C═O)O—, —C(═O)—, —O—, —O(CR⁸⁰¹R⁸⁰²)ₛO—, —S—, —C(═O)S—, —SC(═O)—, —NR⁸⁰¹C(═O)—, —C(═O)NR⁸⁰¹—, —NR⁸⁰¹C(═O)NR⁸⁰²—, —NR⁸⁰¹C(═S)—, —C(═S)NR⁸⁰¹—, —NR⁸⁰¹C(═S)NR⁸⁰²—, —OC(═O)NR⁸⁰¹—, —NR⁸⁰¹C(═O)O—, —SC(═O)NR⁸⁰¹— or —NR⁸⁰¹C(═O)S—.

L⁹ is a bond, —O(C═O)—, —(C═O)O—, —O(C═O)O—, —C(═O)—, —O—, —O(CR⁹⁰¹R⁹⁰²)ₛO—, —S—, —C(═O)S—, —SC(═O)—, —NR⁹⁰¹C(═O)—, —C(═O)NR⁹⁰¹—, —NR⁹⁰¹C(═O)NR⁹⁰²—, —NR⁹⁰¹C(═S)—, —C(═S)NR⁹⁰¹—, —NR⁹⁰¹C(═S)NR⁹⁰²—, —OC(═O)NR⁹⁰¹—, —NR⁹⁰¹C(═O)O—, —SC(═O)NR⁹⁰¹— or —NR⁹⁰¹C(═O)S—.

L¹⁰ is a bond, —O(C═O)—, —(C═O)O—, —O(C═O)O—, —C(═O)—, —O—, —O(CR¹¹⁰R¹¹¹)ₛO—, —S—, —C(═O)S—, —SC(═O)—, —NR¹¹⁰C(═O)—, —C(═O)NR¹¹⁰—, —NR¹¹⁰C(═O)NR¹¹¹—, —NR¹¹⁰C(═S)—, —C(═S)NR¹¹⁰—, —NR¹¹⁰C(═S)NR¹¹¹—, —OC(═O)NR¹¹⁰—, —NR¹¹⁰C(═O)O—, —SC(═O)NR¹¹⁰— or —NR¹¹⁰C(═O)S—.

R⁷, R⁸, and R⁹ are each independently H, substituted or unsubstituted C₁-C₃₀ alkyl, or substituted or unsubstituted 2 to 30 membered heteroalkyl.

each R^{a31} and R^{a32} is independently H, substituted or unsubstituted C₁-C₁₂ alkyl, or substituted or unsubstituted 2 to 12 membered heteroalkyl.

each R⁸⁰¹, R⁸⁰², R⁹⁰¹, R⁹⁰², R¹¹⁰, and R¹¹¹ is independently H, substituted or unsubstituted C₁-C₁₂ alkyl, or substituted or unsubstituted 2 to 12 membered heteroalkyl.

each s is independently an integer from 1 to 4.

In embodiments, W⁷ and W⁸ are each independently a bond or substituted (e.g. with a substituent group, a size-limited substituent group or a lower substituent group) or unsubstituted alkylene (e.g., C₁-C₃₀ alkylene, C₁-C₈ alkylene, or C₁-C₄ alkylene), or substituted (e.g. with a substituent group, a size-limited substituent group or a lower substituent group) or unsubstituted heteroalkylene (e.g., 2 to 30 membered heteroalkylene, 2 to 8 membered heteroalkylene, or 2 to 4 membered heteroalkylene). In embodiments, W⁷ and W⁸ are each independently substituted with one or more substituent groups. In embodiments, W⁷ and W⁸ are each independently substituted with one or more size-limited substituent groups. In embodiments, W⁷ and W⁸ are each independently substituted with one or more lower substituent groups.

In embodiments, W⁷ and W⁸ are each independently substituted (e.g. with a substituent group, a size-limited substituent group or a lower substituent group) alkylene (e.g., C₁-C₃₀ alkylene, C₁-C₈ alkylene, or C₁-C₄ alkylene). In embodiments, W⁷ and W⁸ are each independently unsubstituted alkylene (e.g., C₁-C₃₀ alkylene, C₁-C₈ alkylene, or C₁-C₄ alkylene). In embodiments, W⁷ and W⁸ are each independently substituted (e.g. with a substituent group, a size-limited substituent group or a lower substituent group) heteroalkylene (e.g., 2 to 30 membered heteroalkylene, 2 to 8 membered heteroalkylene, or 2 to 4 membered heteroalkylene). In embodiments, W⁷ and W⁸ are each independently unsubstituted heteroalkylene (e.g., 2 to 30 membered heteroalkylene, 2 to 8 membered heteroalkylene, or 2 to 4 membered heteroalkylene). In embodiments, W⁷ and W⁸ are each independently a bond.

In embodiments, W⁷ and W⁸ are each independently a bond or substituted or unsubstituted C₁-C₈ alkylene. In embodiments, W⁷ and W⁸ are each independently substituted (e.g. with a substituent group, a size-limited substituent group or a lower substituent group) C₁-C₈ alkylene. In embodiments, W⁷ and W⁸ are each independently unsubstituted C₁-C₈ alkylene.

In embodiments, W⁷ and W⁸ are each independently a bond or substituted or unsubstituted C₂-C₄ alkylene. In embodiments, W⁷ and W⁸ are each independently substituted (e.g. with a substituent group, a size-limited substituent group or a lower substituent group) C₂-C₄ alkylene. In embodiments, W⁷ and W⁸ are each independently unsubstituted C₂-C₄ alkylene.

In embodiments, W⁷ and W⁸ are each independently a bond or unsubstituted C₂-C₄ alkylene. In embodiments, W⁷ and W⁸ are each independently a bond, ethylene, propylene, butylene, substituted (e.g. with a substituent group, a size-limited substituent group or a lower substituent group) ethylene, substituted (e.g. with a substituent group, a size-limited substituent group or a lower substituent group) propylene, or substituted (e.g. with a substituent group, a size-limited substituent group or a lower substituent group) butylene.

In embodiments, L³ is a bond, —O(C═O)—, —(C═O)O—, —O(C═O)O—, or —C(═O)—. In embodiments, L³ is a bond. In embodiments, L³ is —O(C═O)—. In embodiments, L^{a3} is —(C═O)O—. In embodiments, L³ is —O(C═O)O—. In embodiments, L³ is —C(═O)—.

In embodiments, R¹⁰ and R¹¹ are each independently H, substituted (e.g. with a substituent group, a size-limited substituent group or a lower substituent group) or unsubstituted alkyl (e.g., C₁-C₃₀ alkyl, C₁-C₈ alkyl, or C₁-C₄ alkyl), substituted (e.g. with a substituent group, a size-limited substituent group or a lower substituent group) or unsubstituted heteroalkyl (e.g., 2 to 30 membered heteroalkyl, 2 to 8 membered heteroalkyl, or 2 to 4 membered heteroalkyl), or R¹⁰ and R¹¹ together with the nitrogen atom to which they are connected form a substituted (e.g. with a substituent group, a size-limited substituent group or a lower substituent group) or unsubstituted heterocycloalkyl (e.g., 3 to 8 membered heterocycloalkyl, 3 to 6 membered heterocycloalkyl, or 5 to 6 membered heterocycloalkyl) or substituted (e.g. with a substituent group, a size-limited substituent group or a lower substituent group) or unsubstituted heteroaryl (e.g., 5 to 10 membered heteroaryl, 5 to 9 membered heteroaryl, or 5 to 6 membered heteroaryl). In embodiments, R¹⁰ and R¹¹ are each independently substituted with one or more substituent groups. In embodiments, R¹⁰ and R¹¹ are each independently substituted with one or more size-limited substituent groups. In embodiments, R¹⁰ and R¹¹ are each independently substituted with one or more lower substituent groups.

In embodiments, R¹⁰ and R¹¹ are each independently substituted (e.g. with a substituent group, a size-limited substituent group or a lower substituent group) alkyl (e.g., C₁-C₃₀ alkyl, C₁-C₈ alkyl, or C₁-C₄ alkyl). In embodiments, R¹⁰ and R¹¹ are each independently unsubstituted alkyl (e.g., C₁-C₃₀ alkyl, C₁-C₈ alkyl, or C₁-C₄ alkyl). In embodiments, R¹⁰ and R¹¹ are each independently substituted (e.g. with a substituent group, a size-limited substituent group or a lower substituent group) heteroalkyl (e.g., 2 to 30 membered heteroalkyl, 2 to 8 membered heteroalkyl, or 2 to 4 membered heteroalkyl). In embodiments, R¹⁰ and R¹¹ are each independently unsubstituted heteroalkyl (e.g., 2 to 30 membered heteroalkyl, 2 to 8 membered heteroalkyl, or 2 to 4 membered heteroalkyl). In embodiments, a substituted heterocycloalkyl or substituted heteroaryl formed by the joining of R¹⁰ and R¹¹ groups bonded to the same nitrogen atom is substituted with at least one substituent group, size-limited substituent group, or lower substituent group. If the substituted heterocycloalkyl or substituted heteroaryl is substituted with a plurality of groups selected from substituent groups, size-limited substituent groups, and lower substituent groups; each substituent group, size-limited substituent group, and/or lower substituent group may optionally be different. In embodiments, when a heterocycloalkyl formed by the joining of R¹⁰ and R¹¹ groups bonded to the same nitrogen atom is substituted, it is substituted with at least one substituent group. In embodiments, when a heterocycloalkyl formed by the joining of R¹⁰ and R¹¹ groups bonded to the same nitrogen atom is substituted, it is substituted with at least one size-limited substituent group. In embodiments, when a heterocycloalkyl formed by the joining of R¹⁰ and R¹¹ groups bonded to the same nitrogen atom is substituted, it is substituted with at least one lower substituent group. In embodiments, when a heteroaryl formed by the joining of R¹⁰ and R¹¹ groups bonded to the same nitrogen atom is substituted, it is substituted with at least one substituent group. In embodiments, when a heteroaryl formed by the joining of R¹⁰ and R¹¹ groups bonded to the same nitrogen atom is substituted, it is substituted with at least one size-limited substituent group. In embodiments, when a heteroaryl formed by the joining of R¹⁰ and R¹¹ groups bonded to the same nitrogen atom is substituted, it is substituted with at least one lower substituent group.

In embodiments, R¹⁰ and R¹¹ together with the nitrogen atom to which they are connected form substituted (e.g. with a substituent group, a size-limited substituent group or a lower substituent group) heterocycloalkyl (e.g., 3 to 8 membered heterocycloalkyl, 3 to 6 membered heterocycloalkyl, or 5 to 6 membered heterocycloalkyl). In embodiments, R¹⁰ and R¹¹ together with the nitrogen atom to which they are connected form unsubstituted heterocycloalkyl (e.g., 3 to 8 membered heterocycloalkyl, 3 to 6 membered heterocycloalkyl. or 5 to 6 membered heterocycloalkyl). In embodiments, R¹⁰ and R¹¹ together with the nitrogen atom to which they are connected form substituted (e.g. with a substituent group, a size-limited substituent group or a lower substituent group) heteroaryl (e.g., 5 to 10 membered heteroaryl, 5 to 9 membered heteroaryl, or 5 to 6 membered heteroaryl). In embodiments, R¹⁰ and R¹¹ together with the nitrogen atom to which they are connected form unsubstituted heteroaryl (e.g., 5 to 10 membered heteroaryl, 5 to 9 membered heteroaryl, or 5 to 6 membered heteroaryl).

In embodiments, R¹⁰ and R¹¹ are each independently H, substituted or unsubstituted alkyl or R¹⁰ and R¹¹ together with the nitrogen atom to which they are connected form a substituted or unsubstituted heterocycloalkyl. In embodiments, R¹⁰ and R¹¹ are each independently H, substituted (e.g. with a substituent group, a size-limited substituent group or a lower substituent group) or unsubstituted alkyl (e.g., C₁-C₃₀ alkyl, C₁-C₈ alkyl, or C₁-C₄ alkyl) or R¹⁰ and R¹¹ together with the nitrogen atom to which they are connected form a substituted (e.g. with a substituent group, a size-limited substituent group or a lower substituent group) or unsubstituted heterocycloalkyl. In embodiments, R¹⁰ and R¹¹ are each independently H

In embodiments, R¹⁰ and R¹¹ are each independently substituted or unsubstituted alkyl or R¹⁰ and R¹¹ together with the nitrogen atom to which they are connected form a substituted or unsubstituted heterocycloalkyl. In embodiments, R¹⁰ and R¹¹ are each independently substituted (e.g. with a substituent group, a size-limited substituent group or a lower substituent group) or unsubstituted alkyl (e.g., C₁-C₃₀ alkyl, C₁-C₈ alkyl, or C₁-C₄ alkyl) or R¹⁰ and R¹¹ together with the nitrogen atom to which they are connected form a substituted or unsubstituted heterocycloalkyl (e.g., 3 to 8 membered heterocycloalkyl, 3 to 6 membered heterocycloalkyl, or 5 to 6 membered heterocycloalkyl).

In embodiments, R¹⁰ and R¹¹ are each independently substituted or unsubstituted methyl, ethyl, propyl, isopropyl, butyl, pentyl or hexyl. In embodiments, R¹⁰ and R¹¹ are each independently substituted or unsubstituted methyl, ethyl or propyl.

In embodiments, R¹⁰ and R¹¹ are each independently substituted or unsubstituted methyl, ethyl, propyl, isopropyl, or R¹⁰ and R¹¹ together with the nitrogen atom to which they are connected form a substituted or unsubstituted 3 to 8 membered heterocycloalkyl.

In embodiments, R¹⁰ and R¹¹ are each independently substituted or unsubstituted methyl, ethyl, propyl, isopropyl, or R¹⁰ and R¹¹ together with the nitrogen atom to which they are connected form a substituted (e.g. with a substituent group, a size-limited substituent group or a lower substituent group) or unsubstituted 3 to 8 membered heterocycloalkyl.

In embodiments, R¹⁰ and R¹¹ are each independently substituted or unsubstituted methyl, ethyl, propyl, isopropyl, or R¹⁰ and R¹¹ together with the nitrogen atom to which they are connected form a substituted or unsubstituted 5 to 6 membered heterocycloalkyl.

In embodiments, R¹⁰ and R¹¹ are each independently substituted or unsubstituted methyl, ethyl, propyl, isopropyl, or R¹⁰ and R¹¹ together with the nitrogen atom to which they are connected form a substituted (e.g. with a substituent group, a size-limited substituent group or a lower substituent group) or unsubstituted 5 to 6 membered heterocycloalkyl.

In embodiments, R¹⁰ and R¹¹ together with the nitrogen atom to which they are connected form a substituted (e.g. with a substituent group, a size-limited substituent group or a lower substituent group) 5 to 6 membered heterocycloalkyl. In embodiments, R¹⁰ and R¹¹ together with the nitrogen atom to which they are connected form unsubstituted 5 to 6 membered heterocycloalkyl.

In embodiments, B⁵, B⁶, and B⁷ are each independently a bond, substituted (e.g. with a substituent group, a size-limited substituent group or a lower substituent group) or unsubstituted alkylene (e.g., C₁-C₃₀ alkylene, C₁-C₈ alkylene, or C₁-C₄ alkylene), or substituted (e.g. with a substituent group, a size-limited substituent group or a lower substituent group) or unsubstituted heteroalkylene (e.g., 2 to 30 membered heteroalkylene, 2 to 8 membered heteroalkylene, or 2 to 4 membered heteroalkylene). In embodiments, B⁵, B⁶, and B⁷ are each independently substituted with one or more substituent groups. In embodiments, B⁵, B⁶, and B⁷ are each independently substituted with one or more size-limited substituent groups. In embodiments, B⁵, B⁶, and B⁷ are each independently substituted with one or more lower substituent groups.

In embodiments, B⁵, B⁶, and B¹ are each independently a bond. In embodiments, B⁵, B⁶, and B⁷ are each independently substituted (e.g. with a substituent group, a size-limited substituent group or a lower substituent group) alkylene (e.g., C₁-C₃₀ alkylene, C₁-C₈ alkylene, or C₁-C₄ alkylene). In embodiments, B⁵, B⁶, and B⁷ are each independently unsubstituted alkylene (e.g., C₁-C₃₀ alkylene, C₁-C₈ alkylene, or C₁-C₄ alkylene). In embodiments, B⁵, B⁶, and B⁷ are each independently substituted (e.g. with a substituent group, a size-limited substituent group or a lower substituent group) heteroalkylene (e.g., 2 to membered heteroalkylene, 2 to 8 membered heteroalkylene, or 2 to 4 membered heteroalkylene). In embodiments, B⁵, B⁶, and B⁷ are each independently unsubstituted heteroalkylene (e.g., 2 to 30 membered heteroalkylene, 2 to 8 membered heteroalkylene, or 2 to 4 membered heteroalkylene).

In embodiments, B⁵ is a bond.

In embodiments, B⁶ and B⁷ are each independently a bond or substituted or unsubstituted alkylene. In embodiments, B⁶ and B⁷ are each independently a bond or substituted (e.g. with a substituent group, a size-limited substituent group or a lower substituent group) or unsubstituted alkylene (e.g., C₁-C₃₀ alkylene, C₁-C₈ alkylene, or C₁-C₄ alkylene).

In embodiments, B⁶ and B¹ are each independently a bond or substituted or unsubstituted C₁-C₈ alkylene. In embodiments, B⁶ and B⁷ are each independently a bond or substituted (e.g. with a substituent group, a size-limited substituent group or a lower substituent group) or unsubstituted C₁-C₈ alkylene. In embodiments, B⁶ and B⁷ are each independently a bond. In embodiments, B⁶ and B⁷ are each independently substituted (e.g. with a substituent group, a size-limited substituent group or a lower substituent group) C₁-C₈ alkylene. In embodiments, B⁶ and B⁷ are each independently unsubstituted C₁-C₈ alkylene.

In embodiments, B⁶ and B⁷ are each independently a bond or substituted or unsubstituted C₂-C₄ alkylene. In embodiments, B⁶ and B⁷ are each independently a bond or substituted (e.g. with a substituent group, a size-limited substituent group or a lower substituent group) or unsubstituted C₂-C₄ alkylene. In embodiments, B⁶ and B⁷ are each independently substituted (e.g. with a substituent group, a size-limited substituent group or a lower substituent group) C₂-C₄ alkylene. In embodiments, B⁶ and B⁷ are each independently unsubstituted C₂-C₄ alkylene.

In embodiments, B⁶ and B⁷ are each independently a bond or unsubstituted C₂-C₄ alkylene. In embodiments, B⁶ and B⁷ are each independently a bond, ethylene, propylene, or butylene. In embodiments, B⁶ and B⁷ are each independently a bond. In embodiments, B⁶ and B⁷ are each independently ethylene. In embodiments, B⁶ and B⁷ are each independently propylene. In embodiments, B⁶ and B⁷ are each independently butylene.

In embodiments, L⁸ is a bond, —O(C═O)—, —(C═O)O—, or —C(═O)—. In embodiments, L¹ is a bond. In embodiments, L⁸ is —O(C═O)—. In embodiments, L¹ is —(C═O)O—. In embodiments, L⁸ is —C(═O)—.

In embodiments, L⁹ is a bond, —O(C═O)—, —(C═O)O—, or —C(═O)—. In embodiments, L⁹ is

—O(C═O)— or —(C═O)O—. In embodiments, L⁹ is a bond. In embodiments, L⁹ is —O(C═O)—. In embodiments, L⁹ is —(C═O)O—. In embodiments, L⁹ is —C(═O)—.

In embodiments, L¹⁰ is a bond, —O(C═O)—, —(C═O)O—, or —C(═O)—. In embodiments, L¹⁰ is

—O(C═O)— or —(C═O)O—. In embodiments, L¹⁰ is a bond. In embodiments, L¹⁰ is —O(C═O)—. In embodiments, L¹⁰ is —(C═O)O—. In embodiments, L¹⁰ is —C(═O)—.

In embodiments, R⁷, R, and R⁹ are each independently H, substituted (e.g. with a substituent group, a size-limited substituent group or a lower substituent group) or unsubstituted C₁-C₃₀ alkyl, or substituted (e.g. with a substituent group, a size-limited substituent group or a lower substituent group) or unsubstituted 2 to 30 membered heteroalkyl. In embodiments, R⁷, R⁸, and R⁹ are each independently substituted with one or more substituent groups. In embodiments, R⁷, R⁸, and R⁹ are each independently substituted with one or more size-limited substituent groups. In embodiments, R⁷, R⁸, and R⁹ are each independently substituted with one or more lower substituent groups.

In embodiments, R⁷, R⁸, and R⁹ are each independently H. In embodiments, R⁷, R⁸, and R⁹ are each independently substituted (e.g. with a substituent group, a size-limited substituent group or a lower substituent group) C₁-C₃₀ alkyl. In embodiments, R⁷, R⁸, and R⁹ are each independently unsubstituted C₁-C₃₀ alkyl. In embodiments, R¹, R⁸, and R⁹ are each independently substituted (e.g. with a substituent group, a size-limited substituent group or a lower substituent group) 2 to 30 membered heteroalkyl. In embodiments, R¹, R⁸, and R⁹ are each independently unsubstituted 2 to 30 membered heteroalkyl.

In embodiments, R⁷, R⁸, and R⁹ are each independently H or substituted or unsubstituted C₁-C₃₀ alkyl. In embodiments, R⁷, R⁸, and R⁹ are each independently H or substituted (e.g. with a substituent group, a size-limited substituent group or a lower substituent group) or unsubstituted C₁-C₃₀ alkyl. In embodiments, R¹, R⁸, and R⁹ are each independently substituted (e.g. with a substituent group, a size-limited substituent group or a lower substituent group) or unsubstituted C₁-C₃₀ alkyl.

In embodiments, R¹, R⁸, and R⁹ are each independently substituted or unsubstituted C₁-C₂₀ alkyl. In embodiments, R⁷, R⁸, and R⁹ are each independently substituted (e.g. with a substituent group, a size-limited substituent group or a lower substituent group) or unsubstituted C₁-C₂₀ alkyl. In embodiments, R¹, R⁸, and R⁹ are each independently substituted (e.g. with a substituent group, a size-limited substituent group or a lower substituent group) C₁-C₂₀ alkyl. In embodiments, R¹, R⁸, and R⁹ are each independently unsubstituted C₁-C₂₀ alkyl.

In embodiments, each R^{a31} and R^{a32} is independently H, substituted (e.g. with a substituent group, a size-limited substituent group or a lower substituent group) or unsubstituted C₁-C₁₂ alkyl, or substituted (e.g. with a substituent group, a size-limited substituent group or a lower substituent group) or unsubstituted 2 to 12 membered heteroalkyl. In embodiments, each R^{a31} and R^{a32} is independently H. In embodiments, each R^{a31} and R^{a32} is independently substituted (e.g. with a substituent group, a size-limited substituent group or a lower substituent group) C₁-C₁₂ alkyl. In embodiments, each R^{a31} and R^{a32} is independently unsubstituted C₁-C₁₂ alkyl. In embodiments, each R^{a31} and R^{a32} is independently substituted (e.g. with a substituent group, a size-limited substituent group or a lower substituent group) 2 to 12 membered heteroalkyl. In embodiments, each R³¹ and R³² is independently unsubstituted 2 to 12 membered heteroalkyl.

In embodiments, each R⁸⁰¹, R⁸⁰², R⁹⁰¹, R⁹⁰², R¹¹⁰, and R¹¹¹ is independently H, substituted (e.g. with a substituent group, a size-limited substituent group or a lower substituent group) or unsubstituted C₁-C₁₂ alkyl, or substituted (e.g. with a substituent group, a size-limited substituent group or a lower substituent group) or unsubstituted 2 to 12 membered heteroalkyl. In embodiments, each R⁸⁰¹, R⁸⁰², R⁹⁰¹, R⁹⁰², R¹¹⁰, and R¹¹¹ is independently H. In embodiments, each R⁸⁰¹, R⁸⁰², R⁹⁰¹, R⁹⁰², R¹¹⁰, and R¹¹¹ is independently substituted (e.g. with a substituent group, a size-limited substituent group or a lower substituent group) C₁-C₁₂ alkyl. In embodiments, each R⁸⁰¹, R⁸⁰², R⁹⁰¹, R⁹⁰², R¹¹⁰, and R¹¹¹ is independently unsubstituted C₁-C₁₂ alkyl. In embodiments, each R⁸⁰¹, R⁸⁰², R⁹⁰¹, R⁹⁰², R¹¹⁰, and R¹¹¹ is independently substituted (e.g. with a substituent group, a size-limited substituent group or a lower substituent group) 2 to 12 membered heteroalkyl. In embodiments, each R⁸⁰¹, R⁸⁰², R⁹⁰¹, R⁹⁰², R¹¹⁰, and R¹¹¹ is independently unsubstituted 2 to 12 membered heteroalkyl.

In embodiments, each s is independently an integer from 1 to 4. In embodiments, each s is 1. In embodiments, each s is 2. In embodiments, each s is 3. In embodiments, each s is 4.

In embodiments, W⁷ and W⁸ are each independently a bond or substituted or unsubstituted alkylene. L^{a3} is a bond.

R¹⁰ and R¹¹ are each independently H, substituted or unsubstituted alkyl or R¹⁰ and R¹¹ together with the nitrogen atom to which they are connected form a substituted or unsubstituted heterocycloalkyl. B⁵ is a bond. B⁶ and B⁷ are each independently a bond or substituted or unsubstituted alkylene. L¹ is a bond. L⁹ is a bond, —O(C═O)—, —(C═O)O—, or —C(═O)—.

L¹⁰ is a bond, —O(C═O)—, —(C═O)O—, or —C(═O)—, and R⁷, R⁸, and R⁹ are each independently H or substituted or unsubstituted C₁-C₃₀ alkyl.

In embodiments, W⁷ and W⁸ are each independently a bond or substituted or unsubstituted C₁-C₈ alkylene. L^{a3} is a bond.

R¹⁰ and R¹¹ are each independently substituted or unsubstituted alkyl or R¹⁰ and R¹¹ together with the nitrogen atom to which they are connected form a substituted or unsubstituted heterocycloalkyl. B⁵ is a bond. B⁶ and B⁷ are each independently a bond or substituted or unsubstituted C₁-C₈ alkylene. L⁸ is a bond. L⁹ is —O(C═O)— or —(C═O)O—. L¹⁰-O(C═O)— or —(C═O)O—, and R⁷, R⁸, and R⁹ are each independently substituted or unsubstituted C₁-C₂₀ alkyl.

In embodiments, W⁷ and W⁸ are each independently a bond or substituted or unsubstituted C₂-C₄ alkylene. L^{a3} is a bond.

R¹⁰ and R¹¹ are each independently substituted or unsubstituted methyl, ethyl, propyl, isopropyl, or R¹⁰ and R¹¹ together with the nitrogen atom to which they are connected form a substituted or unsubstituted 3 to 8 membered heterocycloalkyl. B⁵ is a bond. B⁶ and B⁷ are each independently a bond or substituted or unsubstituted C₂-C₄ alkylene. L⁸ is a bond. L⁹ is —O(C═O)— or —(C═O)O—.

L¹⁰ —O(C═O)— or —(C═O)O—. R⁷ is H or methyl, and R⁸, and R⁹ are each independently substituted or unsubstituted C₁-C₂₀ alkyl.

In embodiments, W⁷ and W⁸ are each independently a bond or unsubstituted C₂-C₄ alkylene. L³ is a bond.

R¹⁰ and R¹¹ are each independently substituted or unsubstituted methyl, ethyl, propyl, isopropyl, or R¹⁰ and R¹¹ together with the nitrogen atom to which they are connected form a substituted or unsubstituted 5 to 6 membered heterocycloalkyl. B⁵ is a bond. B⁶ and B⁷ are each independently a bond or unsubstituted C₂-C₄ alkylene. L⁸ is a bond. L⁹ is —O(C═O)— or —(C═O)O—. L¹⁰ is

—O(C═O)— or —(C═O)O—. R⁷ is H or methyl, and R⁸ and R⁹ are each independently substituted or unsubstituted C₁-C₂₀ alkyl.

In embodiments, W⁷ and W⁸ are each independently a bond or unsubstituted C₂-C₄ alkylene. L³ is a bond.

R¹⁰ and R¹¹ are each independently substituted or unsubstituted methyl, ethyl, propyl, isopropyl, or R¹⁰ and R¹¹ together with the nitrogen atom to which they are connected form a substituted or unsubstituted 5 to 6 membered heterocycloalkyl. B⁵, B⁶, and B⁷ are each independently a bond.

L⁸ is a bond. L⁹ is a bond. L¹⁰ is a bond. R⁷ is H or methyl, and R⁸ and R⁹ are each independently substituted or unsubstituted C₁-C₃₀ alkyl.

In embodiments, the cationic lipid of formula (II) is:

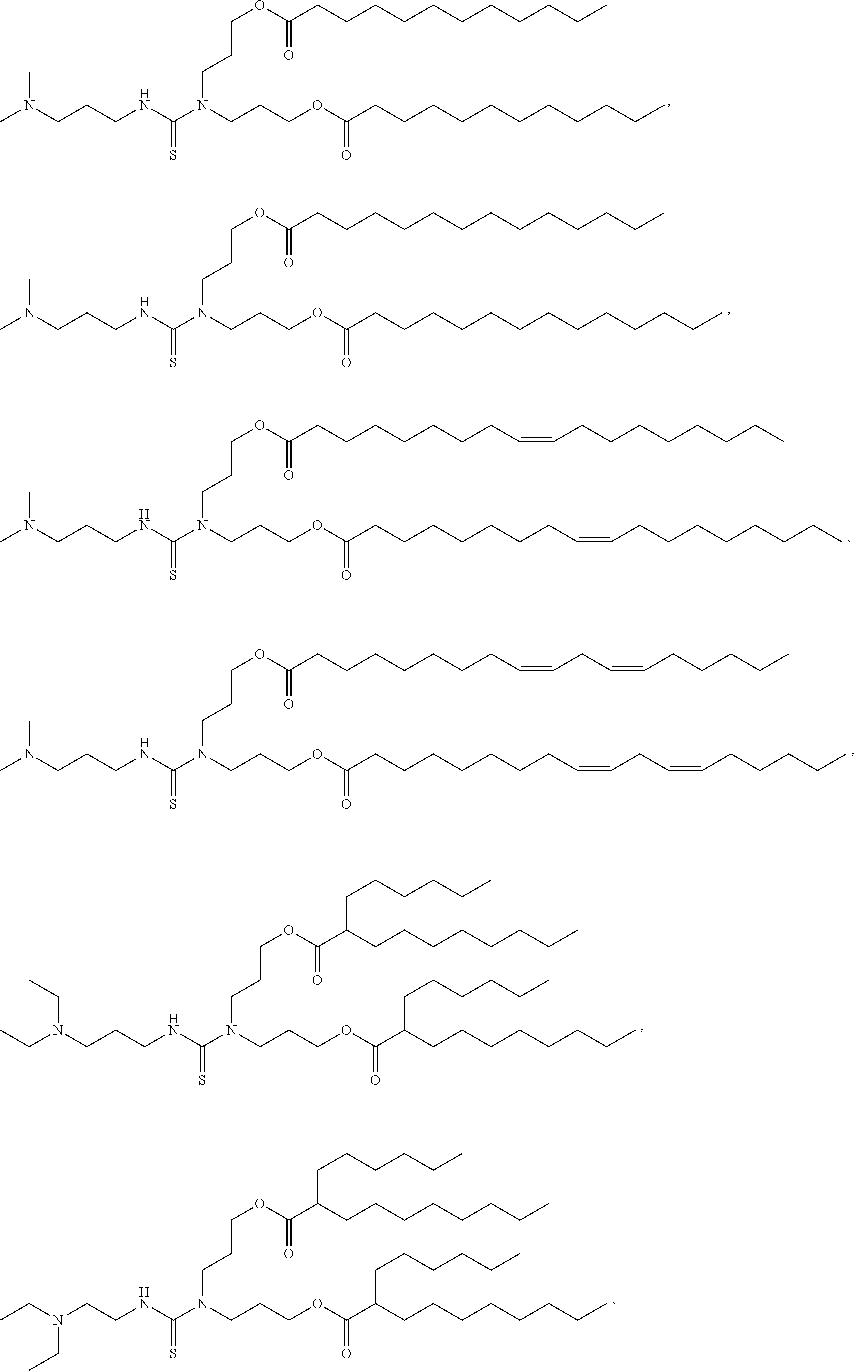

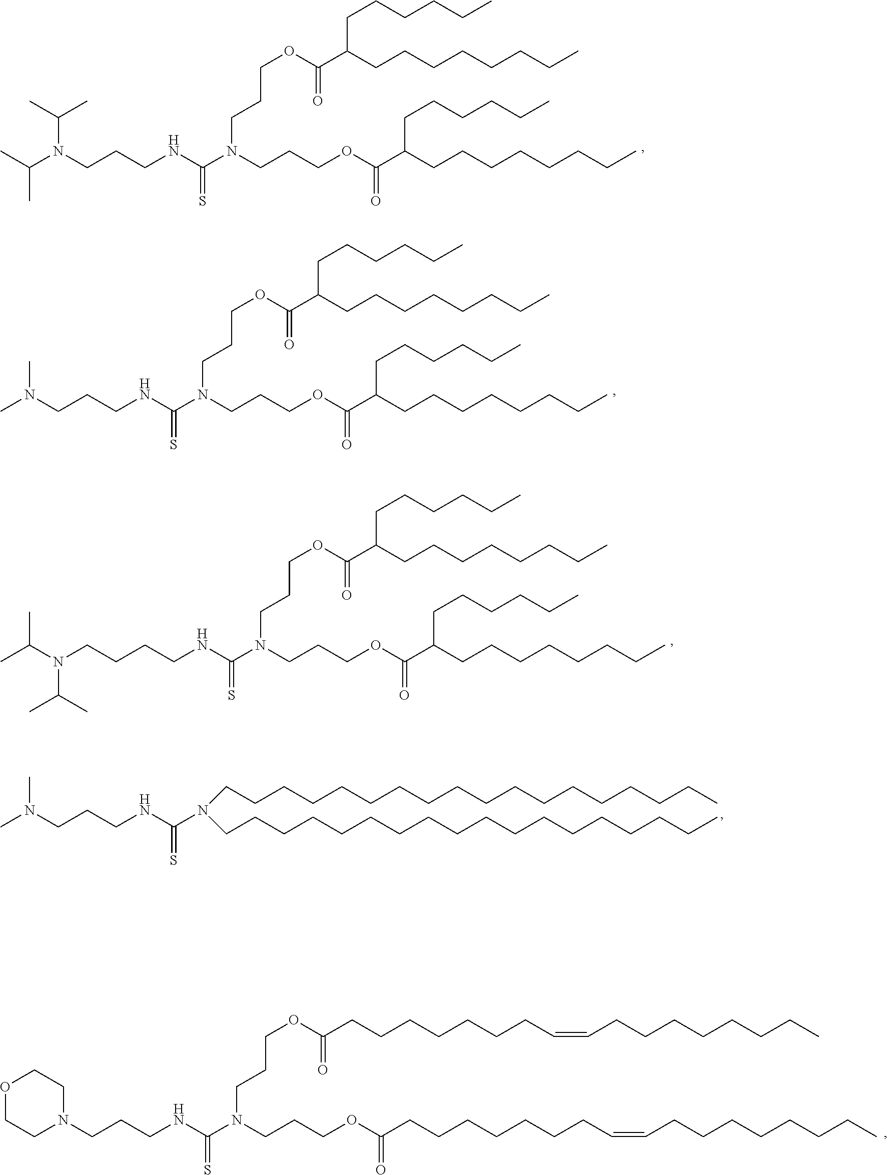

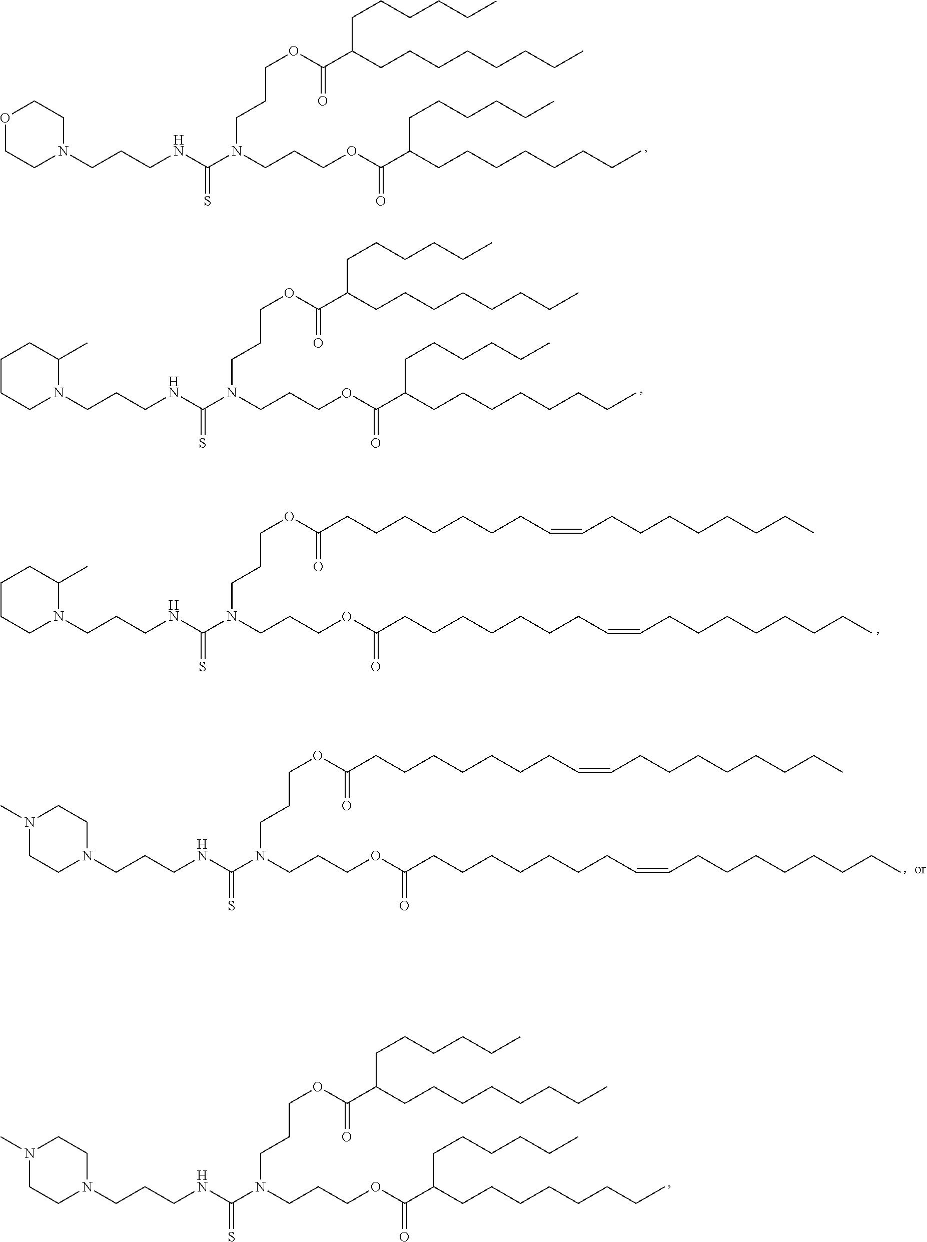

or a pharmaceutically acceptable salt thereof.

In an aspect, provided herein is cationic lipid of formula (III):

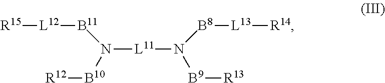

or a pharmaceutically acceptable salt, solvate, hydrate, stereoisomer, or prodrug thereof.

L¹¹ is

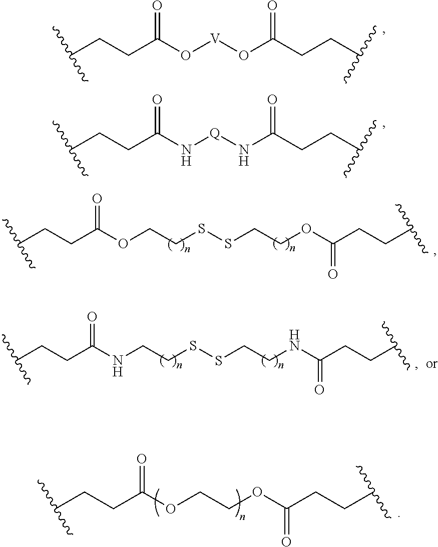

Q is substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroarylene.

V is substituted or unsubstituted alkylene, substituted or unsubstituted cycloalkylene, substituted or unsubstituted arylene.

B⁸, B⁹, B¹⁰, and B¹¹ are each independently a bond, substituted or unsubstituted alkylene, or substituted or unsubstituted heteroalkylene.

L¹² is a bond, —O(C═O)—, —(C═O)O—, —O(C═O)O—, —C(═O)—, —O—, —O(CR²¹⁰R²¹¹)ₛO—, —S—, —C(═O)S—, —SC(═O)—, —NR²¹⁰C(═O)—, —C(═O)NR²¹⁰—, —NR²¹⁰C(═O)NR²¹¹—, —NR²¹⁰C(═S)—, —C(═S)NR²¹⁰—, —NR²¹⁰C(═S)NR²¹¹—, —OC(═O)NR²¹⁰—, —NR²¹⁰C(═O)O—, —SC(═O)NR²¹⁰— or —NR²¹⁰C(═O)S—.

L¹³ is a bond, —O(C═O)—, —(C═O)O—, —O(C═O)O—, —C(═O)—, —O—, —O(CR³¹⁰R³¹¹)ₛO—, —S—, —C(═O)S—, —SC(═O)—, —NR³¹⁰C(═O)—, —C(═O)NR³¹⁰—, —NR³¹⁰C(═O)NR³¹¹—, —NR³¹⁰C(═S)—, —C(═S)NR³¹⁰—, —NR³¹⁰C(═S)NR³¹¹, —OC(═O)NR³¹⁰—, —NR³¹⁰C(═O)O—, —SC(═O)NR³¹⁰— or —NR³¹⁰C(═O)S—.

R¹² is H, —OR^{12A}, —SR^{12A}, —NR^{12A}, —CN, —(C═O)R^{12A}, —O(C═O)R^{12A}, —(C═O)OR¹²A,

—NR^{12A}(C═O)—R^{12B}, —(C═O)NR^{12A}R^{12B}.

R¹³ is H, —OR^{13A}, —SR^{13A}, —NR^{13A}, —CN, —(C═O)R^{13A}, —O(C═O)R^{13A}, —(C═O)OR^{13A},

—NR^{13A}(C═O)—R^{13B}, —(C═O)NR^{13A}R^{13B}.

R¹⁴ and R¹⁵ are each independently substituted or unsubstituted C₂-C₃₀ alkyl, or substituted or unsubstituted 2 to 30 membered heteroalkyl.

R^{12A}, R^{12B}, R^{13A}, and R^{13B} are each independently H, substituted or unsubstituted C₁-C₂₀ alkyl, or substituted or unsubstituted 2 to 20 membered heteroalkyl.

each R²¹⁰, R²¹¹, R³¹⁰, and R³¹¹ is independently H, substituted or unsubstituted C₁-C₁₂ alkyl, or substituted or unsubstituted 2 to 12 membered heteroalkyl.

each n is independently an integer from 0 to 8, and

each s is independently an integer from 1 to 4.

In embodiments, L¹¹ is

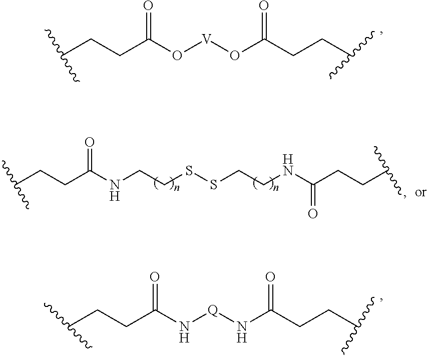

where n is an integer from 0 to 8, V is substituted or unsubstituted alkylene, and Q is substituted or unsubstituted alkylene.

In embodiments, L¹¹ is

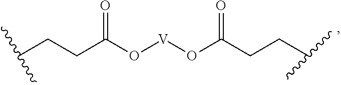

where V is substituted or unsubstituted alkylene. In embodiments, L¹¹ is

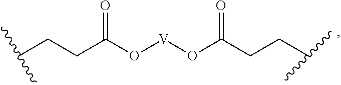

where V is substituted (e.g., with a substituent group, a size-limited substituent group or a lower substituent group) or unsubstituted alkylene (e.g., C₁-C₃₀ alkylene, C₁-C₈ alkylene, or C₁-C₄ alkylene).

In embodiments, L¹¹ is

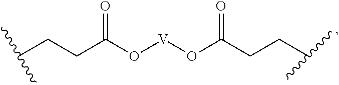

where V is substituted (e.g., with a substituent group, a size-limited substituent group or a lower substituent group) alkylene (e.g., C₁-C₃₀ alkylene, C₁-C₈ alkylene, or C₁-C₄ alkylene). In embodiments, L¹¹ is

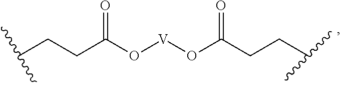

where V is unsubstituted alkylene (e.g., C₁-C₃₀ alkylene, C₁-C₈ alkylene, or C₁-C₄ alkylene).

In embodiments, L¹¹ is

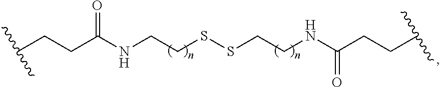

where n is an integer from 0 to 8. In embodiments, L¹¹ is

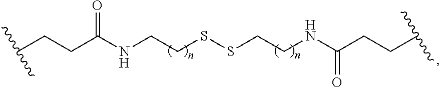

where n is an integer from 0 to 4.

In embodiments, L¹¹ is

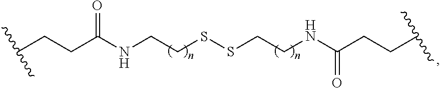

where n is 0. In embodiments, L¹¹ is

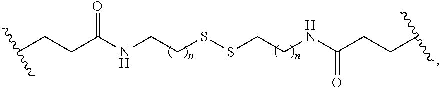

where n is 1.

In embodiments, L¹¹ is

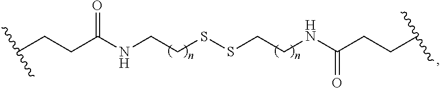

where n is 2.

In embodiments, L¹¹ is

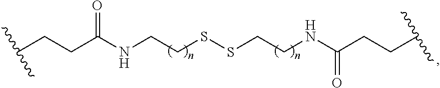

where n is 3.

In embodiments, L¹¹ is

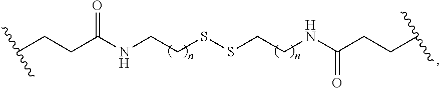

where n is 4.

In embodiments, L¹¹ is

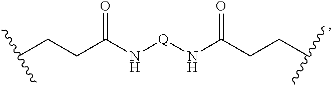

where Q is substituted or unsubstituted alkylene. In embodiments, L¹¹ is

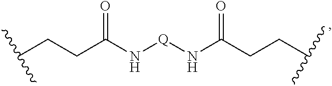

where Q is substituted (e.g., with a substituent group, a size-limited substituent group or a lower substituent group) or unsubstituted alkylene (e.g., C₁-C₃₀ alkylene, C₁-C₈ alkylene, or C₁-C₄ alkylene).

In embodiments, L¹¹ is

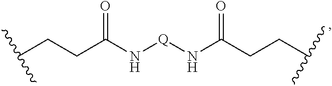

where Q is substituted (e.g. with a substituent group, a size-limited substituent group or a lower substituent group) alkylene (e.g., C₁-C₃₀ alkylene, C₁-C₈ alkylene, or C₁-C₄ alkylene). In embodiments, L¹¹ is

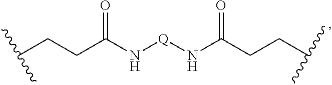

where Q is unsubstituted alkylene (e.g., C₁-C₃₀ alkylene, C₁-C₈ alkylene, or C₁-C₄ alkylene).

In embodiments, L¹¹ is

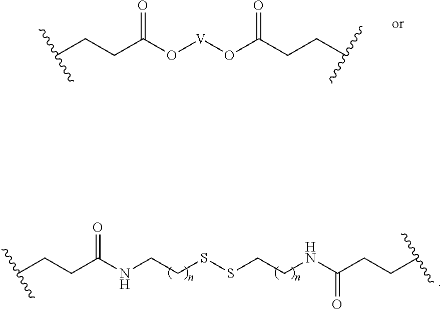

In embodiments, Q is substituted (e.g. with a substituent group, a size-limited substituent group or a lower substituent group) or unsubstituted alkylene (e.g., C₁-C₃₀ alkylene, C₁-C₈ alkylene, or C₁-C₄ alkylene), substituted (e.g. with a substituent group, a size-limited substituent group or a lower substituent group) or unsubstituted heteroalkylene (e.g., 2 to 30 membered heteroalkylene, 2 to 8 membered heteroalkylene, or 2 to 4 membered heteroalkylene), substituted (e.g. with a substituent group, a size-limited substituent group or a lower substituent group) or unsubstituted cycloalkylene (e.g., C₃-C₈ cycloalkylene, C₃-C₆ cycloalkylene, or C₅-C₆ cycloalkylene), substituted (e.g. with a substituent group, a size-limited substituent group or a lower substituent group) or unsubstituted heterocycloalkylene (e.g., 3 to 8 membered heterocycloalkylene, 3 to 6 membered heterocycloalkylene, or 5 to 6 membered heterocycloalkylene), substituted (e.g. with a substituent group, a size-limited substituent group or a lower substituent group) or unsubstituted arylene (e.g., C₆-C₁₀ arylene, C₁₀ arylene, or phenylene), or substituted (e.g. with a substituent group, a size-limited substituent group or a lower substituent group) or unsubstituted heteroarylene (e.g., 5 to 10 membered heteroarylene, 5 to 9 membered heteroarylene, or 5 to 6 membered heteroarylene). In embodiments, Q is substituted with one or more substituent groups. In embodiments, Q is substituted with one or more size-limited substituent groups. In embodiments, Q is substituted with one or more lower substituent groups.

In embodiments, Q is substituted (e.g. with a substituent group, a size-limited substituent group or a lower substituent group) alkylene (e.g., C₁-C₃₀ alkylene, C₁-C₈ alkylene, or C₁-C₄ alkylene). In embodiments, Q is unsubstituted alkylene (e.g., C₁-C₃₀ alkylene, C₁-C₈ alkylene, or C₁-C₄ alkylene). In embodiments, Q is substituted (e.g. with a substituent group, a size-limited substituent group or a lower substituent group) heteroalkylene (e.g., 2 to 30 membered heteroalkylene, 2 to 8 membered heteroalkylene, or 2 to 4 membered heteroalkylene). In embodiments, Q is unsubstituted heteroalkylene (e.g., 2 to membered heteroalkylene, 2 to 8 membered heteroalkylene, or 2 to 4 membered heteroalkylene). In embodiments, Q is substituted (e.g. with a substituent group, a size-limited substituent group or a lower substituent group) cycloalkylene (e.g., C₃-C₈ cycloalkylene, C₃-C₆ cycloalkylene, or C₅-C₆ cycloalkylene). In embodiments, Q is unsubstituted cycloalkylene (e.g., C₃-C₈ cycloalkylene, C₃-C₆ cycloalkylene, or C₅-C₆ cycloalkylene). In embodiments, Q is substituted (e.g. with a substituent group, a size-limited substituent group or a lower substituent group) heterocycloalkylene (e.g., 3 to 8 membered heterocycloalkylene, 3 to 6 membered heterocycloalkylene, or 5 to 6 membered heterocycloalkylene). In embodiments, Q is unsubstituted heterocycloalkylene (e.g., 3 to 8 membered heterocycloalkylene, 3 to 6 membered heterocycloalkylene, or 5 to 6 membered heterocycloalkylene). In embodiments, Q is substituted (e.g. with a substituent group, a size-limited substituent group or a lower substituent group) arylene (e.g., C₆-C₁₀ arylene, C₁₀ arylene, or phenylene). In embodiments, Q is unsubstituted arylene (e.g., C₆-C₁₀ arylene, C₁₀ arylene, or phenylene). In embodiments, Q is substituted (e.g. with a substituent group, a size-limited substituent group or a lower substituent group) heteroarylene (e.g., 5 to 10 membered heteroarylene, 5 to 9 membered heteroarylene, or 5 to 6 membered heteroarylene). In embodiments, Q is unsubstituted heteroarylene (e.g., 5 to 10 membered heteroarylene, 5 to 9 membered heteroarylene, or 5 to 6 membered heteroarylene).

In embodiments, Q is substituted or unsubstituted alkylene. In embodiments, Q is substituted (e.g. with a substituent group, a size-limited substituent group or a lower substituent group) alkylene (e.g., C₁-C₃₀ alkylene, C₁-C₈ alkylene, or C₁-C₄ alkylene).

In embodiments, V is substituted (e.g. with a substituent group, a size-limited substituent group or a lower substituent group) or unsubstituted alkylene (e.g., C₁-C₃₀ alkylene, C₁-C₈ alkylene, or C₁-C₄ alkylene), substituted (e.g. with a substituent group, a size-limited substituent group or a lower substituent group) or unsubstituted cycloalkylene (e.g., C₃-C₈ cycloalkylene, C₃-C₆ cycloalkylene, or C₅-C₆ cycloalkylene), or substituted (e.g. with a substituent group, a size-limited substituent group or a lower substituent group) or unsubstituted arylene (e.g., C₆-C₁₀ arylene, C₁₀ arylene, or phenylene). In embodiments, V is substituted with one or more substituent groups. In embodiments, V is substituted with one or more size-limited substituent groups. In embodiments, V is substituted with one or more lower substituent groups.

In embodiments, V is substituted (e.g. with a substituent group, a size-limited substituent group or a lower substituent group) alkylene (e.g., C₁-C₃₀ alkylene, C₁-C₈ alkylene, or C₁-C₄ alkylene). In embodiments, V is unsubstituted alkylene (e.g., C₁-C₃₀ alkylene, C₁-C₈ alkylene, or C₁-C₄ alkylene). In embodiments, V is substituted (e.g. with a substituent group, a size-limited substituent group or a lower substituent group) cycloalkylene (e.g., C₃-C₈ cycloalkylene, C₃-C₆ cycloalkylene, or C₅-C₆ cycloalkylene). In embodiments, V is unsubstituted cycloalkylene (e.g., C₃-C₈ cycloalkylene, C₃-C₆ cycloalkylene, or C₅-C₆ cycloalkylene). In embodiments, V is substituted (e.g. with a substituent group, a size-limited substituent group or a lower substituent group) arylene (e.g., C₆-C₁₀ arylene, C₁₀ arylene, or phenylene). In embodiments, V is unsubstituted arylene (e.g., C₆-C₁₀ arylene, C₁₀ arylene, or phenylene).

In embodiments, V is substituted or unsubstituted alkylene. In embodiments, V is substituted (e.g. with a substituent group, a size-limited substituent group or a lower substituent group) alkylene (e.g., C₁-C₃₀ alkylene, C₁-C₈ alkylene, or C₁-C₄ alkylene).

In embodiments, B⁸, B⁹, B¹⁰, and B¹¹ are each independently a bond, substituted (e.g. with a substituent group, a size-limited substituent group or a lower substituent group) or unsubstituted alkylene (e.g., C₁-C₃₀ alkylene, C₁-C₈ alkylene, or C₁-C₄ alkylene), or substituted (e.g. with a substituent group, a size-limited substituent group or a lower substituent group) or unsubstituted heteroalkylene (e.g., 2 to 30 membered heteroalkylene, 2 to 8 membered heteroalkylene, or 2 to 4 membered heteroalkylene). In embodiments, B⁸, B⁹, B¹⁰, and B¹¹ are each independently substituted with one or more substituent groups. In embodiments, B⁸, B⁹, B¹⁰, and B¹¹ are each independently substituted with one or more size-limited substituent groups. In embodiments, B⁸, B⁹, B¹⁰, and B¹¹ are each independently substituted with one or more lower substituent groups.

In embodiments, B⁸, B⁹, B¹⁰, and B¹¹ are each independently a bond. In embodiments, B⁸, B⁹, B¹⁰, and B¹¹ are each independently substituted (e.g. with a substituent group, a size-limited substituent group or a lower substituent group) alkylene (e.g., C₁-C₃₀ alkylene, C₁-C₈ alkylene, or C₁-C₄ alkylene). In embodiments, B⁸, B⁹, B¹⁰, and B¹¹ are each independently unsubstituted alkylene (e.g., C₁-C₃₀ alkylene, C₁-C₈ alkylene, or C₁-C₄ alkylene). In embodiments, B⁸, B⁹, B¹⁰, and B¹¹ are each independently substituted (e.g. with a substituent group, a size-limited substituent group or a lower substituent group) heteroalkylene (e.g., 2 to 30 membered heteroalkylene, 2 to 8 membered heteroalkylene, or 2 to 4 membered heteroalkylene). In embodiments, B⁸, B⁹, B¹⁰, and B¹¹ are each independently unsubstituted heteroalkylene (e.g., 2 to 30 membered heteroalkylene, 2 to 8 membered heteroalkylene, or 2 to 4 membered heteroalkylene).

In embodiments, B⁸, B⁹, B¹⁰, and B¹¹ are each independently substituted or unsubstituted alkylene. In embodiments, B⁸, B⁹, B¹⁰, and B¹¹ are each independently substituted (e.g. with a substituent group, a size-limited substituent group or a lower substituent group) or unsubstituted alkylene (e.g. with a substituent group, a size-limited substituent group or a lower substituent group).

In embodiments, B⁸, B⁹, B¹⁰, and B¹¹ are each independently substituted or unsubstituted C₁-C₂₀ alkylene. In embodiments, B⁸, B⁹, B¹⁰, and B¹¹ are each independently substituted (e.g. with a substituent group, a size-limited substituent group or a lower substituent group) or unsubstituted C₁-C₂₀ alkylene. In embodiments, B⁸, B⁹, B¹⁰, and B¹¹ are each independently substituted (e.g. with a substituent group, a size-limited substituent group or a lower substituent group) C₁-C₂₀ alkylene. In embodiments, B⁸, B⁹, B¹⁰, and B¹¹ are each independently unsubstituted C₁-C₂₀ alkylene.

In embodiments, B⁸, B⁹, B¹⁰, and B¹¹ are each independently substituted or unsubstituted C₁-C₈ alkylene. In embodiments, B⁸, B⁹, B¹⁰, and B¹¹ are each independently substituted (e.g. with a substituent group, a size-limited substituent group or a lower substituent group) or unsubstituted C₁-C₈ alkylene. In embodiments, B⁸, B⁹, B¹⁰, and B¹¹ are each independently substituted (e.g. with a substituent group, a size-limited substituent group or a lower substituent group) C₁-C₈ alkylene. In embodiments, B⁸, B⁹, B¹⁰, and B¹¹ are each independently unsubstituted C₁-C₈ alkylene.

In embodiments, L¹² is a bond, —O(C═O)—, —(C═O)O—, —O(C═O)O—, —C(═O)—, or —O—. In embodiments, L¹² is a bond. In embodiments, L¹² is —O(C═O)—. In embodiments, L¹² is —(C═O)O—. In embodiments, L¹² is —O(C═O)O—. In embodiments, L¹² is-C(═O)—. In embodiments, L¹² is —O—.

In embodiments, L¹² is —O(C═O)— or —(C═O)O—.

In embodiments, L¹³ is a bond, —O(C═O)—, —(C═O)O—, —O(C═O)O—, —C(═O)—, or —O—. In embodiments, L¹³ is a bond. In embodiments, L¹³ is —O(C═O)—. In embodiments, L¹³ is —(C═O)O—. In embodiments, L¹³ is —O(C═O)O—. In embodiments, L¹³ is-C(═O)—. In embodiments, L¹³ is —O—.

In embodiments, L¹³ is —O(C═O)— or —(C═O)O—.

In embodiments, R¹² is H, —OR^{12A}, —SR^{12A}, —NR^{12A}, —CN, or —(C═O)R^{12A}. In embodiments, R¹² is H, —OR^{12A}, or —NR^{12A}. In embodiments, R¹² is H or —OR^{12A}.

In embodiments, R¹² is H. In embodiments, R¹² is —OR^{12A}. In embodiments, R¹² is —SR^{12A}. In embodiments, R¹² is —NR^{12A}. In embodiments, R¹² is CN. In embodiments, R¹² is —(C═O)R^{12A}.

In embodiments, R¹² is —OH, methoxy, or ethoxy.

In embodiments, R¹³ is H, —OR^{13A}, —SR^{13A}, —NR^{13A}, —CN, or —(C═O)R^{13A}. In embodiments, R¹³ is H, —OR^{13A}, or —NR^{13A} In embodiments, R¹³ is H or —OR^{13A}.

In embodiments, R¹³ is H. In embodiments, R¹³ is —OR^{13A}. In embodiments, R¹³ is —SR^{13A}. In embodiments, R¹³ is —NR^{13A}. In embodiments, R¹³ is CN. In embodiments, R¹³ is —(C═O)R^{13A}.

In embodiments, R¹³ is —OH, methoxy, or ethoxy.

In embodiments, R^{12A} and R^{13A} are each independently H, substituted (e.g. with a substituent group, a size-limited substituent group or a lower substituent group) or unsubstituted C₁-C₂₀ alkyl, or substituted (e.g. with a substituent group, a size-limited substituent group or a lower substituent group) or unsubstituted 2 to 30 membered heteroalkyl. In embodiments, R^{12A} and R^{13A} are each independently substituted with one or more substituent groups. In embodiments, R^{12A} and R^{13A} are each independently substituted with one or more size-limited substituent groups. In embodiments, R^{12A} and R^{13A} are each independently substituted with one or more lower substituent groups.

In embodiments, R^{12A} and R^{13A} are each independently H. In embodiments, R^{12A} and R^{13A} are each independently substituted (e.g. with a substituent group, a size-limited substituent group or a lower substituent group) C₁-C₂₀ alkyl. In embodiments, R^{12A} and R^{13A} are each independently unsubstituted C₁-C₂₀ alkyl. In embodiments, R^{12A} and R^{13A} are each independently substituted (e.g. with a substituent group, a size-limited substituent group or a lower substituent group) 2 to 30 membered heteroalkyl. In embodiments, R^{12A} and R^{13A} are each independently unsubstituted 2 to 30 membered heteroalkyl.

In embodiments, R^{12A} and R^{13A} are each independently H, substituted or unsubstituted C₁-C₂₀ alkyl. In embodiments, R^{12A} and R^{13A} are each independently H, substituted (e.g. with a substituent group, a size-limited substituent group or a lower substituent group) or unsubstituted C₁-C₂₀ alkyl. In embodiments, R^{12A} and R^{13A} are each independently substituted (e.g. with a substituent group, a size-limited substituent group or a lower substituent group) C₁-C₂₀ alkyl. In embodiments, R^{12A} and R^{13A} are each independently unsubstituted C₁-C₂₀ alkyl.

In embodiments, R^{12A} and R^{13A} are each independently H, substituted or unsubstituted C₁-C₈ alkyl. In embodiments, R^{12A} and R^{13A} are each independently H, substituted (e.g. with a substituent group, a size-limited substituent group or a lower substituent group) or unsubstituted C₁-C₈ alkyl. In embodiments, R^{12A} and R^{13A} are each independently substituted (e.g. with a substituent group, a size-limited substituent group or a lower substituent group) C₁-C₈ alkyl. In embodiments, R^{12A} and R^{13A} are each independently unsubstituted C₁-C₈ alkyl.

In embodiments, R^{12B} and R^{13B} are each independently H, substituted (e.g. with a substituent group, a size-limited substituent group or a lower substituent group) or unsubstituted C₁-C₂₀ alkyl, or substituted (e.g. with a substituent group, a size-limited substituent group or a lower substituent group) or unsubstituted 2 to 30 membered heteroalkyl.

In embodiments, R^{12B} and R^{13B} are each independently H. In embodiments, R^{12B} and R^{13B} are each independently substituted (e.g. with a substituent group, a size-limited substituent group or a lower substituent group) C₁-C₂₀ alkyl. In embodiments, R^{12B} and R^{13B} are each independently unsubstituted C₁-C₂₀ alkyl. In embodiments, R^{12B} and R^{13B} are each independently substituted (e.g. with a substituent group, a size-limited substituent group or a lower substituent group) 2 to 30 membered heteroalkyl. In embodiments, R^{12B} and R^{13B} are each independently unsubstituted 2 to 30 membered heteroalkyl.

In embodiments, R¹⁴ and R¹⁵ are each independently substituted (e.g. with a substituent group, a size-limited substituent group or a lower substituent group) or unsubstituted C₂-C₃₀ alkyl, or substituted (e.g. with a substituent group, a size-limited substituent group or a lower substituent group) or unsubstituted 2 to 30 membered heteroalkyl. In embodiments, R¹⁴ and R¹⁵ are each independently substituted with one or more substituent groups. In embodiments, R¹⁴ and R¹⁵ are each independently substituted with one or more size-limited substituent groups. In embodiments, R¹⁴ and R¹⁵ are each independently substituted with one or more lower substituent groups.

In embodiments, R¹⁴ and R¹⁵ are each independently substituted (e.g. with a substituent group, a size-limited substituent group or a lower substituent group) C₂-C₃₀ alkyl. In embodiments, R¹⁴ and R¹⁵ are each independently unsubstituted C₂-C₃₀ alkyl. In embodiments, R¹⁴ and R¹⁵ are each independently substituted (e.g. with a substituent group, a size-limited substituent group or a lower substituent group) 2 to 30 membered heteroalkyl. In embodiments, R¹⁴ and R¹⁵ are each independently unsubstituted 2 to 30 membered heteroalkyl.

In embodiments, R¹⁴ and R¹⁵ are each independently substituted or unsubstituted C₂-C₃₀ alkyl. In embodiments, R¹⁴ and R¹⁵ are each independently substituted (e.g. with a substituent group, a size-limited substituent group or a lower substituent group) or unsubstituted C₂-C₃₀ alkyl.

In embodiments, each R²¹⁰, R²¹¹, R³¹⁰, and R³¹¹ is independently H, substituted (e.g. with a substituent group, a size-limited substituent group or a lower substituent group) or unsubstituted C₁-C₁₂ alkyl, or substituted (e.g. with a substituent group, a size-limited substituent group or a lower substituent group) or unsubstituted 2 to 12 membered heteroalkyl. In embodiments, each R²¹⁰, R²¹¹, R³¹⁰, and R³¹¹ is independently substituted with one or more substituent groups. In embodiments, each R²¹⁰, R²¹¹, R³¹⁰, and R³¹¹ is independently substituted with one or more size-limited substituent groups. In embodiments, each R²¹⁰, R²¹¹, R³¹⁰, and R³¹¹ is independently substituted with one or more lower substituent groups.

In embodiments, each R²¹⁰, R²¹¹, R³¹⁰, and R³¹¹ is independently H. In embodiments, each R²¹⁰, R²¹¹, R³¹⁰, and R³¹¹ is independently substituted (e.g. with a substituent group, a size-limited substituent group or a lower substituent group) C₁-C₁₂ alkyl. In embodiments, each R²¹⁰, R²¹¹, R³¹⁰, and R³¹¹ is independently unsubstituted C₁-C₁₂ alkyl. In embodiments, each R²¹⁰, R²¹¹, R³¹⁰, and R³¹¹ is independently substituted (e.g. with a substituent group, a size-limited substituent group or a lower substituent group) 2 to 12 membered heteroalkyl. In embodiments, each R²¹⁰, R²¹¹, R³¹⁰, and R³¹¹ is independently unsubstituted 2 to 12 membered heteroalkyl.

In embodiments, each n is independently an integer from 0 to 8. In embodiments, each n is independently an integer from 0 to 4. In embodiments, each n is independently 8. In embodiments, each n is independently 7. In embodiments, each n is independently 6. In embodiments, each n is independently 5. In embodiments, each n is independently 4. In embodiments, each n is independently 3. In embodiments, each n is independently 2. In embodiments, each n is independently 1. In embodiments, each n is independently 0.

In embodiments, each s is an integer from 1 to 4. In embodiments, each s is 4. In embodiments, each s is 3. In embodiments, each s is 2. In embodiments, each s is 1.

In embodiments,
L¹¹ is

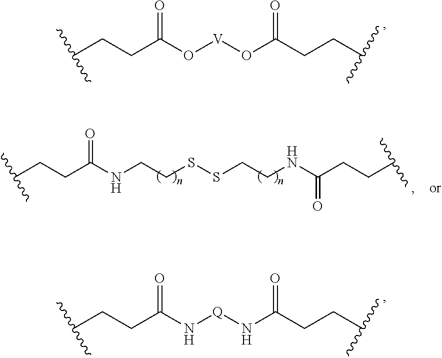

where Q is substituted or unsubstituted alkylene, V is substituted or unsubstituted alkylene and each n is independently an integer from 0 to 8.

B⁸, B⁹, B¹⁰, and B¹¹ are each independently substituted or unsubstituted alkylene.

L¹² is —O(C═O)— or —(C═O)O—. L¹³ is —O(C═O)— or —(C═O)O—. R¹² is H—, OR^{12A}, or —NR^{12A}.

R¹³ is H, —OR^{13A}, or —NR^{13A}. R¹⁴ and R¹⁵ are each independently substituted or unsubstituted C₂-C₃₀ alkyl. R^{12A} and R^{13A} are each independently H, substituted or unsubstituted C₁-C₂₀ alkyl.

In embodiments,
L¹¹ is

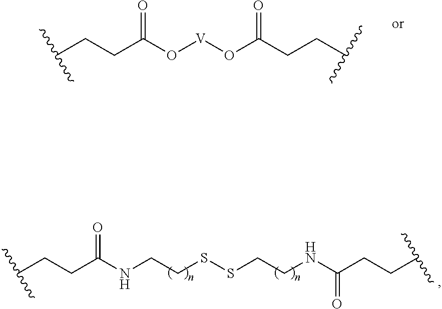

where V is substituted or unsubstituted alkylene and each n is independently an integer from 0 to 4.

B⁸, B⁹, B¹⁰, and B¹¹ are each independently substituted or unsubstituted C₁-C₂₀ alkylene.

L¹² is —O(C═O)— or —(C═O)O—. L¹³ is —O(C═O)— or —(C═O)O—. R¹² is H or —OR^{12A}. R¹³ is H or —OR^{13A}. R¹⁴ and R¹⁵ are each independently substituted or unsubstituted C₂-C₂₀ alkyl.

R^{12A} and R^{13A} are each independently H, substituted or unsubstituted C₁-C₈ alkyl.

In embodiments,

L¹¹ is

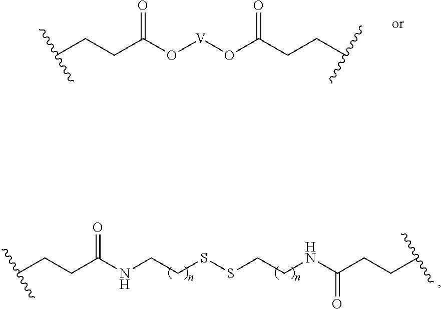

where V is substituted or unsubstituted alkylene and each n is independently an integer from 0 to 4.

B⁸, B⁹, B¹⁰, and B¹¹ are each independently substituted or unsubstituted C₁-C₈ alkylene.

L¹² is —O(C═O)— or —(C═O)O—. L¹³ is —O(C═O)— or —(C═O)O—. R¹² is —OH, methoxy, or ethoxy.

R¹³ is —OH, methoxy, or ethoxy. R¹⁴ and R¹⁵ are each independently substituted or unsubstituted C₂-C₂₀ alkyl.

In embodiments, the cationic lipid of formula (III) is:

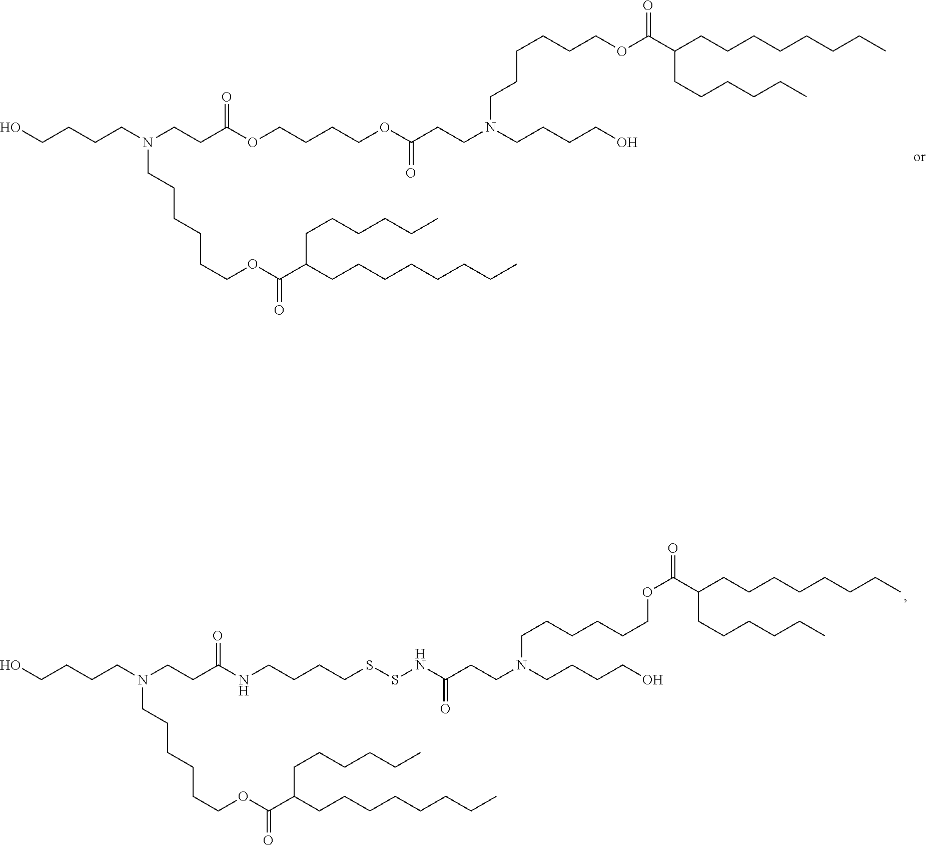 [SMILES: CCCCCCCCC(CCCCCC)C(=O)OCCCCCCN(CCCCO)CCC(=O)OCCCCOC(=O)CCN(CCCCO)CCCCCCOC(=O)C(CCCCCC)CCCCCCCC ; CCCCCCCCC(CCCCCC)C(=O)OCCCCCCN(CCCCO)CCC(=O)NCCCCSSNC(=O)CCN(CCCCO)CCCCCCOC(=O)C(CCCCCC)CCCCCCCC]

or a pharmaceutically acceptable salt thereof.

In an aspect, provided herein is cationic lipid of formula (IV):

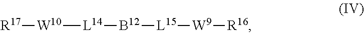

or a pharmaceutically acceptable salt, solvate, hydrate, stereoisomer, or prodrug thereof.

B¹² is —W⁷-L^{a3}-W⁸—.

W⁷ and W⁸ are each independently a bond, substituted or unsubstituted C₁-C₁₂ alkylene, or substituted or unsubstituted 2 to 12 membered heteroalkylene.

L^{a3} is a bond, —S—S—, —O—(CH₂O)ₘ—,

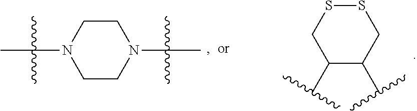 [SMILES: *N1CCN(*)CC1 ; *C1CSSCC1*]

W⁹ and W¹⁰ are each independently a bond, substituted or unsubstituted C₁-C₁₂ alkylene, substituted or unsubstituted 2 to 12 membered heteroalkylene, substituted or unsubstituted cycloalkylene, substituted or unsubstituted heterocycloalkylene, or any combination thereof.

L¹⁴ is a bond, —O(C═O)—, —(C═O)O—, —O(C═O)O—, —C(═O)—, —O—, —O(CR⁴¹⁰R⁴¹¹)ₛO—, —S—, —C(═O)S—, —SC(═O)—, —NR⁴¹⁰C(═O)—, —C(═O)NR⁴¹⁰—, —NR⁴¹⁰C(═O)NR⁴¹⁰—, —NR⁴¹⁰C(═S)—, —C(═S)NR⁴¹⁰—, —NR⁴¹⁰C(═S)NR⁴¹⁰—, —OC(═O)NR⁴¹⁰—, —NR⁴¹⁰C(═O)O—, —SC(═O)NR⁴¹⁰— or —NR⁴¹⁰C(═O)S—.

L¹⁵ is a bond, —O(C═O)—, —(C═O)O—, —O(C═O)O—, —C(═O)—, —O—, —O(CR⁵¹⁰R⁵¹¹)ₛO—, —S—, —C(═O)S—, —SC(═O)—, —NR⁵¹⁰C(═O)—, —C(═O)NR⁵¹⁰—, —NR⁵¹⁰C(═O)NR⁵¹¹—, —NR⁵¹⁰C(═S)—, —C(═S)NR⁵¹⁰—, —NR⁵¹⁰C(═S)NR⁵¹¹—, —OC(═O)NR⁵¹⁰—, —NR⁵¹⁰C(═O)O—, —SC(═O)NR⁵¹⁰— or —NR⁵¹⁰C(═O)S—.

R¹⁶ and R¹⁷ are each independently

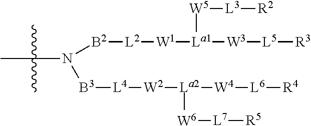

a fragment of cationic lipid of formula (I),

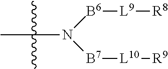

a fragment of cationic lipid of formula (II),

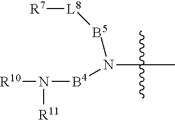

fragment of cationic lipid of formula (II),

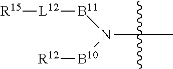

a fragment of cationic lipid of formula (III),

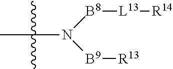

a fragment of cationic lipid of formula (III).

each R⁴¹⁰, R⁴¹¹, R⁵¹⁰, and R⁵¹¹ is independently H, substituted or unsubstituted C₁-C₁₂ alkyl, or substituted or unsubstituted 2 to 12 membered heteroalkyl.

each m is independently an integer from 0 to 8, and

each s is independently an integer from 1 to 4.

In embodiments, W⁷ and W⁸ are each independently a bond, substituted (e.g. with a substituent group, a size-limited substituent group or a lower substituent group) or unsubstituted C₁-C₁₂ alkylene, or substituted (e.g. with a substituent group, a size-limited substituent group or a lower substituent group) or unsubstituted 2 to 12 membered heteroalkylene. In embodiments, W⁷ and W⁸ are each independently substituted with one or more substituent groups. In embodiments, W⁷ and W⁸ are each independently substituted with one or more size-limited substituent groups. In embodiments, W⁷ and W⁸ are each independently substituted with one or more lower substituent groups.

In embodiments, W⁷ and W⁸ are each independently a bond. In embodiments, W⁷ and W⁸ are each independently substituted (e.g. with a substituent group, a size-limited substituent group or a lower substituent group) C₁-C₁₂ alkylene. In embodiments, W⁷ and W⁸ are each independently unsubstituted C₁-C₁₂ alkylene. In embodiments, W⁷ and W⁸ are each independently substituted (e.g. with a substituent group, a size-limited substituent group or a lower substituent group) 2 to 12 membered heteroalkylene. In embodiments, W⁷ and W⁸ are each independently unsubstituted 2 to 12 membered heteroalkylene.

In embodiments, W⁷ and W⁸ are each independently a bond or substituted or unsubstituted C₁-C₁₂ alkylene. W⁷ and W⁸ are each independently a bond or substituted (e.g. with a substituent group, a size-limited substituent group or a lower substituent group) or unsubstituted C₁-C₁₂ alkylene

In embodiments, W⁷ and W⁸ are each independently a bond or unsubstituted C₁-C₁₂ alkylene.

In embodiments, W⁷ and W⁸ are each independently a bond or unsubstituted C₁-C₈ alkylene. In embodiments, W⁷ and W⁸ are each independently unsubstituted C₁-C₈ alkylene.

In embodiments, L^{a3} is a bond, —S—S—,

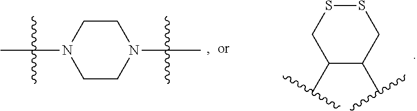 [SMILES: *N1CCN(*)CC1 ; *C1CSSCC1*]

In embodiments, L^{a3} is a bond. In embodiments, L^{a3} is —S—S—. In embodiments, L^{a3} is

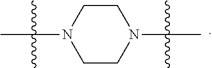 [SMILES: *N1CCN(*)CC1]

In embodiments, L^{a3} is

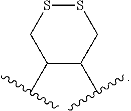 [SMILES: *C1CSSCC1*]

In embodiments, W⁹ and W¹⁰ are each independently a bond, substituted (e.g. with a substituent group, a size-limited substituent group or a lower substituent group) or unsubstituted C₁-C₁₂ alkylene, substituted (e.g. with a substituent group, a size-limited substituent group or a lower substituent group) or unsubstituted 2 to 12 membered heteroalkylene, substituted (e.g. with a substituent group, a size-limited substituent group or a lower substituent group) or unsubstituted cycloalkylene (e.g., C₃-C₈ cycloalkylene, C₃-C₆ cycloalkylene, or C₅-C₆ cycloalkylene), substituted (e.g. with a substituent group, a size-limited substituent group or a lower substituent group) or unsubstituted heterocycloalkylene(e.g., 3 to 8 membered heterocycloalkylene, 3 to 6 membered heterocycloalkylene, or 5 to 6 membered heterocycloalkylene), or any combination thereof. In embodiments, W⁹ and W¹⁰ are each independently substituted with one or more substituent groups. In embodiments, W⁹ and W¹⁰ are each independently substituted with one or more size-limited substituent groups. In embodiments, W⁹ and W¹⁰ are each independently substituted with one or more lower substituent groups.

In embodiments, W⁹ and W¹⁰ are each independently a bond. In embodiments, W⁹ and W¹⁰ are each independently substituted (e.g. with a substituent group, a size-limited substituent group or a lower substituent group) C₁-C₁₂ alkylene. In embodiments, W⁹ and W¹⁰ are each independently unsubstituted C₁-C₁₂ alkylene. In embodiments, W⁹ and W¹⁰ are each independently substituted (e.g. with a substituent group, a size-limited substituent group or a lower substituent group) 2 to 12 membered heteroalkylene. In embodiments, W⁹ and W¹⁰ are each independently unsubstituted 2 to 12 membered heteroalkylene. In embodiments, W⁹ and W¹⁰ are each independently substituted (e.g. with a substituent group, a size-limited substituent group or a lower substituent group) cycloalkylene (e.g., C₃-C₈ cycloalkylene, C₃-C₆ cycloalkylene, or C₅-C₆ cycloalkylene). In embodiments, W⁹ and W¹⁰ are each independently unsubstituted cycloalkylene (e.g., C₃-C₈ cycloalkylene, C₃-C₆ cycloalkylene, or C₅-C₆ cycloalkylene). In embodiments, W⁹ and W¹⁰ are each independently substituted (e.g. with a substituent group, a size-limited substituent group or a lower substituent group) heterocycloalkylene(e.g., 3 to 8 membered heterocycloalkylene, 3 to 6 membered heterocycloalkylene, or 5 to 6 membered heterocycloalkylene). In embodiments, W⁹ and W¹⁰ are each independently unsubstituted heterocycloalkylene(e.g., 3 to 8 membered heterocycloalkylene, 3 to 6 membered heterocycloalkylene, or 5 to 6 membered heterocycloalkylene).

In embodiments, W⁹ and W¹⁰ are each independently a bond or substituted or unsubstituted C₁-C₁₂ alkylene. In embodiments, W⁹ and W¹⁰ are each independently a bond or substituted (e.g. with a substituent group, a size-limited substituent group or a lower substituent group) or unsubstituted C₁-C₁₂ alkylene. In embodiments, W⁹ and W¹⁰ are each independently substituted (e.g. with a substituent group, a size-limited substituent group or a lower substituent group) C₁-C₁₂ alkylene. In embodiments, W⁹ and W¹⁰ are each independently unsubstituted C₁-C₁₂ alkylene.

In embodiments, W⁹ and W¹⁰ are each independently a bond or unsubstituted C₁-C₈ alkylene. In embodiments, W⁹ and W¹⁰ are each independently unsubstituted C₁-C₈ alkylene.

In embodiments, L¹⁴ is-O(C═O)—, —(C═O)O—, —C(═O)—, —NR⁴¹⁰C(═O)—, —C(═O)NR⁴¹⁰—,

—NR⁴¹⁰C(═S)—, —C(═S)NR⁴¹⁰—, —OC(═O)NR⁴¹⁰—, or —NR⁴¹⁰C(═O)O—. In embodiments, L¹⁴ is —O(C═O)—, —(C═O)O—, —NR⁴¹⁰C(═S)—, —C(═S)NR⁴¹⁰—, —OC(═O)NR⁴¹⁰—, or —NR⁴¹⁰C(═O)O—.

In embodiments, L¹⁴ is —O(C═O)—. In embodiments, L¹⁴ is —(C═O)O—. In embodiments, L¹⁴ is-C(═O)—. In embodiments, L¹⁴ is —NR⁴¹⁰C(═O)—. In embodiments, L¹⁴ is —C(═O)NR⁴¹⁰—. In embodiments, L¹⁴ is —NR⁴¹⁰C(═S)—. In embodiments, L¹⁴ is —C(═S)NR⁴¹⁰—. In embodiments, L¹⁴ is —OC(═O)NR⁴¹⁰—. In embodiments, L¹⁴ is —NR⁴¹⁰C(═O)O—.

In embodiments, L¹⁵ is-O(C═O)—, —(C═O)O—, —C(═O)—, —NR⁵¹⁰C(═O)—, —C(═O)NR⁵¹⁰—,

—NR⁵¹⁰C(═S)—, —C(═S)NR⁵¹⁰—, —OC(═O)NR⁵¹⁰—, or —NR⁵¹⁰C(═O)O—. In embodiments, L¹⁵ is —O(C═O)—, —(C═O)O—, —NR⁵¹⁰C(═S)—, —C(═S)NR⁵¹⁰—, —OC(═O)NR⁵¹⁰—, or —NR⁵¹⁰C(═O)O—.

In embodiments, L¹⁵ is —O(C═O)—. In embodiments, L¹⁵ is —(C═O)O—. In embodiments, L¹⁵ is-C(═O)—. In embodiments, L¹⁵ is —NR⁵¹⁰C(═O)—. In embodiments, L¹⁵ is —C(═O)NR⁵¹⁰—. In embodiments, L¹⁵ is —NR⁵¹⁰C(═S)—. In embodiments, L¹⁵ is —C(═S)NR⁵¹⁰—. In embodiments, L¹⁵ is —OC(═O)NR⁵¹⁰—. In embodiments, L⁵ is —NR⁵¹⁰C(═O)O—.

In embodiments, each R⁴¹⁰, R⁴¹¹, R⁵¹⁰, and R⁵¹¹ is independently H, substituted (e.g. with a substituent group, a size-limited substituent group or a lower substituent group) or unsubstituted C₁-C₁₂ alkyl, or substituted (e.g. with a substituent group, a size-limited substituent group or a lower substituent group) or unsubstituted 2 to 12 membered heteroalkyl. In embodiments, each R⁴¹⁰, R⁴¹¹, R⁵¹⁰, and R⁵¹¹ is independently substituted with one or more substituent groups. In embodiments, each R⁴¹⁰, R⁴¹¹, R⁵¹⁰, and R⁵¹¹ is independently substituted with one or more size-limited substituent groups. In embodiments, each R⁴¹⁰, R⁴¹¹, R⁵¹⁰, and R⁵¹¹ is independently substituted with one or more lower substituent groups.

In embodiments, each R⁴¹⁰, R⁴¹¹, R⁵¹⁰, and R⁵¹¹ is independently H. In embodiments, each R⁴¹⁰, R⁴¹¹, R⁵¹⁰, and R⁵¹¹ is independently substituted (e.g. with a substituent group, a size-limited substituent group or a lower substituent group) C₁-C₁₂ alkyl. In embodiments, each R⁴¹⁰, R⁴¹¹, R⁵¹⁰, and R⁵¹¹ is independently unsubstituted C₁-C₁₂ alkyl. In embodiments, each R⁴¹⁰, R⁴¹¹, R⁵¹⁰, and R⁵¹¹ is independently substituted (e.g. with a substituent group, a size-limited substituent group or a lower substituent group) 2 to 12 membered heteroalkyl. In embodiments, each R⁴¹⁰, R⁴¹¹, R⁵¹⁰, and R⁵¹¹ is independently unsubstituted 2 to 12 membered heteroalkyl.

In embodiments, each R⁴¹⁰ and R¹⁰⁰ is independently H or substituted or unsubstituted C₁-C₁₂ alkyl. In embodiments, each R⁴¹⁰ and R⁵¹⁰ is independently H or substituted (e.g. with a substituent group, a size-limited substituent group or a lower substituent group) or unsubstituted C₁-C₁₂ alkyl.

In embodiments, each R⁴¹⁰ and R¹⁰⁰ is independently H or unsubstituted C₁-C₈ alkyl. In embodiments, each R⁴¹⁰ and R⁵¹⁰ is independently unsubstituted C₁-C₈ alkyl.

In embodiments, each R⁴¹⁰ and R⁵¹⁰ is independently H, methyl, ethyl, propyl, butyl, pentyl, hexyl, heptyl, or octyl. In embodiments, each R⁴¹⁰ and R⁵¹⁰ is independently H or methyl. In embodiments, each R⁴¹⁰ and R⁵¹⁰ is independently H. In embodiments, each R⁴¹⁰ and R⁵¹⁰ is independently methyl.

In embodiments, R¹⁶ and R¹⁷ are each independently

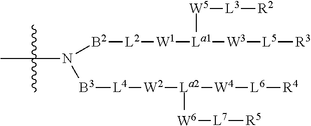

a fragment of cationic lipid of formula (I),

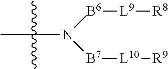

a fragment of cationic lipid of formula (II),

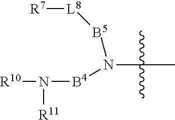

a fragment of cationic lipid of formula (II),

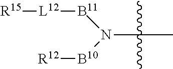

a fragment of cationic lipid of formula (III), or

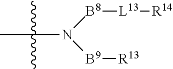

a fragment of cationic lipid of formula (III), where B², B³, B⁴, B⁵, B⁶, B⁷, B⁸, B⁹, B¹⁰, B¹¹, L⁹, L², L³, L⁴, L⁵, L⁶, L⁷, L⁹, L⁹, L¹⁰, L¹², L¹³, R², R³, R⁴, R⁵, R⁷, R⁸, R⁹, R¹⁰, R¹¹, R¹², R¹³, R¹⁴, and R¹⁵ are as described herein including embodiments.

In embodiments, R¹⁶ and R¹⁷ are each independently

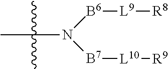

a fragment of cationic lipid of formula (II), where B⁶, B⁷, L⁹, L¹⁰, R⁸, and R⁹ are as described herein including embodiments.

In embodiments, R¹⁶ and R¹⁷ are each independently

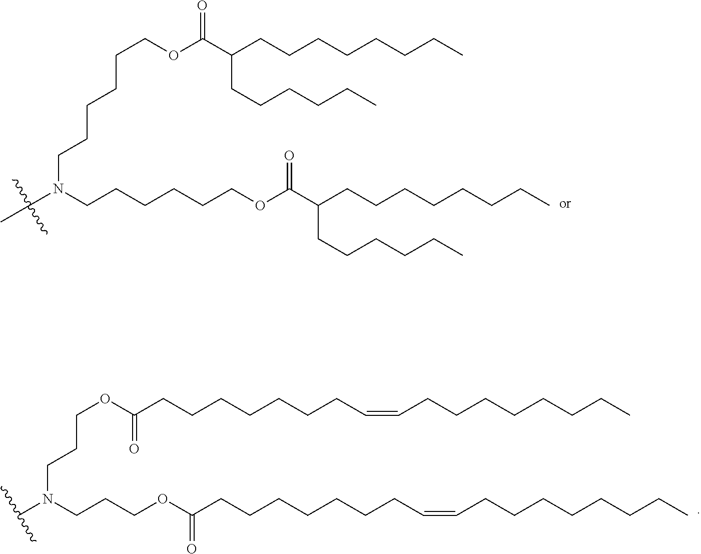 [SMILES: *N(CCCCCCOC(=O)C(CCCCCC)CCCCCCCC)CCCCCCOC(=O)C(CCCCCC)CCCCCCCC ; *N(CCCOC(=O)CCCCCCC/C=C\CCCCCCCC)CCCOC(=O)CCCCCCC/C=C\CCCCCCCC]

In embodiments, R¹⁶ and R¹⁷ are each independently

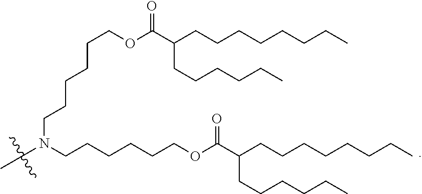 [SMILES: *N(CCCCCCOC(=O)C(CCCCCC)CCCCCCCC)CCCCCCOC(=O)C(CCCCCC)CCCCCCCC]

In embodiments, R¹⁶ and R¹⁷ are each independently

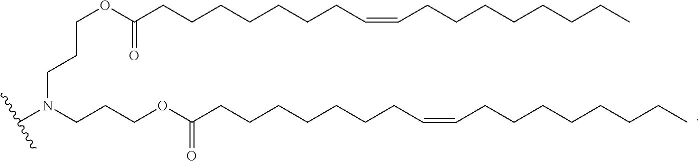 [SMILES: *N(CCCOC(=O)CCCCCCC/C=C\CCCCCCCC)CCCOC(=O)CCCCCCC/C=C\CCCCCCCC]

In embodiments, each m is independently an integer from 0 to 8. In embodiments, each m is independently 8. In embodiments, each m is independently 7. In embodiments, each m is independently 6. In embodiments, each m is independently 5. In embodiments, each m is independently 4. In embodiments, each m is independently 3. In embodiments, each m is independently 2. In embodiments, each m is independently 1. In embodiments, each m is independently 0.

In embodiments, each s is an integer from 1 to 4. In embodiments, each s is 4. In embodiments, each s is 3. In embodiments, each s is 2. In embodiments, each s is 1.

In embodiments, the cationic lipid of formula (IV) is:

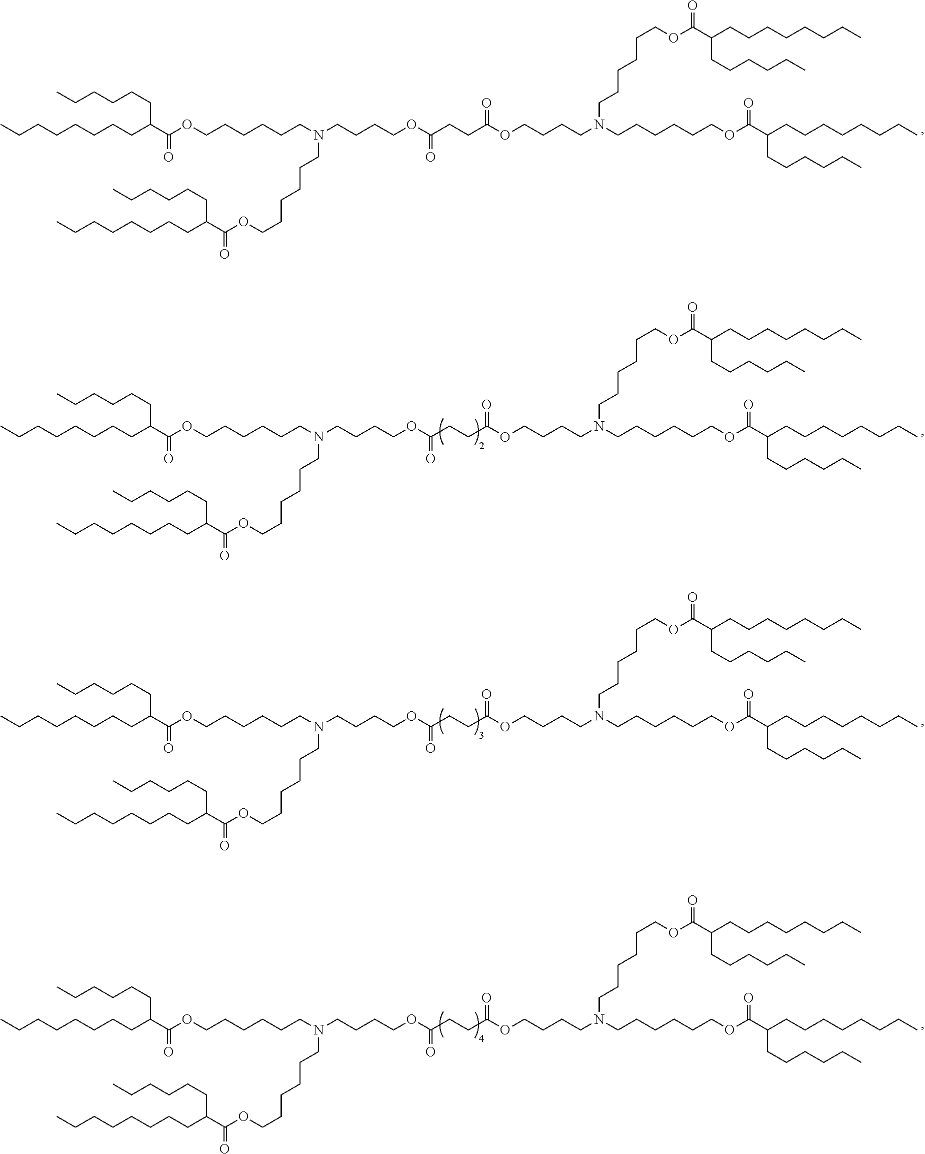

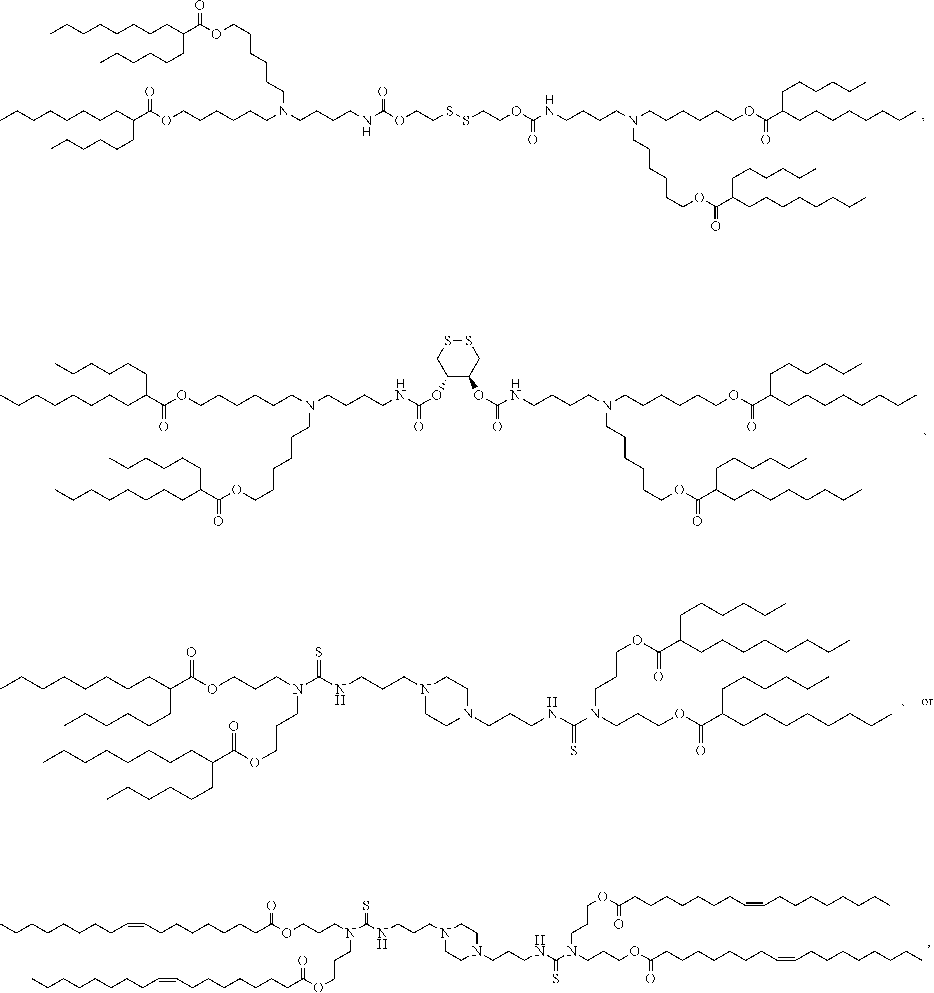

or a pharmaceutically acceptable salt thereof.

In some embodiments, the lipid is KT-001, ALC-0315, TU-001, TU-002, BAE-001, DS-001, or JK-0315-CA.

In some embodiments, the lipid is KT-001.

### Lipid Nanoparticles

In an aspect, provided herein are lipid nanoparticles comprising one or more of the ionizable cationic lipids or salts thereof described herein. In embodiments, the lipid nanoparticles described herein further include one or more non-cationic lipids. In embodiments, the lipid nanoparticles described herein further include one or more conjugated lipids capable of reducing or inhibiting particle aggregation. In other embodiments, the lipid nanoparticles described herein further include one or more therapeutic agents such as nucleic acids (e.g., mRNA).

In embodiments, lipid nanoparticles comprising one or more ionizable cationic lipids described herein are used to encapsulate nucleic acids (e.g., mRNA) within the lipid nanoparticles.

In embodiments, the lipid nanoparticles include a therapeutic agent such as nucleic acid (e.g., mRNA), a cationic lipid (one or more ionizable cationic lipids of formula I-IV or salts thereof, as described herein, or cationic lipids known in the art), a non-cationic lipid (e.g., mixtures of one or more phospholipids and cholesterol), and a conjugated lipid that inhibits aggregation of particles (e.g., one or more PEG-lipid conjugates).

In embodiments, non-cationic lipids that can be used in the lipid nanoparticles described herein include, without limitation, neutral, zwitterionic or anionic lipids, for example:
phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyolphosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, palmitoyl, stearoyl, or oleoyl.

In embodiments, non-cationic lipids may be sterols such as cholesterol and derivatives thereof. Non-limiting examples of cholesterol derivatives include polar analogues such as 5α-cholestanol, 5β-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5β-cholestanone, and cholesteryl decanoate; and mixtures thereof. In embodiments, the cholesterol derivative is a polar analogue such as cholesteryl-(4′-hydroxy)-butyl ether.

In embodiments, the non-cationic lipids included in the lipid nanoparticles include a mixture of one or more phospholipids and cholesterol or a derivative thereof.

In embodiments, non-cationic lipids suitable for use in the lipid nanoparticles include stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, sphingomyelin, and the like.

In embodiments, lipid conjugates that can be used in the lipid nanoparticles described herein include, without limitation, PEG-lipid conjugates, POZ-lipid conjugates, ATTA-lipid conjugates, cationic-polymer-lipid conjugates (CPLs), and mixtures thereof. In embodiments, the nanoparticles comprise PEG-lipid conjugate.

In embodiments, lipid conjugates that can be used in the lipid nanoparticles described herein include, PEG coupled to dialkyloxypropyls (PEG-DAA), PEG coupled to diacylglycerol (PEG-DAG), PEG coupled to phospholipids such as phosphatidylethanolamine (PEG-PE), PEG conjugated to ceramides, mPEG2000-1,2-di-O-alkyl-sn3-carbomoylglyceride (PEG-C-DOMG), 1-[8′-(1,2-dimyristoyl-3-propanoxy)-carboxamido-3′,6′-dioxaoctanyl]carbamoyl-co-methyl-poly(ethylene glycol) (2 KPEG-DMG), 1,2-Dimyristoyl-rac-glycero-3-methylpolyoxyethylene (DMG-PEG), PEG conjugated to cholesterol or a derivative thereof, and mixtures thereof.

In embodiments, lipid nanoparticles described herein are useful for the introduction of therapeutic agents such as nucleic acids (e.g., mRNA) into cells.

In an aspect, provided herein is a method for the in vivo delivery of a therapeutic agent comprising administering the lipid nanoparticle, composed of the ionizable cationic lipids of formula I-IV as described herein, to a mammal.

In embodiments, the lipid nanoparticles described herein can be administered either alone or in a mixture with a pharmaceutically acceptable carrier. Non-limiting examples of pharmaceutically acceptable carriers include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like.

The pharmaceutically acceptable carrier is usually added following lipid nanoparticle formation. Thus, after the lipid nanoparticle is formed, the nanoparticle can be diluted into pharmaceutically acceptable carriers such as normal buffered saline.

For in vivo administration, administration can be in any manner known in the art, e.g., by injection, oral administration, inhalation (e.g., intransal or intratracheal), transdermal application, or rectal administration.

In embodiments, the pharmaceutical compositions can be administered parenterally, i.e., intraarticularly, intravenously, intraperitoneally, subcutaneously, or intramuscularly. In embodiments, the pharmaceutical compositions are administered intravenously or intraperitoneally by a bolus injection.

In an aspect, provided herein is a method for preventing or treating a disease in a mammal in need thereof by administering to the mammal a therapeutically effective amount of a lipid nanoparticle composed of the ionizable cationic lipids of formula I-IV as described herein. In embodiments, provided herein is a method for preventing a disease in a mammal by administering to the mammal a therapeutically effective amount of a lipid nanoparticle composed of the ionizable cationic lipids of formula I-IV as described herein. In embodiments, provided herein is a method for treating a disease in a mammal, in need thereof, by administering to the mammal a therapeutically effective amount of a lipid nanoparticle composed of the ionizable cationic lipids of formula I-IV as described herein.

In embodiments the mammal is a dog, a cat or a human. In embodiments, the mammal is a dog. In embodiments, the mammal is a cat. In embodiments, the mammal is a human.

### Methods for Treating or Preventing Diseases or a Disorders

In still another aspect, the present invention is directed to a method for treating or preventing a disease or a disorder, e.g., a proliferation disease or disorder, an autoimmune or inflammatory disease or disorder, or an infectious disease or disorder, in a subject in need comprising administering to said subject an effective amount of a polynucleotide comprising a nucleic acid sequence encoding a fusion protein comprising a modified interleukin 2 (IL-2) polypeptide, e.g., DNA, RNA or vector, or a pharmaceutical composition such a polynucleotide, as described above.

The present method can be used for treating or preventing a disease or a disorder, e.g., a proliferation disease or disorder, in any suitable subject. For example, the present method can be used for treating or preventing a disease or a disorder, e.g., a proliferation disease or disorder, in a human. In another example, the present method can be used for treating or preventing a disease or a disorder, e.g., a proliferation disease or disorder, in a non-human mammal.

In one embodiment, the present method can be used to treat a proliferation disorder in a subject. In another embodiment, the present method can be used to prevent a proliferation disorder in a subject.

The present method can be used for treating or preventing any suitable proliferation disease or disorder in a subject. For example, the present method can be used for treating or preventing a tumor in a subject. In another example, the present method can be used for treating or preventing a cancer in a subject.

In one embodiment, the present method can be used to treat or prevent a solid tumor or cancer in a subject. The present method can be used to treat or prevent any suitable solid tumor or cancer in a subject. For example, the solid tumor or cancer can be Chondrosarcoma, Ewing's sarcoma, Malignant fibrous histiocytoma of bone/osteosarcoma, Osteosarcoma, Rhabdomyosarcoma, Heart cancer, Astrocytoma, Brainstem glioma, Pilocytic astrocytoma, Ependymoma, Primitive neuroectodermal tumor, Cerebellar astrocytoma, Cerebral astrocytoma, Glioma, Medulloblastoma, Neuroblastoma, Oligodendroglioma, Pineal astrocytoma, Pituitary adenoma, Visual pathway and hypothalamic glioma, Breast cancer, Invasive lobular carcinoma, Tubular carcinoma, Invasive cribriform carcinoma, Medullary carcinoma, Male breast cancer, Phyllodes tumor, Inflammatory Breast Cancer, Adrenocortical carcinoma, Islet cell carcinoma (endocrine pancreas), Multiple endocrine neoplasia syndrome, Parathyroid cancer, Pheochromocytoma, Thyroid cancer, Merkel cell carcinoma, Uveal melanoma, Retinoblastoma, Anal cancer, Appendix cancer, cholangiocarcinoma, Carcinoid tumor, gastrointestinal, Colon cancer, Extrahepatic bile duct cancer, Gallbladder cancer, Gastric (stomach) cancer, Gastrointestinal carcinoid tumor, Gastrointestinal stromal tumor (GIST), Hepatocellular cancer, Pancreatic cancer islet cell, Rectal cancer, Bladder cancer, Cervical cancer, Endometrial cancer, Extragonadal germ cell tumor, Ovarian cancer, Ovarian epithelial cancer (surface epithelial-stromal tumor), Ovarian germ cell tumor, Penile cancer, Renal cell carcinoma, Renal pelvis and ureter, transitional cell cancer, Prostate cancer, Testicular cancer, Gestational trophoblastic tumor, Ureter and renal pelvis, transitional cell cancer, Urethral cancer, Uterine sarcoma, Vaginal cancer, Vulvar cancer, Wilms tumor, Esophageal cancer, Head and neck cancer, Nasopharyngeal carcinoma, Oral cancer, Oropharyngeal cancer, Paranasal sinus and nasal cavity cancer, Pharyngeal cancer, Salivary gland cancer, Hypopharyngeal cancer, Basal-cell carcinoma, Melanoma, Skin cancer (non-melanoma), Bronchial adenomas/carcinoids, Small cell lung cancer, Mesothelioma, Non-small cell lung cancer, Pleuropulmonary blastoma, Laryngeal cancer, Thymoma and thymic carcinoma, AIDS-related cancers, Kaposi sarcoma, Epithelioid hemangioendothelioma (EHE), Desmoplastic small round cell tumor or Liposarcoma.

In another embodiment, the present method can be used to treat or prevent a hematological malignancy in a subject. The present method can be used to treat or prevent any suitable hematological malignancy in a subject. For example, the hematological malignancy can be myeloid neoplasms, Leukemias, Lymphomas, Hodgkin lymphoma, Non-Hodgkin lymphoma, Anaplastic large cell lymphoma, Angioimmunoblastic T-cell lymphoma, Hepatosplenic T-cell lymphoma, B-cell lymphoma reticuloendotheliosis, Reticulosis, Microglioma, Diffuse large B-cell lymphoma, Follicular lymphoma, Mucosa-associated lymphatic tissue lymphoma, B-cell chronic lymphocytic leukemia, Mantle cell lymphoma, Burkitt lymphoma, Mediastinal large B cell lymphoma, Waldenstrom's macroglobulinemia, Nodal marginal zone B cell lymphoma, Splenic marginal zone lymphoma, Intravascular large B-cell lymphoma, Primary effusion lymphoma, Lymphomatoid granulomatosis, Nodular lymphocyte predominant Hodgkin's lymphoma, plasma cell leukemia, Acute erythraemia and erythroleukaemia, Acute erythremic myelosis, Acute erythroid leukemia, Heilmeyer-Schoner disease, Acute megakaryoblastic leukemia, Mast cell leukemia, Panmyelosis, Acute panmyelosis with myelofibrosis, Lymphosarcoma cell leukemia, Acute leukaemia of unspecified cell type, Blastic phase chronic myelogenous leukemia, Stem cell leukemia, Chronic leukaemia of unspecified cell type, Subacute leukaemia of unspecified cell type, Accelerated phase chronic myelogenous leukemia, Acute myeloid leukemia, Polycythemia vera, Acute promyelocytic leukemia, Acute basophilic leukemia, Acute eosinophilic leukemia, Acute lymphoblastic leukemia, Acute monocytic leukemia, Acute myeloblastic leukemia with maturation, Acute myeloid dendritic cell leukemia, Adult T-cell leukemia/lymphoma, Aggressive NK-cell leukemia, B-cell prolymphocytic leukemia, B-cell chronic lymphocytic leukemia, B-cell leukemia, Chronic myelogenous leukemia, Chronic myelomonocytic leukemia, Chronic neutrophilic leukemia, Chronic lymphocytic leukemia, Hairy cell leukemia, Chronic idiopathic myelofibrosis, Multiple myeloma, Kahler's disease, Myelomatosis, Solitary myeloma, Plasma cell leukemia, Plasmacytoma, extramedullary, Malignant plasma cell tumour NOS, Plasmacytoma NOS, Monoclonal gammopathy, Multiple Myeloma, Angiocentric immunoproliferative lesion, Lymphoid granulomatosis, Angioimmunoblastic lymphadenopathy, T-gamma lymphoproliferative disease, Waldenstrom's macroglobulinaemia, Alpha heavy chain disease, Gamma heavy chain disease, Franklin's disease, Immunoproliferative small intestinal disease, Mediterranean disease, Malignant immunoproliferative disease, unspecified, or Immunoproliferative disease NOS.

In still another embodiment, the present method can be used to treat or prevent an immune deficiency disease or disorder in a subject. The present method can be used to treat or prevent any suitable an immune deficiency disease or disorder in a subject. For example, the immune deficiency disease or disorder can be Agammaglobulinemia: X-Linked and Autosomal Recessive, Ataxia Telangiectasia, Chronic Granulomatous Disease and Other Phagocytic Cell Disorders, Common Variable Immune Deficiency, Complement Deficiencies, DiGeorge Syndrome, Hemophagocytic Lymphohistiocytosis (HLH), Hyper IgE Syndrome, Hyper IgM Syndromes, IgG Subclass Deficiency, Innate Immune Defects, NEMO Deficiency Syndrome, Selective IgA Deficiency, Selective IgM Deficiency, Severe Combined Immune, Deficiency and Combined Immune Deficiency, Specific Antibody Deficiency, Transient Hypogammaglobulinemia of Infancy, WHIM Syndrome (Warts, Hypogammaglobulinemia, Infections, and Myelokathexis), Wiskott-Aldrich Syndrome, Other Antibody Deficiency Disorders, Other Primary Cellular Immunodeficiencies, Severe combined immune deficiency (SCID), Common variable immune deficiency (CVID), Human immunodeficiency virus/acquired immune deficiency syndrome (HIV/AIDS), Drug-induced immune deficiency, Graft versus host syndrome, Primary Immune Deficiency Diseases (PIDDs), or Lymphopenia.

In still another embodiment, the present method can be used to treat or prevent an autoimmune disease or disorder. For example, the present method can be used to treat or prevent inflammation, autoimmune disease, paraneoplastic autoimmune diseases, cartilage inflammation, fibrotic disease and/or bone degradation, arthritis, rheumatoid arthritis, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reter's Syndrome, SEA Syndrome (Seronegativity, Enthesopathy, Arthropathy Syndrome), juvenile dermatomyositis, juvenile psoriatic arthritis, juvenile scleroderma, juvenile systemic lupus erythematosus, juvenile vasculitis, pauciarticular rheumatoid arthritis, polyarticular rheumatoid arthritis, systemic onset rheumatoid arthritis, ankylosing spondylitis, enteropathic arthritis, reactive arthritis, Reter's Syndrome, SEA Syndrome (Seronegativity, Enthesopathy, Arthropathy Syndrome), dermatomyositis, psoriatic arthritis, scleroderma, systemic lupus erythematosus, vasculitis, myolitis, polymyolitis, dermatomyolitis, osteoarthritis, polyarteritis nodossa, Wegener's granulomatosis, arteritis, ploymyalgia rheumatica, sarcoidosis, scleroderma, sclerosis, primary biliary sclerosis, sclerosing cholangitis, Sjogren's syndrome, psoriasis, plaque psoriasis, guttate psoriasis, inverse psoriasis, pustular psoriasis, erythrodermic psoriasis, dermatitis, atopic dermatitis, atherosclerosis, lupus, Still's disease, Systemic Lupus Erythematosus (SLE), myasthenia gravis, inflammatory bowel disease (IBD), Crohn's disease, ulcerative colitis, celiac disease, multiple schlerosis (MS), asthma, COPD, Guillain-Barre disease, Type I diabetes mellitus, thyroiditis (e.g., Graves' disease), Addison's disease, Raynaud's phenomenon, autoimmune hepatitis, GVHD, transplantation rejection, and/or the like. However, autoimmune disease or disorder is a very active area of research, and further diseases or disorder may be identified as the present invention can be obtained by the treatment. In some embodiments, an autoimmune disease or disorder refers to a disease or disorder in which the immune system attacks its own proteins, cells, tissues and organs, etc. For example, in some human autoimmune diseases or disorders, human immune system attacks its own proteins, cells, tissues and organs, etc, including diseased proteins, cells, tissues and organs. A review of some autoimmune diseases or disorders and their list can be found in The Autoimmune Diseases (Rose and Mackay, 6^{th} Edition, 2019, Academic Press). The present method can further comprise administering an effective amount of a second therapeutic agent for treating or preventing a proliferation disorder in a subject. For example, the present method can be used for treating or preventing a proliferation disease or disorder, e.g., a tumor or a cancer, in a subject and further comprise administering an anti-neoplasm substance to the subject.

To practice the method of the present invention, a modified IL-2 polypeptide, a polynucleotide, e.g., DNA, RNA or viral vector, a modified IL-2 polypeptide conjugate or a pharmaceutical composition, as described above, may be administered via any suitable route. For example, a modified IL-2 polypeptide, a polynucleotide, e.g., DNA, RNA or viral vector, a modified IL-2 polypeptide conjugate or a pharmaceutical composition, as described above, may be administered orally, parenterally, by inhalation, topically, rectally, nasally, buccally, vaginally, via an implanted reservoir, or other drug administration methods. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

A sterile injectable composition, such as a sterile injectable aqueous or oleaginous suspension, may be formulated according to techniques known in the art using suitable dispersing or wetting agents and suspending agents. The sterile injectable preparation may also be a sterile injectable solution or suspension in a non-toxic parenterally acceptable diluent or solvent. Among the acceptable vehicles and solvents that may be employed include mannitol, water, Ringer's solution and isotonic sodium chloride solution. Suitable carriers and other pharmaceutical composition components are typically sterile.

In addition, sterile, fixed oils are conventionally employed as a solvent or suspending medium (e.g., synthetic mono- or diglycerides). Fatty acids, such as oleic acid and its glyceride derivatives, are useful in the preparation of injectables, as are pharmaceutically acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Various emulsifying agents or bioavailability enhancers which are commonly used in the manufacture of pharmaceutically acceptable solid, liquid, or other dosage forms can also be used for the purpose of formulation.

A composition for oral administration may be any orally acceptable dosage form including, but not limited to, tablets, capsules, emulsions and aqueous suspensions, dispersions and solutions. In the case of tablets for oral use, commonly used carriers include lactose and corn starch. Lubricating agents, such as magnesium stearate, can also be added. For oral administration in a capsule form, useful diluents include lactose and dried corn starch. When aqueous suspensions or emulsions are administered orally, the active ingredient can be suspended or dissolved in an oily phase combined with emulsifying or suspending agents. If needed, certain sweetening, flavoring, or coloring agents can be added. A nasal aerosol or inhalation compositions can be prepared according to techniques well-known in the art of pharmaceutical formulation and can be prepared as solutions in, for example saline, employing suitable preservatives (for example, benzyl alcohol), absorption promoters to enhance bioavailability, and/or other solubilizing or dispersing agents known in the art.

In some embodiments, are provided uses of an effective amount of a polynucleotide comprising a nucleic acid sequence encoding a fusion protein comprising a modified interleukin 2 (IL-2) polypeptide, e.g., DNA, RNA or vector, or a pharmaceutical composition such a polynucleotide, e.g., DNA, RNA or a vector, as described above, for the manufacture of a medicament for treating or preventing a disease or a disorder, e.g., a proliferation disease or disorder, in a subject.

In yet another aspect, the present invention is directed to a method of expanding a CD4⁺ helper cell, CD8⁺ effector naive and memory cell, Natural Killer (NK) cell, or Natural killer T (NKT) cell population, which comprises contacting a cell population with an effective amount of a polynucleotide comprising a nucleic acid sequence encoding a fusion protein comprising a modified interleukin 2 (IL-2) polypeptide, e.g., DNA, RNA or vector, or a pharmaceutical composition such a polynucleotide, as described above, for a time sufficient to induce formation of a complex with an IL-2R βγ, thereby stimulating the expansion of the T cell, NK cell, and/or NKT cell population.

In yet another aspect, the present invention is directed to a method of expanding a CD4⁺ helper cell, CD8⁺ effector naive and memory cell, Treg cells, Natural Killer (NK) cell, or Natural killer T (NKT) cell population, which comprises contacting a cell population with an effective amount of a polynucleotide comprising a nucleic acid sequence encoding a fusion protein comprising a modified interleukin 2 (IL-2) polypeptide, e.g., DNA, RNA or vector, or a pharmaceutical composition such a polynucleotide, as described above, for a time sufficient to induce formation of a complex with an IL-2R βγ, thereby stimulating the expansion of the T cell, Treg cell, NK cell, and/or NKT cell population with reduced cell death by 10% to 100%, e.g., with reduced cell death by 10%, 20%, 30%, 40%, 50%, 60%, 70%, 80%, 90%, 100% or any subrange thereof.

In one embodiment, a polynucleotide comprising a nucleic acid sequence encoding a fusion protein comprising a modified interleukin 2 (IL-2) polypeptide, e.g., DNA, RNA or vector, or a pharmaceutical composition such a polynucleotide, as described above, expands CD4⁺ T regulatory (Treg) cells by less than 20%, 19%, 18%, 17%, 16%, 15%, 14%, 13%, 12%, 11%, 10%, 9%, 8%, 7%, 6%, 5%, 4%, 3%, 2%, 1% or less in the CD3⁺ cell population compared to an expansion of CD4⁺ Treg cells in the CD3⁺ cell population contacted with a comparable IL-2 polypeptide comprising an amino acid sequence set forth in SEQ ID NO:1 or SEQ ID NO:2 without the substitution. In another embodiment, a polynucleotide comprising a nucleic acid sequence encoding a fusion protein comprising a modified interleukin 2 (IL-2) polypeptide, e.g., DNA, RNA or vector, or a pharmaceutical composition such a polynucleotide, as described above, does not expand CD4⁺ Treg cells in the cell population. In still another embodiment, the ratio of the Teff cells to Treg cells in the cell population after incubation with the a polynucleotide comprising a nucleic acid sequence encoding a fusion protein comprising a modified interleukin 2 (IL-2) polypeptide, e.g., DNA, RNA or vector, or a pharmaceutical composition such a polynucleotide, as described above, is about or at least 2:1, 3:1, 4:1, 5:1, 6:1, 7:1, 8:1, 9:1, 10:1, 20:1, 50:1, 100:1 or more.

The present methods can be conducted in any suitable manner. In one embodiment, the present method is conducted in vivo. In another embodiment, the present method is conducted in vitro. In still another embodiment, the present method is conducted ex vivo.

In yet another aspect, the present invention is directed to an use of an effective amount of a polynucleotide comprising a nucleic acid sequence encoding a fusion protein comprising a modified interleukin 2 (IL-2) polypeptide, e.g., DNA, RNA or vector, or a pharmaceutical composition such a polynucleotide, as described above, for the manufacture of a medicament for expanding a CD4⁺ helper cell, CD8⁺ effector naive and memory cell, Treg cell, Natural Killer (NK) cell, or Natural killer T (NKT) cell population in a cell population. In one embodiment, the present use is configured for expanding a CD4⁺ helper cell, CD8⁺ effector naive and memory cell, Treg cell, Natural Killer (NK) cell, or Natural killer T (NKT) cell population in a subject.

### OTHER EXEMPLARY EMBODIMENTS

Embodiment 1. A polynucleotide comprising a nucleic acid sequence encoding a fusion protein comprising a modified interleukin 2 (IL-2) polypeptide, wherein the modified IL-2 polypeptide comprises an amino acid having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100% sequence identity to the amino acid sequence set forth in SEQ ID NO:1 or SEQ ID NO:2, wherein the modified IL-2 polypeptide comprises at least one substitution with a natural amino acid or an unnatural amino acid at one or more positions selected from the group consisting of Q13, L19, N29, N30, Y31, K32, N33, P34, K35, T37, R38, T41, F42, K43, Y45, K48, K49, E62, K64, P65, N71, Q74, K76, R81, L85, S87, V91, I92, V93, and combinations thereof.

Embodiment 2. The polynucleotide of Embodiment 1, wherein the modified IL-2 polypeptide:
a) is configured to be unconjugated or is conjugated to a water-soluble polymer, a lipid, a polypeptide, a protein or a peptide; and/or
b) has reduced binding to an interleukin 2 receptor α (IL-2Rα) compared to a comparable IL-2 polypeptide comprising an amino acid sequence set forth in SEQ ID NO:1 or SEQ ID NO:2 without the at least one substitution; and/or
c) has reduced receptor signaling potency to IL-2Rαβγ compared to a comparable IL-2 polypeptide comprising an amino acid sequence set forth in SEQ ID NO:1 or SEQ ID NO:2 without the at least one substitution, and/or
d) has increased ratio of signaling potency to IL-2Rγ over signaling potency to IL-2Rαβγ (increased ratio of signaling potency to IL-2Rβγ/signaling potency to IL-2Rαβγ) compared to a comparable IL-2 polypeptide comprising an amino acid sequence set forth in SEQ ID NO:1 or SEQ ID NO:2 without the at least one substitution, and/or
e) has enhanced receptor signaling potency to IL-2Rβγ compared to a comparable IL-2 polypeptide comprising an amino acid sequence set forth in SEQ ID NO:1 or SEQ ID NO:2 without the at least one substitution, and/or
provided that when the modified IL-2 polypeptide comprises at least one substitution with an unnatural amino acid, the modified IL-2 polypeptide comprises at least one substitution at a position selected from the group consisting of N29, N30, Y31, K32, N33, P34, K35, R38, T41, F42, K43, Y45, K48, K49, E62, K64, P65, N71, Q74, K76, and combinations thereof, and/or at least one substitution with a natural amino acid or an unnatural amino acid at at least one position within IL-2Rα interaction region, IL-2Rβ interaction region and/or IL-2Rγ interaction region; f) combinations of a) through e).

Embodiment 3. The polynucleotide of Embodiment 1 or Embodiment 2, wherein the modified IL-2 polypeptide has at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity in one or more regions of amino acid positions 10-25, 80-100 and/or 100-134 to the corresponding one or more regions of an IL-2 polypeptide comprising an amino acid sequence set forth in SEQ ID NO:1 or SEQ ID NO:2.

Embodiment 4. The polynucleotide of any one of Embodiments 1-3, wherein the modified IL-2 polypeptide has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100% sequence identity sequence identity to an IL-2 polypeptide comprising the amino acid sequence set forth in SEQ ID NO:1 or SEQ ID NO:2.

Embodiment 5. The polynucleotide of any one of Embodiments 1-4, wherein the modified IL-2 polypeptide comprises at least one substitution with lysine, cysteine, histidine, arginine, aspartic acid, glutamic acid, serine, threonine, alanine, tryptophan, isoleucine, phenylalanine, or tyrosine at a position selected from the group consisting of Q13, L19, N29, N30, Y31, K32, N33, P34, K35, T37, R38, T41, F42, K43, Y45, K48, K49, E62, K64, P65, N71, Q74, K76, R81, L85, S87, V91, I92, V93, and combinations thereof.

Embodiment 6. The polynucleotide of any one of Embodiments 1-5, wherein the modified IL-2 polypeptide comprises:
a) at least one substitution with a natural amino acid at a position selected from the group consisting of N29, N30, Y31, K32, N33, P34, K35, R38, T41, F42, K43, Y45, K48, K49, E62, K64, P65, N71, Q74, K76 and combinations thereof, and is configured to be conjugated to a water-soluble polymer, a lipid, a protein, or a peptide at at least one position selected from the group consisting of N29, N30, Y31, K32, N33, P34, K35, R38, T41, F42, K43, Y45, K48, K49, E62, K64, P65, N71, Q74, K76, and combinations thereof, and/or
b) at least one substitution with a natural amino acid at at least one position selected from the group consisting of N29, N30, Y31, K32, N33, P34, K35, R38, T41, F42, K43, Y45, K48, K49, E62, K64, P65, N71, Q74, K76 and combinations thereof, and is configured to be conjugated to a water-soluble polymer, a lipid, a protein, or a peptide at the N terminal and/or C terminal of the modified IL-2 polypeptide.

Embodiment 7. The polynucleotide of any one of Embodiments 1-6, wherein the modified IL-2 polypeptide comprises:
a) at least one substitution with lysine, cysteine, histidine, arginine, aspartic acid, glutamic acid, serine, threonine, alanine, tryptophan, isoleucine, phenylalanine, or tyrosine at at least one position selected from the group consisting of N29, N30, Y31, K32, N33, P34, K35, R38, T41, F42, K43, Y45, K48, K49, E62, K64, P65, N71, Q74, K76, and combinations thereof, and/or
b) comprises at least one substitution with lysine, cysteine, histidine, arginine, aspartic acid, glutamic acid, serine, threonine, alanine, tryptophan, isoleucine, phenylalanine, or tyrosine at a position selected from the group consisting of N30, Y31, N33, P34, K35, R38, T41, K43, K48, K49, K64, P65, N71, Q74, K76, and combinations thereof.

Embodiment 8. The polynucleotide of any one of Embodiments 1-7, wherein the modified IL-2 polypeptide comprises:
a) at least one substitution with cysteine at a position selected from the group consisting of N29, N30, Y31, N33, P34, K35, R38, T41, K43, K48, K49, K64, P65, N71, Q74, K76 and a combination thereof,
b) at least one substitution with cysteine at a position selected from the group consisting of N29, Y31, K35, P65, N71, Q74 and a combination thereof,
c) at least one substitution with any amino acid at a position of Y31, N29 or a combination thereof,
d) at least one substitution with cysteine, serine or alanine at a position of Y31, N29 or a combination thereof,
e) at least one substitution with cysteine at a position of Y31;
f) at least one substitution with cysteine at a position of N29; and/or
g) at least one substitution with cysteine at a position of P65.

Embodiment 9. The polynucleotide of any one of Embodiments 1-8, wherein the modified IL-2 polypeptide comprises at least one substitution with a natural amino acid at at least one position selected from the group consisting of R38, F42, Y45, E62, P65, and combinations thereof.

Embodiment 10. The polynucleotide of any one of Embodiments 1-9, wherein the modified IL-2 polypeptide comprises at least one substitution with lysine, cysteine, histidine, arginine, aspartic acid, glutamic acid, serine, threonine, alanine, tryptophan, isoleucine, phenylalanine, or tyrosine at at least one position selected from the group consisting of R38, F42, Y45, E62, P65 and a combination thereof.

Embodiment 11. The polynucleotide of any one of Embodiments 1-10, wherein the modified IL-2 polypeptide comprises:
a) at least one substitution with cysteine at a position selected from the group consisting of R38, F42, Y45, E62, P65, and combinations thereof, and/or
b) a substitution with alanine, lysine or serine at position F42; and/or
c) a substitution with alanine at position F42; and/or
d) a substitution with serine at position F42; and/or
e) a substitution with lysine at position F42; and/or
f) a substitution with alanine, histidine or serine at position Y45; and/or
g) a substitution with alanine at position Y45; and/or
h) a substitution with histidine at position Y45; and/or
i) a substitution with alanine, aspartic acid or serine at position R38; and/or
j) a substitution with aspartic acid at position R38; and/or
k) a substitution with alanine at position P65; and/or
l) a substitution with serine at position P65; and/or
m) a substitution with alanine at position E62; and/or
n) a substitution with lysine at position F42, and/or
o) a substitution with cysteine at position Y31, and/or
p) combinations of a) through o).

Embodiment 12. The polynucleotide of any one of Embodiments 1-11, wherein the modified IL-2 polypeptide comprises at least one substitution with a natural amino acid at at least one position selected from the group consisting of Q13, L19, R81, L85, S87, V91, 192, V93, and combinations thereof.

Embodiment 13. The polynucleotide of any one of Embodiments 1-12, wherein the modified IL-2 polypeptide comprises at least one substitution with lysine, cysteine, histidine, arginine, aspartic acid, glutamic acid, serine, threonine, alanine, tryptophan, isoleucine, phenylalanine, or tyrosine at a position selected from the group consisting of Q13, L19, R81, L85, S87, V91, I92, V93, and combinations thereof.

Embodiment 14. The polynucleotide of any one of Embodiments 1-13, wherein the modified IL-2 polypeptide comprises at least one substitution with cysteine at a position selected from the group consisting of Q13, L19, R81, L85, S87, V91, I92, V93, and combinations thereof.

Embodiment 15. The polynucleotide of any one of Embodiments 1-14, wherein the modified IL-2 polypeptide comprises:
a) a substitution at position Y31; and/or
c) a substitution at position F42; and/or
d) a substitution at position C125; and/or
e) a substitution at positions Y31, F42, and C125.

Embodiment 16. The polynucleotide of any one of Embodiments 1-15, wherein the modified IL-2 polypeptide comprises:
a) a substitution with cysteine at position Y31; and/or
c) a substitution with phenylalanine at position F42; and/or
d) a substitution with serine at position C125; and/or e) a substitution with cysteine at position Y31, a substitution with phenylalanine at position Y31, and a substitution with serine at position C125.

Embodiment 17. The polynucleotide of any one of Embodiments 1-16, further comprising at least one substitution with a natural amino acid or an unnatural amino acid at at least one position within IL-2Rα interaction region, IL-2Rβ interaction region and/or IL-2Rγ interaction region.

Embodiment 18. The polynucleotide of any one of Embodiments 1-17, further comprising at least one substitution with a natural amino acid at a position within IL-2Rα interaction region.

Embodiment 19. The polynucleotide of any one of Embodiments 1-18, further comprising at least one substitution with a natural amino acid at a position within IL-2Rβ interaction region.

Embodiment 20. The polynucleotide of any one of Embodiments 1-19, further comprising: a) a substitution with a natural amino acid at a position within IL-2Rα interaction region and a substitution with a natural amino acid at a position within IL-2Rβ interaction region; b) a substitution with a natural amino acid at a position within IL-2Rα interaction region and a substitution with a natural amino acid at a position within IL-2Rγ interaction region; or c) a substitution with a natural amino acid at a position within IL-2Rα interaction region, a substitution with a natural amino acid at a position within IL-2Rβ interaction region and a substitution with a natural amino acid at a position within IL-2Rγ interaction region.

Embodiment 21. The polynucleotide of any one of Embodiments 1-20, wherein the modified IL-2 polypeptide has reduced binding to an IL-2Rα compared to an IL-2 polypeptide comprising an amino acid sequence set forth in SEQ ID NO:1 or SEQ ID NO:2 without the at least one substitution.

Embodiment 22. The polynucleotide of any one of Embodiments 1-21, wherein the modified IL-2 polypeptide has an N-terminal deletion and/or a C-terminal deletion.

Embodiment 23. The polynucleotide of any one of Embodiments 1-22, wherein the modified IL-2polypeptide has an N-terminal deletion of amino acid residues 1-30, and/or a C terminal deletion of amino acid residues 114-134.

Embodiment 24. The polynucleotide of any one of Embodiments 1-23, wherein the fusion protein comprises the modified IL-2 polypeptide and an additional amino acid sequence.

Embodiment 25. The polynucleotide of any one of Embodiments 1-24, wherein the fusion protein comprises the modified IL-2 polypeptide linked to an additional amino acid sequence via a linker.

Embodiment 26. The polynucleotide of any one of Embodiments 1-25, wherein the additional amino acid sequence confers an enhanced and/or extended pharmacokinetic (PK) profile on the modified IL-2 polypeptide or on the fusion protein compared to an IL-2 polypeptide comprising an amino acid sequence set forth in SEQ ID NO:1 or SEQ ID NO:2 without the at least one substitution

Embodiment 27. The polynucleotide of any one of Embodiments 1-26, wherein additional amino acid sequence is fused to the additional amino acid sequence via the N-terminus of the modified IL-2 polypeptide or via the C-terminus of the modified IL-2 polypeptide.

Embodiment 28. The polynucleotide of any one of Embodiments 1-27, wherein the additional amino acid sequence comprises an antibody sequence or a portion or a fragment thereof.

Embodiment 29. The polynucleotide of any one of Embodiments 1-28, wherein the additional amino acid sequence comprises an Fc portion of an antibody.

Embodiment 30. The polynucleotide of any one of Embodiments 1-29, wherein the additional amino acid sequence comprises a serum albumin or a PK-extending fragment or analog thereof.

Embodiment 31. The polynucleotide of any one of Embodiments 1-30, wherein the additional amino acid sequence comprises:
a) a human serum albumin (HSA);
b) a murine serum albumin (MSA);
c) or a PK-extending fragment or analog of a) or b).

Embodiment 32. The polynucleotide of any one of Embodiments 1-31, wherein the modified IL-2 polypeptide comprises the amino acid sequence set forth in SEQ ID NO: 3

Embodiment 33. The polynucleotide of any one of Embodiments 1-32, wherein the modified IL-2 polypeptide and/or the additional amino acid sequence is further configured to be conjugated to a water-soluble polymer, a lipid, a protein, or a peptide via a single amino acid residue of the modified IL-2 polypeptide.

Embodiment 34. The polynucleotide of any one of Embodiments 1-33, wherein the modified IL-2 polypeptide and/or the additional amino acid sequence is further configured to be conjugated to a water-soluble polymer, a lipid, a protein, or a peptide via:
i) the alpha amino group of the N-terminal amino acid residue of the fusion polypeptide;
ii) the epsilon amino group of a lysine amino acid residue of the fusion polypeptide; or
iii) an N-glycosylation site or O-glycosylation site of the fusion polypeptide.

Embodiment 35. The polynucleotide of any one of Embodiments 1-34, wherein the IL-2 polypeptide or the fusion protein has increased binding to an IL-2Rγ compared to an IL-2 polypeptide comprising an amino acid sequence set forth in SEQ ID NO:1 or SEQ ID NO:2 without the at least one substitution.

Embodiment 36. The polynucleotide of any one of Embodiments 1-35, wherein the IL-2 polypeptide or the fusion protein has: (i) reduced binding to an IL-2Rα and (ii) increased binding to an IL-2Rβγ; compared to an IL-2 polypeptide comprising an amino acid sequence set forth in SEQ ID NO:1 or SEQ ID NO:2 without the at least one substitution.

Embodiment 37. The polynucleotide of any one of Embodiments 1-36, wherein the binding affinity of the IL-2 polypeptide or the fusion protein to an IL-2Rα is decreased: from about 10%, about 20%, about 30%, about 40%, about 50%, about 60, about 70%, about 80%, about 90%, about 100 about 100%; or is decreased from about 1 fold to about 100,000 fold or more compared to the binding affinity of an IL-2 polypeptide comprising an amino acid sequence set forth in SEQ ID NO:1 or SEQ ID NO:2 without the at least one substitution.

Embodiment 38. The polynucleotide of any one of Embodiments 1-37, wherein the IL-2 polypeptide or the fusion protein has no detectable binding to an IL-2Rα.

Embodiment 39. The polynucleotide of any one of Embodiments 1-38, wherein the IL-2 polypeptide or the fusion protein has reduced receptor signaling potency to IL-2Rαβγ compared to an IL-2 polypeptide comprising an amino acid sequence set forth in SEQ ID NO:1 or SEQ ID NO:2 without the at least one substitution.

Embodiment 40. The polynucleotide of any one of Embodiments 1-39, wherein the ratio between the signaling potency of the IL-2 polypeptide or the fusion protein to IL-2Rαβγ and the signaling potency to IL-2Rαβγ of the an IL-2 polypeptide comprising an amino acid sequence set forth in SEQ ID NO:1 or SEQ ID NO:2 without the at least one substitution is from about 1/2 to about 1/100,000.

Embodiment 41. The polynucleotide of any one of Embodiments 1-40, wherein the IL-2 polypeptide or the fusion protein has no detectable receptor signaling potency to IL-2Rαβγ.

Embodiment 42. The polynucleotide of any one of Embodiments 1-41, wherein the IL-2 polypeptide or the fusion protein: (i) has reduced binding to an IL-2Rα compared to an IL-2 polypeptide comprising an amino acid sequence set forth in SEQ ID NO:1 or SEQ ID NO:2 without the at least one substitution; and (ii) has reduced receptor signaling potency to IL-2Rαβγ compared to an IL-2 polypeptide comprising an amino acid sequence set forth in SEQ ID NO:1 or SEQ ID NO:2 without the at least one substitution.

Embodiment 43. The polynucleotide of any one of Embodiments 1-42, wherein the IL-2 polypeptide or the fusion protein has no detectable binding to an IL-2Rα and has no detectable receptor signaling potency to IL-2Rαβγ.

Embodiment 44. The polynucleotide of any one of Embodiments 1-43, wherein the IL-2 polypeptide or the fusion protein, wherein the modified IL-2 polypeptide or the fusion protein: has comparable has increased binding to an interleukin 2 receptor β (IL-2R β) and/or an interleukin 2 receptor γ (IL-2R γ) compared to an IL-2 polypeptide comprising an amino acid sequence set forth in SEQ ID NO:1 or SEQ ID NO:2 without the at least one substitution; and/or has comparable or has increased receptor signaling potency to IL-2R βγ compared to an IL-2 polypeptide comprising an amino acid sequence set forth in SEQ ID NO:1 or SEQ ID NO:2 without the at least one substitution.

Embodiment 45. The polynucleotide of any one of Embodiments 1-44, wherein the IL-2 polypeptide or the fusion protein has comparable or has higher binding level to an IL-2R β or an IL-2R γ compared to an n IL-2 polypeptide comprising an amino acid sequence set forth in SEQ ID NO:1 or SEQ ID NO:2 without the at least one substitution.

Embodiment 46. The polynucleotide of any one of Embodiments 1-45, wherein the IL-2 polypeptide or the fusion protein has comparable or has higher receptor signaling potency to IL-2R βγ compared to an IL-2 polypeptide comprising an amino acid sequence set forth in SEQ ID NO:1 or SEQ ID NO:2 without the at least one substitution.

Embodiment 47. The polynucleotide of any one of Embodiments 1-46, wherein the IL-2 polypeptide or the fusion protein has comparable or has higher binding level to an IL-2R β or an IL-2R γ compared to an comparable IL-2 polypeptide comprising an amino acid sequence set forth in SEQ ID NO:1 or SEQ ID NO:2 without the at least one substitution, and has comparable or has higher receptor signaling potency to IL-2R βγ compared to an IL-2 polypeptide comprising an amino acid sequence set forth in SEQ ID NO:1 or SEQ ID NO:2 without the at least one substitution.

Embodiment 48. The polynucleotide of any one of Embodiments 1-47, wherein the IL-2 polypeptide or the fusion protein has increased ratio of signaling potency to IL-2Rβγ over signaling potency to IL-2Rαβγ (increased ratio of signaling potency to IL-2Rβγ/signaling potency to IL-2Rαβγ) compared to an IL-2 polypeptide comprising an amino acid sequence set forth in SEQ ID NO:1 or SEQ ID NO:2 without the at least one substitution.

Embodiment 49. The polynucleotide of any one of Embodiments 1-48, further comprising a promoter operably linked to the nucleic acid sequence encoding the fusion protein.

Embodiment 50. The polynucleotide of any one of Embodiments 1-49, further comprising a promoter operably linked to the nucleic acid sequence encoding the fusion protein, wherein the promoter drives tissue-specific expression of the fusion protein.

Embodiment 51. The polynucleotide of any one of Embodiments 1-50, further comprising a promoter operably linked to the nucleic acid sequence encoding the fusion protein, wherein the promoter drives muscle-specific expression of the fusion protein.

Embodiment 52. The polynucleotide of any one of Embodiments 1-50, further comprising a promoter operably linked to the nucleic acid sequence encoding the fusion protein, wherein the promoter drives cancer-specific expression or tumor-specific expression of the fusion protein.

Embodiment 53. The polynucleotide of any one of Embodiments 1-50, further comprising a promoter operably linked to the nucleic acid sequence encoding the fusion protein, wherein the promoter drives liver-specific expression of the fusion protein.

Embodiment 54. The polynucleotide of any one of Embodiments 1-53, further comprising a promoter operably linked to the nucleic acid sequence encoding the fusion protein, wherein the promoter is constitutively active.

Embodiment 55. The polynucleotide of any one of Embodiments 1-53, further comprising a promoter operably linked to the nucleic acid sequence encoding the fusion protein, wherein the promoter is conditionally active.

Embodiment 56. The polynucleotide any one of Embodiments 1-55, further comprising a promoter operably linked to the nucleic acid sequence, wherein the promoter comprises an SP6, T3, or T7 promoter sequence.

Embodiment 57. The polynucleotide any one of Embodiments 1-56, wherein at least a portion of the nucleic acid sequence encoding the fusion protein has been codon-optimized.

Embodiment 58. The polynucleotide of any one of Embodiments 1-57, wherein the polynucleotide includes at least one modified nucleotide.

Embodiment 59. The polynucleotide of any one of Embodiments 1-58, wherein the polynucleotide includes at least one modified nucleotide, wherein the at least one modified nucleotide is pseudouridine, N1-methyl-pseudouridine, or 2-thiouridine.

Embodiment 60. The polynucleotide of any one of Embodiments 1-59, wherein the nucleic acid molecule comprises a 5′ cap structure.

Embodiment 61. The polynucleotide of any one of Embodiments 1-60, wherein the nucleic acid molecule comprises a 3′ polyA sequence.

Embodiment 62. The polynucleotide of any one of Embodiments 1-61, wherein the polynucleotide comprises: DNA; RNA; or a DNA/RNA hybrid.

Embodiment 63. The polynucleotide of any one of Embodiments 1-62, wherein the polynucleotide comprises RNA.

Embodiment 64. The polynucleotide of any one of Embodiments 1-63 wherein the polynucleotide comprises: a viral vector; a nonviral vector; a plasmid; or a Nanoplasmid™ vector.

Embodiment 65. The polynucleotide of any one of Embodiments 1-64 wherein the polynucleotides configured to express the fusion protein in vitro.

Embodiment 66. The polynucleotide of any one of Embodiments 1-64 wherein the polynucleotide is configured to express the fusion protein in vivo.

Embodiment 67. The polynucleotide of any one of Embodiments 1-66 wherein the polynucleotide is in an isolated form.

Embodiment 68. The polynucleotide of any one of Embodiments 1-67 wherein the polynucleotide wherein the modified IL-2 polypeptide or the fusion protein has a half-life in vivo from about 5 minutes to about 10 days, from about 5 minutes to about 9 days, from about 5 minutes to about 8 days, from about 5 minutes to about 8 days, from about 5 minutes to about 7 days, from about 5 minutes to about 6 days, from about 5 minutes to about 5 days, from about 5 minutes to about 4 days, from about 5 minutes to about 3 days, from about 5 minutes to about 2 days, from about 5 minutes to about 1 day, from about 1 hour to about 10 days, about 1 hour to about 9 days, from out 1 hour to about 8 days, from about 1 hour to about 7 days, from out 1 hours to about 6 days, from about 1 hour to about 5 days, from about 1 hour to about 4 days, about 1 hour to about 3 days, from out 1 hour to about 2 days, from about 1 hour to about 1 day, from about 5 minutes, from about 10 minutes, from about 20 minutes, from about 30 minutes, from about 40 minutes, from about 50 minutes, from about 1 hour, from about 2 hours, from about 3 hours, from about 4 hours, from about 5 hours, from about 6 hours, from about 7 hours, from about 8 hours, from about 9 hours, from about 10 hours, from about 11 hours, from about 12 hours, from about 13 hours, from about 14 hours, from about 15 hours, from about 16 hours, from about 17 hours, from about 18 hours, from about 19 hours, from about 20 hours, from about 21 hours, from about 22 hours, from about 23 hours, from about 1 day, from about 2 days, from about 3 days, from about 4 days, from about 5 days, from about 6 days, from about 7 days, from about 8 days, from about 9 days, from about 10 days or a value or a range in between.

Embodiment 69. A pharmaceutical composition comprising an effective amount of a polynucleotide of any one of Embodiments 1-68 and a pharmaceutically acceptable carrier.

Embodiment 70. The pharmaceutical composition of Embodiment 69, wherein the pharmaceutically acceptable carrier comprises a lipid.

Embodiment 71. The pharmaceutical composition of Embodiment 69 or Embodiment 70, wherein the lipid comprises a cationic lipid of formula (I):

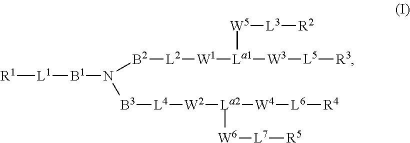

or a pharmaceutically acceptable salt, solvate, hydrate, stereoisomer, or prodrug thereof, wherein:
R¹ is H, —OR^{1A}, —YOR^{1A}, —NR^{1A}R^{1B}, —YNR^{1A}R^{1B}, —SR^{1A}, —YSR^{1A}, —(C═O)R^{1A}, —Y(C═O)R^{1A}, —(C═O)OR^{1A}, —Y(C═O)OR^{1A}, —O(C═O)R^{1A}, —YO(C═O)R^{1A}, —O(C═O)OR^{1A}, —YO(C═O)OR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Y is substituted or unsubstituted C₀-C₁₂ alkylene or substituted or unsubstituted 0 to 12 membered heteroalkylene;
R² is H, —OR^{2A}, —SR^{2A}, —(C═O)R^{2A}, —(C═O)OR^{2A}, —O(C═O)R^{2A}, —O(C═O)OR^{2A}, —(C═O)NHR^{2A}, —NH(C═O)R^{2A}, substituted or unsubstituted alkyl, or substituted or unsubstituted heteroalkyl;
R³ is H, —OR^{3A}, —SR^{3A}, —(C═O)R^{3A}, —(C═O)OR^{3A}, —O(C═O)R^{3A}, —O(C═O)OR^{3A}, —(C═O)NHR^{3A}, —NH(C═O)R^{3A}, substituted or unsubstituted alkyl, or substituted or unsubstituted heteroalkyl;
R⁴ is H, —OR^{4A}, —SR^{4A}, —(C═O)R^{4A}, —(C═O)OR^{4A}, —O(C═O)R^{4A}, —O(C═O)OR^{4A}, —(C═O)NHR^{4A}, —NH(C═O)R^{4A}, substituted or unsubstituted alkyl, or substituted or unsubstituted heteroalkyl;
R⁵ is H, —OR^{5A}, —SR^{5A}, —(C═O)R^{5A}, —(C═O)OR^{5A}, —O(C═O)R^{5A}, —O(C═O)OR^{5A}, —(C═O)NHR^{5A}, —NH(C═O)R^{5A}, substituted or unsubstituted alkyl, or substituted or unsubstituted heteroalkyl;
B¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
B² and B³ are each independently a bond, substituted or unsubstituted alkylene, or substituted or unsubstituted heteroalkylene;
L¹ is a bond, —O(C═O)—, —(C═O)O—, —O(C═O)O—, —C(═O)—, —O—, —O(CR¹⁰¹R¹⁰²)ₛO—, —S—, —C(═O)S—, —SC(═O)—, —NR¹⁰¹C(═O)—, —C(═O)NR¹⁰¹—, —NR¹⁰¹C(═S)—, —C(═S)NR¹⁰¹—, —NR¹⁰¹C(═O)NR¹⁰²—, —NR¹⁰¹C(═S)NR¹⁰²—, —OC(═O)NR¹⁰¹—, —NR¹⁰¹C(═O)O—, —SC(═O)NR¹⁰¹— or —NR¹⁰¹C(═O)S—;
L² is a bond, —O(C═O)—, —(C═O)O—, —O(C═O)O—, —C(═O)—, —O—, —O(CR²⁰¹R²⁰²)ₛO—, —S—, —C(═O)S—, —SC(═O)—, —NR²⁰¹C(═O)—, —C(═O)NR²⁰¹—, —NR²⁰¹C(═O)NR²⁰²—, —NR²⁰¹C(═S)—, —C(═S)NR²⁰¹—, —NR²⁰¹C(═S)NR²⁰²—, —OC(═O)NR²⁰¹—, —NR²⁰¹C(═O)O—, —SC(═O)NR²⁰¹— or —NR²⁰¹C(═O)S—;
L³ is a bond, —O(C═O)—, —(C═O)O—, —O(C═O)O—, —C(═O)—, —O—, —O(CR³⁰¹R³⁰²)ₛO—, —S—, —C(═O)S—, —SC(═O)—, —NR³⁰¹C(═O)—, —C(═O)NR³⁰¹—, —NR³⁰¹C(═O)NR³⁰²—, —NR³⁰¹C(═S)—, —C(═S)NR³⁰¹—, —NR³⁰¹C(═S)NR³⁰²—, —OC(═O)NR³⁰¹—, —NR³⁰¹C(═O)O—, —SC(═O)NR³⁰¹— or —NR³⁰¹C(═O)S—;
L⁴ is a bond, —O(C═O)—, —(C═O)O—, —O(C═O)O—, —C(═O)—, —O—, —O(CR⁴⁰¹R⁴⁰²)ₛO—, —S—, —C(═O)S—, —SC(═O)—, —NR⁴⁰¹C(═O)—, —C(═O)NR⁴⁰¹—, —NR⁴⁰¹C(═O)NR⁴⁰²—, —NR⁴⁰¹C(═S)—, —C(═S)NR⁴⁰¹—, —NR⁴⁰¹C(═S)NR⁴⁰²—, —OC(═O)NR⁴⁰¹—, —NR⁴⁰¹C(═O)O—, —SC(═O)NR⁴⁰¹— or —NR⁴⁰¹C(═O)S—;
L⁵ is a bond, —O(C═O)—, —(C═O)O—, —O(C═O)O—, —C(═O)—, —O—, —O(CR⁵⁰¹R⁵⁰²)ₛO—, —S—, —C(═O)S—, —SC(═O)—, —NR⁵⁰¹C(═O)—, —C(═O)NR⁵⁰¹—, —NR⁵⁰¹C(═O))NR⁵⁰²—, —NR⁵⁰¹C(═S)—, —C(═S)NR⁵⁰¹—, —NR⁵⁰¹C(═S)NR⁵⁰²—, —OC(═O)NR⁵⁰¹—, —NR⁵⁰¹C(═O)O—, —SC(═O)NR⁵⁰¹— or —NR⁵⁰¹C(═O)S—;
L⁶ is a bond, —O(C═O)—, —(C═O)O—, —O(C═O)O—, —C(═O)—, —O—, —O(CR⁶⁰¹R⁶⁰²)ₛO—, —S—, —C(═O)S—, —SC(═O)—, —NR⁶⁰¹C(═O)—, —C(═O)NR⁶⁰¹—, —NR⁶⁰¹C(═O)NR⁶⁰²—, —NR⁶⁰¹C(═S)—, —C(═S)NR⁶⁰¹—, —NR⁶⁰¹C(═S)NR⁶⁰²—, —OC(═O)NR⁶⁰¹—, —NR⁶⁰¹C(═O)O—, —SC(═O)NR⁶⁰¹— or —NR⁶⁰¹C(═O)S—;
L⁷ is a bond, —O(C═O)—, —(C═O)O—, —O(C═O)O—, —C(═O)—, —O—, —O(CR⁷⁰¹R⁷⁰²)ₛO—, —S—, —C(═O)S—, —SC(═O)—, —NR⁷⁰¹C(═O)—, —C(═O)NR⁷⁰¹—, —NR⁷⁰¹C(═O)NR⁷⁰²—, —NR⁷⁰¹C(═S)—, —C(═S)NR⁷⁰¹—, —NR⁷⁰¹C(═S)NR⁷⁰²—, —OC(═O)NR⁷⁰¹—, —NR⁷⁰¹C(═O)O—, —SC(═O)NR⁷⁰¹— or —NR⁷⁰¹C(═O)S—;
L^{a1} and L^{a2} are each independently

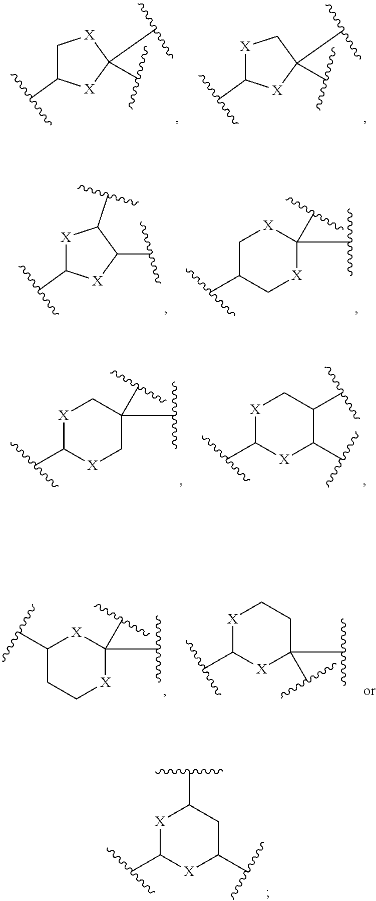

each X is independently O, S, or CH₂;
W¹, W², W³, W⁴, W⁵, and W⁶ are each independently a bond, substituted or unsubstituted C₁-C₁₂ alkylene, or substituted or unsubstituted 2 to 12 membered heteroalkylene;
each R^{1A} and R^{1B} is independently H, substituted or unsubstituted C₁-C₁₂ alkyl, or substituted or unsubstituted 2 to 12 membered heteroalkyl;
each R^{2A}, R^{3A}, R^{4A}, and R^{5A} is independently H, substituted or unsubstituted C₁-C₃₀ alkyl, or substituted or unsubstituted 2 to 30 membered heteroalkyl;
each R¹⁰¹, R¹⁰², R²⁰¹, R²⁰², R³⁰¹, R³⁰², R⁴⁰¹, R⁴⁰², R³⁰¹, R⁵⁰², R⁶⁰¹, R⁶⁰², R⁷⁰¹, and R⁷⁰² is independently H, substituted or unsubstituted C₁-C₁₂ alkyl, or substituted or unsubstituted 2 to 12 membered heteroalkyl; and each s is independently an integer from 1 to 4.

Embodiment 72. The pharmaceutical composition of Embodiment 71, wherein the cationic lipid is a lipid wherein: R¹ is H, —OR^{1A} or substituted or unsubstituted heteroalkyl;
L¹ is a bond, —NR¹⁰¹C(═S)—, —C(═S)NR¹⁰¹—, —O(C═O)—, —(C═O)O—, or —O—;
B¹ is a bond or a substituted or unsubstituted alkylene;
B² and B³ are each independently a bond or substituted or unsubstituted alkylene;
L² is a bond, —O(C═O)—, —(C═O)O—, —O(C═O)O—, —C(═O)—, —O—, or —S—;
L⁴ is a bond, —O(C═O)—, —(C═O)O—, —O(C═O)O—, —C(═O)—, —O—, or —S—;
W¹, W², W³, W⁴, W⁵, and W⁶ are each independently a bond or substituted or unsubstituted C₁-C₁₂ alkylene;
L^{a1} and L^{a2} are each independently

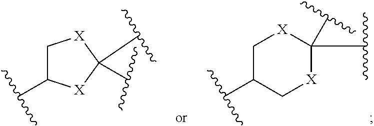

each X is independently O or S;
L³ is a bond, —O(C═O)—, —(C═O)O—, —O(C═O)O—, —C(═O)—, —O—, or —S—;
L⁵ is a bond, —O(C═O)—, —(C═O)O—, —O(C═O)O—, —C(═O)—, —O—, or —S—;
L⁶ is a bond, —O(C═O)—, —(C═O)O—, —O(C═O)O—, —C(═O)—, —O—, or —S—;
L⁷ is a bond, —O(C═O)—, —(C═O)O—, —O(C═O)O—, —C(═O)—, —O—, or —S—;
R² is H or substituted or unsubstituted alkyl;
R³ is H or substituted or unsubstituted alkyl;
R⁴ is H or substituted or unsubstituted alkyl;
R⁵ is H or substituted or unsubstituted alkyl;
each R^{1A} is independently H or substituted or unsubstituted C₁-C₁₂ alkyl; and
each R¹⁰¹ is independently H or substituted or unsubstituted 2 to 12 membered heteroalkyl.

Embodiment 73. The pharmaceutical composition of Embodiment 71, wherein the cationic lipid is a lipid wherein: R¹ is H, —OH, methoxy, ethoxy, or substituted or unsubstituted heteroalkyl;
L¹ is a bond, —NR¹⁰¹C(═S)—, or —C(═S)NR¹⁰¹—;
B¹ is a bond or an unsubstituted C₁-C₈ alkylene;
B² and B³ are each independently a bond or substituted or unsubstituted C₁-C₈ alkylene;
L² is a bond, —O(C═O)—, or —(C═O)O—;
L⁴ is a bond, —O(C═O)—, or —(C═O)O—;
W¹, W², W³, W⁴, W⁵, and W⁶ are each independently a bond or substituted or unsubstituted C₁-C₁₂ alkylene;
L^{a1} and L^{a2} are each independently

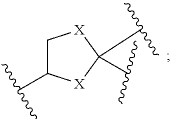

each X is independently O or S;
L³ is a bond, —O(C═O)—, or —(C═O)O—;
L⁵ is a bond, —O(C═O)—, or —(C═O)O—;
L⁶ is a bond, —O(C═O)—, or —(C═O)O—;
L⁷ is a bond, —O(C═O)—, or —(C═O)O—;
R² is H or substituted or unsubstituted C₁-C₁₂ alkyl;
R³ is H or substituted or unsubstituted C₁-C₁₂ alkyl;
R⁴ is H or substituted or unsubstituted C₁-C₁₂ alkyl;
R⁵ is H or substituted or unsubstituted C₁-C₁₂ alkyl; and
each R¹⁰¹ is independently substituted or unsubstituted 2 to 12 membered heteroalkyl.

Embodiment 74. The pharmaceutical composition of Embodiment 71, wherein the cationic lipid is a lipid wherein: R¹ is —OH or methoxy;
L¹ is a bond;
B¹ is an unsubstituted C₁-C₈ alkylene;
B² and B³ are each independently a bond or substituted or unsubstituted C₁-C₈ alkylene;
L² is a bond;
L⁴ is a bond;
W¹, W², W³, W⁴, W⁵, and W⁶ are each independently a bond or substituted or unsubstituted C₁-C₁₂ alkylene;
L^{a1} and L^{a2} are each independently

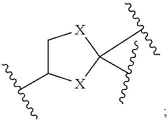

each X is independently O;
L³ is a bond;
L⁵ is a bond;
L⁶ is a bond;
L⁷ is a bond;
R² is H or substituted or unsubstituted C₁-C₁₂ alkyl;
R³ is H or substituted or unsubstituted C₁-C₁₂ alkyl;
R⁴ is H or substituted or unsubstituted C₁-C₁₂ alkyl; and
R⁵ is H or substituted or unsubstituted C₁-C₁₂ alkyl.

Embodiment 75. The pharmaceutical composition of Embodiment 71, wherein the cationic lipid is a lipid wherein: R¹ is substituted or unsubstituted heteroalkyl;
L¹ is —C(═S)NR¹⁰¹—, where the carbon atom is connected to the nitrogen atom in formula (I);
B¹ is a bond;
B² and B³ are each independently a bond or substituted or unsubstituted C₁-C₈ alkylene;
L² is a bond, —O(C═O)—, or —(C═O)O—;
L⁴ is a bond, —O(C═O)—, or —(C═O)O—;
W¹, W², W³, W⁴, W⁵, and W⁶ are each independently a bond or substituted or unsubstituted C₁-C₁₂ alkylene;
L^{a1} and L^{a2} are each independently

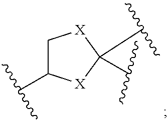

each X is independently O;
L³ is a bond;
L⁵ is a bond;
L⁶ is a bond;
L⁷ is a bond;
R² is H or substituted or unsubstituted C₁-C₁₂ alkyl;
R³ is H or substituted or unsubstituted C₁-C₁₂ alkyl;
R⁴ is H or substituted or unsubstituted C₁-C₁₂ alkyl; and
R⁵ is H or substituted or unsubstituted C₁-C₁₂ alkyl.

Embodiment 76. The pharmaceutical composition of Embodiment 71, wherein the cationic lipid is:

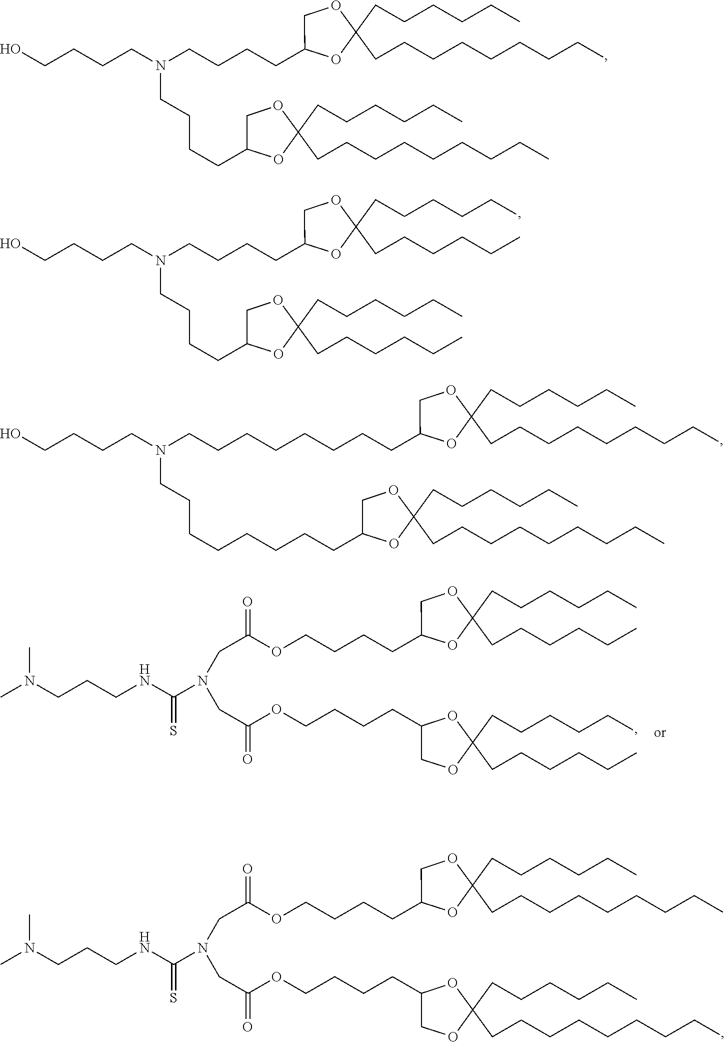

or a pharmaceutically acceptable salt thereof.

Embodiment 77. The pharmaceutical composition of Embodiment 69 or Embodiment 70, wherein the lipid comprises a cationic lipid of formula (II):

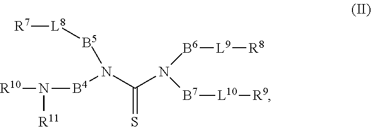

or a pharmaceutically acceptable salt, solvate, hydrate, stereoisomer, or prodrug thereof, wherein:
B⁴ is W⁷-L^{a3}-W⁸;
W⁷ and W⁸ are each independently a bond, substituted or unsubstituted alkylene, or substituted or unsubstituted heteroalkylene;
L^{a3} is a bond, —O(C═O)—, —(C═O)O—, —O(C═O)O—, —C(═O)—, —O—, —O(CR^{a31}R^{a32})ₛO—, —S—, —C(═O)S—, —SC(═O)—, —NR^{a31}C(═O)—, —C(═O))NR^{a31}—, —NR^{a31}C(═O))NR^{a32}—, —NR^{a31}C(═S)—, —C(═S)NR^{a31}—, —NR^{a31}C(═S)NR^{a32}—, —OC(═O)NR^{a31}—, —NR^{a31}C(═O)O—, —SC(═O)NR^{a31}— or —NR^{a31}C(═O)S—;
R¹⁰ and R¹¹ are each independently H, substituted or unsubstituted alkyl, substituted or unsubstituted heteroalkyl, or R¹⁰ and R¹¹ together with the nitrogen atom to which they are connected form a substituted or unsubstituted heterocycloalkyl or substituted or unsubstituted heteroaryl;
B⁵, B⁶, and B⁷ are each independently a bond, substituted or unsubstituted alkylene, or substituted or unsubstituted heteroalkylene;
L⁸ is a bond, —O(C═O)—, —(C═O)O—, —O(C═O)O—, —C(═O)—, —O—, —O(CR⁸⁰¹R⁸⁰²)ₛO—, —S—, —C(═O)S—, —SC(═O)—, —NR⁸⁰¹C(═O)—, —C(═O)NR⁸⁰¹—, —NR⁸⁰¹C(═O))NR⁸⁰²—, —NR⁸⁰¹C(═S)—, —C(═S)NR⁸⁰¹—, —NR⁸⁰¹C(═S)NR⁸⁰²—, —OC(═O)NR⁸⁰¹—, —NR⁸⁰¹C(═O)O—, —SC(═O)NR⁸⁰¹— or —NR⁸⁰¹C(═O)S—;
L⁹ is a bond, —O(C═O)—, —(C═O)O—, —O(C═O)O—, —C(═O)—, —O—, —O(CR⁹⁰¹R⁹⁰²)ₛO—, —S—, —C(═O)S—, —SC(═O)—, —NR⁹⁰¹C(═O)—, —C(═O)NR⁹⁰¹—, —NR⁹⁰¹C(═O)NR⁹⁰²—, —NR⁹⁰¹C(═S)—, —C(═S)NR⁹⁰¹—, —NR⁹⁰¹C(═S)NR⁹⁰²—, —OC(═O)NR⁹⁰¹—, —NR⁹⁰¹C(═O)O—, —SC(═O)NR⁹⁰¹— or —NR⁹⁰¹C(═O)S—;
L¹⁰ is a bond, —O(C═O)—, —(C═O)O—, —O(C═O)O—, —C(═O)—, —O—, —O(CR¹¹⁰R¹¹¹)ₛO—, —S—, —C(═O)S—, —SC(═O)—, —NR¹¹⁰C(═O)—, —C(═O)NR¹¹⁰—, —NR¹¹⁰C(═O)NR¹¹¹—, —NR¹¹⁰C(═S)—, —C(═S)NR¹¹⁰—, —NR¹¹⁰C(═S)NR¹¹¹—, —OC(═O)NR¹¹⁰—, —NR¹¹⁰C(═O)O—, —SC(═O)NR¹¹⁰— or —NR¹¹⁰C(═O)S—;
R⁷, R⁸, and R⁹ are each independently H, substituted or unsubstituted C₁-C₃₀ alkyl, or substituted or unsubstituted 2 to 30 membered heteroalkyl;
each R^{a31} and R^{a32} is independently H, substituted or unsubstituted C₁-C₁₂ alkyl, or substituted or unsubstituted 2 to 12 membered heteroalkyl;
each R⁸⁰¹, R⁸⁰², R⁹⁰¹, R⁹⁰², R¹¹⁰, and R¹¹¹ is independently H, substituted or unsubstituted C₁-C₁₂ alkyl, or substituted or unsubstituted 2 to 12 membered heteroalkyl; and
each s is independently an integer from 1 to 4.

Embodiment 78. The pharmaceutical composition of Embodiment 77, wherein the cationic lipid is a lipid wherein:
W⁷ and W⁸ are each independently a bond or substituted or unsubstituted alkylene;
L^{a3} is a bond;
R¹⁰ and R¹¹ are each independently H, substituted or unsubstituted alkyl or R¹⁰ and R¹¹ together with the nitrogen atom to which they are connected form a substituted or unsubstituted heterocycloalkyl;
B⁵ is a bond;
B⁶ and B⁷ are each independently a bond or substituted or unsubstituted alkylene;
L⁸ is a bond;
L⁹ is a bond, —O(C═O)—, —(C═O)O—, or —C(═O)—;
L¹⁰ is a bond, —O(C═O)—, —(C═O)O—, or —C(═O)—; and
R⁷, R⁸, and R⁹ are each independently H or substituted or unsubstituted C₁-C₃₀ alkyl.

Embodiment 79. The cationic lipid of Embodiment 77, wherein W⁷ and W⁸ are each independently a bond or substituted or unsubstituted C₁-C₈ alkylene;
L^{a3} is a bond;
R¹⁰ and R¹¹ are each independently substituted or unsubstituted alkyl or R¹⁰ and R¹¹ together with the nitrogen atom to which they are connected form a substituted or unsubstituted heterocycloalkyl;
B⁵ is a bond;
B⁶ and B⁷ are each independently a bond or substituted or unsubstituted C₁-C₈ alkylene;
L⁸ is a bond;
L⁹ is —O(C═O)— or —(C═O)O—;
L¹⁰ —O(C═O)— or —(C═O)O—; and
R⁷, R⁸, and R⁹ are each independently substituted or unsubstituted C₁-C₂₀ alkyl.

Embodiment 80. The pharmaceutical composition of Embodiment 77, wherein the cationic lipid is a lipid wherein: W⁷ and W⁸ are each independently a bond or substituted or unsubstituted C₂-C₄ alkylene;
L^{a3} is a bond;
R¹⁰ and R¹¹ are each independently substituted or unsubstituted methyl, ethyl, propyl, isopropyl, or R¹⁰ and R¹¹ together with the nitrogen atom to which they are connected form a substituted or unsubstituted 3 to 8 membered heterocycloalkyl;
B⁵ is a bond;
B⁶ and B⁷ are each independently a bond or substituted or unsubstituted C₂-C₄ alkylene;
L¹ is a bond;
L⁹ is —O(C═O)— or —(C═O)O—;
L¹⁰ —O(C═O)— or —(C═O)O—;
R⁷ is H or methyl; and
R⁸, and R⁹ are each independently substituted or unsubstituted C₁-C₂₀ alkyl.

Embodiment 81. The pharmaceutical composition of Embodiment 77, wherein the cationic lipid is a lipid wherein: W⁷ and W⁸ are each independently a bond or unsubstituted C₂-C₄ alkylene;
L^{a3} is a bond;
R¹⁰ and R¹¹ are each independently substituted or unsubstituted methyl, ethyl, propyl, isopropyl, or R¹⁰ and R¹¹ together with the nitrogen atom to which they are connected form a substituted or unsubstituted 5 to 6 membered heterocycloalkyl;
B⁵ is a bond;
B⁶ and B⁷ are each independently a bond or unsubstituted C₂-C₄ alkylene;
L¹ is a bond;
L⁹ is —O(C═O)— or —(C═O)O—;
L¹⁰ is —O(C═O)— or —(C═O)O—;
R⁷ is H or methyl; and
R⁸ and R⁹ are each independently substituted or unsubstituted C₁-C₂₀ alkyl.

Embodiment 82. The pharmaceutical composition of Embodiment 77, wherein the cationic lipid is a lipid wherein: W⁷ and W⁸ are each independently a bond or unsubstituted C₂-C₄ alkylene;
L^{a3} is a bond;
R¹⁰ and R¹¹ are each independently substituted or unsubstituted methyl, ethyl, propyl, isopropyl, or R¹⁰ and R¹¹ together with the nitrogen atom to which they are connected form a substituted or unsubstituted 5 to 6 membered heterocycloalkyl;
B5, B⁶, and B⁷ are each independently a bond;
L⁸ is a bond;
L⁹ is a bond;
L¹⁰ is a bond;
R⁷ is H or methyl; and
R⁸ and R⁹ are each independently substituted or unsubstituted C₁-C₃₀ alkyl.

Embodiment 83. The pharmaceutical composition of Embodiment 77, wherein the cationic lipid is a lipid wherein the cationic lipid is:

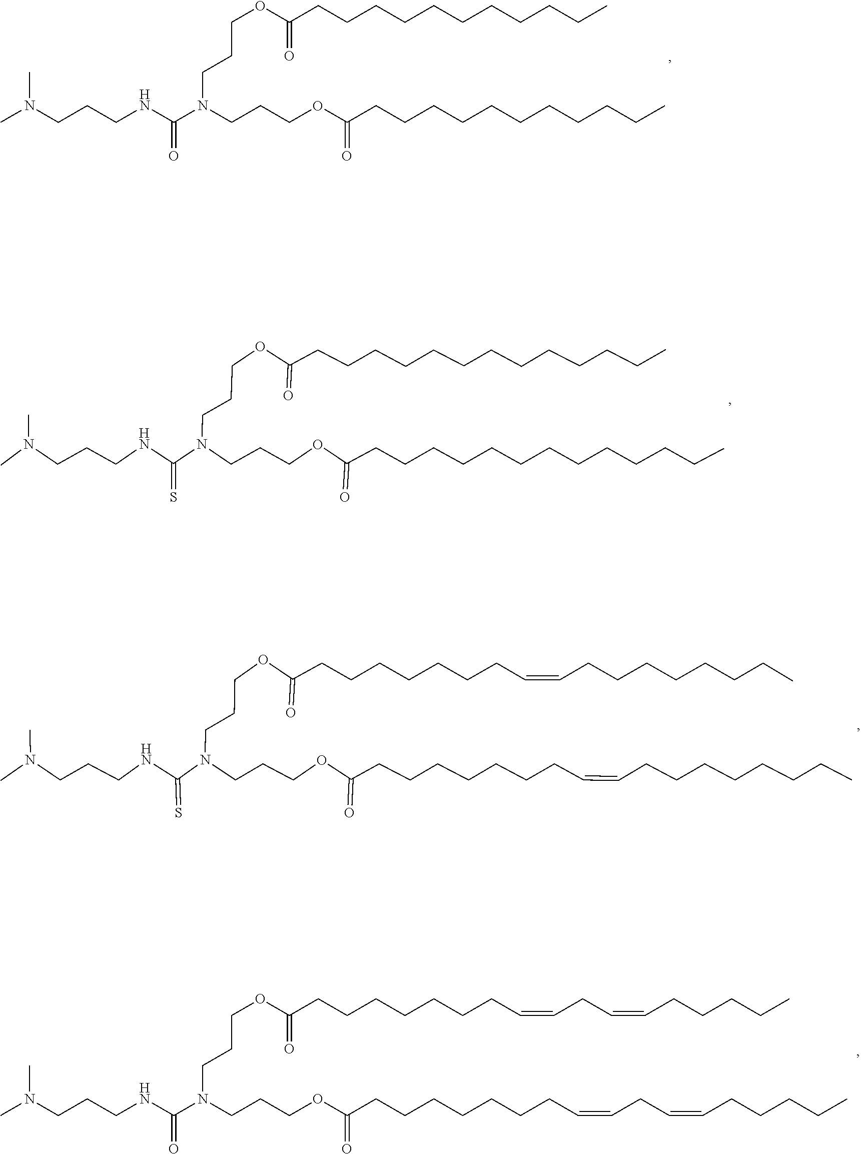

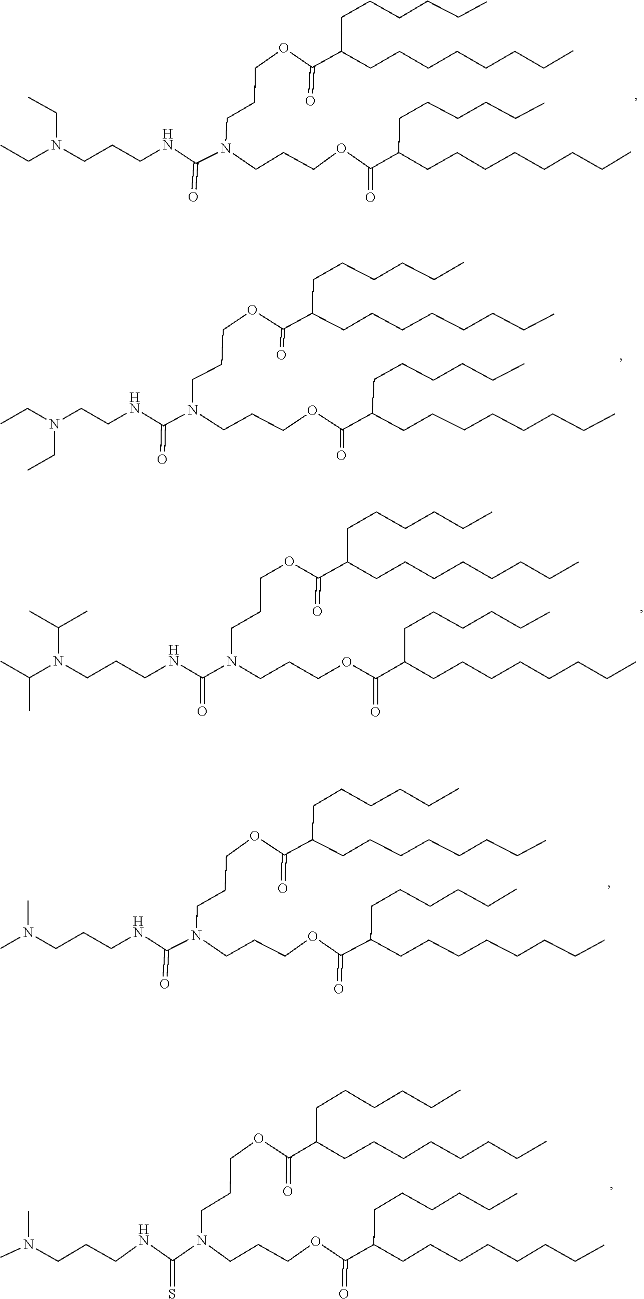

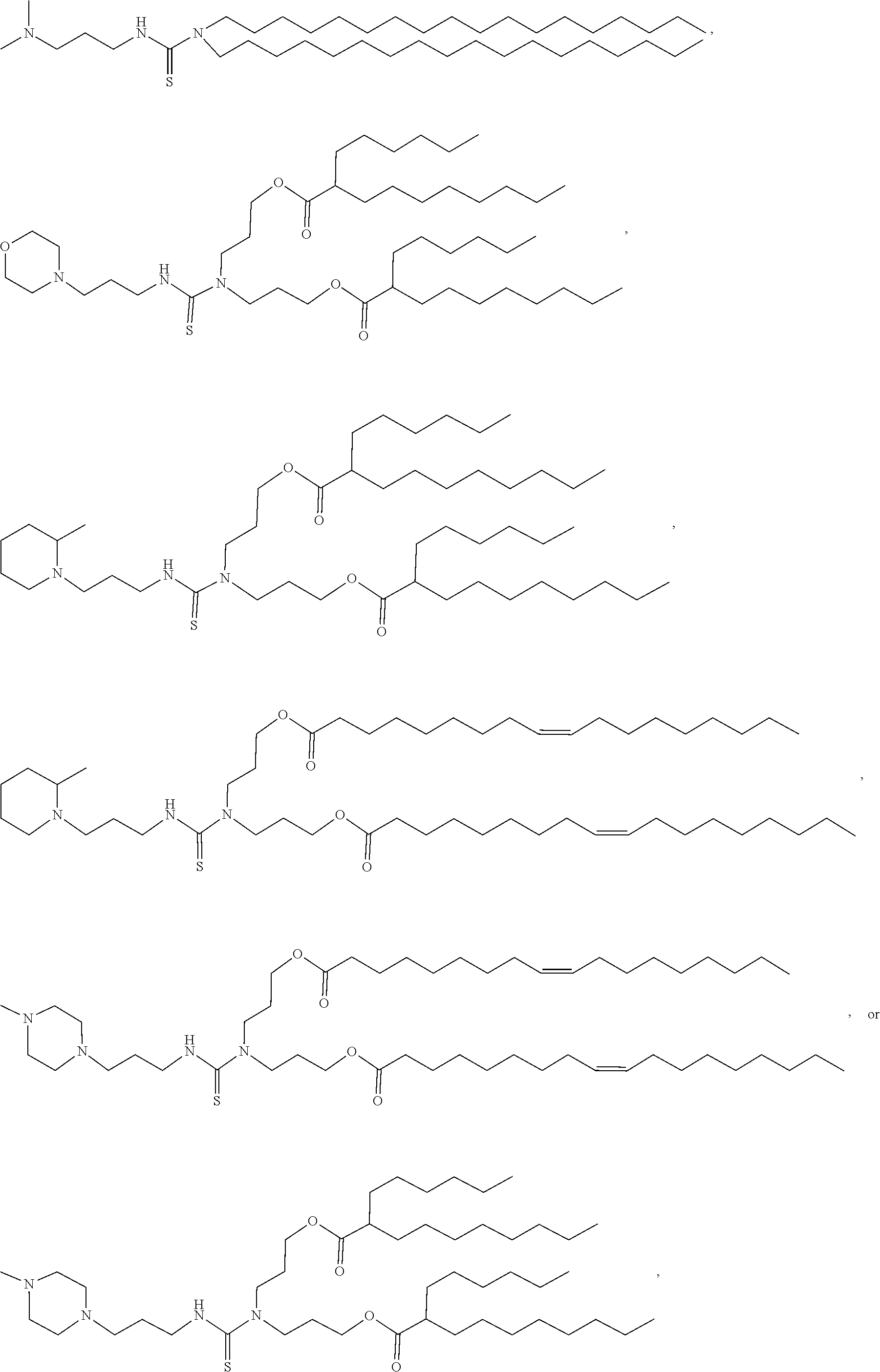

or a pharmaceutically acceptable salt thereof.

Embodiment 84. The pharmaceutical composition of Embodiment 69 or Embodiment 70, wherein the lipid comprises a cationic lipid of formula (III):

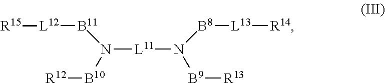

or a pharmaceutically acceptable salt, solvate, hydrate, stereoisomer, or prodrug thereof, wherein:
   L¹¹ is

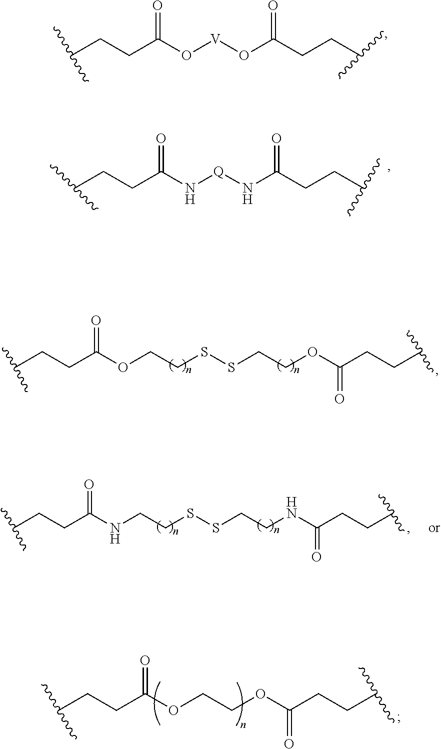

Q is substituted or unsubstituted alkylene, substituted or unsubstituted heteroalkylene, substituted or unsubstituted cycloalkylene, substituted or unsubstituted heterocycloalkylene, substituted or unsubstituted arylene, substituted or unsubstituted heteroaylenearylene;
V is substituted or unsubstituted alkylene, substituted or unsubstituted cycloalkylene, substituted or unsubstituted arylene;
B⁸, B⁹, B¹⁰, and B¹¹ are each independently a bond, substituted or unsubstituted alkylene, or substituted or unsubstituted heteroalkylene;
L¹² is a bond, —O(C═O)—, —(C═O)O—, —O(C═O)O—, —C(═O)—, —O—, —O(CR²¹⁰R²¹¹)ₛO—, —S—, —C(═O)S—, —SC(═O)—, —NR²¹⁰C(═O)—, —C(═O)NR²¹⁰—, —NR²¹⁰C(═O)NR²¹¹—, —NR²¹⁰C(═S)—, —C(═S)NR²¹⁰—, —NR²¹⁰C(═S)NR²¹¹—, —OC(═O)NR²¹⁰—, —NR²¹⁰C(═O)O—, —SC(═O)NR²¹⁰— or —NR²¹⁰C(═O)S—;
L¹³ is a bond, —O(C═O)—, —(C═O)O—, —O(C═O)O—, —C(═O)—, —O—, —O(CR³¹⁰R³¹¹)ₛO—, —S—, —C(═O)S—, —SC(═O)—, —NR³¹⁰C(═O)—, —C(═O)NR³¹⁰—, —NR³¹⁰C(═O)NR³¹¹—, —NR³¹⁰C(═S)—, —C(═S)NR³¹⁰—, —NR³¹⁰C(═S)NR³¹¹—, —O(═O)NR³¹⁰—, —NR³¹⁰C(═O)O—, —SC(═O)NR³¹⁰— or —NR³¹⁰C(═O)S—;
R¹² is H, —OR^{12A}, —SR^{12A}, —NR¹²A, —CN, —(C═O)R^{12A}, —O(C═O)R^{12A}, —(C═O)OR^{12A}, —NR^{12A}(C═O)—R^{12B}, —(C═O)NR^{12A}R^{12B};
R¹³ is H, —OR^{13A}, —SR^{13A}, —NR^{13A}, —CN, —(C═O)R^{13A}, —O(C═O)R^{13A}, —(C═O)OR^{13A}, —NR^{13A}(C═O)—R^{13B}, (C═O)NR^{13A}R^{13B};
R¹⁴ and R¹⁵ are each independently substituted or unsubstituted C₂-C₃₀ alkyl, or substituted or unsubstituted 2 to 30 membered heteroalkyl;
R^{12A}, R^{12B}, R^{13A}, and R^{13B} are each independently H, substituted or unsubstituted C₁-C₂₀ alkyl, or substituted or unsubstituted 2 to 20 membered heteroalkyl;
each R²¹⁰, R²¹¹, R³¹⁰, and R³¹¹ is independently H, substituted or unsubstituted C₁-C₁₂ alkyl, or substituted or unsubstituted 2 to 12 membered heteroalkyl; each n is independently an integer from 0 to 8; and each s is independently an integer from 1 to 4.

Embodiment 85. The pharmaceutical composition of Embodiment 84, wherein the cationic lipid is a lipid wherein:
L¹¹ is

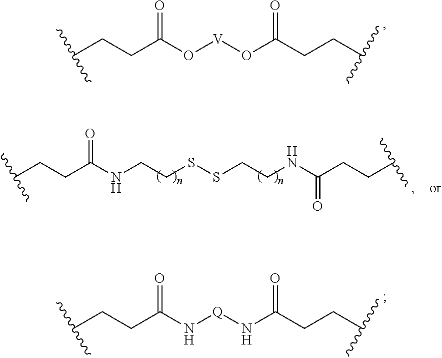

Q is substituted or unsubstituted alkylene;
V is substituted or unsubstituted alkylene;
B⁸, B⁹, B¹⁰, and B¹¹ are each independently substituted or unsubstituted alkylene;
L¹² is —O(C═O)— or —(C═O)O—;
L¹³ is —O(C═O)— or —(C═O)O—;
R¹² is H, —OR^{12A}, or —NR¹²A;
R¹³ is H, —OR^{13A}, or —NR^{13A};
R¹⁴ and R¹⁵ are each independently substituted or unsubstituted C₂-C₃₀ alkyl;
R^{12A} and R^{13A} are each independently H, substituted or unsubstituted C₁-C₂₀ alkyl; and
each n is independently an integer from 0 to 8.

Embodiment 86. The pharmaceutical composition of Embodiment 84, wherein the cationic lipid is a lipid wherein:

L¹¹ is

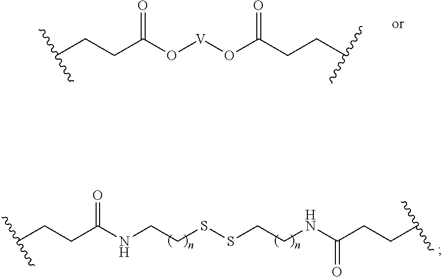

V is substituted or unsubstituted alkylene;
B⁸, B⁹, B¹⁰, and B¹¹ are each independently substituted or unsubstituted C₁-C₂₀ alkylene;
L¹² is —O(C═O)— or —(C═O)O—;
L¹³ is —O(C═O)— or —(C═O)O—;
R¹² is H or —OR¹²A;
R¹³ is H or —OR^{13A};
R¹⁴ and R¹⁵ are each independently substituted or unsubstituted C₂-C₂₀ alkyl;
R^{12A} and R^{13A} are each independently H, substituted or unsubstituted C₁-C₈ alkyl; and each
n is independently an integer from 0 to 4.

Embodiment 87. The pharmaceutical composition of Embodiment 84, wherein the cationic lipid is a lipid wherein:
L¹¹ is

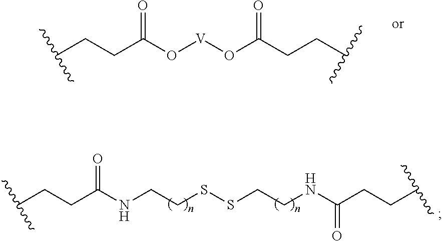

V is unsubstituted alkylene;
B⁸, B⁹, B¹⁰, and B¹¹ are each independently substituted or unsubstituted C₁-C₈ alkylene;
L¹² is —O(C═O)— or —(C═O)O—;
L¹³ is —O(C═O)— or —(C═O)O—;
R¹² is —OH, methoxy, or ethoxy;
R¹³ is —OH, methoxy, or ethoxy;
R¹⁴ and R¹⁵ are each independently substituted or unsubstituted C₂-C₂₀ alkyl; and
each n is independently an integer from 0 to 4.

Embodiment 88. The pharmaceutical composition of Embodiment 84, wherein the cationic lipid is a lipid wherein the cationic lipid is:

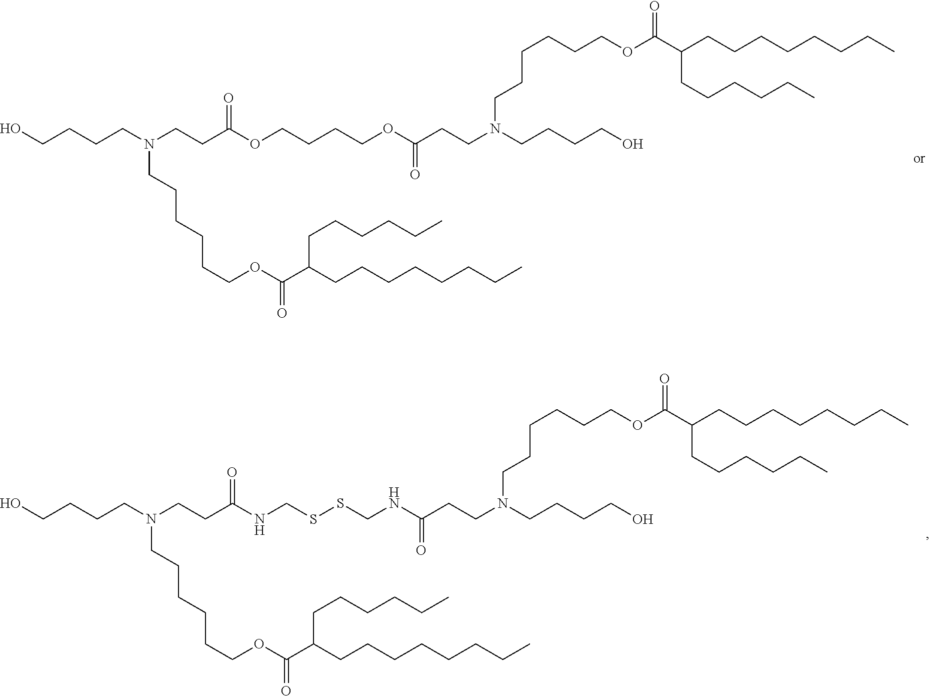 [SMILES: CCCCCCCCC(CCCCCC)C(=O)OCCCCCCN(CCCCO)CCC(=O)OCCCCOC(=O)CCN(CCCCO)CCCCCCOC(=O)C(CCCCCC)CCCCCCCC ; CCCCCCCCC(CCCCCC)C(=O)OCCCCCCN(CCCCO)CCC(=O)NCSSCNC(=O)CCN(CCCCO)CCCCCCOC(=O)C(CCCCCC)CCCCCCCC]

or a pharmaceutically acceptable salt thereof.

Embodiment 89. The pharmaceutical composition of Embodiment 69 or Embodiment 70, wherein the lipid comprises a cationic lipid of formula (IV):

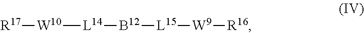

or a pharmaceutically acceptable salt, solvate, hydrate, stereoisomer, or prodrug thereof, wherein:

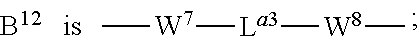

W⁷ and W⁸ are each independently a bond, substituted or unsubstituted C₁-C₁₂ alkylene, or substituted or unsubstituted 2 to 12 membered heteroalkylene;
L^{a3} is a bond, —S—S—, —O—(CH₂O)ₘ—,

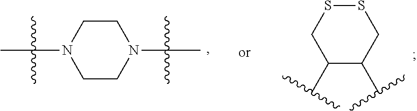 [SMILES: *N1CCN(*)CC1 ; *C1CSSCC1*]

W⁹ and W¹⁰ are each independently a bond, substituted or unsubstituted C₁-C₁₂ alkylene, substituted or unsubstituted 2 to 12 membered heteroalkylene, substituted or unsubstituted cycloalkylene, substituted or unsubstituted heterocycloalkylene, or any combination thereof;
L¹⁴ is a bond, —O(C═O)—, —(C═O)O—, —O(C═O)O—, —C(═O)—, —O—, —O(CR⁴¹⁰R⁴¹¹)ₛO—, —S—, —C(═O)S—, —SC(═O)—, —NR⁴¹⁰C(═O)—, —C(═O)NR⁴¹⁰—, —NR⁴¹⁰C(═O)NR⁴¹¹—, —NR⁴¹⁰C(═S)—, —C(═S)NR⁴¹⁰—, —NR⁴¹⁰C(═S)NR⁴¹¹—, —OC(═O)NR⁴¹⁰—, —NR⁴¹⁰C(═O)O—, —SC(═O)NR⁴¹⁰— or —NR⁴¹⁰C(═O)S—;
L¹⁵ is a bond, —O(C═O)—, —(C═O)O—, —O(C═O)O—, —C(═O)—, —O—, —O(CR⁵¹⁰R⁵¹¹)ₛO—, —S—, —C(═O)S—, —SC(═O)—, —NR⁵¹⁰C(═O)—, —C(═O)NR⁵¹⁰—, —NR⁵¹⁰C(═O)NR⁵¹⁰—, —NR⁵¹⁰C(═S)—, —C(═S)NR⁵¹⁰—, —NR⁵¹⁰C(═S)NR⁵¹⁰—, —OC(═O)NR⁵¹⁰—, —NR⁵¹⁰C(═O)O—, —SC(═O)NR⁵¹⁰— or —NR⁵¹⁰C(═O)S—;
R¹⁶ and R¹⁷ are each independently

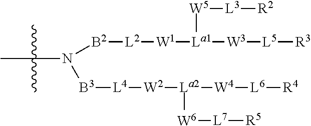

a fragment of cationic lipid of formula (I),

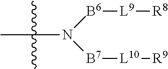

a fragment of cationic lipid of formula (II),

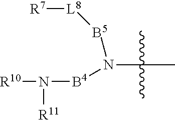

a fragment of cationic lipid of formula (II),

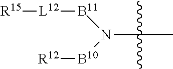

a fragment of cationic lipid of formula (III), or

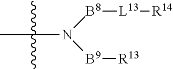

a fragment of cationic lipid of formula (III);
each R⁴¹⁰, R⁴¹¹, R⁵¹⁰, and R⁵¹¹ is independently H, substituted or unsubstituted C₁-C₁₂ alkyl, or substituted or unsubstituted 2 to 12 membered heteroalkyl; each m is independently an integer from 0 to 8; and each s is independently an integer from 1 to 4.

Embodiment 90. The pharmaceutical composition of Embodiment 89, wherein the cationic lipid is a lipid wherein:
L^{a3} is a bond, —S—S—,

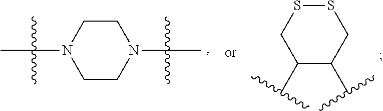 [SMILES: *N1CCN(*)CC1 ; *C1CSSCC1*]

W⁷ and W⁸ are each independently a bond or substituted or unsubstituted C₁-C₁₂ alkylene;
L¹⁴ is —O(C═O)—, —(C═O)O—, —C(═O)—, —NR⁴¹⁰C(═O)—, —C(═O)NR⁴¹⁰—, —NR⁴¹⁰C(═S)—, —C(═S)NR⁴¹⁰—, —OC(═O)NR⁴¹⁰—, or —NR⁴¹⁰C(═O)O—;
L¹⁵ is —O(C═O)—, —(C═O)O—, —C(═O)—, —NR⁵¹⁰C(═O)—, —C(═O)NR⁵¹⁰—, —NR⁵¹⁰C(═S)—, —C(═S)NR⁵¹⁰—, —OC(═O)NR⁵¹⁰—, or —NR⁵¹⁰C(═O)O—;
W⁹ and W¹⁰ are each independently a bond or substituted or unsubstituted C₁-C₁₂ alkylene;
R¹⁶ and R¹⁷ are each independently

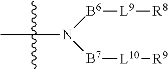

a fragment of cationic lipid of formula (II); and
each R⁴¹⁰ and R⁵¹⁰ is independently H or substituted or unsubstituted C₁-C₁₂ alkyl.

Embodiment 91. The pharmaceutical composition of Embodiment 89, wherein the cationic lipid is a lipid wherein:
L^{a3} is a bond, —S—S—,

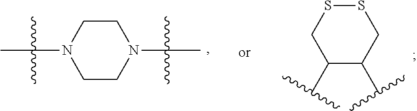 [SMILES: *N1CCN(*)CC1 ; *C1CSSCC1*]

W⁷ and W⁸ are each independently a bond or unsubstituted C₁-C₁₂ alkylene;
L¹⁴ is —O(C═O)—, —(C═O)O—, —NR⁴¹⁰C(═S)—, —C(═S)NR⁴¹⁰—, —OC(═O)NR⁴¹⁰—, or —NR⁴¹⁰C(═O)O—;
L¹⁵ is —O(C═O)—, —(C═O)O—, —NR⁵¹⁰C(═S)—, —C(═S)NR⁵¹⁰—, —OC(═O)NR⁵¹⁰—, or —NR⁵¹⁰C(═O)O—;
W⁹ and W¹⁰ are each independently a bond or substituted or unsubstituted C₁-C₁₂ alkylene;
R¹⁶ and R¹⁷ are each independently

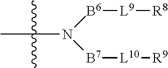

a fragment of cationic lipid of formula (II); and
each R⁴¹⁰ and R⁵¹⁰ is independently H or substituted or unsubstituted C₁-C₁₂ alkyl.

Embodiment 92. The pharmaceutical composition of Embodiment 89, wherein the cationic lipid is a lipid wherein:
L^{a3} is a bond, —S—S—,

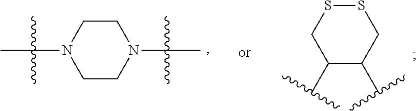 [SMILES: *N1CCN(*)CC1 ; *C1CSSCC1*]

W⁷ and W⁸ are each independently a bond or unsubstituted C₁-C₈ alkylene;
L¹⁴ is —O(C═O)—, —(C═O)O—, —NR⁴¹⁰C(═S)—, —C(═S)NR⁴¹⁰—, —OC(═O)NR⁴¹⁰—, or —NR⁴¹⁰C(═O)O—;
L¹⁵ is —O(C═O)—, —(C═O)O—, —NR⁵¹⁰C(═S)—, —C(═S)NR⁵¹⁰—, —OC(═O)NR⁵¹⁰—, or —NR⁵¹⁰C(═O)O—;
W⁹ and W¹⁰ are each independently a bond or unsubstituted C₁-C₈ alkylene;
R¹⁶ and R¹⁷ are each independently

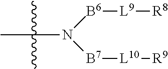

a fragment of cationic lipid of formula (II); and
each R⁴¹⁰ and R⁵¹⁰ is independently H or unsubstituted C₁-C₈ alkyl.

Embodiment 93. The pharmaceutical composition of Embodiment 89, wherein the cationic lipid is a lipid wherein:
L^{a3} is a bond, —S—S—,

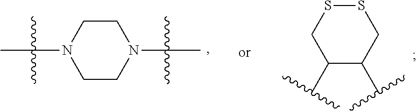 [SMILES: *N1CCN(*)CC1 ; *C1CSSCC1*]

W⁷ and W⁸ are each independently a bond or unsubstituted C₁-C₈ alkylene;
L¹⁴ is —O(C═O)—, —(C═O)O—, —NR⁴¹⁰C(═S)—, —C(═S)NR⁴¹⁰—, —OC(═O)NR⁴¹⁰—, or —NR⁴⁰C(═O)O—;
L¹⁵ is —O(C═O)—, —(C═O)O—, —NR⁵¹⁰C(═S)—, —C(═S)NR⁵¹⁰—, —OC(═O)NR⁵¹⁰—, or —NR⁵¹⁰C(═O)O—;
W⁹ and W¹⁰ are each independently a bond or unsubstituted C₁-C₈ alkylene;
R¹⁶ and R¹⁷ are each independently

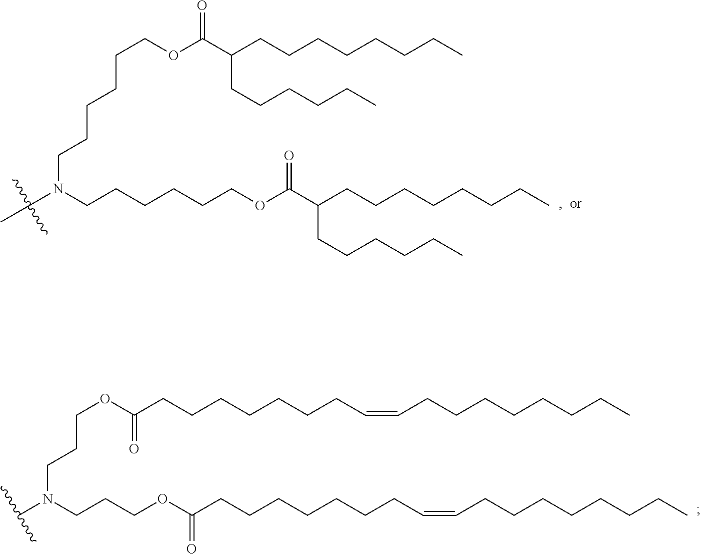 [SMILES: *N(CCCCCCOC(=O)C(CCCCCC)CCCCCCCC)CCCCCCOC(=O)C(CCCCCC)CCCCCCCC ; *N(CCCOC(=O)CCCCCCC/C=C\CCCCCCCC)CCCOC(=O)CCCCCCC/C=C\CCCCCCCC]

and each R⁴¹⁰ and R⁵¹⁰ is independently H or methyl.

Embodiment 94. The pharmaceutical composition of Embodiment 89, wherein the cationic lipid is:

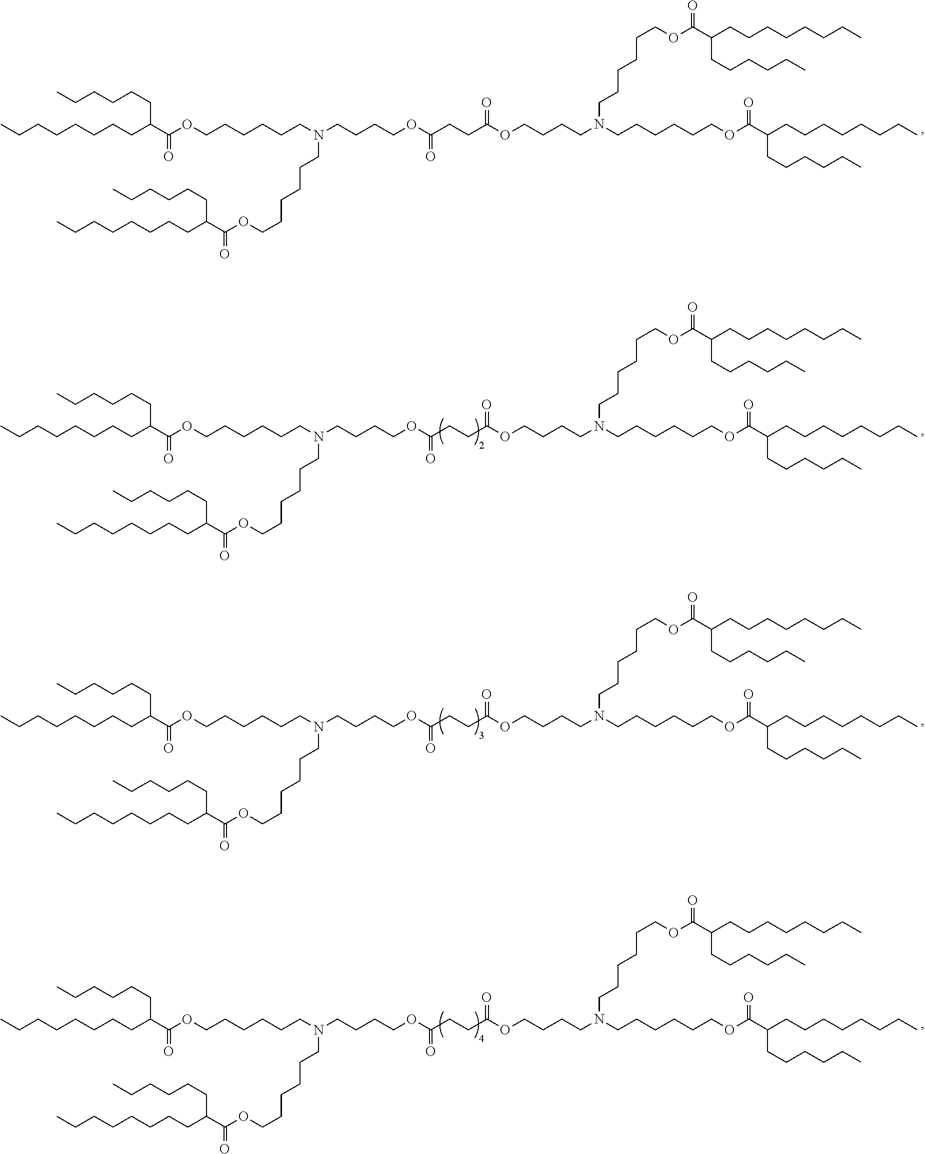

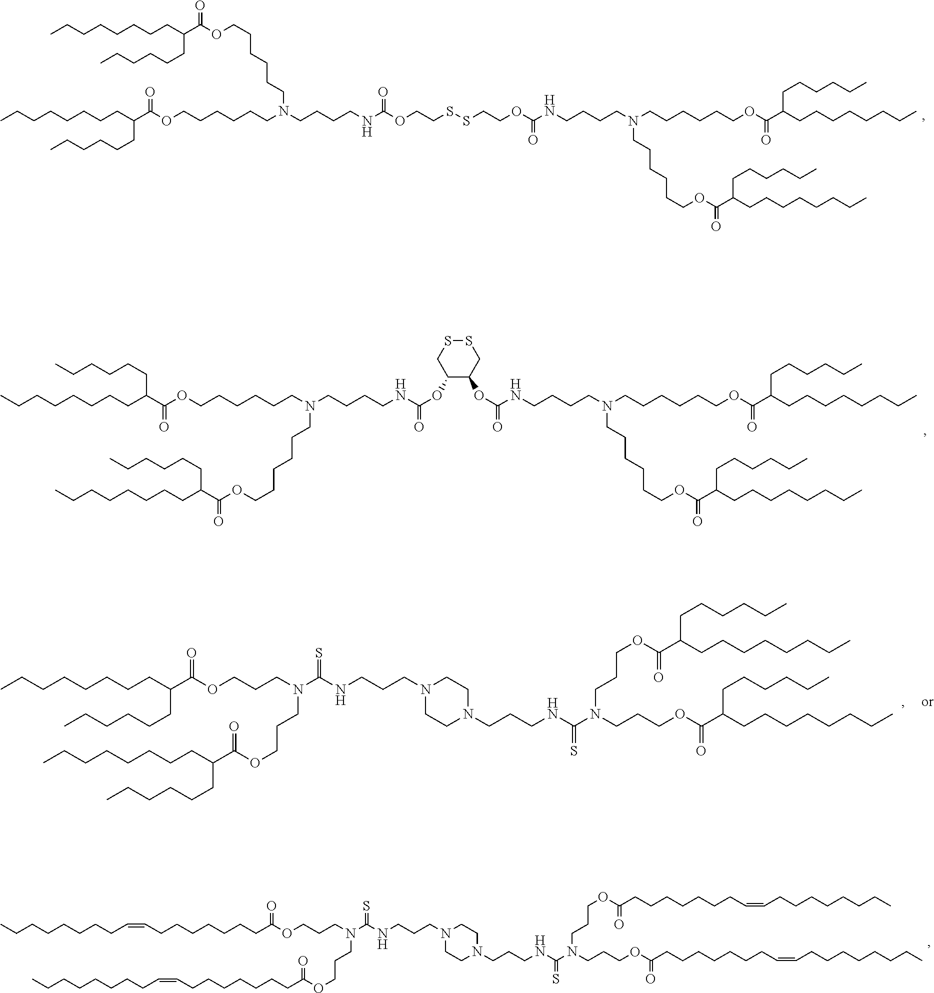

or a pharmaceutically acceptable salt thereof.

Embodiment 95. The pharmaceutical composition of any of Embodiments 69-94, wherein the pharmaceutical composition comprises the following compound:

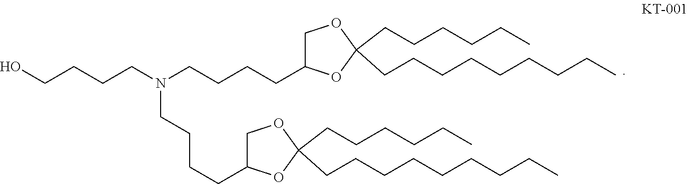 [SMILES: CCCCCCCCCC1(CCCCCC)OCC(CCCCN(CCCCO)CCCCC2COC(CCCCCC)(CCCCCCCCC)O2)O1]

Embodiment 96. The pharmaceutical composition of any of Embodiments 69-95, wherein the pharmaceutical composition further comprises lipid nanoparticles.

Embodiment 97. The pharmaceutical composition of any of Embodiments 69-96, further comprising at least a second therapeutic agent.

Embodiment 98. The pharmaceutical composition of Embodiment 97, wherein the at least a second therapeutic agent comprises an anti-neoplasm agent or substance, immune-oncology (I-0) agent, an immune checkpoint inhibitor, and/or an antibody-dependent cell-mediated cytotoxicity agent, and/or a cell therapy agent.

Embodiment 99. The pharmaceutical composition of Embodiment 97 or Embodiment 98, wherein the at least a second therapeutic agent comprises an anti-PD1 antibody, and anti-PDL1 antibody, an anti-EGFR antibody, and anti-Her2 antibody, and anti-CD20 antibody, an anti-CD38 antibody, an anti-CD47 antibody, an anti-CD123 antibody, an anti-cMET antibody, an anti-CCR4 antibody, an anti-CTLA4 antibody, a chimeric antigen receptor T cell (CAR-T), or a dimeric antigen receptor T cell (DAR-T).

Embodiment 100. The polynucleotide of any one of Embodiments 1-68 or the pharmaceutical composition of any one of Embodiments 69-99, wherein the polynucleotide or the pharmaceutical composition is for use in a method of treating or preventing a proliferation disorder in a subject.

Embodiment 101. A method for treating or preventing a proliferation disease or disorder in a subject having, suspected of having, or at risk of having the proliferation disease or disorder, the method comprising administering to the subject an effective amount of the polynucleotide of any one of Embodiments 1-68 or the pharmaceutical composition of any one of Embodiments 69-99.

Embodiment 102. The method of Embodiment 100 or Embodiment 101, wherein the proliferation disease or disorder comprises a tumor.

Embodiment 103. The method of any one of Embodiments 100-102, wherein the proliferation disease or disorder comprises a cancer.

Embodiment 104. The method of any one of Embodiments 100-103, wherein the proliferation disease or disorder comprises a solid tumor.

Embodiment 105. The method of any one of Embodiments 100-104, wherein the proliferation disease or disorder is selected from the group consisting of: Chondrosarcoma, Ewing's sarcoma, Malignant fibrous histiocytoma of bone/osteosarcoma, Osteosarcoma, Rhabdomyosarcoma, Heart cancer, Astrocytoma, Brainstem glioma, Pilocytic astrocytoma, Ependymoma, Primitive neuroectodermal tumor, Cerebellar astrocytoma, Cerebral astrocytoma, Glioma, Medulloblastoma, Neuroblastoma, Oligodendroglioma, Pineal astrocytoma, Pituitary adenoma, Visual pathway and hypothalamic glioma, Breast cancer, Invasive lobular carcinoma, Tubular carcinoma, Invasive cribriform carcinoma, Medullary carcinoma, Male breast cancer, Phyllodes tumor, Inflammatory Breast Cancer, Adrenocortical carcinoma, Islet cell carcinoma (endocrine pancreas), Multiple endocrine neoplasia syndrome, Parathyroid cancer, Pheochromocytoma, Thyroid cancer, Merkel cell carcinoma, Uveal melanoma, Retinoblastoma, Anal cancer, Appendix cancer, cholangiocarcinoma, Carcinoid tumor, gastrointestinal, Colon cancer, Extrahepatic bile duct cancer, Gallbladder cancer, Gastric (stomach) cancer, Gastrointestinal carcinoid tumor, Gastrointestinal stromal tumor (GIST), Hepatocellular cancer, Pancreatic cancer, Islet cell cancer, Rectal cancer, Colorectal cancer, Bladder cancer, Cervical cancer, Endometrial cancer, Extragonadal germ cell tumor, Ovarian cancer, Ovarian epithelial cancer (surface epithelial-stromal tumor), Ovarian germ cell tumor, Fallopian Tube cancer, Peritoneal cancer, Penile cancer, Renal cell carcinoma (RCC), Renal pelvis and ureter cancer, transitional cell cancer, Prostate cancer, Testicular cancer, Gestational trophoblastic tumor, Ureter and renal pelvis, transitional cell cancer, Urethral cancer, Uterine sarcoma, Vaginal cancer, Vulvar cancer, Wilms tumor, Esophageal cancer, Head and neck cancer, Nasopharyngeal carcinoma, Oral cancer, Oropharyngeal cancer, Paranasal sinus and nasal cavity cancer, Pharyngeal cancer, Salivary gland cancer, Hypopharyngeal cancer, Basal-cell carcinoma, Melanoma, Skin cancer (non-melanoma), Bronchial adenomas/carcinoids, Small cell lung cancer, Mesothelioma, Non-small cell lung cancer (NSCLC), Pleuropulmonary blastoma, Laryngeal cancer, Thymoma and thymic carcinoma, AIDS-related cancers, Kaposi sarcoma, Epithelioid hemangioendothelioma (EHE), Desmoplastic small round cell tumor, and Liposarcoma.

Embodiment 106. The method of any one of Embodiments 102-104, wherein the tumor or the cancer comprises a hematological malignancy.

Embodiment 107. The method of any one of Embodiments 100-103 and 106, wherein the proliferation disease or disorder comprises a hematological malignancy selected from the group consisting of: myeloid neoplasms, Leukemias, Lymphomas, Hodgkin lymphoma, classic Hodgkin Lymphoma, Non-Hodgkin lymphoma, Anaplastic large cell lymphoma, Angioimmunoblastic T-cell lymphoma, Hepatosplenic T-cell lymphoma, B-cell lymphoma reticuloendotheliosis, Reticulosis, Microglioma, Diffuse large B-cell lymphoma, Follicular lymphoma, Mucosa-associated lymphatic tissue lymphoma, B-cell chronic lymphocytic leukemia, Mantle cell lymphoma, Burkitt lymphoma, Mediastinal large B cell lymphoma, Waldenstrom's macroglobulinemia, Nodal marginal zone B cell lymphoma, Splenic marginal zone lymphoma, Intravascular large B-cell lymphoma, Primary effusion lymphoma, Lymphomatoid granulomatosis, Nodular lymphocyte predominant Hodgkin's lymphoma, plasma cell leukemia, Acute erythraemia and erythroleukaemia, Acute erythremic myelosis, Acute erythroid leukemia, Heilmeyer-Schoner disease, Acute megakaryoblastic leukemia, Mast cell leukemia, Panmyelosis, Acute panmyelosis with myelofibrosis, Lymphosarcoma cell leukemia, Acute leukaemia of unspecified cell type, Blastic phase chronic myelogenous leukemia, Stem cell leukemia, Chronic leukaemia of unspecified cell type, Subacute leukaemia of unspecified cell type, Accelerated phase chronic myelogenous leukemia, Acute myeloid leukemia, Polycythemia vera, Acute promyelocytic leukemia, Acute basophilic leukemia, Acute eosinophilic leukemia, Acute lymphoblastic leukemia, Acute monocytic leukemia, Acute myeloblastic leukemia with maturation, Acute myeloid dendritic cell leukemia, Adult T-cell leukemia/lymphoma, Aggressive NK-cell leukemia, B-cell prolymphocytic leukemia, B-cell chronic lymphocytic leukemia, B-cell leukemia, Chronic myelogenous leukemia, Chronic myelomonocytic leukemia, Chronic neutrophilic leukemia, Chronic lymphocytic leukemia, Hairy cell leukemia, Chronic idiopathic myelofibrosis, Multiple myeloma, Kahler's disease, Myelomatosis, Solitary myeloma, Plasma cell leukemia, Plasmacytoma, extramedullary, Malignant plasma cell tumour NOS, Plasmacytoma NOS, Monoclonal gammopathy, Multiple Myeloma, Angiocentric immunoproliferative lesion, Lymphoid granulomatosis, Angioimmunoblastic lymphadenopathy, T-gamma lymphoproliferative disease, Waldenstrom's macroglobulinaemia, Alpha heavy chain disease, Gamma heavy chain disease, Franklin's disease, Immunoproliferative small intestinal disease, Mediterranean disease, Malignant immunoproliferative disease, unspecified, and Immunoproliferative disease NOS.

Embodiment 108. A method for treating or preventing an immune deficiency disease or disorder in a subject having, suspected of having, or at risk of having the immune deficiency disease or disorder, the method comprising administering to the subject an effective amount of the polynucleotide of any one of Embodiments 1-68 or the pharmaceutical composition of any one of Embodiments 69-99.

Embodiment 109. The method of Embodiment 108, wherein the immune deficiency disease or disorder is selected from the group consisting of: Agammaglobulinemia: X-Linked and Autosomal Recessive, Ataxia Telangiectasia, Chronic Granulomatous Disease and Other Phagocytic Cell Disorders, Common Variable Immune Deficiency, Complement Deficiencies, DiGeorge Syndrome, Hemophagocytic Lymphohistiocytosis (HLH), Hyper IgE Syndrome, Hyper IgM Syndromes, IgG Subclass Deficiency, Innate Immune Defects, NEMO Deficiency Syndrome, Selective IgA Deficiency, Selective IgM Deficiency, Severe Combined Immune, Deficiency and Combined Immune Deficiency, Specific Antibody Deficiency, Transient Hypogammaglobulinemia of Infancy, WHIM Syndrome (Warts, Hypogammaglobulinemia, Infections, and Myelokathexis), Wiskott-Aldrich Syndrome, Other Antibody Deficiency Disorders, Other Primary Cellular Immunodeficiencies, Severe combined immune deficiency (SCID), Common variable immune deficiency (CVID), Human immunodeficiency virus/acquired immune deficiency syndrome (HIV/AIDS), Drug-induced immune deficiency, Graft versus host syndrome, Primary Immune Deficiency Diseases (PIDDs), and Lymphopenia.

Embodiment 110. A method for treating or preventing an autoimmune or inflammatory disease or disorder in a subject having, suspected of having, or at risk of having the autoimmune or inflammatory disease or disorder, the method comprising administering to the subject an effective amount of the polynucleotide of any one of Embodiments 1-68 or the pharmaceutical composition of any one of Embodiments 69-99.

Embodiment 111. The method of Embodiment 110, wherein the autoimmune or inflammatory disease or disorder is selected from the group consisting of: inflammation, autoimmune disease, paraneoplastic autoimmune diseases, cartilage inflammation, fibrotic disease and/or bone degradation, arthritis, rheumatoid arthritis, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reter's Syndrome, SEA Syndrome (Seronegativity, Enthesopathy, Arthropathy Syndrome), juvenile dermatomyositis, juvenile psoriatic arthritis, juvenile scleroderma, juvenile systemic lupus erythematosus, juvenile vasculitis, pauciarticular rheumatoid arthritis, polyarticular rheumatoid arthritis, systemic onset rheumatoid arthritis, ankylosing spondylitis, enteropathic arthritis, reactive arthritis, Reter's Syndrome, SEA Syndrome (Seronegativity, Enthesopathy, Arthropathy Syndrome), dermatomyositis, psoriatic arthritis, scleroderma, systemic lupus erythematosus, vasculitis, myolitis, polymyolitis, dermatomyolitis, osteoarthritis, polyarteritis nodossa, Wegener's granulomatosis, arteritis, ploymyalgia rheumatica, sarcoidosis, scleroderma, sclerosis, primary biliary sclerosis, sclerosing cholangitis, Sjogren's syndrome, psoriasis, plaque psoriasis, guttate psoriasis, inverse psoriasis, pustular psoriasis, erythrodermic psoriasis, dermatitis, atopic dermatitis, atherosclerosis, lupus, Still's disease, Systemic Lupus Erythematosus (SLE), myasthenia gravis, inflammatory bowel disease (IBD), Crohn's disease, ulcerative colitis, celiac disease, multiple schlerosis (MS), asthma, COPD, Guillain-Barre disease, Type I diabetes mellitus, thyroiditis (e.g., Graves' disease), Addison's disease, Raynaud's phenomenon, autoimmune hepatitis, GVHD, and transplantation rejection.

Embodiment 112. A method for treating or preventing an infectious disease or disorder in a subject in a subject having, suspected of having, or at risk of having an infectious disease or, the method comprising administering to the subject an effective amount of the polynucleotide of any one of Embodiments 1-68 or the pharmaceutical composition of any one of Embodiments 69-99.

Embodiment 113. The method of Embodiment 112, wherein the infectious disease or disorder is selected from the group consisting of: Acinetobacter infections, Actinomycosis, African sleeping sickness (African trypanosomiasis), AIDS (acquired immunodeficiency syndrome), Amoebiasis, Anaplasmosis, Angiostrongyliasis Anisakiasis, Anthrax, Arcanobacterium haemolyticum infection, Argentine hemorrhagic fever, Ascariasis, Aspergillosis Astrovirus infection, Babesiosis, Bacillus cereus infection, Bacterial meningitis, Bacterial pneumonia, Bacterial vaginosis, Bacteroides infection, Balantidiasis, Bartonellosis, Baylisascaris infection, BK virus infection, Black piedra, Blastocystosis, Blastomycosis, Bolivian hemorrhagic fever, Botulism (and Infant botulism), Brazilian hemorrhagic fever, Brucellosis, Bubonic plague, Burkholderia infection, Buruli ulcer, Calicivirus infection (Norovirus and Sapovirus), Campylobacteriosis, Candidiasis (Moniliasis; Thrush), Capillariasis, Carrion's disease, Cat-scratch disease, Cellulitis, Chagas disease (American trypanosomiasis), Chancroid, Chickenpox, Chikungunya, Chlamydia, Chlamydophila pneumoniae infection (Taiwan acute respiratory agent or TWAR), Cholera, Chromoblastomycosis, Chytridiomycosis, Clonorchiasis, Clostridium difficile colitis, Coccidioidomycosis, Colorado tick fever (CTF), Common cold (Acute viral rhinopharyngitis; Acute coryza), Coronavirus disease 2019 (COVID-19), Creutzfeldt-Jakob disease (CJD), Crimean-Congo hemorrhagic fever (CCHF), Cryptococcosis, Cryptosporidiosis, Cutaneous larva migrans (CLM), Cyclosporiasis, Cysticercosis, Cytomegalovirus infection, Dengue fever, Desmodesmus infection, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum (Sixth disease), Fasciolasis, Fasciolopsiasis, Fatal familial insomnia (FFI), Filariasis, Food poisoning by Clostridium perfringens, Free-living amebic infection, Fusobacterium infection, Gas gangrene (Clostridial myonecrosis), Geotrichosis, Gerstmann-Strtussler-Scheinker syndrome (GSS), Giardiasis, Glanders, Gnathostomiasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome (HPS), Heartland virus disease, Helicobacter pylori infection, Hemolytic-uremic syndrome (HUS), Hemorrhagic fever with renal syndrome (HFRS), Hendra virus infection, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis (HGA), Human metapneumovirus infection, Human monocytic ehrlichiosis, Human papillomavirus (HPV) infection, Human parainfluenza virus infection, Hymenolepiasis, Epstein-Barr virus infectious mononucleosis (Mono), Influenza (flu), Isosporiasis, Kawasaki disease, Keratitis, Kingella kingae infection, Kuru, Lassa fever, Legionellosis (Legionnaires' disease), Pontiac fever, Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever (MHF), Measles, Middle East respiratory syndrome (MERS), Melioidosis (Whitmore's disease), Meningitis, Meningococcal disease, Metagonimiasis, Microsporidiosis, Molluscum contagiosum (MC), Monkeypox, Mumps, Murine typhus (Endemic typhus), Mycoplasma pneumonia, Mycoplasma genitalium infection, Mycetoma, Myiasis, Neonatal conjunctivitis (Ophthalmia neonatorum), Nipah virus infection, Norovirus (children and babies), (New) Variant Creutzfeldt-Jakob disease (vCJD, nvCJD), Nocardiosis, Onchocerciasis (River blindness), Opisthorchiasis, Paracoccidioidomycosis (South American blastomycosis), Paragonimiasis, Pasteurellosis, Pediculosis capitis (Head lice), Pediculosis corporis (Body lice), Pediculosis pubis (pubic lice, crab lice), Pelvic inflammatory disease (PID), Pertussis (whooping cough), Plague, Pneumococcal infection, Pneumocystis pneumonia (PCP), Pneumonia, Poliomyelitis, Prevotella infection, Primary amoebic meningoencephalitis (PAM), Progressive multifocal leukoencephalopathy, Psittacosis, Q fever, Rabies, Relapsing fever, Respiratory syncytial virus infection, Rhinosporidiosis, Rhinovirus infection, Rickettsial infection, Rickettsialpox, Rift Valley fever (RVF), Rocky Mountain spotted fever (RMSF), Rotavirus infection, Rubella, Salmonellosis, SARS (severe acute respiratory syndrome), Scabies, Scarlet fever, Schistosomiasis, Sepsis, Shigellosis (bacillary dysentery), Shingles (Herpes zoster), Smallpox (variola), Sporotrichosis, Staphylococcal food poisoning, Staphylococcal infection, Strongyloidiasis, Subacute sclerosing panencephalitis, Bejel, Syphilis, and Yaws, Taeniasis, Tetanus (lockjaw), Tinea barbae (barber's itch), Tinea capitis (ringworm of the scalp), Tinea corporis (ringworm of the body), Tinea cruris (Jock itch), Tinea manum (ringworm of the hand), Tinea nigra, Tinea pedis (athlete's foot), Tinea unguium (onychomycosis), Tinea versicolor (Pityriasis versicolor), Toxocariasis (ocular larva migrans (OLM)), Toxocariasis (visceral larva migrans (VLM)), Toxoplasmosis, Trachoma, Trichinosis, Trichomoniasis, Trichuriasis (whipworm infection), Tuberculosis, Tularemia, Typhoid fever, Typhus fever, Ureaplasma urealyticum infection, Valley fever, Venezuelan equine encephalitis, Venezuelan hemorrhagic fever, Vibrio vulnificus infection, Vibrio parahaemolyticus enteritis, Viral pneumonia, West Nile fever, White piedra (tinea blanca), Yersinia pseudotuberculosis infection, Yersiniosis, Yellow fever, Zeaspora, Zika fever, and Zygomycosis.

Embodiment 114. Use of an effective amount of the polynucleotide of any one of Embodiments 1-68 or the pharmaceutical composition of any one of Embodiments 69-99 for the manufacture of a medicament for treating or preventing a proliferation disease or disorder, an autoimmune or inflammatory disease or disorder, or an infectious disease or disorder, in a subject.

Embodiment 115. The method of any one of Embodiments 100-113 or the use of Embodiment 114, wherein the subject is a human.

Embodiment 116. The method of any one of Embodiments 100-113 or the use of Embodiment 114, wherein the subject is a non-human mammal.

Embodiment 117. A method of expanding a CD4⁺ helper cell, CD8⁺ effector naive and memory cell, T regulatory (Treg) cell, Natural Killer (NK) cell, or Natural killer T (NKT) cell population, comprising contacting a cell population with an effective amount of the polynucleotide of any one of Embodiments 1-68 or the pharmaceutical composition of any one of Embodiments 69-99 for a time sufficient to induce formation of a complex with an IL-2R βγ, thereby stimulating the expansion of the T cell, NK cell, and/or NKT cell population.

Embodiment 118. A method of expanding a CD4⁺ helper cell, CD8⁺ effector naive and memory cell, Treg cell, Natural Killer (NK) cell, or Natural killer T (NKT) cell population, comprising contacting a cell population with an effective amount of the polynucleotide of any one of Embodiments 1-68 or the pharmaceutical composition of any one of Embodiments 69-99 for a time sufficient to induce formation of a complex with an IL-2R γ, thereby stimulating the expansion of the T cell, Treg cell, NK cell, and/or NKT cell population with reduced cell death by 10% to 100%.

Embodiment 119. The method of Embodiment 117 or Embodiment 118, wherein the polynucleotide of any one of Embodiments 1-68 or the pharmaceutical composition of any one of Embodiments 69-99 expands CD4⁺ T Treg cells by less than 20%, 15%, 10%, 5%, 1% or less in the CD3⁺ cell population compared to an expansion of CD4⁺ Treg cells in the CD3⁺ cell population contacted with a comparable IL-2 polypeptide comprising an amino acid sequence set forth in SEQ ID NO:1 or SEQ ID NO:2 without the at least one substitution.

Embodiment 120. The method of any one of Embodiments 117-119, wherein the polynucleotide of any one of Embodiments 1-68 or the pharmaceutical composition of any one of Embodiments 69-99 does not substantially expand CD4⁺ Treg cells in the cell population.

Embodiment 121. The method of any one of Embodiments 117-120, wherein the ratio of effector T cells to Treg cells in the cell population after incubation with the polynucleotide of any one of Embodiments 1-68 or the pharmaceutical composition of any one of Embodiments 69-99 is about or at least 2:1, 3:1, 4:1, 5:1, 6:1, 7:1, 8:1, 9:1, 10:1, 20:1, 50:1, 100:1 or more.

Embodiment 122. The method of any one of Embodiments 117-121, wherein the method is conducted in vivo.

Embodiment 123. The method of any of Embodiments 117-121, wherein the method is conducted in vitro.

Embodiment 124. The method of any of Embodiments 117-121, wherein the method is conducted ex vivo.

Embodiment 125. Use of an effective amount of the polynucleotide of any one of Embodiments 1-68 or the pharmaceutical composition of any one of Embodiments 69-99 for the manufacture of a medicament for expanding a CD4⁺ helper cell, CD8⁺ effector naive and memory cell, Treg cell, Natural Killer (NK) cell, or Natural killer T (NKT) cell population in a cell population.

Embodiment 126. Use of an effective amount of the polynucleotide of any one of Embodiments 1-68 or the pharmaceutical composition of any one of Embodiments 69-99 for expanding a CD4⁺ helper cell, CD8⁺ effector naive and memory cell, Treg cell, Natural Killer (NK) cell, or Natural killer T (NKT) cell population in a subject.

Embodiment 127. The polynucleotide of any one of Embodiments 1-68 or the pharmaceutical composition of any one of Embodiments 69-99, wherein the fusion protein comprises the murine serum albumin (MSA) amino acid sequence set forth in SEQ ID NO:6.

Embodiment 128. The polynucleotide of any one of Embodiments 1-68 or the pharmaceutical composition of any one of Embodiments 69-99, wherein the fusion protein comprises the human serum albumin (HSA) amino acid sequence set forth in SEQ ID NO:7.

Embodiment 129. The polynucleotide of any one of Embodiments 1-68 or the pharmaceutical composition of any one of Embodiments 69-99, wherein the fusion protein comprises the murine serum albumin (MSA) amino acid sequence set forth in SEQ ID NO:6 linked to the N-terminus of the amino acid sequence set forth in SEQ ID NO:3.

Embodiment 130. The polynucleotide of any one of Embodiments 1-68 or the pharmaceutical composition of any one of Embodiments 69-99, wherein the fusion protein comprises the amino acid sequence set forth in SEQ ID NO:3 linked to the N-terminus of the amino acid sequence set forth in the murine serum albumin (MSA) amino acid sequence set forth in SEQ ID NO:6.

Embodiment 131. The polynucleotide of any one of Embodiments 1-68 or the pharmaceutical composition of any one of Embodiments 69-99, wherein the fusion protein comprises the amino acid sequence set forth in the murine serum albumin (MSA) amino acid sequence set forth in SEQ ID NO:6 linked via a linker to the N-terminus of the amino acid sequence set forth in SEQ ID NO:3.

Embodiment 132. The polynucleotide of any one of Embodiments 1-68 or the pharmaceutical composition of any one of Embodiments 69-99, wherein the fusion protein comprises the amino acid sequence set forth in SEQ ID NO:3 linked via a linker to the N-terminus of the amino acid sequence set forth in the murine serum albumin (MSA) amino acid sequence set forth in SEQ ID NO:6.

Embodiment 133. The polynucleotide of any one of Embodiments 1-68 or the pharmaceutical composition of any one of Embodiments 69-99, wherein the fusion protein comprises the amino acid sequence set forth in SEQ ID NO:7 linked to the N-terminus of the amino acid sequence set forth in SEQ ID NO:3.

Embodiment 134. The polynucleotide of any one of Embodiments 1-68 or the pharmaceutical composition of any one of Embodiments 69-99, wherein the fusion protein comprises the amino acid sequence set forth in SEQ ID NO:3 linked to the N-terminus of the amino acid sequence set forth in in SEQ ID NO:7.

Embodiment 135. The polynucleotide of any one of Embodiments 1-68 or the pharmaceutical composition of any one of Embodiments 69-99, wherein the fusion protein comprises the amino acid sequence set forth in in SEQ ID NO:7 linked to the N-terminus of the amino acid sequence set forth in SEQ ID NO:3.

Embodiment 136. The polynucleotide of any one of Embodiments 1-68 or the pharmaceutical composition of any one of Embodiments 69-99, wherein the fusion protein comprises the amino acid sequence set forth in SEQ ID NO:3 linked via a linker to the N-terminus of the amino acid sequence set forth in in SEQ ID NO:7.

Embodiment 137. The polynucleotide of any one of Embodiments 1-68 or the pharmaceutical composition of any one of Embodiments 69-99, wherein the fusion protein comprises the amino acid sequence set forth in SEQ ID NO:4.

Embodiment 138. The polynucleotide of any one of Embodiments 1-68 or the pharmaceutical composition of any one of Embodiments 69-99, wherein the fusion protein comprises the amino acid sequence set forth in SEQ ID NO:5.

Embodiment 139. The pharmaceutical composition of any one of Embodiments 69-99, wherein the polynucleotide comprises the nucleic acid sequence set forth in SEQ ID NO:8, SEQ ID NO:9, SEQ ID NO:10, SEQ ID NO:11, SEQ ID NO:12, SEQ ID NO:13, or SEQ ID NO:14.

Embodiment 140. The pharmaceutical composition of any one of Embodiments 69-99 and 139, wherein the polynucleotide comprises the nucleic acid sequence set forth in SEQ ID NO:11, SEQ ID NO:12, SEQ ID NO:13, or SEQ ID NO:14.

Embodiment 141. The pharmaceutical composition of any one of Embodiments 69-99, and 139, wherein the pharmaceutical composition comprises: a) the nucleic acid sequence set forth in SEQ ID NO:8, SEQ ID NO:9, SEQ ID NO:10, SEQ ID NO:11, SEQ ID NO:12, SEQ ID NO:13, or SEQ ID NO:14; and b) the following compound:

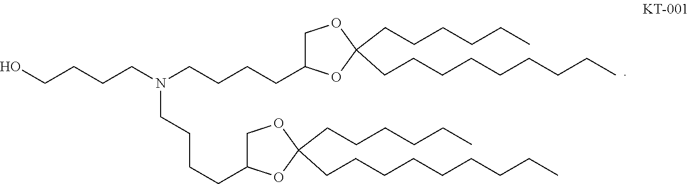 [SMILES: CCCCCCCCCC1(CCCCCC)OCC(CCCCN(CCCCO)CCCCC2COC(CCCCCC)(CCCCCCCCC)O2)O1]

Embodiment 142. The pharmaceutical composition of any one of Embodiments 69-99, 139, and 141, wherein the pharmaceutical composition comprises: a) the nucleic acid sequence set forth in SEQ ID NO:8, SEQ ID NO:9, SEQ ID NO:10, SEQ ID NO:11, SEQ ID NO:12, SEQ ID NO:13, or SEQ ID NO:14; b) the following compound:

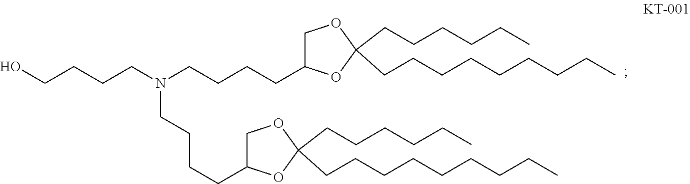 [SMILES: CCCCCCCCCC1(CCCCCC)OCC(CCCCN(CCCCO)CCCCC2COC(CCCCCC)(CCCCCCCCC)O2)O1]

and lipid nanoparticles.

Embodiment 143. The pharmaceutical composition of any one of Embodiments 69-99, and 139-142, wherein the pharmaceutical composition comprises: a) the nucleic acid sequence set forth in SEQ ID NO:11, SEQ ID NO:12, SEQ ID NO:13, or SEQ ID NO:14; b) the following compound:

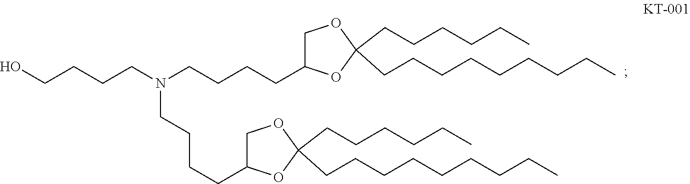 [SMILES: CCCCCCCCCC1(CCCCCC)OCC(CCCCN(CCCCO)CCCCC2COC(CCCCCC)(CCCCCCCCC)O2)O1]

and lipid nanoparticles.

Embodiment 144. The method of any one of Embodiments 101-124, wherein the polynucleotide or the pharmaceutical composition comprises the polynucleotide or the pharmaceutical composition of any one of Embodiments 127-143.

Embodiment 145. The use of Embodiment 125 or Embodiment 126, wherein the polynucleotide or the pharmaceutical composition comprises the polynucleotide or the pharmaceutical composition of any one of Embodiments 127-143.

### EXAMPLES

### Example 1: Preparation of mRNAs Encoding Modified IL-2 Polypeptide Fusion Proteins and LNP Pharmaceutical Compositions Containing them

### Materials and Methods

Generation of mRNAs Encoding HSA-4210 and MSA-4210, Respectively

Two mRNA species, one termed HSA-4210 and the other termed MSA-4210, each encode one of two fusion proteins comprising, in N-terminal to C-terminal order: a signal peptide; either human serum albumin (HSA) (e.g., the amino acid sequence set forth in SEQ ID NO:7) or murine serum albumin (MSA) (e.g., the amino acid sequence set forth in SEQ ID NO:6); three repeats of a G4S linker (e.g, the amino acid sequence set forth in SEQ ID NO:15); and human IL-2 ACT4210 (e.g., the amino acid sequence set forth in SEQ ID NO:3).

To generate each HSA-4210 and MSA-4210 template for RNA synthesis, the codon-optimized DNA for each was independently cloned into pVAX1-based backbone which features 5′-UTR, 3′-UTR and Poly-A tail. Each plasmid DNA was independently produced in bacteria, purified and linearized by a single-site restriction enzyme digestion. Each template DNA was independently purified, spectrophotometrically quantified, and in vitro transcribed by T7 RNA polymerase (Cat: M0251, NEB) in the presence of a trinucleotide cap1 analogue, m7(3′OMeG)(5′)ppp(5′)(2′OMeA)pG (Cat: N-7413, TriLink), and of N1-methylpseudouridine-5′-triphosphate (Cat: NU-890-THR-CSTM, Jena Bioscience) in place of uridine-5′-triphosphate (UTP). After the reaction, DNase I (Cat: M0303, NEB) was added to remove each template DNA, and each respective mRNA was then purified by LiCl precipitation (Cat: AM9480, ThermoFisher).

### Synthesis of Compound KT-001

Compound KT-001 was prepared as shown in Scheme 1 below:

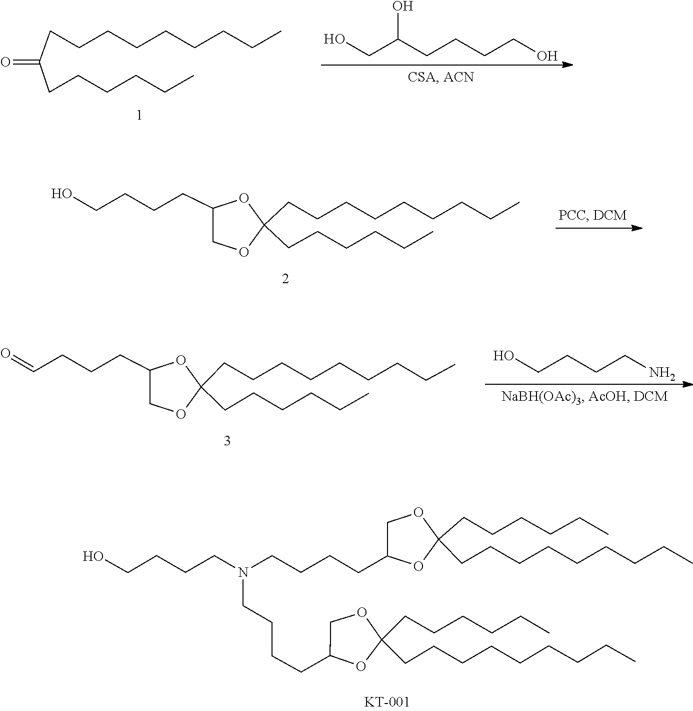 [SMILES: CCCCCCCCCC(=O)CCCCCC ; OCCCCC(O)CO ; CCCCCCCCCC1(CCCCCC)OCC(CCCCO)O1 ; CCCCCCCCCC1(CCCCCC)OCC(CCCC=O)O1 ; NCCCCO ; CCCCCCCCCC1(CCCCCC)OCC(CCCCN(CCCCO)CCCCC2COC(CCCCCC)(CCCCCCCCC)O2)O1]

Compound 2. To a mixture of compound 1 (1.00 g, 4.16 mmol) and 1,2,6-hexanetriol (1.12 g, 8.32 mmol) in anhydrous acetonitrile (20 mL) was added camphorsulfonic acid (CSA) (290 mg, 1.25 mmol). The resulting solution was refluxed for 16 hr until TLC indicated completion of reaction. Compound 2 (650 mg) was obtained by silica gel chromatography.

Compound 3. Pyridinium chlorochromate (PCC) (785 mg, 3.64 mmol) was added to a solution of compound 2 (650 mg, 1.82 mmol) in dichloromethane (30 mL). The reaction was stirred at room temperature for 2 hr until TLC indicated completion of reaction. Compound 3 (320 mg) was obtained by silica gel chromatography.

Compound KT-001. Acetic acid (4 μL, 0.068 mmol) was added to a mixture of compound 3 (300 mg, 0.846 mmol) and 4-amino-1-butanol (30 mg, 0.338 mmol) in dichloromethane (5 mL). The resulting mixture was stirred at r.t. for 15 min followed by addition of sodium triacetoxyborohydride (NaBH(OAc)₃) (215 mg. 1.01 mmol) and stirred at r.t. for another 4 h. Compound KT-001 (165 mg) was obtained by silica gel chromatography. ¹HNMR (500 MHz, CDCl₃): δ=0.85-0.91 (m, 12H), 1.20-1.44 (m, 48H), 1.46-1.72 (m, 20H), 2.31-2.61 (br, 6H), 3.40-3.47 (m, 2H), 3.52-3.60 (m, 2H), 3.99-4.07 (m, 4H).

Generation and Optimization of mRNA-Lipid Nanoparticle Formulations.

Lipid containing formulations, such as lipid nanoparticle formulations that may be employed to prepare lyophilized formulations comprising the nucleic acid sequences, polynucleotides, and pharmaceutical composition, as disclosed herein and throughout were generated and optimized. Such formulations that exhibit, inter alia, stability, nucleic acid molecule integrity, and in vivo efficacy after storage as a lyophilized powder at approximately 2-8 degrees Celsius for over two months. To generate such formulations, parameters such as buffer choice, use of cryoprotectants, ratio of lipids, N/P ration, and pH, were tested and optimized.

### Exemplar Lipid Nanoparticle Preparation Using Precision Ignite Nanoassemblr

### Preparation of TBT Buffer

To a 6 L beaker equipped with a stir bar (3″) was added Tris base (9.688 g, 0.08 mol), trehalose anhydrous (400.00 g), and water for injection (3.8 L). The mixture was allowed to stir at room temperature for 2 hours or until all materials dissolved in aqueous phase. The pH of the solution was adjusted with HCl (1N) to pH 7.3-7.4. The overall volume of the solution was quantum satis-ed to 4 L, sterile filtered using 0.22 uM filtration, kept at room temperature for further use.

### Exemplar Preparation of Lipid Solution

To a 5 mL Eppendorf tube was added JK0315CA (550.5 uL, 20 mg/mL in EtOH), DSPC (105.4 uL, 20 mg/mL in EtOH), Cholesterol (198.7 uL, 20 mg/mL in EtOH), and DMG-PEG2000 (62.8 uL, 20 mg/mL in EtOH) and EtOH (1796 uL) to make 2.713 mL lipid solution (lipid concentration: 10 mM; molar ratio of lipids: JK0315CA/DSPC/Cholesterol/DMG-PEG2000=49.9/10/38.4/0.17)

Exemplar Preparation of mRNA Solutions

mRNAs (600 uL, 1 mg/mL) were dissolved in NaOAc buffer (pH 5, 25 mM) to make an mRNA NaOAc solution (pH 5, 25 mM, mRNA 73.7 ug/mL)

### Exemplar LNP Formulation

mRNA solutions (8.14 mL, 86.4 ug/mL) were drawn in a 10 mL BD syringe, remove air bubble by gently tapping the syringe and loaded onto ignite nanoassembr cartridge. Lipid solution (10 mM) was drawn into a 3 mL BD syringe and air bubbles removed. The lipid solution was loaded onto cartridge. The flow rate was set at 12 mL/min and the ratio of the aqueous solution to EtOH solution as 3/1, 0.1 mL and 0.05 mL waste volume at the beginning and the ending stage. The formulated solution was collected into a dialysis bag (100 KD) for buffer exchange.

### Exemplar Dialysis Procedure

The dialysis bag with the formulation was dialyzed with TBT buffer (1 L) prepared at step 1. Buffer was changed every 6 hours for 3 times. The LNP solution after dialysis was collected into an eppendoff tube (15 mL). The size and zeta was measured on Zetasizer Ultra. The mRNA concentration and encapsulation rate was determined using a Ribogreen-based mRNA assay using plate reader.

### Addition of Cryoprotectant (HP-b-CD)

After determine the mRNA concentration, the solution was added HP-b-CD (40%). The volume of HP-b-CD is dependent on the mRNA/lipid concentration and the ratio of Lipid/HP-b-CD is 1/8.17

### Exemplar Lyophilization Procedure

The LNP solution (with HP-b-CD) was aliquoted into 2 mL serum vial (400 uL each vial) with igloo cap on. The solution was frozen in a −80° C. freezer for 4 hours. The frozen sample was then transferred into the shelf of a freeze dryer (the temperature of the shelf was set and precooled to −20° C.) The frozen sample was lyophilized at −20° C. for 24 hours under vacuum (0.05 mbar), then the shelf temperature was set to 25° C. (1 degree temp increase per min). The sample was then lyophilized at 25° C. for 6 hours, the cap was pushed on under vacuum, allow the freeze dryer chamber pressure to be increased to 1 atm, crimp the serum vial with aluminum cap, then store the capped product at 2-8° C. for long time storage.

### Exemplar Lipid Nanoparticle Preparation Using HPLC Pump

### Exemplar Preparation of TBT Buffer

Same as above for Precision ignite nanoassemblr

### Exemplar Preparation of Lipid Solution

To a 100 mL Nelgene bottle was added JK0315CA (11.560 mL, 20 mg/mL in EtOH), DSPC (2.213 mL, 20 mg/mL in EtOH), Cholesterol (4.172 mL, 20 mg/mL in EtOH), and DMG-PEG2000 (1.319 mL, 20 mg/mL in EtOH) and EtOH (37.716 mL) to make 56.979 mL lipid solution (lipid concentration: 10 mM; molar ratio of lipids: JK0315CA/DSPC/Cholesterol/DMG-PEG2000=49.9/10/38.4/0.17)

Exemplar Preparation of mRNA Solution

To a 200 mL Nelgene bottle was added mRNA (12.6 mL, 1 mg/mL) and NaOAc buffer (158.34 mL, pH 5, 25 mM) to make a mRNA NaOAc solution 170.94 mL (pH 5, 25 mM, mRNA 73.7 ug/mL).

### Preparation of Exemplar LNP Formulations

The lipid solution (organic) and mRNA solution (aqueous) was loaded onto HPLC pump. The pump was purged with lipid solution and mRNA solution until no bubble shows up. Adjust the percentage of organic to 25% and aqueous to 75% and the flow rate was set up to 12.5 mL/min (organic) and 37.5 mL/min (aqueous). The solution coming out of T-mixer was collected into 500 mL nelgene bottle equipped with a stir bar. After collecting 100 mL of the solution, the pump was stopped, and the bottle was put onto a stir plate. TBT buffer (300 mL) was slowly added into the nelgene bottle while stirring (150 rpm) during 5 min.

### Exemplar Tangential Flow Filtration (TFF) Procedure

The diluted LNP solution was transferred into Pall minimate EVO system equipped with Repligen hollow fiber filter (mPES, 100KD, 115 cm²). The flow rate of the TFF was set to 60 mL/min, and the permeate flow rate was set to less than 5 mL/min. The solution was concentrated to 20 mL, then diluted to 100 mL with TBT buffer. This process was repeated 5 times and the final mRNA concentration of the LNP was adjusted to 100 ug/mL using TBT buffer.

### Addition of Cryoprotectant (HP-b-CD)

Same as above for Precision ignite nanoassemblr

### Exemplar Lyophilization Procedure

Same as above for Precision ignite nanoassemblr

### Characterization of KT-001, TU-001, TU-002, BAE-001, and DS-001

Lipid nanoparticles were prepared with the following compositions:
ALC-0315:DSPC:Cholesterol:GM020=50:10:38.5:1.5 (and buffer) (positive control);
TU-001:DSPC:Cholesterol:GM020=50:10:38.5:1.5 (and buffer);
TU-002:DSPC:Cholesterol:GM020=50:10:38.5:1.5 (and buffer);
BAE-001:DSPC:Cholesterol:GM020=50:10:38.5:1.5 (and buffer);
DS-001:DSPC:Cholesterol:GM020=25:10:38.5:1.5 (and buffer); and
KT-001:DSPC:Cholesterol:GM020=50:10:38.5:1.5 (and buffer);
   where “buffer” is a solution of water, trehalose, Tris Base, and sodium chloride. DSPC is 1,2-distearoyl-sn-glycero-3-phosphocholine. GM020 is DMG-PEG (1,2-Dimyristoyl-rac-glycero-3-phosphocholine. GM020 is DMG-PEG (1,2-Dimyristoyl-rac-glycero-3-methylpolyoxyethylene).

Table 1 below summarizes the lipid nanoparticle characterization with respect to various parameters including nanoparticle size (Z avg), polydispersity index (PDI), charge (Zeta potential), encapsulation efficiency and yield.

TABLE 1

| | | Fresh | | | | | | |
|---|---|---|---|---|---|---|---|---|
| | Amount | | | | | Conc. | | |
| | of | Z | | Zeta | Encapsulation | of | Total | |
| | mRNA | Avg. | | Potential | Efficiency | mRNA | volume | Yield |
| Composition | (μg) | (nm) | PDI | (mV) | (%) | (μg/mL) | (mL) | (%) |
| ALC-0315 | 250 | 78.67 | 0.223 | −4.74 | 83.16 | 116.16 | 1.82 | 84.6 |
| (rsF352) | | | | | | | | |
| TU-001 | 250 | 73.81 | 0.208 | 0.0305 | 66.04 | 86.16 | 1.87 | 64.4 |
| (rsF348) | | | | | | | | |
| TU-002 | 250 | 76.99 | 0.133 | −2.55 | 80.64 | 118.38 | 1.26 | 59.7 |
| (rsF350) | | | | | | | | |
| BAE-001 | 250 | 111.7 | 0.141 | −0.28 | 83.32 | 107.06 | 2.04 | 87.4 |
| (rsF359) | | | | | | | | |
| DS-001 | 250 | 63.0 | 0.12 | −3.74 | 84.50 | 91.42 | 1.85 | 67.6 |
| (rsF373) | | | | | | | | |
| KT-001 | 250 | 84.7 | 0.08 | −3.30 | 80.60 | 98.20 | 1.90 | 74.6 |
| (rsF377) | | | | | | | | |

### Example 2: In Vitro Characterization of HSA-4210 and MSA-4210 Fusion Protein-Encoding mRNAs

### Materials and Methods

### ACT421H and ACT421M Expression In Vitro

HEK293T cells were cultured DMEM with 10% FBS with 80-90% confluent in a T-75 flask. Cells were passaged at one day before the transfection as indicated density and incubated at 37 C, 5% C02 incubator for 24 hours. On the day of transfection, the cells have more than 95% viability and be about 70% confluence, then transfected with either human serum albumin (HSA)-4210 or murine serum albumin (MSA)-4210 fusion protein-encoding mRNA-LNP pharmaceutical compositions (also referred to herein and throughout as ACT421H and ACT421M, respectively), each with a concentration of 1 to 5 μg/ml. After 4 hours of transfection, cells were added 10% FBS into medium, and incubated at 37 C 5% C02 incubator for 3 days. Then, the supernatant was harvested for ELISA analysis and characterization assay in vitro.

### ACT421H and ACT421M Expression In Vivo

ACT421H (LNP-mRNA-HSA-4210) or ACT421M (LNP-mRNA-MSA-4210) was injected into tail vein of C57 mouse each at a concentration of 30 μg per mouse. Then blood samples were collected via check vein post 6 hours injection.

### Sandwich ELISA Assay

For sandwich ELISA assays, reagents were as follows:
Capture reagent: anti-IL2 monoclonal antibody clone MQ1-17H12;
Detection reagent: biotinylated anti-IL2 polyclonal antibody+Avidin-HRP;
Signal generation: TMB with sulfuric acid to stop.

Procedure was performed as follows. High bind-treated well plates were coated with anti-IL2 monoclonal antibody clone MQ1-17H12. All standards and samples were diluted in same BSA buffer. Samples originating from mRNA encoding for ACT4210 fusion protein (MSA-4210 or HSA-4210) were plated alongside serially diluted purified ACT4210 standard. Anti-IL2 biotinylated polyclonal antibody and avidin-HRP were diluted in same BSA buffer and plated. TMB was used to show signal and sulfuric acid was used to stop the signal generation. Plate was read at 450 nm. Standard curve was generated using 4PL sigmoidal regression. Experimental samples were interpolated. Readings outside signal range of standard curve were removed. Remaining values were interpolated using standard curve, multiplied by dilution factor, and averaged. Reported concentration was not considered absolute due to difference in protein structure of samples and standard.

### ELISA Based Binding to Human, Ynomolgus and Mouse IL-2R

Anti-Human IgG1 Fc (abcam, ab1927) was immobilized on high binding 96 well plate (CORNING) by coating the pleat with 100 μL of antibody (5 ug/ml in coating buffer) per well and incubation at 4° C. overnight. Plates were washed three times with 300 μL per well of wash buffer in microplate washer. Non-specific binding sites were blocked with Elisa buffer, 200 μL per well for 30 mins at room temperature, then wash plate one time. FC tagged IL-2R was captured by adding 100 ul of Human IL-2RA, Fc Tag&Fc (1000 ng/ml), Human IL-2RB&IL-2RG Tag&Fc (1000 ng/ml) or Human IL-2RB&IL-2RA&IL-2RG Tag&Fc (1000 ng/ml) into anti-FC coated plat at room temperature for 60 mins, then wash 1× with 300 μL per well of Wash buffer in Microplate washer. ACT4210-his or IL-2-his was serially diluted in Elisa buffer, then add 100 ul of samples per well and incubate it for 2 hours at room temperature, then wash 1× with 300 μL per well of Wash buffe in Microplate washer. 100 μL of detection antibody solution (HRP-anti-His; 2 μg/ml) was added into each well. The plate was sealed and incubated for 1 hour at room temperature, then washed 3 times and soaked with 300 μL per well of Wash buffer. The plate was again washed 3 times with 300 ul in Microplate washer. 100 μL of TMB substrate solution was then added to each well and incubated at room temperature not more than 15 minutes. 100 μL of Stop solution was then added to each well when the color in the wells of standard turned to diamond blue. Absorbance was measured at 450 nm within 30 minutes of adding Stop solution.

### ELISA Based Binding to Rat IL-2R

Coating Solution (5 ug/ml) was prepared by diluting the Capture antibody (Anti-Human IL-2) in Coating buffer. Plates were coated with 100 μL per well of Coating Solution. The plate was sealed and incubated at 4° C. overnight, then washed 3 times with 300 μL per well of Wash buffer in Microplate washer. Non-specific binding sites were blocked with 200 μL per well with Blocking buffer. The plate was sealed and incubates for 30 mins at room temperature, then washed 1× with 300 μL per well of Wash buffer in Microplate washer. 100 ul of His-tagged IL-2R (1000 ng/ml) was added at room temperature for 60 mins. The plate was then washed 1× with 300 μL per well of Wash buffer in Microplate washer. Samples were prepared by using 1000 ng/ml as starting concentration, then by making 1:2 dilution. 100 ul of samples were added per well and the plate sealed and incubated for 2 hours at room temperature. The plate was then washed 1× with 300 μL per well of Wash buffer in Microplate washer. 100 μL of detection antibody solution (HRP-anti-His; 2 μg/ml) was then added to each well. The plate was then sealed and incubated for 1 hour at room temperature. The plate was then washed 3 times and soaked with 300 μL per well of Wash buffer, then washed 3 times with 300 ul again in Microplate washer. 100 μL of TMB substrate solution was then added to each well, and the plate incubated at room temperature for not more than 15 minutes. 100 μL of Stop solution was then added to each well when the color in the wells of standard turned to diamond blue. Absorbance was then measured at 450 nm within 30 minutes of adding Stop solution.

### HEK Blue IL-2R Reporter Assay

HEK-Blue IL-2R reporter cells (InvivoGen) were grown in DMEM with 15% Hi FBS (Sigma) and selection solution (InvivoGen). Cells were collected by rinsing cells twice with PBS and detaching the cells by tapping. Cells were resuspended at 5×10⁵ cells/ml of test medium (DMEM with 15% Hi FBS) and then seeded at 5×10⁴ cells per well in 96-well cell culture plates. Samples of supernatant were collected from 293T cells transfected with mRNA-encoded albumin (HSA or MSA)-4210 fusion proteins. The serial dilutions of samples were added alongside serially diluted purified ACT4210 standard, and incubated at 37° C., in a CO₂ incubator for 20-24 hours, as per manufacturer's protocol. Afterwards, 20 μL cell supernatant was collected and incubated with 180 μL Quanti-Blue reagent (InvivoGen) for 90 minutes. The mixture was read at 630 nm using the spectrophotometer (TECAN).

### HEK-Blue CD122/CD132 Reporter Assay

HEK-Blue CD122/CD132 reporter cells have been specifically designed to detect bioactive human (h) interleukin-2 (hIL-2) by monitoring the activation of the JAKSTAT pathway. HEK-Blue CD122/CD132 cells were generated by stable transfection of HEK293 cells with the human CD122 (IL-2Rβ) and CD132 (IL-2Rγ) genes, along with the human JAK3 and STAT5 genes to obtain a fully active IL-2 signaling pathway. In addition, a STAT5-inducible secreted embryonic alkaline phosphatase (SEAP) reporter gene was also introduced. Upon hIL-2 stimulation, HEK-Blue CD122/CD132 cells trigger the JAK/STAT5 activation and the subsequent secretion of SEAP, which can be readily monitored using QUANTI-Blue™ Solution, a SEAP detection medium.

HEK-Blue IL-2R CD122/CD123 reporter cells were grown in DMEM with 15% Hi FBS (Sigma) and selection solution (InvivoGen). Cells were collected by rinsing cells twice with PBS and detaching the cells by tapping. Resuspend cells at 5×10⁵ cells/ml of test medium (DMEM with 15% Hi FBS) and seeded cells (5×10⁴ cells per well) to 96-wells cell culture plate. Serum samples were obtained from ACT421H treated mice. The serial dilutions of samples were added alongside serially diluted purified ACT4210 standard, and incubated at 37° C., a C02 incubator for 22 hours as per manufacturer's protocol. Afterwards, 20 μL cell supernatant was collected and incubated with 180 μL Quanti-Blue reagent (InvivoGen) for 60 minutes. The mixture was read at 630 nm using the spectrophotometer.

### CTLL-2 Proliferation Assay

CTLL-2 cell line is a clone of cytotoxic T cells derived from a C57BL/6 mouse (ATCC). CTLL-2 cells were cultured in RPMI 1640 supplemented with 10% fetal bovine serum (FBS), and 10% T-Stim with ConA, rIL-2 10 ng/ml. The cells were harvested in their logarithmic phase (Cell passage 5 after thawing; Cell viability: ≥95%) and washed three times with the medium (by centrifugation at 1000 rpm, 5 min) and incubated for 4 h in assay medium (RPMI 1640 supplement with 10% FBS without Con A and IL-2) at 37° C., 5% CO2. During this period, a 96-well tissue culture plate was set up. The IL-2 samples were diluted to an initial concentration of 1000 ng/ml in the assay medium and followed by serial two-fold or three-fold dilutions and added to the wells in 100 μl of the assay medium in triplicates. After the 4 hours incubation, the prepared cell suspension was transferred to a sterile reservoir and seeded immediately in the wells of the 96-well plate [final cell density: (2×104) cells/well](containing 100 μl of test cytokines at different concentrations) in 100 μl of the assay medium added to cells and incubated at 37° C., 5% C02 for 48 h. After the 48 hours incubation period, CCK-8 reagent was added in 10% concentration (20 μl/well) and incubated for another 2-4 h at 37° C. and 5% C02, the plate was then read at 450 nm.

### Data Analysis

For data analysis, GraphPad Prism software was used. For quantification, a standard curve was generated using 4PL sigmoidal regression. Experimental samples were interpolated. Also, the non-linear fit curves were generated using the agonist concentration vs. response model and EC50s are calculate.

### Results

### ELISA Assay of Post Transfection Supernatants

High bind treated well plates are coated with anti-IL2 monoclonal antibody. All standards and samples are diluted in same BSA buffer. Samples (supernatant of mRNA encoded IL-2 variants albumin fusion transfected 293 cells) were plated alongside serially diluted purified ACT4210 standard. Anti-IL2 biotinylated antibody was used as detection. The quantification of HSA-4210 or MSA4210 was measured with absorbance-based microplate reader at OD 450 nm. The result, illustrated in FIG. 1, showed that mRNA encoded IL-2 and IL-2 albumin fusions have good expression yield in 293T cells in vitro.

HEK-Blue IL-2 Reporter Assay of mRNA Encoded 4210 Albumin Fusions

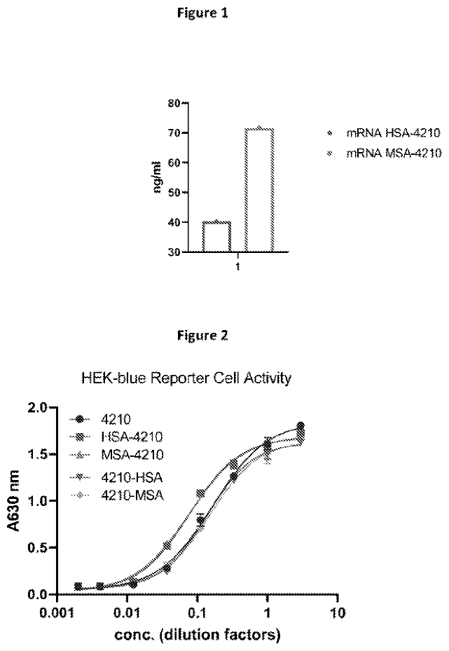

HEK-Blue IL-2R reporter cells (InvivoGen) were grown in DMEM with 15% Hi FBS (Sigma) and selection solution (InvivoGen). Cells were collected by rinsing cells twice with PBS and detaching the cells by tapping. Cells were resuspended at 5×10⁵ cells/ml of test medium (DMEM with 15% Hi FBS) and seeded (5×10⁴ cells per well) to 96-wells cell culture plate. Serial-diluted post transfection supernatants of mRNA encoded 4210 albumin fusion protein variants were incubated at 37° C., a CO2 incubator for 20-24 hours as per manufacturer's protocol. Afterwards, 20 μL cell supernatant was collected and incubated with 180 μL Quanti-Blue reagent (InvivoGen) for 90 minutes. The mixture was read at 630 nm using the spectrophotometer (TECAN). As illustrated in FIG. 2, mRNA-encoded 4210 albumin fusion proteins have good bioactivity in a dose dependence manner in HEK-BLUE IL-2 reporter cells.

Interaction of mRNA-Encoded 4210 Albumin Fusion Proteins with IL-2Rα and IL-2Rβγ

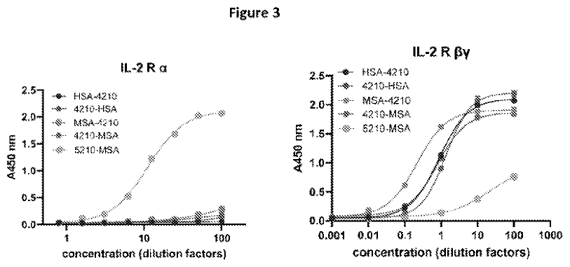

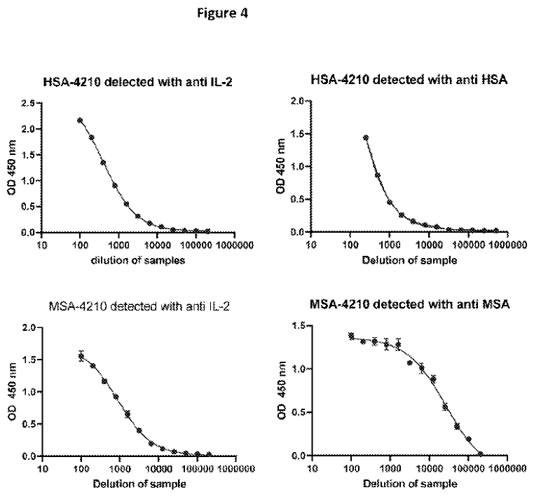

Human IL-2Rα and IL-2Rβγ in human Fc fusion protein format were immobilized by anti-Human IgG Fc, which were captured to a microplate. Then, the serial dilutions of mRNA-encoded 4210 albumin fusion protein variants were added to wells with IL-2Rα or IL-2Rβγ and IL-2Rαβγ. The binding was detected by anti-HSA or MSA antibody conjugated with HRP and its substrate. The quantification of binding was measured with absorbance-based microplate reader. The results, illustrated in FIG. 3, showed that both mRNA encoding either HSA and MSA-fused 4210 protein fusions expressed fusions proteins that displayed essentially no appreciable interaction with IL-2Rα (left panel). Conversely, these fusions proteins displayed marked ability to interact with IL-2Rβγ (right panel).

In Vivo Expression In Vivo of HSA-4210 and MSA-4210 from Pharmaceutical Compositions Comprising mRNA Encoding the Fusions Proteins

ACT421H (LNP-mRNA-HSA-4210) or ACT421M (LNP-mRNA-MSA-4210) pharmaceutical compositions were each independently injected into tail veins of C57 mice at 30 μg per mouse. Then blood samples were collected via check vein post 24 hours drug administration and were centrifuged at 10000 rpm for 10 minutes to get serum for analysis. High bind treated plates were coated with anti-IL2 monoclonal antibody, then the serially diluted serum samples were added to plate. Anti-IL2 and anti HSA or MSA biotinylated antibodies were used as detection. The quantification of HSA-4210 or MSA-4210 fusion protein was measured with absorbance-based microplate reader at OD 450 nm. As illustrated in FIG. 4, both HSA-4210 and MSA-4210 fusion proteins were detected by an anti-IL-2 antibody and either an anti-HSA antibody or an anti-MSA antibody, respectively, demonstrating in vivo expression of the mRNA components of the tested pharmaceutical compositions.

In Vivo-Expressed mRNA Encoding HSA-4210 or MSA-4210 Incorporated in ACT421H or ACT421M Pharmaceutical Compositions Each Bind to Human IL-2Rα not IL-2Rβγ

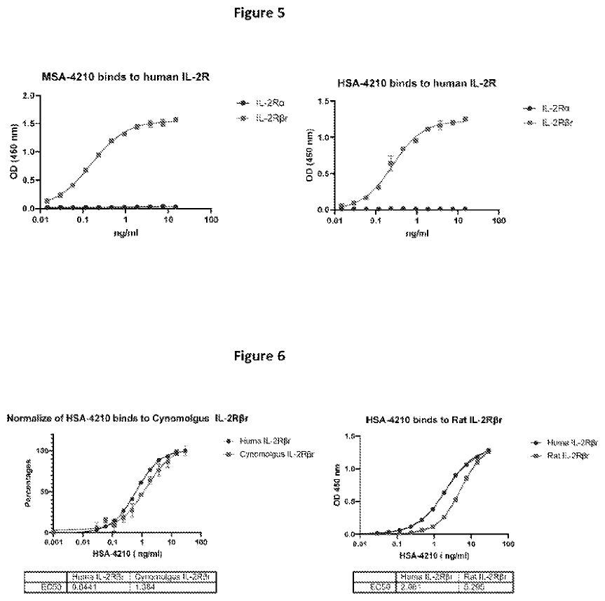

ACT421H (LNP-mRNA-HAS-4210) or ACT421M (LNP-mRNA-MSA-4210) was injected into tail vein of C57 mouse, 30 μg per mouse. Then blood samples were collected via check vein post 24 hours drug administration and were centrifuged at 10000 rpm 10 minutes to get serum for analysis. IL-2Rα, IL-2Rβγ in human Fc fusion protein format were immobilized by anti-Human IgG Fc, which were captured to a microplate. Then the serial diluted serums were added to wells with IL-2Rα or IL-2Rβγ. The binding was detected by anti-HSA or Anti-MSA antibody conjugated with HRP and its substrate. The quantification of binding was measured with absorbance-based microplate reader. The results, illustrated in FIG. 5, demonstrate that both HSA-4210 and MSA-4210 expressed in vivo from mRNA encoding the fusion proteins and incorporated into pharmaceutical compositions ACT421H or ACT421M, respectively, bind to human IL-2Rβγ, but not appreciably to IL-2Rα.

HSA-4210 Binds to IL-2R βγ from Different Species

ACT421H (LNP-mRNA-HAS-4210) was injected into tail vein of C57 mouse, 15 μg per mouse. Then blood samples were collected via check vein post 24 hours drug administration and were centrifuged at 10000 rpm 10 minutes to get serum for analysis. The 96 wells plate was coated with capture antibody (Human IL-2, 5 μg/ml) in Coating buffer. Human, Cynomolgus or ray IL-2Rβr were immobilized to human IL-2 coated microplate. Then the serial diluted serums of ACT421H treated mice were added to wells. The binding was detected by anti-HSA or Anti-MSA antibody conjugated with HRP and its substrate. The quantification of binding was measured with absorbance-based microplate reader. The results, illustrated in FIG. 6, demonstrate that HSA-4210 binds to IL-2Rβγ of rat and Cynomolgus.

### MSA-4210 Binds to IL-2Rβγ in Different Species

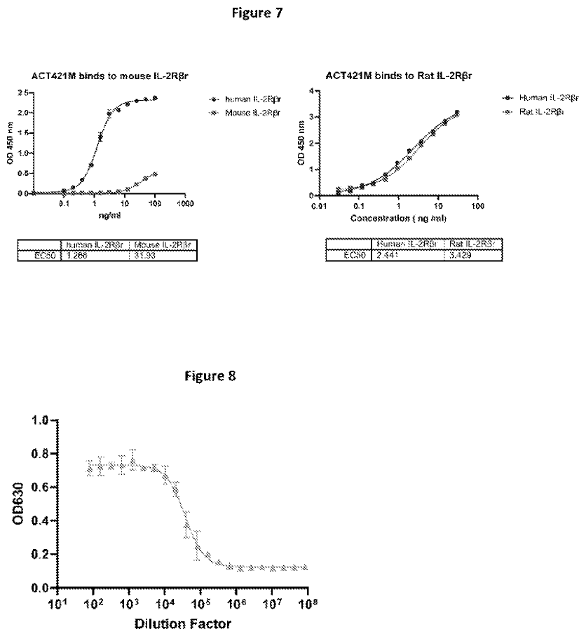

ACT421M (LNP-mRNA-MSA-4210) was injected into tail vein of C57 mouse, 30 μg per mouse. Then blood samples were collected via check vein post 24 hours drug administration and were centrifuged at 10000 rpm 10 minutes to get serum for analysis. Elisa based binding assays, illustrated in FIG. 7, demonstrate that MSA-4210 has the similar ability in binding to IL-2Rβγ of human, Cynomolgus and rat. However, MSA-4210 has lower affinity to mouse IL-2Rβγ compared to human IL-2Rβγ.

mRNA Encoded 4210 Albumin Fusions Showed Bioactivity in HEK Blue IL-2R CD122/CD132 Reporter Cells

HEK-Blue IL-2R CD122/CD123 reporter cells were grown in DMEM with 15% Hi FBS (Sigma) and selection solution (InvivoGen). Cells were collected by using 0.25% EDTA trypsin to detach the cells. Resuspend cells at 5×10³ cells/ml of test medium (DMEM with 15% Hi FBS) and seeded cells (5×10⁴ cells per well) to 96-wells cell culture plate. The serial dilutions of serum from ACT421H-treated mice were added and incubated at 37° C., a CO₂ incubator for 22 hours as per manufacturer's protocol. Afterwards, 20 μL cell supernatant was collected and incubated with 180 μL Quanti-Blue reagent (InvivoGen) for 60 minutes. The mixture was read at 630 nm using the spectrophotometer. As illustrated in FIG. 8, mRNA-encoded 4210 albumin fusions demonstrate good bioactivity in a dose-dependent manner in HEK Blue IL-2R CD122/CD132 reporter cells.

CTLL-2 Proliferation when Treated with HSA-4210

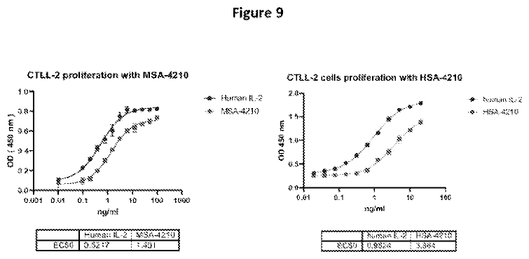

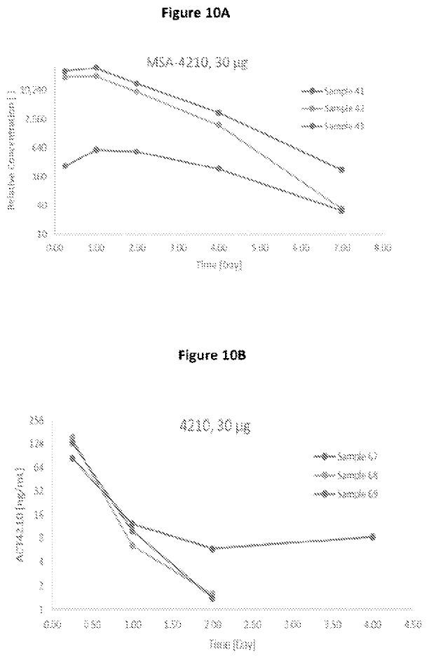

CTLL-2 cells were cultured in RPMI 1640 supplemented with 10% fetal bovine serum (FBS), and 10% T-Stim with ConA, rIL-2 10 ng/ml. The cells were harvested in their logarithmic phase (Cell passage 5 after thawing; cell viability: ≥95%) and washed three times with the and incubated for 5 h in assay medium (RPMI 1640 supplement with 10%) at 37° C., 5% CO₂. During this period, a 96-well tissue culture plate was set up. The samples were diluted to an initial concentration of 100 ng/ml in the assay medium and followed by serial two- or three-fold dilutions. After the 4 hours incubation, 100 ul of prepared cell suspension was seeded in the wells of the 96-well plate [final cell density: (5×10⁴) cells/well](containing 100 μl of test cytokines at different concentrations) and incubated at 37° C., 5% CO₂ for 48 h. After the 48 hours incubation period with either human IL-2, HSA-4210, or MSA-4210, CCK-8 reagent was added in 10% concentration (20 μl/well) and incubated for another 2 h at 37° C. and 5% CO₂, The plate was then read at 450 nm using the spectrophotometer. The results, illustrated in FIG. 9, demonstrate that both MSA-4210 and HSA-4210 can indice proliferation of CTLL-2 cells in a dose-dependent manner.

### Example 3: Pharmacokinetic and Pharmacodynamic Study of HSA-4210 and MSA-4210 Fusion Protein-Encoding mRNAs in C57BL Mice

### Materials and Methods

All mRNA-encoding protein, including fusion proteins, and pharmaceutical compositions comprising them, and methods preparing and administering them, were as described in Example 2 above. Additional Materials and methods employed in experiments described in this Example 3 were as follows.

### Pharmacokinetic and Pharmacodynamic Study in C57BL/6 Mice

All in vivo protocols and housing in mice were approved by the Institutional Animal Care and Use Committee (IACUC) prior to conduct. Female C57BL/6 mice received a single IV dose of 0, 1, 3, 10, 30 μg/per mouse of MSA-4210 encoding mRNA, 4210-MSA mRNA or 4210 mRNA via the tail vein (n-3/group) to evaluate pharmacokinetics, pharmacodynamics, PK/PD relationship over a broad dose range. Blood samples were collected via mice cheek bleeds performed on each mouse in each group at following time points: 6 hours, 1 d, 2d, 4d and 7 d, and processed for serum for PK analysis and whole blood for PD analysis.

### Results

### ELISA Conducted for PK Study

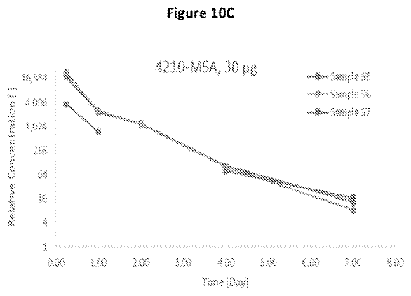

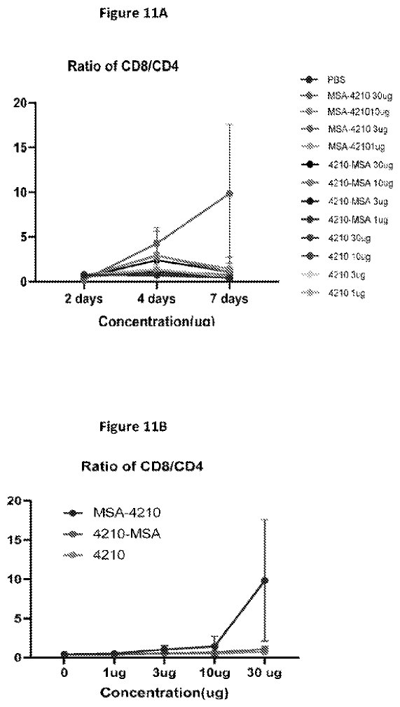

ELISA plates (Corning, Cat:3690) were coated with 50 ul coating buffer (Biolegend, Cat 421701) and 2 μg/ml anti-IL-2 antibody (Biolegend, Cat: 500302) overnight. Blood samples were diluted for 1:1 to 1:1000 times depending on estimated concentration of the samples. Diluted samples were added to the coated ELISA plate, which were detected by mixture of biotin conjugated anti-IL-2 antibody 2 μg/ml (ThemoFisher, Cat 13-7028-85) and Avidin-HRP (1:2500). Three mice were used for each time point and all the tests were done in duplicates. ELISA readings were calculated by standard curve of IL-2 mutein. The results, illustrated in FIGS. 10A-10C, demonstrate that both HSA-4210 and MSA-4210 concentration maxima were approximately 4-80-fold greater than that observed with 4210 alone (4210=modified IL-2 mutein not expressed as a fusion protein).

### Pharmacodynamics Study for MSA-4210, 4210-MSA and 4210 in C57BL/6 Mice

After collection of blood samples, the red blood cells were eliminated by RBC lysis buffer (biolegend, Cat 420301) and the rest of cells were seeded in V-shape 96 wells plate, 5×10⁵ cells/per well. Cells were stained with 50 ul of 1% BSA PBS, Sul of true stain (Biolegend, Cat 101319) and 1 ul of different anti-surface fluorophores antibody, such as PE conjugated anti-CD3 (Biolegend, cat 100206), BV conjugated CD8 (biolegend, Cat100750), BV421 conjugated CD4 (biolegend, Cat 100443) for 30 minutes at 4° C. in the dark. After centrifugation, the cells were fixed with fixation buffer (Fisher scientific, Cat 00-5523-00) for 30 minutes and permeabilized with permeabilization buffer. Cells were then stained with foxp3 staining buffer and 1 ul of Eflour 660 conjugated anti-mouse Foxp3 antibody (Invitrogen, Cat 50-5773-82) for 30 minutes at 4° C. 200 ul of 1% BSA PBS buffer were added to each well of the plate. Cells were spun and resuspended with 200 ul of 1% BSA PBS. CD4, CD8 and Foxp3 expressing cells were assessed by flow cytometry (Novocyte). Three mice were used for each time point and each group.

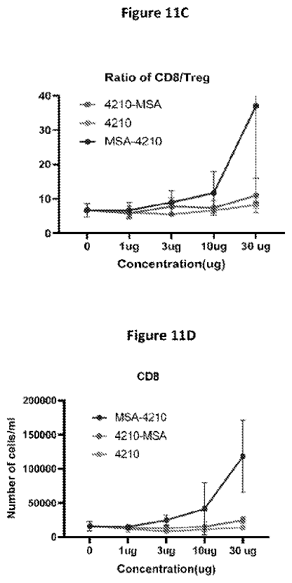

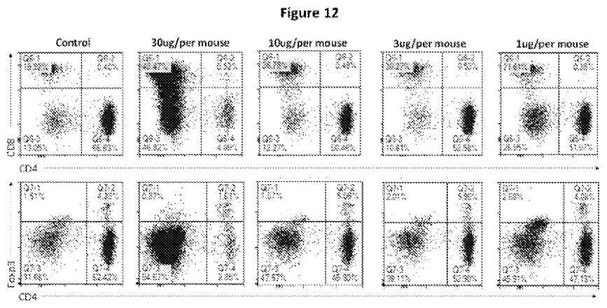

As illustrated in FIG. 11A, a single dose of MSA-4210, 4210-MSA or 4210 administrations via tail vein significantly increased ratio of CD8/CD4 in T cells of mouse blood cells on day 4 or day 7 with its peak on day 4 except group of MSA-4210 30 ug, which continuously increased the ratio of CD8/CD4 to 10 on day 7. In contrast, ratio of CD8/CD4 on control group remained the same at about 0.6.

Next, the T cell profile among these groups on day 7 were compared. Each group displayed an increased ratio of CD8 cells/CD4 cells in dose-dependent manner. Notably, MSA-4210 is strongest one in promoting ratio of CD8/CD4 (See FIG. 11B). Similar results were also found in ratio of CD8 cells/Treg cells (FIG. 11C) and number of CD8 cells in peripheral blood cells (FIG. 11D) indicating that.

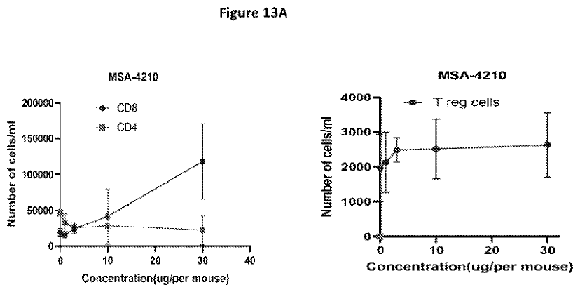

Next, we compared percentage of CD8 cells to that of CD4 cells or Treg cells on day 7 after single dose of MSA-4210 administration. MSA-4210 showed increased percentage of CD8 T cells and reduced percentage of CD4 T cells in dose-dependent manner while it showed no significant effect on percentage of T reg cells (FIG. 12). Similar results were also found on cell number of sub-type of T cells. MSA-4210 increased number of CD8 T cells but decreased the number of CD4 cells while showing no effect on T reg cells. (FIG. 13). Collectively, these results suggested that MSA-4210, 4210-MSA, HSA-4210, and 4210-HSA fusion proteins, each expressed in vivo from mRNA encoding these fusion proteins, are strong activators of CD8 T cells.

### Efficacy Study of MSA-4210 on B16F10 Metastasis Mouse Model

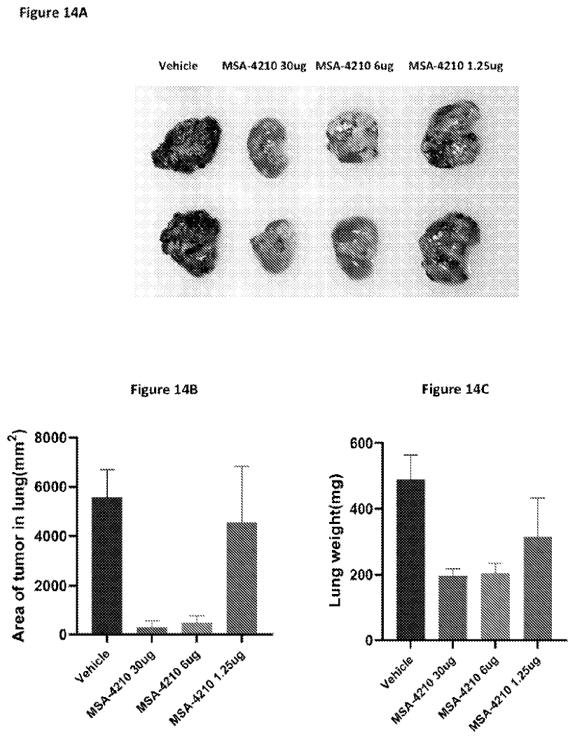

MSA-4210 was tested in this B16F10 mouse lung metastasis model. Tumor cells were routinely cultured in DMEM containing 10% FCS at 37° C. and 5% CD2 in a water saturated tissue culture incubator. 5×10³ cells in 200 ul of DMEM without serum was injected into mice by tail vein. One day after tumor injection, mice were administrated with MSA-4210 30 ug/per mouse for single dose and MSA-4210 6 ug/per mouse or 1.25 ug/per mouse (QW) for two weeks. 18 days after tumor injection, mice were euthanized, and mouse lungs were removed. Efficacy of metastasis was evaluated by measuring the area of lung foci and calculating mouse lung weight. Bright field mages representing metastasis, illustrated in FIG. 14A, demonstrate massive lung metastases observed in mice treated with vehicle. However, treatment with MSA-4210 markedly inhibited lung metastasis in a dose-dependent manner. Lung foci area (See FIG. 14B) and lung weight measurement data (See FIG. 14C) were consistent with the lung metastasis image data.

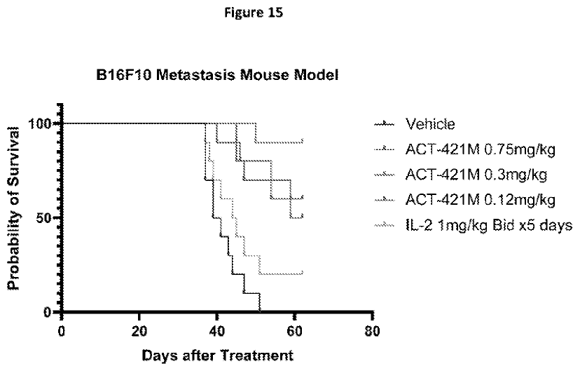

Similar effects were also observed in a B16F10 mouse lung metastasis survival model. Overall survival was significantly extended after treatment with one dose of MSA-4210, which was much longer than that of mice treated with EL-2 (Miltenyli Biotech, Cat 130-097-748). (see FIG. 15). Collectively, these results demonstrate that MSA-4210 effectively inhibited metastasis in this metastasis model.

Informal Sequence Listing

| SEQ | | |
|---|---|---|
| ID NO | Sequence | Description |
| 1 | APTSSSTKKTQLQLEHLLLDLQMILNGIN | Amino acid sequence of |
| | NYKNPKLTRMLTFKFYMPKKATELKHL | human rhIL-2 (contains |
| | QCLEEELKPLEEVLNLAQSKNFHLRPRD | C125S mutation) |
| | LISNINVIVLELKGSETTFMCEYADETATI | |
| | VEFLNRWITFSQSIISTLT | |
| 2 | MPTSSSTKKTQLQLEHLLLDLQMILNGI | Amino acid sequence of |
| | NNYKNPKLTRMLTFKFYMPKKATELKH | wild-type human IL-2 |
| | LQCLEEELKPLEEVLNLAQSKNFHLRPR | |
| | DLISNINVIVLELKGSETTFMCEYADETA | |
| | TIVEFLNRWITFCQSIISTLT | |
| 3 | APTSSSTKKTQLQLEHLLLDLQMILNGIN | Amino acid sequence of |
| | NCKNPKLTRMLTKKFYMPKKATELKHL | ACT4210, a modified |
| | QCLEEELKPLEEVLNLAQSKNFHLRPRD | IL-2 polypeptide |
| | LISNINVIVLELKGSETTFMCEYADETATI | |
| | VEFLNRWITFSQSIISTLT | |
| 4 | RGVFRREAHKSEIAHRYNDLGEQHFKGL | Amino acid sequence of |
| | VLIAFSQYLQKCSYDEHAKLVQEVTDFA | MSA-ACT4210, also |
| | KTCVADESAANCDKSLHTLFGDKLCAIP | known as MSA-4210 |
| | NLRENYGELADCCTKQEPERNECFLQH | (includes G4S linker) |
| | KDDNPSLPPFERPEAEAMCTSFKENPTTF | |
| | MGHYLHEVARRHPYFYAPELLYYAEQY | |
| | NEILTQCCAEADKESCLTPKLDGVKEKA | |
| | LVSSVRQRMKCSSMQKFGERAFKAWA | |
| | VARLSQTFPNADFAEITKLATDLTKVNK | |
| | ECCHGDLLECADDRAELAKYMCENQAT | |
| | ISSKLQTCCDKPLLKKAHCLSEVEHDTM | |
| | PADLPAIAADFVEDQEVCKNYAEAKDV | |
| | FLGTFLYEYSRRHPDYSVSLLLRLAKKY | |
| | EATLEKCCAEANPPACYGTVLAEFQPLV | |
| | EEPKNLVKTNCDLYEKLGEYGFQNAILV | |
| | RYTQKAPQVSTPTLVEAARNLGRVGTK | |
| | CCTLPEDQRLPCVEDYLSAILNRVCLLH | |
| | EKTPVSEHVTKCCSGSLVERRPCFSALT | |
| | VDETYVPKEFKAETFTFHSDICTLPEKEK | |
| | QIKKQTALAELVKHKPKATAEQLKTVM | |
| | DDFAQFLDTCCKAADKDTCFSTEGPNLV | |
| | TRCKDALAGGGGSGGGGSGGGGSAPTS | |
| | SSTKKTQLQLEHLLLDLQMILNGINNCK | |
| | NPKLTRMLTKKFYMPKKATELKHLQCL | |
| | EEELKPLEEVLNLAQSKNFHLRPRDLISN | |
| | INVIVLELKGSETTFMCEYADETATIVEF | |
| | LNRWITFSQSIISTLT | |
| 5 | DAHKSEVAHRFKDLGEENFKALVLIAFA | Amino acid sequence of |
| | QYLQQCPFEDHVKLVNEVTEFAKTCVA | HSA-ACT4210, also |
| | DESAENCDKSLHTLFGDKLCTVATLRET | known as HSA-4210 |
| | YGEMADCCAKQEPERNECFLQHKDDNP | (includes G4S linker) |
| | NLPRLVRPEVDVMCTAFHDNEETFLKK | |
| | YLYEIARRHPYFYAPELLFFAKRYKAAF | |
| | TECCQAADKAACLLPKLDELRDEGKAS | |
| | SAKQRLKCASLQKFGERAFKAWAVARL | |
| | SQRFPKAEFAEVSKLVTDLTKVHTECCH | |
| | GDLLECADDRADLAKYICENQDSISSKL | |
| | KECCEKPLLEKSHCIAEVENDEMPADLP | |
| | SLAADFVESKDVCKNYAEAKDVFLGMF | |
| | LYEYARRHPDYSVVLLLRLAKTYETTLE | |
| | KCCAAADPHECYAKVFDEFKPLVEEPQ | |
| | NLIKQNCELFEQLGEYKFQNALLVRYTK | |
| | KVPQVSTPTLVEVSRNLGKVGSKCCKHP | |
| | EAKRMPCAEDYLSVVLNQLCVLHEKTP | |
| | VSDRVTKCCTESLVNRRPCFSALEVDET | |
| | YVPKEFNAETFTFHADICTLSEKERQIKK | |
| | QTALVELVKHKPKATKEQLKAVMDDFA | |
| | AFVEKCCKADDKETCFAEEGKKLVAAS | |
| | QAALGLGGGGSGGGGSGGGGSAPTSSS | |
| | TKKTQLQLEHLLLDLQMILNGINNCKNP | |
| | KLTRMLTKKFYMPKKATELKHLQCLEE | |
| | ELKPLEEVLNLAQSKNFHLRPRDLISNIN | |
| | VIVLELKGSETTFMCEYADETATIVEFLN | |
| | RWITFSQSIISTLT | |
| 6 | RGVFRREAHKSEIAHRYNDLGEQHFKGL | Amino acid sequence of |
| | VLIAFSQYLQKCSYDEHAKLVQEVTDFA | murine serum albumin |
| | KTCVADESAANCDKSLHTLFGDKLCAIP | (MSA) |
| | NLRENYGELADCCTKQEPERNECFLQH | |
| | KDDNPSLPPFERPEAEAMCTSFKENPTTF | |
| | MGHYLHEVARRHPYFYAPELLYYAEQY | |
| | NEILTQCCAEADKESCLTPKLDGVKEKA | |
| | LVSSVRQRMKCSSMQKFGERAFKAWA | |
| | VARLSQTFPNADFAEITKLATDLTKVNK | |
| | ECCHGDLLECADDRAELAKYMCENQAT | |
| | ISSKLQTCCDKPLLKKAHCLSEVEHDTM | |
| | PADLPAIAADFVEDQEVCKNYAEAKDV | |
| | FLGTFLYEYSRRHPDYSVSLLLRLAKKY | |
| | EATLEKCCAEANPPACYGTVLAEFQPLV | |
| | EEPKNLVKTNCDLYEKLGEYGFQNAILV | |
| | RYTQKAPQVSTPTLVEAARNLGRVGTK | |
| | CCTLPEDQRLPCVEDYLSAILNRVCLLH | |
| | EKTPVSEHVTKCCSGSLVERRPCFSALT | |
| | VDETYVPKEFKAETFTFHSDICTLPEKEK | |
| | QIKKQTALAELVKHKPKATAEQLKTVM | |
| | DDFAQFLDTCCKAADKDTCFSTEGPNLV | |
| | TRCKDALA | |
| 7 | DAHKSEVAHRFKDLGEENFKALVLIAFA | Amino acid sequence of |
| | QYLQQCPFEDHVKLVNEVTEFAKTCVA | human serum albumin |
| | DESAENCDKSLHTLFGDKLCTVATLRET | (HSA) |
| | YGEMADCCAKQEPERNECFLQHKDDNP | |
| | NLPRLVRPEVDVMCTAFHDNEETFLKK | |
| | YLYEIARRHPYFYAPELLFFAKRYKAAF | |
| | TECCQAADKAACLLPKLDELRDEGKAS | |
| | SAKQRLKCASLQKFGERAFKAWAVARL | |
| | SQRFPKAEFAEVSKLVTDLTKVHTECCH | |
| | GDLLECADDRADLAKYICENQDSISSKL | |
| | KECCEKPLLEKSHCIAEVENDEMPADLP | |
| | SLAADFVESKDVCKNYAEAKDVFLGMF | |
| | LYEYARRHPDYSVVLLLRLAKTYETTLE | |
| | KCCAAADPHECYAKVFDEFKPLVEEPQ | |
| | NLIKQNCELFEQLGEYKFQNALLVRYTK | |
| | KVPQVSTPTLVEVSRNLGKVGSKCCKHP | |
| | EAKRMPCAEDYLSVVLNQLCVLHEKTP | |
| | VSDRVTKCCTESLVNRRPCFSALEVDET | |
| | YVPKEFNAETFTFHADICTLSEKERQIKK | |
| | QTALVELVKHKPKATKEQLKAVMDDFA | |
| | AFVEKCCKADDKETCFAEEGKKLVAAS | |
| | QAALGL | |
| 8 | GCCCCCACAAGCAGCAGCACCAAGAA | DNA sequence of |
| | GACACAGCTGCAGCTGGAGCACCTGCT | ACT4210, a modified |
| | GCTGGACCTGCAGATGATCCTGAACGG | IL-2 polypeptide |
| | CATCAACAACTGCAAGAACCCCAAGC | |
| | TGACAAGAATGCTGACCAAGAAGTTCT | |
| | ACATGCCCAAGAAGGCCACCGAGCTG | |
| | AAGCACCTGCAGTGCCTGGAGGAGGA | |
| | GCTGAAGCCCCTGGAAGAGGTGCTGA | |
| | ACCTGGCTCAGAGCAAGAACTTCCACC | |
| | TGAGACCTAGAGACCTGATCAGCAAC | |
| | ATCAACGTGATCGTGCTGGAGCTGAAG | |
| | GGCAGCGAGACCACCTTTATGTGCGAG | |
| | TACGCCGACGAGACCGCCACCATCGTG | |
| | GAGTTCCTGAACAGATGGATCACCTTT | |
| | TCTCAGAGCATCATTAGCACCCTCACC | |
| 9 | AGAGGCGTGTTCAGAAGAGAGGCCCA | DNA sequence of MSA- |
| | CAAGAGCGAGATCGCCCACAGATACA | ACT4210, also known as |
| | ACGACCTGGGCGAGCAGCACTTCAAG | MSA-4210 (includes |
| | GGCCTGGTGCTGATCGCCTTCTCTCAG | G4S linker) |
| | TACCTGCAGAAGTGCAGCTACGACGA | |
| | GCACGCCAAGCTGGTGCAAGAGGTGA | |
| | CCGACTTCGCCAAGACCTGCGTGGCCG | |
| | ACGAGAGCGCCGCCAACTGCGACAAG | |
| | AGCCTGCACACCCTGTTCGGCGACAAG | |
| | CTGTGCGCCATCCCCAACCTGAGAGAG | |
| | AACTACGGCGAGCTGGCCGACTGCTGC | |
| | ACCAAGCAAGAGCCCGAGAGAAACGA | |
| | GTGCTTCCTGCAGCACAAGGACGACA | |
| | ACCCTAGCCTGCCCCCCTTCGAGAGAC | |
| | CCGAGGCCGAGGCCATGTGCACAAGC | |
| | TTCAAGGAGAACCCCACCACCTTTATG | |
| | GGACACTACCTGCACGAGGTGGCCCGT | |
| | AGACATCCCTATTTTTACGCCCCCGAG | |
| | CTGCTGTACTACGCCGAGCAGTACAAC | |
| | GAGATCCTGACCCAATGCTGTGCCGAA | |
| | GCTGACAAGGAGAGCTGCCTGACCCC | |
| | CAAGCTGGACGGCGTGAAGGAGAAGG | |
| | CCCTGGTGAGCAGCGTGAGACAGAGA | |
| | ATGAAGTGCAGCAGCATGCAGAAGTT | |
| | CGGCGAGAGAGCCTTCAAGGCCTGGG | |
| | CCGTGGCTAGACTGTCTCAGACCTTCC | |
| | CCAACGCCGACTTCGCCGAGATCACCA | |
| | AGCTGGCCACCGACCTGACCAAGGTG | |
| | AACAAGGAGTGCTGCCACGGCGACCT | |
| | GCTGGAGTGCGCCGACGACAGAGCCG | |
| | AGCTGGCCAAGTACATGTGCGAGAAC | |
| | CAAGCCACCATCAGCAGCAAGCTGCA | |
| | GACCTGCTGCGACAAGCCCCTGCTGAA | |
| | GAAGGCCCACTGCCTGTCGGAGGTGG | |
| | AACACGACACCATGCCCGCAGATCTGC | |
| | CCGCCATCGCCGCCGACTTCGTGGAGG | |
| | ACCAAGAGGTGTGCAAGAACTACGCC | |
| | GAAGCCAAGGATGTGTTCCTGGGCACC | |
| | TTCCTGTACGAGTACAGCAGACGGCAT | |
| | CCCGACTACAGCGTGAGCCTGCTGCTG | |
| | AGACTGGCCAAGAAGTATGAGGCCAC | |
| | TCTGGAGAAATGCTGTGCGGAAGCCA | |
| | ACCCGCCGGCCTGCTACGGTACCGTCC | |
| | TGGCCGAGTTTCAGCCCCTGGTGGAGG | |
| | AGCCCAAGAACCTGGTGAAGACCAAC | |
| | TGCGACCTGTACGAGAAGCTGGGCGA | |
| | GTACGGCTTTCAGAACGCCATCCTGGT | |
| | GAGATACACACAGAAGGCCCCCCAAG | |
| | TGAGCACCCCCACCCTAGTGGAGGCCG | |
| | CAAGAAACCTGGGCAGAGTGGGCACA | |
| | AAATGCTGCACGCTCCCCGAGGATCAG | |
| | AGACTGCCCTGCGTGGAGGACTACCTG | |
| | AGCGCCATCCTGAACAGAGTGTGCCTG | |
| | CTGCACGAGAAGACCCCCGTGAGCGA | |
| | GCACGTGACCAAATGCTGCAGCGGAA | |
| | GCCTGGTGGAGAGAAGACCCTGCTTCA | |
| | GCGCCCTGACCGTGGACGAGACCTAC | |
| | GTGCCCAAGGAGTTCAAGGCCGAGAC | |
| | CTTCACCTTCCACAGCGACATCTGCAC | |
| | CCTGCCCGAGAAGGAAAAGCAGATCA | |
| | AGAAGCAGACCGCCCTGGCCGAGCTG | |
| | GTGAAGCACAAGCCCAAGGCCACCGC | |
| | CGAGCAGCTGAAGACCGTGATGGACG | |
| | ACTTCGCTCAGTTCCTGGACACCTGCT | |
| | GCAAGGCCGCCGACAAGGACACCTGC | |
| | TTCAGCACCGAGGGACCCAATTTAGTA | |
| | ACAAGATGCAAAGATGCATTGGCCGG | |
| | TGGTGGCGGCTCGGGCGGGGGCGGGT | |
| | CCGGTGGTGGGGGTAGCGCCCCCACA | |
| | AGCAGCAGCACCAAGAAGACACAGCT | |
| | GCAGCTGGAGCACCTGCTGCTGGACCT | |
| | GCAGATGATCCTGAACGGCATCAACA | |
| | ACTGCAAGAACCCCAAGCTGACAAGA | |
| | ATGCTGACCAAGAAGTTCTACATGCCC | |
| | AAGAAGGCCACCGAGCTGAAGCACCT | |
| | GCAGTGCCTGGAGGAGGAGCTGAAGC | |
| | CCCTGGAAGAGGTGCTGAACCTGGCTC | |
| | AGAGCAAGAACTTCCACCTGAGACCT | |
| | AGAGACCTGATCAGCAACATCAACGT | |
| | GATCGTGCTGGAGCTGAAGGGCAGCG | |
| | AGACTACCTTCATGTGCGAATACGCCG | |
| | ACGAGACCGCCACCATCGTGGAGTTCC | |
| | TGAACAGATGGATCACCTTCTCTCAGA | |
| | GCATCATCAGCACCCTGACCTGATAA | |
| 10 | GACGCCCACAAGAGCGAGGTGGCCCA | DNA sequence of HSA- |
| | CAGATTCAAGGACCTGGGCGAGGAGA | ACT4210, also known as |
| | ACTTCAAGGCCCTGGTGCTGATCGCCT | HSA-4210 (includes |
| | TCGCTCAGTACCTGCAGCAGTGCCCCT | G4S linker) |
| | TCGAGGACCACGTGAAGCTGGTGAAC | |
| | GAGGTGACCGAGTTCGCCAAGACCTG | |
| | CGTGGCCGACGAGAGCGCCGAGAACT | |
| | GCGACAAGAGCCTGCACACCCTGTTCG | |
| | GCGACAAGCTGTGCACCGTGGCCACCC | |
| | TGAGAGAGACCTACGGCGAGATGGCC | |
| | GACTGCTGCGCCAAGCAAGAGCCCGA | |
| | GAGAAACGAGTGCTTCCTGCAGCACA | |
| | AGGACGACAACCCCAACCTGCCTAGA | |
| | CTGGTGAGACCCGAGGTGGACGTGAT | |
| | GTGCACCGCCTTCCACGACAACGAGG | |
| | AGACCTTCCTGAAGAAGTACCTGTACG | |
| | AGATCGCAAGACGGCACCCGTATTTCT | |
| | ACGCCCCCGAGCTGCTGTTCTTCGCCA | |
| | AGAGATACAAGGCGGCTTTTACCGAAT | |
| | GCTGTCAAGCCGCCGACAAAGCGGCG | |
| | TGTCTGCTGCCCAAGCTGGACGAGCTG | |
| | AGAGACGAGGGCAAGGCTAGCAGCGC | |
| | CAAGCAGAGACTGAAGTGCGCTAGCC | |
| | TGCAGAAGTTCGGCGAGAGAGCCTTC | |
| | AAGGCCTGGGCCGTGGCTAGACTGTCT | |
| | CAGAGATTCCCCAAGGCCGAGTTCGCC | |
| | GAGGTGAGCAAGCTGGTGACCGACCT | |
| | GACCAAGGTGCACACCGAGTGCTGCC | |
| | ACGGCGACCTGCTGGAGTGCGCCGAC | |
| | GACAGAGCCGACCTGGCCAAGTACAT | |
| | CTGCGAGAACCAAGACAGCATCAGCA | |
| | GCAAGCTGAAGGAGTGCTGCGAGAAG | |
| | CCCCTGCTGGAGAAGAGCCACTGCATC | |
| | GCCGAGGTGGAGAACGACGAGATGCC | |
| | CGCCGACCTGCCTAGCCTGGCCGCCGA | |
| | CTTCGTGGAGAGCAAGGACGTGTGCA | |
| | AGAACTACGCCGAGGCCAAGGACGTG | |
| | TTCCTGGGCATGTTCCTGTACGAGTAC | |
| | GCTAGAAGACATCCGGACTACAGCGT | |
| | GGTGCTGTTACTGAGACTGGCCAAGAC | |
| | CTACGAAACTACCTTAGAGAAGTGTTG | |
| | TGCCGCTGCCGACCCCCATGAGTGCTA | |
| | TGCCAAGGTGTTCGACGAGTTCAAGCC | |
| | CCTAGTGGAAGAGCCTCAGAACCTGAT | |
| | CAAGCAGAACTGCGAGCTGTTCGAGC | |
| | AGCTGGGCGAGTACAAGTTTCAGAAC | |
| | GCCCTGCTGGTGAGATACACCAAGAA | |
| | GGTGCCCCAAGTGAGCACCCCCACGTT | |
| | GGTGGAGGTGAGCAGAAACCTGGGCA | |
| | AGGTGGGCAGCAAGTGCTGCAAGCAC | |
| | CCCGAGGCCAAGAGAATGCCCTGCGC | |
| | CGAGGACTACCTGTCCGTGGTTCTGAA | |
| | TCAGCTGTGCGTGCTGCACGAGAAGAC | |
| | CCCCGTGAGCGACAGAGTGACCAAGT | |
| | GCTGCACCGAGAGCCTGGTGAACAGA | |
| | AGACCCTGCTTCAGCGCCCTGGAGGTG | |
| | GACGAGACCTACGTGCCCAAGGAGTT | |
| | CAACGCCGAGACCTTCACCTTCCACGC | |
| | CGACATCTGCACCCTGAGCGAGAAGG | |
| | AGAGACAGATCAAGAAGCAGACCGCC | |
| | CTGGTGGAGCTGGTGAAGCACAAGCC | |
| | CAAGGCCACCAAGGAGCAGCTGAAGG | |
| | CCGTGATGGACGACTTCGCCGCCTTCG | |
| | TGGAAAAATGTTGTAAAGCCGATGAC | |
| | AAGGAGACCTGCTTCGCCGAGGAGGG | |
| | CAAGAAGCTGGTGGCCGCTAGCCAAG | |
| | CCGCCCTGGGCCTGGGGGGTGGGGGA | |
| | AGCGGCGGTGGCGGTAGCGGGGGAGG | |
| | CGGTAGCGCCCCCACAAGCAGCAGCA | |
| | CCAAGAAGACACAGCTGCAGCTGGAG | |
| | CACCTGCTGCTGGACCTGCAGATGATC | |
| | CTGAACGGCATCAACAACTGCAAGAA | |
| | CCCCAAGCTGACAAGAATGCTGACCA | |
| | AGAAGTTCTACATGCCCAAGAAGGCC | |
| | ACCGAGCTGAAGCACCTGCAGTGCCTG | |
| | GAGGAGGAGCTGAAGCCCCTGGAAGA | |
| | GGTGCTGAACCTGGCTCAGAGCAAGA | |
| | ACTTCCACCTGAGACCTAGAGACCTGA | |
| | TCAGCAACATCAACGTGATCGTGCTGG | |
| | AGCTGAAGGGCAGCGAGACCACCTTT | |
| | ATGTGCGAGTACGCCGACGAGACCGC | |
| | CACCATCGTGGAGTTCCTGAACAGATG | |
| | GATCACCTTTTCTCAGAGCATCATTAG | |
| | CACCCTCACC | |
| 11 | AGAGGCGUGUUCAGAAGAGAGGCCCA | mRNA sequence of |
| | CAAGAGCGAGAUCGCCCACAGAUACA | MSA-ACT4210, also |
| | ACGACCUGGGCGAGCAGCACUUCAAG | known as MSA-4210 |
| | GGCCUGGUGCUGAUCGCCUUCUCUCA | (includes G4S linker; |
| | GUACCUGCAGAAGUGCAGCUACGACG | without 5′ and 3′ non- |
| | AGCACGCCAAGCUGGUGCAAGAGGUG | coding sequences)) |
| | ACCGACUUCGCCAAGACCUGCGUGGC | |
| | CGACGAGAGCGCCGCCAACUGCGACA | |
| | AGAGCCUGCACACCCUGUUCGGCGAC | |
| | AAGCUGUGCGCCAUCCCCAACCUGAG | |
| | AGAGAACUACGGCGAGCUGGCCGACU | |
| | GCUGCACCAAGCAAGAGCCCGAGAGA | |
| | AACGAGUGCUUCCUGCAGCACAAGGA | |
| | CGACAACCCUAGCCUGCCCCCCUUCG | |
| | AGAGACCCGAGGCCGAGGCCAUGUGC | |
| | ACAAGCUUCAAGGAGAACCCCACCAC | |
| | CUUUAUGGGACACUACCUGCACGAGG | |
| | UGGCCCGUAGACAUCCCUAUUUUUAC | |
| | GCCCCCGAGCUGCUGUACUACGCCGA | |
| | GCAGUACAACGAGAUCCUGACCCAAU | |
| | GCUGUGCCGAAGCUGACAAGGAGAGC | |
| | UGCCUGACCCCCAAGCUGGACGGCGU | |
| | GAAGGAGAAGGCCCUGGUGAGCAGCG | |
| | UGAGACAGAGAAUGAAGUGCAGCAGC | |
| | AUGCAGAAGUUCGGCGAGAGAGCCUU | |
| | CAAGGCCUGGGCCGUGGCUAGACUGU | |
| | CUCAGACCUUCCCCAACGCCGACUUC | |
| | GCCGAGAUCACCAAGCUGGCCACCGA | |
| | CCUGACCAAGGUGAACAAGGAGUGCU | |
| | GCCACGGCGACCUGCUGGAGUGCGCC | |
| | GACGACAGAGCCGAGCUGGCCAAGUA | |
| | CAUGUGCGAGAACCAAGCCACCAUCA | |
| | GCAGCAAGCUGCAGACCUGCUGCGAC | |
| | AAGCCCCUGCUGAAGAAGGCCCACUG | |
| | CCUGUCGGAGGUGGAACACGACACCA | |
| | UGCCCGCAGAUCUGCCCGCCAUCGCC | |
| | GCCGACUUCGUGGAGGACCAAGAGGU | |
| | GUGCAAGAACUACGCCGAAGCCAAGG | |
| | AUGUGUUCCUGGGCACCUUCCUGUAC | |
| | GAGUACAGCAGACGGCAUCCCGACUA | |
| | CAGCGUGAGCCUGCUGCUGAGACUGG | |
| | CCAAGAAGUAUGAGGCCACUCUGGAG | |
| | AAAUGCUGUGCGGAAGCCAACCCGCC | |
| | GGCCUGCUACGGUACCGUCCUGGCCG | |
| | AGUUUCAGCCCCUGGUGGAGGAGCCC | |
| | AAGAACCUGGUGAAGACCAACUGCGA | |
| | CCUGUACGAGAAGCUGGGCGAGUACG | |
| | GCUUUCAGAACGCCAUCCUGGUGAGA | |
| | UACACACAGAAGGCCCCCCAAGUGAG | |
| | CACCCCCACCCUAGUGGAGGCCGCAA | |
| | GAAACCUGGGCAGAGUGGGCACAAAA | |
| | UGCUGCACGCUCCCCGAGGAUCAGAG | |
| | ACUGCCCUGCGUGGAGGACUACCUGA | |
| | GCGCCAUCCUGAACAGAGUGUGCCUG | |
| | CUGCACGAGAAGACCCCCGUGAGCGA | |
| | GCACGUGACCAAAUGCUGCAGCGGAA | |
| | GCCUGGUGGAGAGAAGACCCUGCUUC | |
| | AGCGCCCUGACCGUGGACGAGACCUA | |
| | CGUGCCCAAGGAGUUCAAGGCCGAGA | |
| | CCUUCACCUUCCACAGCGACAUCUGC | |
| | ACCCUGCCCGAGAAGGAAAAGCAGAU | |
| | CAAGAAGCAGACCGCCCUGGCCGAGC | |
| | UGGUGAAGCACAAGCCCAAGGCCACC | |
| | GCCGAGCAGCUGAAGACCGUGAUGGA | |
| | CGACUUCGCUCAGUUCCUGGACACCU | |
| | GCUGCAAGGCCGCCGACAAGGACACC | |
| | UGCUUCAGCACCGAGGGACCCAAUUU | |
| | AGUAACAAGAUGCAAAGAUGCAUUG | |
| | GCCGGUGGUGGCGGCUCGGGCGGGGG | |
| | CGGGUCCGGUGGUGGGGGUAGCGCCC | |
| | CCACAAGCAGCAGCACCAAGAAGACA | |
| | CAGCUGCAGCUGGAGCACCUGCUGCU | |
| | GGACCUGCAGAUGAUCCUGAACGGCA | |
| | UCAACAACUGCAAGAACCCCAAGCUG | |
| | ACAAGAAUGCUGACCAAGAAGUUCUA | |
| | CAUGCCCAAGAAGGCCACCGAGCUGA | |
| | AGCACCUGCAGUGCCUGGAGGAGGAG | |
| | CUGAAGCCCCUGGAAGAGGUGCUGAA | |
| | CCUGGCUCAGAGCAAGAACUUCCACC | |
| | UGAGACCUAGAGACCUGAUCAGCAAC | |
| | AUCAACGUGAUCGUGCUGGAGCUGAA | |
| | GGGCAGCGAGACUACCUUCAUGUGCG | |
| | AAUACGCCGACGAGACCGCCACCAUC | |
| | GUGGAGUUCCUGAACAGAUGGAUCAC | |
| | CUUCUCUCAGAGCAUCAUCAGCACCC | |
| | UGACC | |
| 12 | AGGAGACCCAAGCUGGCUAGCGUUUA | mRNA sequence of |
| | AACUUAAGCUUGGUACCGAGCUCGGA | MSA-ACT4210, also |
| | UCCACUAGUCCAGUGUGGUGGAAUUC | known as MSA-4210 |
| | UGCAGAUACUCUUCUGGUCCCCACAG | (includes G4S linker; |
| | ACUCAGAGAGAAGCCACCAUGGAGUU | with 5′ and 3′ non- |
| | CGGCCUGAGCUGGGUGUUCCUGGUGG | coding sequences)) |
| | CCCUGUUCAGAGGCGUGCAGAGCAGA | |
| | GGCGUGUUCAGAAGAGAGGCCCACAA | |
| | GAGCGAGAUCGCCCACAGAUACAACG | |
| | ACCUGGGCGAGCAGCACUUCAAGGGC | |
| | CUGGUGCUGAUCGCCUUCUCUCAGUA | |
| | CCUGCAGAAGUGCAGCUACGACGAGC | |
| | ACGCCAAGCUGGUGCAAGAGGUGACC | |
| | GACUUCGCCAAGACCUGCGUGGCCGA | |
| | CGAGAGCGCCGCCAACUGCGACAAGA | |
| | GCCUGCACACCCUGUUCGGCGACAAG | |
| | CUGUGCGCCAUCCCCAACCUGAGAGA | |
| | GAACUACGGCGAGCUGGCCGACUGCU | |
| | GCACCAAGCAAGAGCCCGAGAGAAAC | |
| | GAGUGCUUCCUGCAGCACAAGGACGA | |
| | CAACCCUAGCCUGCCCCCCUUCGAGA | |
| | GACCCGAGGCCGAGGCCAUGUGCACA | |
| | AGCUUCAAGGAGAACCCCACCACCUU | |
| | UAUGGGACACUACCUGCACGAGGUGG | |
| | CCCGUAGACAUCCCUAUUUUUACGCC | |
| | CCCGAGCUGCUGUACUACGCCGAGCA | |
| | GUACAACGAGAUCCUGACCCAAUGCU | |
| | GUGCCGAAGCUGACAAGGAGAGCUGC | |
| | CUGACCCCCAAGCUGGACGGCGUGAA | |
| | GGAGAAGGCCCUGGUGAGCAGCGUGA | |
| | GACAGAGAAUGAAGUGCAGCAGCAUG | |
| | CAGAAGUUCGGCGAGAGAGCCUUCAA | |
| | GGCCUGGGCCGUGGCUAGACUGUCUC | |
| | AGACCUUCCCCAACGCCGACUUCGCC | |
| | GAGAUCACCAAGCUGGCCACCGACCU | |
| | GACCAAGGUGAACAAGGAGUGCUGCC | |
| | ACGGCGACCUGCUGGAGUGCGCCGAC | |
| | GACAGAGCCGAGCUGGCCAAGUACAU | |
| | GUGCGAGAACCAAGCCACCAUCAGCA | |
| | GCAAGCUGCAGACCUGCUGCGACAAG | |
| | CCCCUGCUGAAGAAGGCCCACUGCCU | |
| | GUCGGAGGUGGAACACGACACCAUGC | |
| | CCGCAGAUCUGCCCGCCAUCGCCGCC | |
| | GACUUCGUGGAGGACCAAGAGGUGUG | |
| | CAAGAACUACGCCGAAGCCAAGGAUG | |
| | UGUUCCUGGGCACCUUCCUGUACGAG | |
| | UACAGCAGACGGCAUCCCGACUACAG | |
| | CGUGAGCCUGCUGCUGAGACUGGCCA | |
| | AGAAGUAUGAGGCCACUCUGGAGAAA | |
| | UGCUGUGCGGAAGCCAACCCGCCGGC | |
| | CUGCUACGGUACCGUCCUGGCCGAGU | |
| | UUCAGCCCCUGGUGGAGGAGCCCAAG | |
| | AACCUGGUGAAGACCAACUGCGACCU | |
| | GUACGAGAAGCUGGGCGAGUACGGCU | |
| | UUCAGAACGCCAUCCUGGUGAGAUAC | |
| | ACACAGAAGGCCCCCCAAGUGAGCAC | |
| | CCCCACCCUAGUGGAGGCCGCAAGAA | |
| | ACCUGGGCAGAGUGGGCACAAAAUGC | |
| | UGCACGCUCCCCGAGGAUCAGAGACU | |
| | GCCCUGCGUGGAGGACUACCUGAGCG | |
| | CCAUCCUGAACAGAGUGUGCCUGCUG | |
| | CACGAGAAGACCCCCGUGAGCGAGCA | |
| | CGUGACCAAAUGCUGCAGCGGAAGCC | |
| | UGGUGGAGAGAAGACCCUGCUUCAGC | |
| | GCCCUGACCGUGGACGAGACCUACGU | |
| | GCCCAAGGAGUUCAAGGCCGAGACCU | |
| | UCACCUUCCACAGCGACAUCUGCACC | |
| | CUGCCCGAGAAGGAAAAGCAGAUCAA | |
| | GAAGCAGACCGCCCUGGCCGAGCUGG | |
| | UGAAGCACAAGCCCAAGGCCACCGCC | |
| | GAGCAGCUGAAGACCGUGAUGGACGA | |
| | CUUCGCUCAGUUCCUGGACACCUGCU | |
| | GCAAGGCCGCCGACAAGGACACCUGC | |
| | UUCAGCACCGAGGGACCCAAUUUAGU | |
| | AACAAGAUGCAAAGAUGCAUUGGCCG | |
| | GUGGUGGCGGCUCGGGCGGGGGCGGG | |
| | UCCGGUGGUGGGGGUAGCGCCCCCAC | |
| | AAGCAGCAGCACCAAGAAGACACAGC | |
| | UGCAGCUGGAGCACCUGCUGCUGGAC | |
| | CUGCAGAUGAUCCUGAACGGCAUCAA | |
| | CAACUGCAAGAACCCCAAGCUGACAA | |
| | GAAUGCUGACCAAGAAGUUCUACAUG | |
| | CCCAAGAAGGCCACCGAGCUGAAGCA | |
| | CCUGCAGUGCCUGGAGGAGGAGCUGA | |
| | AGCCCCUGGAAGAGGUGCUGAACCUG | |
| | GCUCAGAGCAAGAACUUCCACCUGAG | |
| | ACCUAGAGACCUGAUCAGCAACAUCA | |
| | ACGUGAUCGUGCUGGAGCUGAAGGGC | |
| | AGCGAGACUACCUUCAUGUGCGAAUA | |
| | CGCCGACGAGACCGCCACCAUCGUGG | |
| | AGUUCCUGAACAGAUGGAUCACCUUC | |
| | UCUCAGAGCAUCAUCAGCACCCUGAC | |
| | CUGAUAAAUCCAGCACAGUGGCGGCC | |
| | CUGGUACUGCAUGCACGCAAUGCUAG | |
| | CUGCCCCUUUCCCGUCCUGGGCACCC | |
| | CGAGUCUCCCCCGACCCCGGGUCCCA | |
| | GGUAUGCUCCCACCUCCACCUGCCCC | |
| | ACUCACCACCUCUGCUAGUUCCAGAC | |
| | ACCUCCCAAGCACGCAGCAAUGCAGC | |
| | UCAAAACGCUUAGCCUAGCCACACCC | |
| | CCACGGGAAACAGCAGUGAUUAACCU | |
| | UUAGCAAUAAACGAAAGUUUAACUA | |
| | AGCUAUACUAACCCCAGGGUUGGUCA | |
| | AUUUCGUGCCAGCCACAAAAAAAAAA | |
| | AAAAAAAAAAAAAAAAAAAAAAAAA | |
| | AAAAAAAAAAAAAAAAAAAAAAAAA | |
| | AAAAAAAAAAAAAAAAAAAAAAAAA | |
| | AAAAAAAAAAAAAAAAAAAAAAAAA | |
| | AAAAAAAAAAU | |
| 13 | GACGCCCACAAGAGCGAGGUGGCCCA | mRNA sequence of |
| | CAGAUUCAAGGACCUGGGCGAGGAGA | HSA-ACT4210, also |
| | ACUUCAAGGCCCUGGUGCUGAUCGCC | known as HSA-4210 |
| | UUCGCUCAGUACCUGCAGCAGUGCCC | (includes G4S linker; |
| | CUUCGAGGACCACGUGAAGCUGGUGA | without 5′ and 3′ non- |
| | ACGAGGUGACCGAGUUCGCCAAGACC | coding sequences)) |
| | UGCGUGGCCGACGAGAGCGCCGAGAA | |
| | CUGCGACAAGAGCCUGCACACCCUGU | |
| | UCGGCGACAAGCUGUGCACCGUGGCC | |
| | ACCCUGAGAGAGACCUACGGCGAGAU | |
| | GGCCGACUGCUGCGCCAAGCAAGAGC | |
| | CCGAGAGAAACGAGUGCUUCCUGCAG | |
| | CACAAGGACGACAACCCCAACCUGCC | |
| | UAGACUGGUGAGACCCGAGGUGGACG | |
| | UGAUGUGCACCGCCUUCCACGACAAC | |
| | GAGGAGACCUUCCUGAAGAAGUACCU | |
| | GUACGAGAUCGCAAGACGGCACCCGU | |
| | AUUUCUACGCCCCCGAGCUGCUGUUC | |
| | UUCGCCAAGAGAUACAAGGCGGCUUU | |
| | UACCGAAUGCUGUCAAGCCGCCGACA | |
| | AAGCGGCGUGUCUGCUGCCCAAGCUG | |
| | GACGAGCUGAGAGACGAGGGCAAGGC | |
| | UAGCAGCGCCAAGCAGAGACUGAAGU | |
| | GCGCUAGCCUGCAGAAGUUCGGCGAG | |
| | AGAGCCUUCAAGGCCUGGGCCGUGGC | |
| | UAGACUGUCUCAGAGAUUCCCCAAGG | |
| | CCGAGUUCGCCGAGGUGAGCAAGCUG | |
| | GUGACCGACCUGACCAAGGUGCACAC | |
| | CGAGUGCUGCCACGGCGACCUGCUGG | |
| | AGUGCGCCGACGACAGAGCCGACCUG | |
| | GCCAAGUACAUCUGCGAGAACCAAGA | |
| | CAGCAUCAGCAGCAAGCUGAAGGAGU | |
| | GCUGCGAGAAGCCCCUGCUGGAGAAG | |
| | AGCCACUGCAUCGCCGAGGUGGAGAA | |
| | CGACGAGAUGCCCGCCGACCUGCCUA | |
| | GCCUGGCCGCCGACUUCGUGGAGAGC | |
| | AAGGACGUGUGCAAGAACUACGCCGA | |
| | GGCCAAGGACGUGUUCCUGGGCAUGU | |
| | UCCUGUACGAGUACGCUAGAAGACAU | |
| | CCGGACUACAGCGUGGUGCUGUUACU | |
| | GAGACUGGCCAAGACCUACGAAACUA | |
| | CCUUAGAGAAGUGUUGUGCCGCUGCC | |
| | GACCCCCAUGAGUGCUAUGCCAAGGU | |
| | GUUCGACGAGUUCAAGCCCCUAGUGG | |
| | AAGAGCCUCAGAACCUGAUCAAGCAG | |
| | AACUGCGAGCUGUUCGAGCAGCUGGG | |
| | CGAGUACAAGUUUCAGAACGCCCUGC | |
| | UGGUGAGAUACACCAAGAAGGUGCCC | |
| | CAAGUGAGCACCCCCACGUUGGUGGA | |
| | GGUGAGCAGAAACCUGGGCAAGGUGG | |
| | GCAGCAAGUGCUGCAAGCACCCCGAG | |
| | GCCAAGAGAAUGCCCUGCGCCGAGGA | |
| | CUACCUGUCCGUGGUUCUGAAUCAGC | |
| | UGUGCGUGCUGCACGAGAAGACCCCC | |
| | GUGAGCGACAGAGUGACCAAGUGCUG | |
| | CACCGAGAGCCUGGUGAACAGAAGAC | |
| | CCUGCUUCAGCGCCCUGGAGGUGGAC | |
| | GAGACCUACGUGCCCAAGGAGUUCAA | |
| | CGCCGAGACCUUCACCUUCCACGCCG | |
| | ACAUCUGCACCCUGAGCGAGAAGGAG | |
| | AGACAGAUCAAGAAGCAGACCGCCCU | |
| | GGUGGAGCUGGUGAAGCACAAGCCCA | |
| | AGGCCACCAAGGAGCAGCUGAAGGCC | |
| | GUGAUGGACGACUUCGCCGCCUUCGU | |
| | GGAAAAAUGUUGUAAAGCCGAUGAC | |
| | AAGGAGACCUGCUUCGCCGAGGAGGG | |
| | CAAGAAGCUGGUGGCCGCUAGCCAAG | |
| | CCGCCCUGGGCCUGGGGGGUGGGGGA | |
| | AGCGGCGGUGGCGGUAGCGGGGGAGG | |
| | CGGUAGCGCCCCCACAAGCAGCAGCA | |
| | CCAAGAAGACACAGCUGCAGCUGGAG | |
| | CACCUGCUGCUGGACCUGCAGAUGAU | |
| | CCUGAACGGCAUCAACAACUGCAAGA | |
| | ACCCCAAGCUGACAAGAAUGCUGACC | |
| | AAGAAGUUCUACAUGCCCAAGAAGGC | |
| | CACCGAGCUGAAGCACCUGCAGUGCC | |
| | UGGAGGAGGAGCUGAAGCCCCUGGAA | |
| | GAGGUGCUGAACCUGGCUCAGAGCAA | |
| | GAACUUCCACCUGAGACCUAGAGACC | |
| | UGAUCAGCAACAUCAACGUGAUCGUG | |
| | CUGGAGCUGAAGGGCAGCGAGACCAC | |
| | CUUUAUGUGCGAGUACGCCGACGAGA | |
| | CCGCCACCAUCGUGGAGUUCCUGAAC | |
| | AGAUGGAUCACCUUUUCUCAGAGCAU | |
| | CAUUAGCACCCUCACC | |
| 14 | AGGAGACCCAAGCUGGCUAGCGUUUA | mRNA sequence of |
| | AACUUAAGCUUGGUACCGAGCUCGGA | HSA-ACT4210, also |
| | UCCACUAGUCCAGUGUGGUGGAAUUC | known as HSA-4210 |
| | UGCAGAUACUCUUCUGGUCCCCACAG | (includes G4S linker; |
| | ACUCAGAGAGAAGCCACCAUGGAGUU | with 5′ and 3′ non- |
| | CGGCCUGAGCUGGGUGUUCCUGGUGG | coding sequences)) |
| | CCCUGUUCAGAGGCGUGCAGAGCGAC | |
| | GCCCACAAGAGCGAGGUGGCCCACAG | |
| | AUUCAAGGACCUGGGCGAGGAGAACU | |
| | UCAAGGCCCUGGUGCUGAUCGCCUUC | |
| | GCUCAGUACCUGCAGCAGUGCCCCUU | |
| | CGAGGACCACGUGAAGCUGGUGAACG | |
| | AGGUGACCGAGUUCGCCAAGACCUGC | |
| | GUGGCCGACGAGAGCGCCGAGAACUG | |
| | CGACAAGAGCCUGCACACCCUGUUCG | |
| | GCGACAAGCUGUGCACCGUGGCCACC | |
| | CUGAGAGAGACCUACGGCGAGAUGGC | |
| | CGACUGCUGCGCCAAGCAAGAGCCCG | |
| | AGAGAAACGAGUGCUUCCUGCAGCAC | |
| | AAGGACGACAACCCCAACCUGCCUAG | |
| | ACUGGUGAGACCCGAGGUGGACGUGA | |
| | UGUGCACCGCCUUCCACGACAACGAG | |
| | GAGACCUUCCUGAAGAAGUACCUGUA | |
| | CGAGAUCGCAAGACGGCACCCGUAUU | |
| | UCUACGCCCCCGAGCUGCUGUUCUUC | |
| | GCCAAGAGAUACAAGGCGGCUUUUAC | |
| | CGAAUGCUGUCAAGCCGCCGACAAAG | |
| | CGGCGUGUCUGCUGCCCAAGCUGGAC | |
| | GAGCUGAGAGACGAGGGCAAGGCUAG | |
| | CAGCGCCAAGCAGAGACUGAAGUGCG | |
| | CUAGCCUGCAGAAGUUCGGCGAGAGA | |
| | GCCUUCAAGGCCUGGGCCGUGGCUAG | |
| | ACUGUCUCAGAGAUUCCCCAAGGCCG | |
| | AGUUCGCCGAGGUGAGCAAGCUGGUG | |
| | ACCGACCUGACCAAGGUGCACACCGA | |
| | GUGCUGCCACGGCGACCUGCUGGAGU | |
| | GCGCCGACGACAGAGCCGACCUGGCC | |
| | AAGUACAUCUGCGAGAACCAAGACAG | |
| | CAUCAGCAGCAAGCUGAAGGAGUGCU | |
| | GCGAGAAGCCCCUGCUGGAGAAGAGC | |
| | CACUGCAUCGCCGAGGUGGAGAACGA | |
| | CGAGAUGCCCGCCGACCUGCCUAGCC | |
| | UGGCCGCCGACUUCGUGGAGAGCAAG | |
| | GACGUGUGCAAGAACUACGCCGAGGC | |
| | CAAGGACGUGUUCCUGGGCAUGUUCC | |
| | UGUACGAGUACGCUAGAAGACAUCCG | |
| | GACUACAGCGUGGUGCUGUUACUGAG | |
| | ACUGGCCAAGACCUACGAAACUACCU | |
| | UAGAGAAGUGUUGUGCCGCUGCCGAC | |
| | CCCCAUGAGUGCUAUGCCAAGGUGUU | |
| | CGACGAGUUCAAGCCCCUAGUGGAAG | |
| | AGCCUCAGAACCUGAUCAAGCAGAAC | |
| | UGCGAGCUGUUCGAGCAGCUGGGCGA | |
| | GUACAAGUUUCAGAACGCCCUGCUGG | |
| | UGAGAUACACCAAGAAGGUGCCCCAA | |
| | GUGAGCACCCCCACGUUGGUGGAGGU | |
| | GAGCAGAAACCUGGGCAAGGUGGGCA | |
| | GCAAGUGCUGCAAGCACCCCGAGGCC | |
| | AAGAGAAUGCCCUGCGCCGAGGACUA | |
| | CCUGUCCGUGGUUCUGAAUCAGCUGU | |
| | GCGUGCUGCACGAGAAGACCCCCGUG | |
| | AGCGACAGAGUGACCAAGUGCUGCAC | |
| | CGAGAGCCUGGUGAACAGAAGACCCU | |
| | GCUUCAGCGCCCUGGAGGUGGACGAG | |
| | ACCUACGUGCCCAAGGAGUUCAACGC | |
| | CGAGACCUUCACCUUCCACGCCGACA | |
| | UCUGCACCCUGAGCGAGAAGGAGAGA | |
| | CAGAUCAAGAAGCAGACCGCCCUGGU | |
| | GGAGCUGGUGAAGCACAAGCCCAAGG | |
| | CCACCAAGGAGCAGCUGAAGGCCGUG | |
| | AUGGACGACUUCGCCGCCUUCGUGGA | |
| | AAAAUGUUGUAAAGCCGAUGACAAG | |
| | GAGACCUGCUUCGCCGAGGAGGGCAA | |
| | GAAGCUGGUGGCCGCUAGCCAAGCCG | |
| | CCCUGGGCCUGGGGGGUGGGGGAAGC | |
| | GGCGGUGGCGGUAGCGGGGGAGGCGG | |
| | UAGCGCCCCCACAAGCAGCAGCACCA | |
| | AGAAGACACAGCUGCAGCUGGAGCAC | |
| | CUGCUGCUGGACCUGCAGAUGAUCCU | |
| | GAACGGCAUCAACAACUGCAAGAACC | |
| | CCAAGCUGACAAGAAUGCUGACCAAG | |
| | AAGUUCUACAUGCCCAAGAAGGCCAC | |
| | CGAGCUGAAGCACCUGCAGUGCCUGG | |
| | AGGAGGAGCUGAAGCCCCUGGAAGAG | |
| | GUGCUGAACCUGGCUCAGAGCAAGAA | |
| | CUUCCACCUGAGACCUAGAGACCUGA | |
| | UCAGCAACAUCAACGUGAUCGUGCUG | |
| | GAGCUGAAGGGCAGCGAGACCACCUU | |
| | UAUGUGCGAGUACGCCGACGAGACCG | |
| | CCACCAUCGUGGAGUUCCUGAACAGA | |
| | UGGAUCACCUUUUCUCAGAGCAUCAU | |
| | UAGCACCCUCACCUGAUAAAUCCAGC | |
| | ACAGUGGCGGCCCUGGUACUGCAUGC | |
| | ACGCAAUGCUAGCUGCCCCUUUCCCG | |
| | UCCUGGGCACCCCGAGUCUCCCCCGA | |
| | CCCCGGGUCCCAGGUAUGCUCCCACC | |
| | UCCACCUGCCCCACUCACCACCUCUG | |
| | CUAGUUCCAGACACCUCCCAAGCACG | |
| | CAGCAAUGCAGCUCAAAACGCUUAGC | |
| | CUAGCCACACCCCCACGGGAAACAGC | |
| | AGUGAUUAACCUUUAGCAAUAAACGA | |
| | AAGUUUAACUAAGCUAUACUAACCCC | |
| | AGGGUUGGUCAAUUUCGUGCCAGCCA | |
| | CAAAAAAAAAAAAAAAAAAAAAAAA | |
| | AAAAAAAAAAAAAAAAAAAAAAAAA | |
| | AAAAAAAAAAAAAAAAAAAAAAAAA | |
| | AAAAAAAAAAAAAAAAAAAAAAAAA | |
| | AAAAAAAAAAAAAAAAAAAAAU | |
| 15 | GGGGSGGGGSGGGGS | Amino acid sequence of |
| | | G4S linker |
| 16 | GGGGGTGGGGGAAGCGGCGGTGGCGG | DNA sequence of G4S |
| | TAGCGGGGGAGGCGGTAGC | linker |
| 17 | GGGGGUGGGGGAAGCGGCGGUGGCG | mRNA sequence of G4S |
| | GUAGCGGGGGAGGCGGUAGC | linker |

1. A polynucleotide comprising a nucleic acid sequence encoding a fusion protein comprising a modified interleukin 2 (IL-2) polypeptide, wherein the modified IL-2 polypeptide comprises at least one substitution with a natural amino acid or an unnatural amino acid at position Y31, or positions Y31 and F42, and wherein
the fusion protein comprises the modified IL-2 polypeptide and an additional amino acid sequence comprising a serum albumin or a PK-extending fragment or analog thereof.

2. The polynucleotide of claim 1, wherein the modified IL-2 polypeptide:
a) is configured to be unconjugated or is conjugated to a water-soluble polymer, a lipid, a polypeptide, a protein or a peptide; and/or
b) has reduced binding to an interleukin 2 receptor α (IL-2Rα) compared to a comparable IL-2 polypeptide comprising an amino acid sequence set forth in SEQ ID NO:1 or SEQ ID NO:2 without the at least one substitution; and/or
c) has reduced receptor signaling potency to IL-2Rαβγ compared to a comparable IL-2 polypeptide comprising an amino acid sequence set forth in SEQ ID NO:1 or SEQ ID NO:2 without the at least one substitution, and/or
d) has increased ratio of signaling potency to IL-2Rβγ over signaling potency to IL-2Rαβγ (increased ratio of signaling potency to IL-2Rβγ/signaling potency to IL-2Rαβγ) compared to a comparable IL-2 polypeptide comprising an amino acid sequence set forth in SEQ ID NO:1 or SEQ ID NO:2 without the at least one substitution, and/or
e) has enhanced receptor signaling potency to IL-2Rβγ compared to a comparable IL-2 polypeptide comprising an amino acid sequence set forth in SEQ ID NO:1 or SEQ ID NO:2 without the at least one substitution, and/or
provided that when the modified IL-2 polypeptide comprises at least one substitution with an unnatural amino acid, the modified IL-2 polypeptide comprises at least one substitution at position Y31, or positions Y31 and F42; and/or
f) combinations of a) through e).

3. The polynucleotide of claim 1, wherein the modified IL-2 polypeptide has at least about 90%, 91%, 92%, 93%, 94%, 95%, 96%, 97%, 98%, 99% or 100% sequence identity in one or more regions of amino acid positions 10-25, 80-100 and/or 100-134 to the corresponding one or more regions of an IL-2 polypeptide comprising an amino acid sequence set forth in SEQ ID NO:1 or SEQ ID NO:2.

4. (canceled)

5. The polynucleotide of claim 1, wherein the modified IL-2 polypeptide comprises at least one substitution with lysine, cysteine, histidine, arginine, aspartic acid, glutamic acid, serine, threonine, alanine, tryptophan, isoleucine, phenylalanine, or tyrosine at a position Y31, or positions Y31 and F42.

6. The polynucleotide of claim 1, wherein the modified IL-2 polypeptide comprises:
a) at least one substitution with a natural amino acid at Y31, positions Y31 and F42.

7. (canceled)

8. The polynucleotide of claim 1, wherein the modified IL-2 polypeptide comprises:
at least one substitution with cysteine at a position of Y31.

9-10. (canceled)

11. The polynucleotide of claim 1, wherein the modified IL-2 polypeptide comprises:
a) at least one substitution with cysteine at a position F42, Y45, E62, P65, and combinations thereof; and/or
b) a substitution with alanine, lysine or serine at position F42; and/or
c) a substitution with alanine at position F42; and/or
d) a substitution with serine at position F42; and/or
e) a substitution with lysine at position F42; and/or
f) a substitution with cysteine at position Y31, and/or
g) combinations of a) through f).

12-14. (canceled)

15. The polynucleotide of claim 1, wherein the modified IL-2 polypeptide comprises:
a) a substitution at position Y31; and/or
c) a substitution at position F42; and/or
d) a substitution at position C125; and/or
e) a substitution at positions Y31, F42, and C125.

16. (canceled)

17. The polynucleotide of claim 1, further comprising at least one substitution with a natural amino acid or an unnatural amino acid at at least one position within IL-2Rα interaction region, IL-2Rβ interaction region and/or IL-2Rγ interaction region.

18-30. (canceled)

31. The polynucleotide of claim 1, wherein the additional amino acid sequence comprises:
a) a human serum albumin (HSA);
b) a murine serum albumin (MSA);
c) or a PK-extending fragment or analog of a) or b).

32. The polynucleotide of claim 1, wherein the modified IL-2 polypeptide comprises the amino acid sequence set forth in SEQ ID NO:3.

33-61. (canceled)

62. The polynucleotide of claim 1, wherein the polynucleotide comprises: DNA; RNA; or a DNA/RNA hybrid.

63. The polynucleotide of claim 1, wherein the polynucleotide comprises RNA.

64-67. (canceled)

68. The polynucleotide ofany claim 1, wherein the fusion protein has a half-life in vivo from about 5 minutes to about 10 days, from about 5 minutes to about 9 days, from about 5 minutes to about 8 days, from about 5 minutes to about 8 days, from about 5 minutes to about 7 days, from about 5 minutes to about 6 days, from about 5 minutes to about 5 days, from about 5 minutes to about 4 days, from about 5 minutes to about 3 days, from about 5 minutes to about 2 days, from about 5 minutes to about 1 day, from about 1 hour to about 10 days, about 1 hour to about 9 days, from out 1 hour to about 8 days, from about 1 hour to about 7 days, from out 1 hours to about 6 days, from about 1 hour to about 5 days, from about 1 hour to about 4 days, about 1 hour to about 3 days, from out 1 hour to about 2 days, from about 1 hour to about 1 day, from about 5 minutes, from about 10 minutes, from about 20 minutes, from about 30 minutes, from about 40 minutes, from about 50 minutes, from about 1 hour, from about 2 hours, from about 3 hours, from about 4 hours, from about 5 hours, from about 6 hours, from about 7 hours, from about 8 hours, from about 9 hours, from about 10 hours, from about 11 hours, from about 12 hours, from about 13 hours, from about 14 hours, from about 15 hours, from about 16 hours, from about 17 hours, from about 18 hours, from about 19 hours, from about 20 hours, from about 21 hours, from about 22 hours, from about 23 hours, from about 1 day, from about 2 days, from about 3 days, from about 4 days, from about 5 days, from about 6 days, from about 7 days, from about 8 days, from about 9 days, from about 10 days or a value or a range in between.

69. A pharmaceutical composition comprising an effective amount of a polynucleotide of claim 1 and a pharmaceutically acceptable carrier.

70. The pharmaceutical composition of claim 69, wherein the pharmaceutically acceptable carrier comprises a lipid.

71. The pharmaceutical composition of claim 70, wherein the lipid comprises a cationic lipid of formula (I): 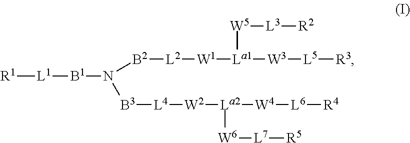
or a pharmaceutically acceptable salt, solvate, hydrate, stereoisomer, or prodrug thereof, wherein:
R¹ is H, —OR^{1A}, —YOR^{1A}, —NR^{1A}R^{1B}, —YNR^{1A}R^{1B}, —SR^{1A}, —YSR^{1A}, —(C═O)R^{1A}, —Y(C═O)R^{1A}, —(C═O)OR^{1A}, —Y(C═O)OR^{1A}, —O(C═O)R^{1A}, —YO(C═O)R^{1A}, —O(C═O)OR^{1A}, —YO(C═O)OR^{1A}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Y is substituted or unsubstituted C₀-C₁₂ alkylene or substituted or unsubstituted 0 to 12 membered heteroalkylene;
R² is H, —OR^{2A}, —SR^{2A}, —(C═O)R^{2A}, —(C═O)OR^{2A}, —O(C═O)R^{2A}, —O(C═O)OR^{2A}, —(C═O)NHR^{2A}, —NH(C═O)R^{2A}, substituted or unsubstituted alkyl, or substituted or unsubstituted heteroalkyl;
R³ is H, —OR^{3A}, —SR^{3A}, —(C═O)R^{3A}, —(C═O)OR^{3A}, —O(C═O)R^{3A}, —O(C═O)OR^{3A}, —(C═O)NHR^{3A}, —NH(C═O)R^{3A}, substituted or unsubstituted alkyl, or substituted or unsubstituted heteroalkyl;
R⁴ is H, —OR^{4A}, —SR^{4A}, —(C═O)R^{4A}, —(C═O)OR^{4A}, —O(C═O)R^{4A}, —O(C═O)OR^{4A}, —(C═O)NHR^{4A}, —NH(C═O)R^{4A}, substituted or unsubstituted alkyl, or substituted or unsubstituted heteroalkyl;
R⁵ is H, —OR^{5A}, —SR^{5A}, —(C═O)R^{5A}, —(C═O)OR^{5A}, —O(C═O)R^{5A}, —O(C═O)OR^{5A}, —(C═O)NHR^{5A}, —NH(C═O)R^{5A}, substituted or unsubstituted alkyl, or substituted or unsubstituted heteroalkyl;
B¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
B² and B³ are each independently a bond, substituted or unsubstituted alkylene, or substituted or unsubstituted heteroalkylene;
L¹ is a bond, —O(C═O)—, —(C═O)O—, —O(C═O)O—, —C(═O)—, —O—, —O(CR¹⁰¹R¹⁰²)ₛO—, —S—, —C(═O)S—, —SC(═O)—, —NR¹⁰¹C(═O)—, —C(═O)NR¹⁰¹—, —NR¹⁰¹C(═S)—, —C(═S)NR¹⁰¹—, —NR¹⁰¹C(═O)NR¹⁰²—, —NR¹⁰¹C(═S)NR¹⁰²—, —OC(═O)NR¹⁰¹—, —NR¹⁰¹C(═O)O—, —SC(═O)NR¹⁰¹— or —NR¹⁰¹C(═O)S—;
L² is a bond, —O(C═O)—, —(C═O)O—, —O(C═O)O—, —C(═O)—, —O—, —O(CR²⁰¹R²⁰²)ₛO—, —S—, —C(═O)S—, —SC(═O)—, —NR²⁰¹C(═O)—, —C(═O)NR²⁰¹—, —NR²⁰¹C(═O)NR²⁰²—, —NR²⁰¹C(═S)—, —C(═S)NR²⁰¹—, —NR²⁰¹C(═S)NR²⁰²—, —OC(═O)NR²⁰¹—, —NR²⁰¹C(═O)O—, —SC(═O)NR²⁰¹— or —NR²⁰¹C(═O)S—;
L³ is a bond, —O(C═O)—, —(C═O)O—, —O(C═O)O—, —C(═O)—, —O—, —O(CR³⁰¹R³⁰²)ₛO—, —S—, —C(═O)S—, —SC(═O)—, —NR³⁰¹C(═O)—, —C(═O)NR³⁰¹—, —NR³⁰¹C(═O)NR³⁰²—, —NR³⁰¹C(═S)—, —C(═S)NR³⁰¹—, —NR³⁰¹C(═S)NR³⁰²—, —OC(═O)NR³⁰¹—, —NR³⁰¹C(═O)O—, —SC(═O)NR³⁰¹— or —NR³⁰¹C(═O)S—;
L⁴ is a bond, —O(C═O)—, —(C═O)O—, —O(C═O)O—, —C(═O)—, —O—, —O(CR⁴⁰¹R⁴⁰²)ₛO—, —S—, —C(═O)S—, —SC(═O)—, —NR⁴⁰¹C(═O)—, —C(═O)NR⁴⁰¹—, —NR⁴⁰¹C(═O)NR⁴⁰²—, —NR⁴⁰¹C(═S)—, —C(═S)NR⁴⁰¹—, —NR⁴⁰¹C(═S)NR⁴⁰²—, —OC(═O)NR⁴⁰¹—, —NR⁴⁰¹C(═O)O—, —SC(═O)NR⁴⁰¹— or —NR⁴⁰¹C(═O)S—;
L⁵ is a bond, —O(C═O)—, —(C═O)O—, —O(C═O)O—, —C(═O)—, —O—, —O(CR⁵⁰¹R⁵⁰²)ₛO—, —S—, —C(═O)S—, —SC(═O)—, —NR⁵⁰¹C(═O)—, —C(═O)NR⁵⁰¹—, —NR⁵⁰¹C(═O)NR⁵⁰²—, —NR⁵⁰¹C(═S)—, —C(═S)NR⁵⁰¹—, —NR⁵⁰¹C(═S)NR⁵⁰²—, —OC(═O)NR⁵⁰¹—, —NR⁵⁰¹C(═O)O—, —SC(═O)NR⁵⁰¹— or —NR⁵⁰¹C(═O)S—;
L⁶ is a bond, —O(C═O)—, —(C═O)O—, —O(C═O)O—, —C(═O)—, —O—, —O(CR⁶⁰¹R⁶⁰²)ₛO—, —S—, —C(═O)S—, —SC(═O)—, —NR⁶⁰¹C(═O)—, —C(═O)NR⁶⁰¹—, —NR⁶⁰¹C(═O)NR⁶⁰²—, —NR⁶⁰¹C(═S)—, —C(═S)NR⁶⁰¹—, —NR⁶⁰¹C(═S)NR⁶⁰²—, —OC(═O)NR⁶⁰¹—, —NR⁶⁰¹C(═O)O—, —SC(═O)NR⁶⁰¹— or —NR⁶⁰¹C(═O)S—;
L⁷ is a bond, —O(C═O)—, —(C═O)O—, —O(C═O)O—, —C(═O)—, —O—, —O(CR⁷⁰¹R⁷⁰²)ₛO—, —S—, —C(═O)S—, —SC(═O)—, —NR⁷⁰¹C(═O)—, —C(═O)NR⁷⁰¹—, —NR⁷⁰¹C(═O)NR⁷⁰²—, —NR⁷⁰¹C(═S)—, —C(═S)NR⁷⁰¹—, —NR⁷⁰¹C(═S)NR⁷⁰²—, —OC(═O)NR⁷⁰¹—, —NR⁷⁰¹C(═O)O—, —SC(═O)NR⁷⁰¹— or —NR⁷⁰¹C(═O)S—;
L^{a1} and L^{a2} are each independently 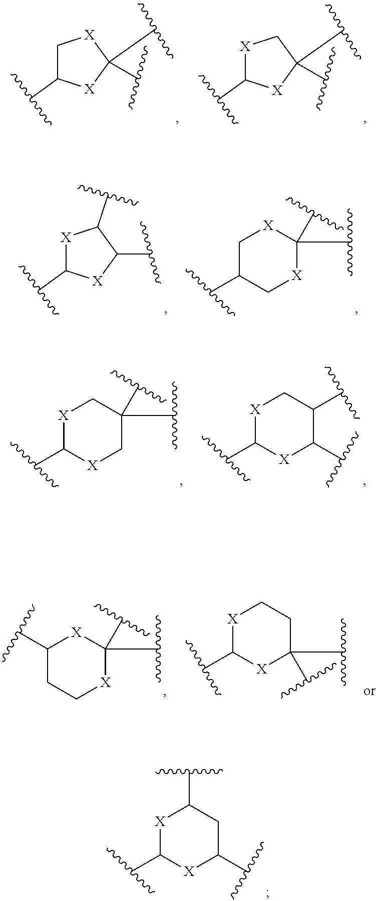
each X is independently O, S, or CH₂;
W¹, W², W³, W⁴, W⁵, and W⁶ are each independently a bond, substituted or unsubstituted C₁-C₁₂ alkylene, or substituted or unsubstituted 2 to 12 membered heteroalkylene;
each R^{1A} and R^{1B} is independently H, substituted or unsubstituted C₁-C₁₂ alkyl, or substituted or unsubstituted 2 to 12 membered heteroalkyl;
each R^{2A}, R^{3A}, R^{4A}, and R^{5A} is independently H, substituted or unsubstituted C₁-C₃₀ alkyl, or substituted or unsubstituted 2 to 30 membered heteroalkyl;
each R¹⁰¹, R¹⁰², R²⁰¹, R²⁰², R³⁰¹, R³⁰², R⁴⁰¹, R⁴⁰², R⁵⁰¹, R⁵⁰², R⁶⁰¹, R⁶⁰², R⁷⁰¹, and R⁷⁰² is independently H, substituted or unsubstituted C₁-C₁₂ alkyl, or substituted or unsubstituted 2 to 12 membered heteroalkyl; and
each s is independently an integer from 1 to 4.

72-75. (canceled)

76. The pharmaceutical composition of claim 71, wherein the cationic lipid is: 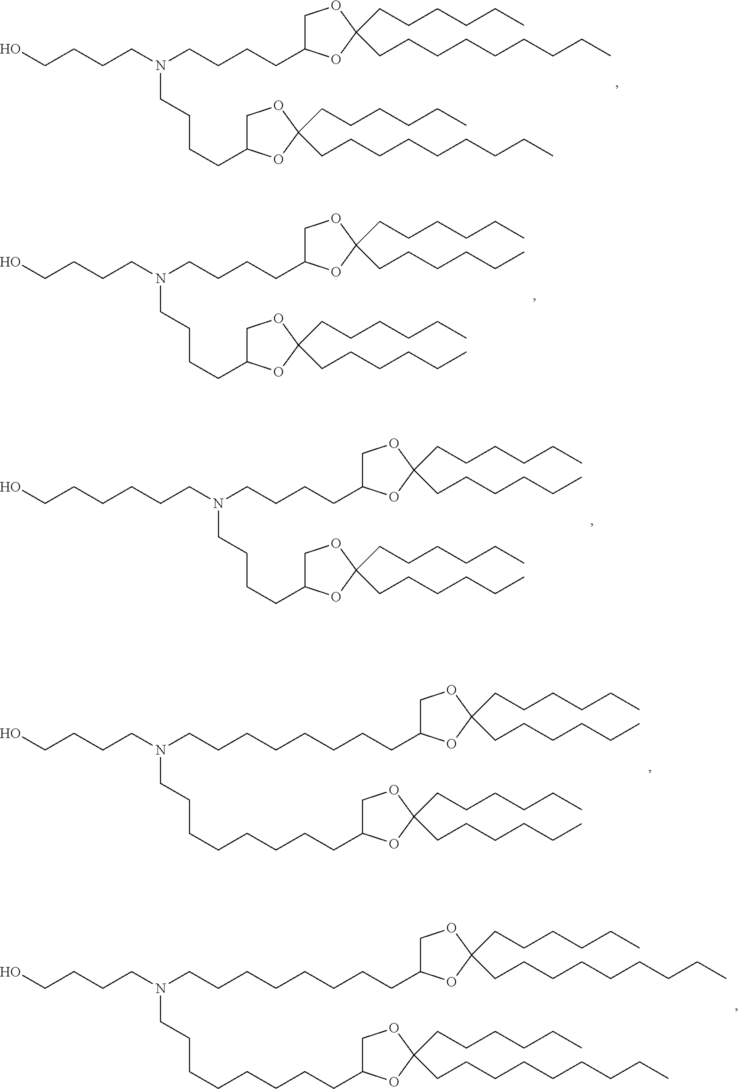 [SMILES: CCCCCCCCCC1(CCCCCC)OCC(CCCCN(CCCCO)CCCCC2COC(CCCCCC)(CCCCCCCCC)O2)O1 ; CCCCCCC1(CCCCCC)OCC(CCCCN(CCCCO)CCCCC2COC(CCCCCC)(CCCCCC)O2)O1 ; CCCCCCC1(CCCCCC)OCC(CCCCN(CCCCCCO)CCCCC2COC(CCCCCC)(CCCCCC)O2)O1 ; CCCCCCC1(CCCCCC)OCC(CCCCCCCCN(CCCCO)CCCCCCCCC2COC(CCCCCC)(CCCCCC)O2)O1 ; CCCCCCCCCC1(CCCCCC)OCC(CCCCCCCCN(CCCCO)CCCCCCCCC2COC(CCCCCC)(CCCCCCCCC)O2)O1] 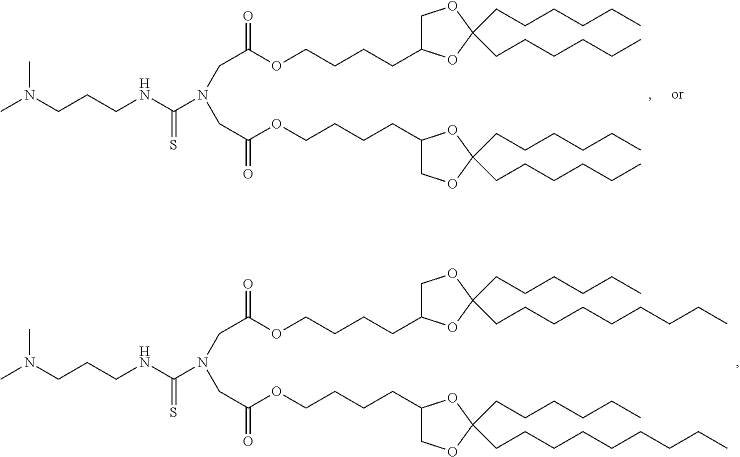
or a pharmaceutically acceptable salt thereof.

77-100. (canceled)

101. A method for treating or preventing a proliferation disease or disorder in a subject having, suspected of having, or at risk of having the proliferation disease or disorder, the method comprising administering to the subject an effective amount of the polynucleotide of claim 1.

102-107. (canceled)

108. A method for treating or preventing an immune deficiency disease or disorder in a subject having, suspected of having, or at risk of having the immune deficiency disease or disorder, the method comprising administering to the subject an effective amount of the polynucleotide of claim 1.

109. (canceled)

110. A method for treating or preventing an autoimmune or inflammatory disease or disorder in a subject having, suspected of having, or at risk of having the autoimmune or inflammatory disease or disorder, the method comprising administering to the subject an effective amount of the polynucleotide of claim 1.

111. (canceled)

112. A method for treating or preventing an infectious disease or disorder in a subject in a subject having, suspected of having, or at risk of having an infectious disease or, the method comprising administering to the subject an effective amount of the polynucleotide of claim 1.

113-145. (canceled)
